(19) United States
(12) Patent Application Publication
(10) Pub. No.: US 2017/0006815 A1
(43) Pub. Date: Jan. 12, 2017

(54) CEREAL SEED STARCH SYNTHASE II ALLELES AND THEIR USES

(71) Applicant: Montana State University, Bozeman, MT (US)

(72) Inventor: Michael J. GIROUX, Bozeman, MT (US)

(21) Appl. No.: 15/205,376

(22) Filed: Jul. 8, 2016

Related U.S. Application Data
(60) Provisional application No. 62/190,381, filed on Jul. 9, 2015.

(51) Int. Cl. A01H 5/10; A01H 1/04; C12N 9/10; A01H 1/02; C12Q 1/68

(52) U.S. Cl. CPC A01H 5/10; A01H 1/02; C12Q 1/6895; C12N 9/1051; C12Y 204/01021; A01H 1/04; C12Q 2600/156; C12Q 2600/158

(57) ABSTRACT

The present invention provides compositions and methods of altering/improving wheat phenotypes. Furthermore, methods of breeding wheat and/or other closely related species to produce plants having altered or improved phenotypes are provided.

### CROSS-REFERENCE TO RELATED APPLICATIONS

This application claims priority to U.S. provisional application No. 62/190,381 filed on Jul. 9, 2015, which is hereby incorporated by reference in its entirety, including all descriptions, references, figures, and claims for all purposes.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: MONT_155_02_SeqList_ST25.txt, date recorded: Jul. 5, 2016; file size: 463 kilobytes).

### TECHNICAL FIELD

The invention generally relates to improving the end product quality characteristics of wheat. More specifically, the present invention relates to compositions and methods for improving one or more end product quality characteristics of wheat by modifying one or more starch synthesis genes.

### BACKGROUND

Starch makes up approximately 70% of the dry weight of cereal grains and is composed of two forms of glucose polymers, straight chained amylose with α-1,4 linkages and branched amylopectin with α-1,4 linkages and α-1,6 branch points. In bread wheat, amylose accounts for approximately 25% of the starch with amylopectin the other 75% (reviewed in Tetlow 2006). The synthesis of starch granules is an intricate process that involves several enzymes which associate in complexes (Tetlow et al. 2008; Tetlow et al. 2004b). In bread wheat, the “waxy” proteins (granule bound starch synthase I) encoded by the genes Wx-A1a, Wx-B1a, and Wx-D1a are solely responsible for amylose synthesis after the production of ADP-glucose by ADP-glucose pyrophosphorylase (AGPase) (Denyer et al. 1995; Miura et al. 1994; Yamamori et al. 1994). In contrast, amylopectin synthesis involves a host of enzymes such as AGPase, starch synthases (SS) I, II, III, IV, starch branching enzymes (SBE) I and II, and starch de-branching enzymes (Tetlow et al. 2004a).

Several starch biosynthetic proteins remain bound to the interior of starch granules with a subset of these proteins designated the starch granule proteins (SGPs). SGP-1 proteins are isoforms of SSII encoded by the genes SSIIa-A, SSIIa-B, SSIIa-D on the short arms of group 7 chromosomes (Li et al., 1999). Much attention has been devoted to creating increased amylose wheat varieties. A survey of hexaploid wheat germplasm identified lines lacking SGP-A1, SGP-B1, or SGP-D1 (Yamamori and Endo, 1996), which were crossed to create an SGP-1 null (Yamamori et al., 2000). The SGP-1 null had a 29% increase in amylose content (37.3% null vs. 28.9% wild-type), deformed starch granules, reduced starch content, and reduced binding of SGP-2 and SGP-3 to starch granules.

The key advantage of SGP-1 null lines is in their increased amylose, protein content, and dietary fiber. The key disadvantage of the SGP-1 nulls however is their reduced seed size and overall reduction in agronomic yield. Therefore, there is a great need for compositions and methods of increasing amylose contents of wheat while mitigating large reductions in seed size and yield.

### SUMMARY OF INVENTION

The present invention provides compositions and methods for producing improved wheat plants through conventional plant breeding and/or molecular methodologies. Among such compositions, the present invention provides high amylose wheat grain. In some embodiments, the grain is produced from a durum wheat plant of the present invention. In some embodiments, the grain is produced from a bread wheat plant of the present invention.

Thus in some embodiments, the wheat plants of the present disclosure are tetraploid, comprising a first and second genome. In other embodiments, the wheat plants of the present disclosure are hexaploid, comprising a first, second, and third genome.

In some embodiments, the grain is produced from wheat comprising one or more mutations of one or more starch synthesis genes.

In some embodiments, the present invention teaches leaky starch synthase II alleles and wheat grain comprising a starch synthase II allele. In some embodiments, the present invention teaches a wheat plant cell comprising one or more leaky starch synthase II alleles.

In some embodiments, the present disclosure teaches SSII leaky alleles comprising a missense mutation encoding for an SSII protein with an amino acid substitution selected from the group consisting of: SSII-D-E656K, SSII-D-A421V, SSII-D-A785V, SSII-B-P251S, SSII-A-P319L, SSII-B-P333L, SSII-B-P333S, SSII-A-E663K, SSII-A-A681T, SSII-A-G721E, and SSII-A-P693S.

In some embodiments, the present disclosure teaches a DNA construct comprising an SSII leaky allele, wherein said leaky alleles comprises a missense mutation encoding for an SSII protein with an amino acid substitution selected from the group consisting of: SSII-D-E656K, SSII-D-A421V, SSII-D-A785V, SSII-B-P251S, SSII-A-P319L, SSII-B-P333L, SSII-B-P333S, SSII-A-E663K, SSII-A-A681T, SSII-A-G721E, and SSII-A-P693S.

Thus, in some embodiments, the present disclosure teaches a DNA construct comprising a sequence encoding a peptide selected from the group consisting of: SEQ ID NO: 40, SEQ ID NO: 44, SEQ ID NO: 42, SEQ ID NO: 26, SEQ ID NO: 11, SEQ ID NO: 45, SEQ ID NO: 48, SEQ ID NO: 68, SEQ ID NO: 70, SEQ ID NO: 72, SEQ ID NO: 86.

In some embodiments, the present disclosure teaches isolated DNA comprising an SSII leaky allele, wherein said leaky alleles comprises a missense mutation encoding for an SSII protein with an amino acid substitution selected from the group consisting of: SSII-D-E656K, SSII-D-A421V, SSII-D-A785V, SSII-B-P251S, SSII-A-P319L, SSII-B-P333L, SSII-B-P333S, SSII-A-E663K, SSII-A-A681T, SSII-A-G721E, and SSII-A-P693S

Thus, in some embodiments, the present disclosure teaches isolated DNA comprising a sequence encoding a peptide selected from the group consisting of: SEQ ID NO: 40, SEQ ID NO: 44, SEQ ID NO: 42, SEQ ID NO: 26, SEQ ID NO: 11, SEQ ID NO: 45, SEQ ID NO: 48, SEQ ID NO: 68, SEQ ID NO: 70, SEQ ID NO: 72, SEQ ID NO: 86.

In some embodiments, the present disclosure teaches wheat plants with low SSII gene activity, above that of SSII null plants, but significantly below wild type levels. Thus in some embodiments, the present disclosure teaches plants in which the only functional SSII alleles are leaky alleles.

In some embodiments, the grain or the wheat plant cell of the present disclosure is produced from wheat comprising one or more mutations of a starch synthase (SSII) gene. In some embodiments, the present invention teaches a high amylose grain produced from a wheat plant comprising a) at least one SSII leaky allele; and b) no SSII wild type functional alleles; wherein the high amylose grain has an increased proportion of starch amylose compared to the proportion of starch amylose of a control grain from an appropriate wild type wheat check variety grown under similar field conditions, and wherein the high amylose grain has higher seed weight compared to grain from an appropriate null wheat check variety grown under similar field conditions, wherein the null wheat check variety comprises only SSII null alleles.

Thus, in some embodiments, the present disclosure teaches a plant cell, plant part, or tissue culture, comprising a) at least one SSII leaky allele; and b) no SSII wild type functional alleles; wherein grain produced from the plant regenerated from said plant cell, plant part, or plant tissue culture has an increased proportion of starch amylose compared to the proportion of starch amylose of a control grain from an appropriate wild type wheat check variety grown under similar field conditions, and wherein the grain also has higher seed weight compared to grain from an appropriate null wheat check variety grown under similar field conditions, wherein the null wheat check variety comprises only SSII null alleles.

In some embodiments, the SSII leaky alleles of the present disclosure are non-naturally occurring alleles. For example, in some embodiments, the leaky alleles of the present disclosure are mutagenized alleles.

In some embodiments, the SSII leaky alleles of the present disclosure comprise one or more i) missense mutations, ii) nonsense mutations, iii) silent mutations (e.g., rare codon usage), iv) splice junction mutations (e.g. affecting transcript processing), v) insertions/or deletions, vi) promoter and or UTR mutations, or a combination thereof.

In some embodiments, the present invention teaches a high amylose grain wherein the wheat plant from which the high amylose grain is produced further comprises one or more SSII null alleles.

In some embodiments, the wheat plant from which the high amylose grain or the plant cell is produced can be, for example, durum or bread wheat plant.

In some embodiments, the present invention teaches a high amylose grain or a wheat plant cell capable of regenerating a plant that produces said high amylose grain, wherein the proportion of amylose in the starch of said grain is at least 25% higher compared to the starch amylose of a control grain from an appropriate wild type wheat check variety grown under similar field conditions.

In some embodiments, the present invention teaches a high amylose grain or a wheat plant cell capable of regenerating a plant that produces said high amylose grain, wherein the high amylose grain has at least a 10% higher seed weight than grain from an appropriate null wheat check variety grown under similar field conditions, wherein the null wheat check variety comprises only null SSII alleles.

In some embodiments, the present invention teaches a high amylose grain or a wheat plant cell, wherein at least one of the SSII leaky alleles comprises a missense mutation encoding for an SSII protein with an amino acid substitution selected from the group consisting of: SSII-D-E656K, SSII-D-A421V, SSII-D-A785V, SSII-B-P251S, SSII-A-P319L, SSII-B-P333L, SSII-B-P333S, SSII-A-E663K, SSII-A-A681T, SSII-A-G721E, and SSII-A-P693S.

In some embodiments, the present invention teaches a high amylose grain or a wheat plant cell, wherein at least one of the SSII leaky alleles comprises a missense mutation encoding for a protein with a SSII-D-E656K and/or SSII-D-A421V amino acid substitution.

In some embodiments, the present invention teaches a high amylose grain or a wheat plant cell, wherein at least one of the SSII leaky alleles comprises a missense mutation encoding for a protein with a SSII-D-E656K amino acid substitution.

In some embodiments, the present invention teaches a high amylose grain or a wheat plant cell, wherein at least one of the SSII leaky alleles comprises a missense mutation encoding for a protein with a SSII-D-A421V amino acid substitution.

In some embodiments, the present invention teaches a high amylose grain or a wheat plant cell, wherein at least one of the SSII leaky alleles comprises a missense mutation encoding for a protein with a SSII-D-A785V amino acid substitution.

In some embodiments, the present invention teaches a high amylose grain or a wheat plant cell, wherein at least one of the SSII leaky alleles comprises a missense mutation encoding for a protein with a SSII-B-P251S amino acid substitution.

In some embodiments, the present invention teaches a high amylose grain or a wheat plant cell, wherein at least one of the SSII leaky alleles comprises a missense mutation encoding for a protein with a SSII-A-P319L amino acid substitution.

In some embodiments, the present invention teaches a high amylose grain or a wheat plant cell, wherein at least one of the SSII leaky alleles comprises a missense mutation encoding for a protein with a SSII-B-P333L amino acid substitution.

In some embodiments, the present invention teaches a high amylose grain or a wheat plant cell, wherein at least one of the SSII leaky alleles comprises a missense mutation encoding for a protein with a SSII-B-P333S L amino acid substitution.

In some embodiments, the present invention teaches a high amylose grain or a wheat plant cell, wherein at least one of the SSII leaky alleles comprises a missense mutation encoding for a protein with a SSII-A-E663K amino acid substitution.

In some embodiments, the present invention teaches a high amylose grain or a wheat plant cell, wherein at least one of the SSII leaky alleles comprises a missense mutation encoding for a protein with a SSII-A-A681T amino acid substitution.

In some embodiments, the present invention teaches a high amylose grain or a wheat plant cell, wherein at least one of the SSII leaky alleles comprises a missense mutation encoding for a protein with a SSII-A-G721E amino acid substitution.

In some embodiments, the present invention teaches a high amylose grain or a wheat plant cell, wherein at least one of the SSII leaky alleles comprises a missense mutation encoding for a protein with a SSII-A-P693S amino acid substitution.

In some embodiments, the present invention teaches a high amylose grain or a wheat plant cell, wherein at least one of the SSII leaky alleles encodes for the protein of SEQ ID No. 40 or SEQ ID No. 44.

In some embodiments, the present invention teaches a high amylose grain or a wheat plant cell, wherein at least one of the SSII leaky alleles comprises a missense mutation encoding for a protein with a SSII-B-P333L and/or SSII-B-P333S amino acid substitution.

In some embodiments, the present invention teaches a high amylose grain or a wheat plant cell, wherein at least one of the SSII leaky alleles comprises a missense mutation encoding for a protein with a SSII-B-P333L amino acid substitution.

In some embodiments, the present invention teaches a high amylose grain or a wheat plant cell, wherein at least one of the SSII leaky alleles comprises a missense mutation encoding for a protein with a SSII-B-P333L amino acid substitution.

In some embodiments, the present invention teaches a high amylose grain or a wheat plant cell, wherein at least one of the SSII leaky alleles encodes for the protein of SEQ ID No. 46 or SEQ ID No. 48.

In some embodiments, the present invention teaches a high amylose grain or a wheat plant cell, wherein at least one of the SSII leaky alleles comprises a missense mutation encoding for a protein with a E656K amino acid substitution.

In some embodiments, the present invention teaches a high amylose grain or a wheat plant cell, wherein at least one of the SSII leaky alleles encodes for the protein of SEQ ID No. 40.

In some embodiments, the present invention teaches a high amylose grain or a wheat plant cell, wherein at least one of the SSII leaky alleles comprises a missense mutation encoding for a protein with a A421V amino acid substitution.

In some embodiments, the present invention teaches a high amylose grain or a wheat plant cell, wherein at least one of the SSII leaky alleles encodes for the protein of SEQ ID No. 44.

In some embodiments, the present invention teaches a high amylose grain or a wheat plant cell, wherein at least one of the SSII leaky alleles encodes for the protein of SEQ ID NO: 42.

In some embodiments, the present invention teaches a high amylose grain or a wheat plant cell, wherein at least one of the SSII leaky alleles encodes for the protein of SEQ ID NO: 26.

In some embodiments, the present invention teaches a high amylose grain or a wheat plant cell, wherein at least one of the SSII leaky alleles encodes for the protein of SEQ ID NO: 11.

In some embodiments, the present invention teaches a high amylose grain or a wheat plant cell, wherein at least one of the SSII leaky alleles encodes for the protein of SEQ ID NO: 45.

In some embodiments, the present invention teaches a high amylose grain or a wheat plant cell, wherein at least one of the SSII leaky alleles encodes for the protein of SEQ ID NO: 48.

In some embodiments, the present invention teaches a high amylose grain or a wheat plant cell, wherein at least one of the SSII leaky alleles encodes for the protein of SEQ ID NO: 68.

In some embodiments, the present invention teaches a high amylose grain or a wheat plant cell, wherein at least one of the SSII leaky alleles encodes for the protein of SEQ ID NO: 70.

In some embodiments, the present invention teaches a high amylose grain or a wheat plant cell, wherein at least one of the SSII leaky alleles encodes for the protein of SEQ ID NO: 72.

In some embodiments, the present invention teaches a high amylose grain or a wheat plant cell, wherein at least one of the SSII leaky alleles encodes for the protein of SEQ ID NO: 86.

In some embodiments, the present invention teaches a high amylose grain or a wheat plant cell capable of regenerating a plant that produces said high amylose grain, wherein the high amylose grain has a flour swelling power (FSP) of less than about 7.5.

In some embodiments, the present invention teaches flour produced from the high amylose grain described herein, and methods of producing the same.

In some embodiments, the present invention teaches starch produced from the high amylose grain described herein, and methods of producing the same.

In some embodiments, the present invention teaches a flour based product comprising the high amylose grain described herein, and methods of producing the same.

In some embodiments, the present invention teaches a high amylose grain or a wheat plant cell, wherein the wheat plant is a hexaploid wheat comprising a first, second, and third genome.

In some embodiments, the hexaploid wheat or a wheat plant cell comprises homozygous SSII null alleles in the first and second genomes, and the SSII leaky allele in the third genome.

In some embodiments, the present invention teaches a high amylose grain or a wheat plant cell wherein the SSII leaky allele is homozygous in the third genome.

In some embodiments, the present invention teaches a method for producing a wheat plant with one or more wheat starch synthase (SSII) leaky alleles, one or more SSII null alleles, and no wild type functional SSII alleles, said method comprising: A) mutagenizing a wheat grain to form a mutagenized population of grain; B) growing one or more wheat plants from said mutagenized wheat grain; C) screening the resulting plants to identify wheat plants with an SSII leaky mutant allele; D) crossing an SSII leaky wheat plant derived from step (c) with a second wheat plant comprising at least one SSII null allele, or at least one SSII leaky allele; E) harvesting the resulting grain; F) growing the harvested grain into a plant; and G) selecting for a wheat plant comprising one or more SSII leaky alleles and no wild type functional SSII alleles.

In some embodiments, the present invention teaches a method for producing a wheat plant with one or more wheat starch synthase (SSII) leaky alleles, and no wild type functional SSII alleles, said method comprising: A) crossing a wheat plant comprising one or more SSII leaky alleles with a second wheat plant in which all the SSII alleles are selected from the group consisting of null genes, leaky alleles, and combinations thereof; B) harvesting the resulting grain; C) growing the harvested grain into a plant; and, D) selecting for a wheat plant comprising one or more SSII leaky alleles, and no wild type functional SSII alleles.

In some embodiments, a method for producing a wheat plant with one or more wheat starch synthase (SSII) leaky alleles, one or more SSII null alleles, and no wild-type SSII alleles, said method comprising: a) crossing a wheat plant comprising one or more SSII leaky alleles with a second durum wheat plant in which all SSII alleles are null; b) harvesting the resulting grain; c) growing the harvested grain into a plant; and d) selecting for a wheat plant comprising one or more wheat starch synthase (SSII) leaky alleles, one or more SSII null alleles, and no wild-type SSII alleles; wherein the selected wheat plant comprises one or more wheat starch synthase (SSII) leaky alleles, one or more SSII null alleles, and no wild-type SSII alleles, and wherein said plant produces high amylose grain.

In some embodiments, the present invention teaches methods of producing high amylose wheat plant, wherein the selected wheat plant further comprises one or more SSII null alleles. In some embodiments, the present invention teaches breeding methods wherein at least one of the SSII leaky alleles comprises a missense mutation encoding for an SSII protein with an amino acid substitution selected from the group consisting of: SSII-D-E656K, SSII-D-A421V, SSII-D-A785V, SSII-B-P251S, SSII-A-P319L, SSII-B-P333L, SSII-B-P333S, SSII-A-E663K, SSII-A-A681T, SSII-A-G721E, and SSII-A-P693S.

In some embodiments, the present invention teaches a method of breeding wheat plants with high amylose grain, the method comprising: a) making a cross between a first plant produced by the methods of the invention with a second plant to produce a F1 plant; b) backcrossing the F1 plant to the second plant; and c) repeating the backcrossing step one or more times to generate a near isogenic or isogenic line; wherein the isogenic or near isogenic wheat plant comprises one or more wheat starch synthase (SSII) leaky alleles, one or more SSII null alleles, and no wild-type functional SSII alleles, and wherein said plant produces high amylose grain.

In some embodiments, the present invention teaches a method of breeding wheat plants with high amylose grain, the method comprising: a) making a cross between a first plant produced by the methods of the invention with a second plant to produce a F1 plant; b) backcrossing the F1 plant to the second plant; and c) repeating the backcrossing step one or more times to generate a near isogenic or isogenic line; wherein the isogenic or near isogenic wheat plant comprises one or more wheat starch synthase (SSII) leaky alleles, and no wild-type functional SSII alleles, and wherein said plant produces high amylose grain.

In some embodiments, the present invention teaches methods of breeding, wherein the isogenic or near isogenic wheat plant further comprises one or more SSII null alleles.

In some embodiments, the present invention teaches a high amylose grain or a wheat plant cell produced from a wheat plant comprising: a) one or more starch synthase a (SSII) null alleles; b) at least one SSII leaky allele, wherein at least one of the SSII leaky alleles comprises a missense mutation encoding for an SGP-1 protein with an amino acid substitution selected from the group consisting of: SSII-D-E656K, SSII-D-A421V, SSII-D-A785V, SSII-B-P251S, SSII-A-P319L, SSII-B-P333L, SSII-B-P333S, SSII-A-E663K, SSII-A-A681T, SSII-A-G721E, and SSII-A-P693S; and c) no SSII wild-type functional alleles; wherein the high amylose grain has an increased proportion of starch amylose compared to the proportion of starch amylose of a control grain from an appropriate wild type wheat check variety grown under similar field conditions, and wherein the grain also has higher seed weight compared to grain from an appropriate null wheat check variety grown under similar field conditions, wherein the null wheat check variety comprises only SSII null alleles. In some embodiments, the present invention teaches a high amylose grain or a wheat plant cell wherein at least one of the SSII leaky alleles comprises a missense mutation encoding for an SGP-1 protein with an amino acid substitution selected from the group consisting of: SSII-D-E656K, SSII-D-A421V, SSII-D-A785V, SSII-B-P251S, SSII-A-P319L, SSII-B-P333L, and SSII-B-P333S.

In some embodiments, the present invention teaches wheat with one or more leaky SSII alleles. In some embodiments, the leaky alleles of the present disclosure are selected for retaining a small amount of starch synthase function. In some embodiments, leaky SSII alleles are selected based on reduced SGP-1 accumulation in purified starch. In some embodiments, leaky SSII alleles are selected for their ability to produce reduced flour swelling power in an SSII null background. In yet other embodiments, leaky SSII alleles of the present disclosure are selected for their ability to produce wheat grain with elevated amylose levels compared to a wild type control plant, but higher seed weights compared to completely SSII null plants.

In some embodiments, the present invention teaches plant cells of high amylose wheat having one or more leaky SSII alleles. In particular embodiments, the wheat plant cells include one or more of the leaky SSII alleles specifically disclosed, including any combination of the disclosed leaky SSII alleles. In some embodiments the plant cells include cells from any plant part such as plant protoplasts, plant cell tissue cultures from which wheat plants can be regenerated, plant calli, embryos, pollen, grain, ovules, fruit, flowers, leaves, seeds, roots, root tips and the like.

In some embodiments, the present disclosure teaches a method of producing a milled product, said method comprising the steps of: a) milling the high amylose grain of the wheat plants of the present disclosure, thereby producing the milled product.

In one aspect of the present invention, there are provided novel bread and durum wheat lines, designated 624, 122, 414, 102, 42, 213, 217, 1174, 1513, 134, and 1704. Thus, one aspect of this invention relates to the grain of any one of wheat lines 624, 122, 414, 102, 42, 213, 217, 1174, 1513, 134, and 1704, to the plants of wheat lines 624, 122, 414, 102, 42, 213, 217, 1174, 1513, 134, and 1704, and parts thereof, for example pollen, ovule, grain, and to methods for producing a wheat plant by crossing the wheat lines 624, 122, 414, 102, 42, 213, 217, 1174, 1513, 134, and 1704 with themselves, or another wheat line. A further aspect relates to wheat seeds produced by crossing the wheat lines 624, 122, 414, 102, 42, 213, 217, 1174, 1513, 134, and 1704 with another wheat line.

Another aspect of the present invention is also directed to wheat lines 624, 122, 414, 102, 42, 213, 217, 1174, 1513, 134, and 1704, into which one or more specific single gene traits, for example transgenes, have been introgressed from another wheat line, and which has essentially all of the morphological and physiological characteristics of wheat lines 624, 122, 414, 102, 42, 213, 217, 1174, 1513, 134, and 1704. Another aspect of the present invention also relates to seeds of wheat lines 624, 122, 414, 102, 42, 213, 217, 1174, 1513, 134, and 1704 into which one or more specific, single gene traits have been introgressed and to plants of wheat lines 624, 122, 414, 102, 42, 213, 217, 1174, 1513, 134, and 1704 into which one or more specific, single gene traits have been introgressed. A further aspect of the present invention relates to methods for producing a wheat plant by crossing plants of wheat lines 624, 122, 414, 102, 42, 213, 217, 1174, 1513, 134, and 1704 into which one or more specific, single gene traits have been introgressed with themselves or with another wheat line.

Another aspect of the present invention relates to hybrid wheat seeds and plants produced by crossing plants of wheat lines 624, 122, 414, 102, 42, 213, 217, 1174, 1513, 134, and 1704 into which one or more specific, single gene traits have been introgressed with another wheat line. A further aspect of the present invention is also directed to a method of producing inbreds comprising planting a collection of hybrid seed, growing plants from the collection, identifying inbreds among the hybrid plants, selecting the inbred plants and controlling their pollination to preserve their homozygosity.

In some embodiments, the present disclosure teaches a tissue culture of cells produced from the plants of the present invention.

Other embodiments of the present invention include high amylose grain, and flour based products from bread and durum wheat grain produced from a wheat plant comprising one or more leaky SSII alleles and no wildtype SSII functional alleles. In some embodiments, the high amylose grain can be used to produce flour based products. In some embodiments, milled products produced from the high amylose grain are flour, starch, semolina, among others. In some embodiments, flour based products produced from the high amylose grain are pasta, and noodles among others. The present invention teaches flour based products produced from the high amylose grain. In some embodiments, the invention teaches flour produced from the high amylose grain. In other embodiments the flour based product produced by the high amylose grain is dried pasta.

In some embodiments, the flour based product has a protein content of at least 17%. In other embodiments the flour based product has a protein content of at least 20%. In some embodiments, the flour based product has a dietary fiber content of at least 3%. In other embodiments the flour based product has a dietary fiber content of at least 7%. In some embodiments, the flour based product has a resistant starch content of at least 2%. In other embodiments the flour based product has a resistant starch content of at least 3%.

In other embodiments the protein, resistant starch and dietary fiber contents of the flour based product are increased when compared to a flour based product from an appropriate durum or bread wheat check line grown under similar field conditions. In some embodiments, of the present invention, when the comparison is to an appropriate durum or bread wheat check line grown under similar field conditions, the wheat lines of the present invention and then check lines are grown at the same time and/or location.

For example, in some embodiments, the flour based product has an increased protein content that is at least 10% higher than a flour based product produced from the grain of an appropriate durum or bread wheat check variety grown under similar field conditions. In other embodiments the flour based product has an increased protein content that is at least 20% higher than a flour based product produced from the grain of an appropriate durum or bread wheat check variety grown under similar field conditions. In other embodiments the flour based product has an increased protein content that is at least 30% higher than a flour based product produced from the grain of an appropriate durum or bread wheat check variety grown under similar field conditions. In some embodiments, the flour based product has an increased dietary fiber content that is at least 50% higher than a flour based product produced from the grain of an appropriate durum or bread wheat check variety grown under similar field conditions. In other embodiments the flour based product has an increased dietary fiber content that is at least 100% higher than a flour based product produced from the grain of an appropriate durum or bread wheat check variety grown under similar field conditions. In other embodiments the flour based product has an increased dietary fiber content that is at least 200% higher than a flour based product produced from the grain of an appropriate durum or bread wheat check variety grown under similar field conditions. In some embodiments, the flour based product has an increased resistant starch content that is at least 50% higher than a flour based product produced from the grain of an appropriate durum or bread wheat check variety grown under similar field conditions. In other embodiments the flour based product has an increased resistant starch content that is at least 100% higher than a flour based product produced from the grain of an appropriate durum or bread wheat check variety grown under similar field conditions. In other embodiments the flour based product has an increased resistant starch content that is at least 200% higher than a flour based product produced from the grain of an appropriate durum or bread wheat check variety grown under similar field conditions. In some embodiments, the flour based product has an increased amylose content that is at least 12% higher than a flour based product produced from the grain of an appropriate durum or bread wheat check variety grown under similar field conditions. In other embodiments the flour based product has an increased amylose content that is at least 25% higher than a flour based product produced from the grain of an appropriate durum or bread wheat check variety grown under similar field conditions. In other embodiments the flour based product has an increased amylose content that is at least 40% higher than a flour based product produced from the grain of an appropriate durum or bread wheat check variety grown under similar field conditions. In some embodiments, the flour based product is dried pasta wherein the pasta has improved firmness after cooking compared to pasta produced from the grain of an appropriate durum or bread wheat check variety grown under similar field conditions.

In some embodiments, the high amylose grain has a flour swelling power (FSP) of less than 8.4. In other embodiments the high amylose grain has an FSP of less than 7.5.

In some embodiments, the proportion of dietary fiber, resistant starch, and protein content that is increased in said high amylose grain is increased when compared to the grain of an appropriate durum or bread wheat check variety grown under similar field conditions. In some embodiments, the amylose content of the starch made from the high amylose grain is at least 12% higher than the amylose content of the starch made from the grain of an appropriate wheat check variety grown under similar field conditions. In other embodiments, the amylose content of the starch made from the high amylose grain is at least 25% higher than the amylose content of the starch made from the grain of an appropriate wheat check variety grown under similar field conditions. In other embodiments, the amylose content of the starch made from the high amylose grain is at least 40% higher than the amylose content of the starch made from the grain of an appropriate wheat check variety grown under similar field conditions. In some embodiments, the appropriate durum wheat check variety is grown at the same time and/or location.

In some embodiments, the starch of the high amylose grain has altered gelatinization properties when compared to starch from the grain of an appropriate durum wheat check variety grown under similar field conditions.

In some embodiments, the pasta or noodles made from the high amylose grain have reduced glycemic index compared to pasta or noodles produced from the grain of an appropriate durum wheat check variety grown under similar field conditions.

In some embodiments, the pasta or noodles made from the high amylose grain have increased firmness compared to pasta or noodles made from grain of the appropriate durum wheat check variety grown under similar field conditions.

In some embodiments, the pasta or noodles made from the high amylose grain have increased tolerance to overcooking compared to pasta or noodles made from grain of the appropriate durum wheat check variety grown under similar field conditions.

In some embodiments, the pasta or noodles made from the high amylose grain have increased protein content compared to pasta or noodles made from grain of the appropriate durum wheat check variety grown under similar field conditions.

Pasta produced from the mutant grain also has increased proportion of dietary fiber, resistant starch and/or protein content when compared to pasta made from the grain of the wild type durum wheat plant.

In some embodiments, the grain has increased amylose content compared to the grain of the wild type durum or bread wheat plant.

In some embodiments, the grain has increased dietary fiber and increased amylose content when compared to the grain of the wild type durum or bread wheat plant.

In some embodiments, the grain has increased protein content and increased amylose content when compared to the grain of the wild type durum or bread wheat plant.

In some embodiments, the grain has increased dietary fiber and decreased endosperm to bran ratio and/or reduced milling yield when compared to the grain of the wild type durum or bread wheat plant.

In some embodiments, the grain has increased dietary fiber and increased ash when compared to the grain of the wild type durum or bread wheat plant.

In some embodiments, the grain has increased protein and reduced starch content when compared to the grain of the wild type durum or bread wheat plant.

### BRIEF DESCRIPTION OF THE DRAWINGS

FIG. 1 depicts the relationship between individual seed weight and average two-row yield for the SSII null and Wild Type Mountrail/55 and Mountrail/175 durum wheat varieties. Mountrail/55 (ab) and Mountrail/175 (ab) SSII null lines exhibit lower seed weight and yields compared to Mountrail/55 and Mountrail/175 (AB) SSII Wild-Type lines.

### DETAILED DESCRIPTION

All publications, patents and patent applications, including any drawings and appendices, and all nucleic acid sequences and polypeptide sequences identified by GenBank Accession numbers, cited herein are incorporated by reference to the same extent as if each individual publication or patent application was specifically and individually indicated to be incorporated by reference.

The following description includes information that may be useful in understanding the present invention. It is not an admission that any of the information provided herein is prior art or relevant to the presently claimed inventions, or that any publication specifically or implicitly referenced is prior art.

### DEFINITIONS

As used herein, the verb “comprise” as is used in this description and in the claims and its conjugations are used in its non-limiting sense to mean that items following the word are included, but items not specifically mentioned are not excluded.

The invention provides compositions and methods for improving the end product quality characteristics of plants. As used herein, the term “plant” refers to wheat (e.g., bread wheat or durum wheat), unless specified otherwise.

As used herein, the term “plant” also includes the whole plant or any parts or derivatives thereof, such as plant cells, plant protoplasts, plant cell tissue cultures from which wheat plants can be regenerated, plant calli, embryos, pollen, grain, ovules, fruit, flowers, leaves, seeds, roots, root tips and the like.

As used herein, the term “appropriate durum wheat check”, “appropriate bread wheat check”, or “appropriate wheat check” is meant to represent a wheat plant which provides a basis for evaluation of the experimental plants of the present invention (e.g. a corresponding durum or bread wheat variety without the genetic change of the experimental variety). An appropriate check is grown under the same environmental conditions, as is the experimental line, and is of approximately the same maturity as the experimental line. The term appropriate wheat check may actually reflect multiple appropriate varieties chosen to represent control lines for the modification or factor being tested in the experimental line. In some embodiments, the appropriate bread or durum wheat check variety can be a corresponding wild type bread or durum wheat variety without the experimental mutation (i.e., a “wild type wheat check variety”). In some embodiments, the appropriate bread or durum wheat check variety can be a corresponding SGP null mutant bread or durum wheat variety (i.e., a “null wheat check variety”. In some embodiments, durum wheat check lines can be ‘Mountrail’, ‘Divide’, ‘Strongfield’, or ‘Alzada’ wild type varieties. In some embodiments, bread wheat check lines can be ‘RJ-597/302’ or other ‘Alpowa’ varieties.

The invention provides plant parts. As used herein, the term “plant part” refers to any part of a plant including but not limited to the shoot, root, stem, seeds, stipules, leaves, petals, flowers, ovules, bracts, branches, petioles, internodes, bark, pubescence, tillers, rhizomes, fronds, blades, pollen, stamen, plant cells, grain and the like.

As used herein, the term “high amylose plant cell” refers to a plant cell capable of regenerating a wheat plant that produces a high amylose grain. In some embodiments the high amylose plant cell comprises at least one leaky SSII alleles.

The term “a” or “an” refers to one or more of that entity; for example, “a gene” refers to one or more genes or at least one gene. As such, the terms “a” (or “an”), “one or more” and “at least one” are used interchangeably herein. In addition, reference to “an element” by the indefinite article “a” or “an” does not exclude the possibility that more than one of the elements are present, unless the context clearly requires that there is one and only one of the elements.

The invention provides selectable markers. As used herein, the phrase “plant selectable or screenable marker” refers to a genetic marker functional in a plant cell. A selectable marker allows cells containing and expressing that marker to grow under conditions unfavorable to growth of cells not expressing that marker. A screenable marker facilitates identification of cells which express that marker.

The invention provides inbred plants. As used herein, the terms “inbred” and “inbred plant” are used in accordance with the context of the present invention. This also includes any single gene conversions of that inbred.

The term “single allele converted plant” as used herein refers to those plants which are developed by a plant breeding technique called backcrossing wherein essentially all of the desired morphological and physiological characteristics of an inbred are recovered in addition to the single allele transferred into the inbred via the backcrossing technique.

The invention provides plant samples. As used herein, the term “sample” includes a sample from a plant, a plant part, a plant cell, or from a transmission vector, or a soil, water or air sample.

The invention provides plant offsprings. As used herein, the term “offspring” refers to any plant resulting as progeny from a vegetative or sexual reproduction from one or more parent plants or descendants thereof. For instance an offspring plant may be obtained by cloning or selfing of a parent plant or by crossing two parent plants and include selfings as well as the F1 or F2 or still further generations. An F1 is a first-generation offspring produced from parents at least one of which is used for the first time as donor of a trait, while offspring of second generation (F2) or subsequent generations (F3, F4, etc.) are specimens produced from selfings of F1's, F2's etc. An F1 may thus be (and usually is) a hybrid resulting from a cross between two true breeding parents (true-breeding is homozygous for a trait), while an F2 may be (and usually is) an offspring resulting from self-pollination of said F1 hybrids.

The invention provides methods for crossing a first plant comprising recombinant sequences with a second plant. As used herein, the term “cross”, “crossing”, “cross pollination” or “cross-breeding” refer to the process by which the pollen of one flower on one plant is applied (artificially or naturally) to the ovule (stigma) of a flower on another plant.

The invention provides plant cultivars. As used herein, the term “cultivar” refers to a variety, strain or race of plant that has been produced by horticultural or agronomic techniques and is not normally found in wild populations.

The invention provides plant genes. As used herein, the term “gene” refers to any segment of DNA associated with a biological function. Thus, genes include, but are not limited to, coding sequences and/or the regulatory sequences required for their expression. Genes can also include nonexpressed DNA segments that, for example, form recognition sequences for other proteins. Genes can be obtained from a variety of sources, including cloning from a source of interest or synthesizing from known or predicted sequence information, and may include sequences designed to have desired parameters.

The invention provides plant genotypes. As used herein, the term “genotype” refers to the genetic makeup of an individual cell, cell culture, tissue, organism (e.g., a plant), or group of organisms.

In some embodiments, the present invention provides homozygotes of plants. As used herein, the term “hemizygous” refers to a cell, tissue or organism in which a gene is present only once in a genotype, as a gene in a haploid cell or organism, a sex-linked gene in the heterogametic sex, or a gene in a segment of chromosome in a diploid cell or organism where its partner segment has been deleted.

In some embodiments, the present invention provides heterologous nucleic acids. As used herein, the terms “heterologous polynucleotide” or a “heterologous nucleic acid” or an “exogenous DNA segment” refer to a polynucleotide, nucleic acid or DNA segment that originates from a source foreign to the particular host cell, or, if from the same source, is modified from its original form. Thus, a heterologous gene in a host cell includes a gene that is endogenous to the particular host cell, but has been modified. Thus, the terms refer to a DNA segment which is foreign or heterologous to the cell, or homologous to the cell but in a position within the host cell nucleic acid in which the element is not ordinarily found. Exogenous DNA segments are expressed to yield exogenous polypeptides.

In some embodiments, the present invention provides heterologous traits. As used herein, the term “heterologous trait” refers to a phenotype imparted to a transformed host cell or transgenic organism by an exogenous DNA segment, heterologous polynucleotide or heterologous nucleic acid.

In some embodiments, the present invention provides heterozygotes. As used herein, the term “heterozygote” refers to a diploid or polyploid individual cell or plant having different alleles (forms of a given gene) present at least at one locus.

In some embodiments, the present invention provides heterozygous traits. As used herein, the term “heterozygous” refers to the presence of different alleles (forms of a given gene) at a particular gene locus.

In some embodiments, the present invention provides homologs. As used herein, the terms “homolog” or “homologue” refer to a nucleic acid or peptide sequence which has a common origin and functions similarly to a nucleic acid or peptide sequence from another species.

In some embodiments, the present invention provides homozygotes. As used herein, the term “homozygote” refers to an individual cell or plant having the same alleles at one or more or all loci. When the term is used with reference to a specific locus or gene, it means at least that locus or gene has the same alleles.

In some embodiments, the present invention provides homozygous traits. As used herein, the terms “homozygous” or “HOMO” refer to the presence of identical alleles at one or more or all loci in homologous chromosomal segments. When the terms are used with reference to a specific locus or gene, it means at least that locus or gene has the same alleles.

In some embodiments, the present invention provides hybrids. As used herein, the term “hybrid” refers to any individual cell, tissue or plant resulting from a cross between parents that differ in one or more genes.

In some embodiments, the present invention provides mutants. As used herein, the terms “mutant” or “mutation” refer to a gene, cell, or organism with an abnormal genetic constitution that may result in a variant phenotype.

The invention provides open-pollinated populations. As used herein, the terms “open-pollinated population” or “open-pollinated variety” refer to plants normally capable of at least some cross-fertilization, selected to a standard, that may show variation but that also have one or more genotypic or phenotypic characteristics by which the population or the variety can be differentiated from others. A hybrid, which has no barriers to cross-pollination, is an open-pollinated population or an open-pollinated variety.

The invention provides plant ovules and pollens. As used herein when discussing plants, the term “ovule” refers to the female gametophyte, whereas the term “pollen” means the male gametophyte.

The invention provides plant phenotypes. As used herein, the term “phenotype” refers to the observable characters of an individual cell, cell culture, organism (e.g., a plant), or group of organisms which results from the interaction between that individual's genetic makeup (i.e., genotype) and the environment.

The invention provides plant tissue. As used herein, the term “plant tissue” refers to any part of a plant. Examples of plant organs include, but are not limited to the leaf, stem, root, tuber, seed, branch, pubescence, nodule, leaf axil, flower, pollen, stamen, pistil, petal, peduncle, stalk, stigma, style, bract, fruit, trunk, carpel, sepal, anther, ovule, pedicel, needle, cone, rhizome, stolon, shoot, pericarp, endosperm, placenta, berry, stamen, and leaf sheath.

The invention provides self-pollination populations. As used herein, the term “self-crossing”, “self pollinated” or “self-pollination” means the pollen of one flower on one plant is applied (artificially or naturally) to the ovule (stigma) of the same or a different flower on the same plant.

As used herein, the term “seed weight” or “kernel weight” refers to the mean weight of seeds produced from a wheat plant. In some embodiments, seed weight is represented in terms of 1,000 kernel seed weight (e.g., 30-50 grams/1000 wheat seeds). In other embodiments, seed weight is represented in terms of the mean weight of individual seeds (e.g., 30-50 mg per seed).

As used herein, the term “amylose content” refers to the amount of amylose in wheat starch. Amylose is a linear polymer of α-1,4 linked D-glucose with relatively few side chains. Amylose is digested more slowly than amylopectin which while also having linear polymers of α-1,4 linked D-glucose has many α-1,6 D-glucose side chains. Amylose absorbs less water upon heating than amylopectin and is digested more slowly. Amylose content can be measured by calorimetric assays involving iodine-potassium iodide assays, by DSC, Con A, or estimated by measuring the water absorbing capacity of flour or starch after heating.

As used herein, the term “starch synthesis genes” refers to any genes that directly or indirectly contribute to, regulate, or affect starch synthesis in a plant. Such genes includes, but are not limited to genes encoding waxy protein (a.k.a., Granule bound starch synthases (GBSS), such as GBSSI, GBSSII), ADP-glucose pyrophosphorylases (AGPases), starch branching enzymes (a.k.a., SBE, such as SBE I and SBE II), starch de-branching enzymes (a.k.a., SDBE), and starch synthases I, II, III, and IV.

As used herein, the term “waxy protein”, “Granule bound starch synthase”, GBSS, or “ADP-glucose:(1->4)-alpha-D-glucan 4-alpha-D-glucosyltransferase” refers to a protein having E.C. number 2.4.1.21, which can catalyze the following reaction:

ADP-glucose+(1,4-alpha-D-glucosyl)n=ADP+(1,4-alpha-D-glucosyl)n+1

As used herein, the term “ADP-glucose pyrophosphorylase”, AGPase, “adenosine diphosphate glucose pyrophosphorylase”, or “adenosine-5′-diphosphoglucose pyrophosphorylase” refers to a protein having E.C. number 2.7.7.27, which can catalyze the following reaction:

ATP+alpha-D-glucose 1-phosphate=diphosphate+ADP-glucose

As used herein, the term “starch branching enzyme”, SBE, “branching enzyme”, BE, “glycogen branching enzyme”, “1,4-alpha-glucan branching enzyme”, “alpha-1,4-glucan:alpha-1,4-glucan 6-glycosyltransferase” or “(1->4)-alpha-D-glucan:(1->4)-alpha-D-glucan 6-alpha-D-[(1->4)-alpha-D-glucano]-transferase” refers to a protein having E.C. number 2.4.1.18, which can catalyze the following reaction:

2 1,4-alpha-D-glucan=alpha-1,4-D-glucan-alpha-1,6-(alpha-1,4-D-glucan)

As used herein, the term “starch de-branching enzymes”, SDBE, or isoamylase refers to a protein having the E.C. number 2.4.1.1, 2.4.1.25, 3.2.1.68 or 3.2.1.41, which can hydrolyze alpha-1,6 glucosidic bonds in glucans containing both alpha-1,4 and alpha-1,6 linkages.

As used herein, the term starch synthase I, II, III, or IV (SSI or SI, SSII or SII, SSIII or SOOO, and SSIV or SIV), refers to a protein of starch synthase class I, class II, class III, or class IV, respectively. Such as protein that is involved in amylopectin synthesis.

As used herein, the term starch granule protein-1 or SGP-1 refers to a protein belonging to starch synthase class II, contained in wheat starch granules (Yamamori and Endo, 1996).

As used herein, the term wheat refers to any wheat species within the genus of Triticum, or the tribe of Triticeae, which includes, but are not limited to, diploid, tetraploid, and hexaploid wheat species.

As used herein, the term “milled product” refers to a product produced from grinding grains (from wheat or other grain producing plants). Non-limiting examples of milled products include: flour, all purpose flour, starch, bread flour, cake flour, self-rising flour, pastry flour, semolina, durum flour, bread wheat flour whole wheat flour, stone ground flour, gluten flour, and graham flour among others.

As used herein, the term “flour based product” refers to products made from flour including: pasta, noodles, bread products, cookies, and pastries among others.

As used herein, the term “high amylose grain” refers to a wheat grain (e.g., bread wheat grain) with starch with high levels of amylose. In some embodiments, the high amylose levels are elevated compared to the amylose content of a wheat grain from a wild type or other appropriate wheat check variety grown at the same time under similar field conditions. In other embodiments, the amylose levels are high in absolute percentage terms as measured by differential scanning calorimetry analysis.

As used herein, the term diploid wheat refers to wheat species that have two homologous copies of each chromosome, such as Einkorn wheat (T. monococcum), having the genome AA.

As used herein, the term tetraploid wheat refers to wheat species that have four homologous copies of each chromosome, such as emmer and durum wheat, which are derived from wild emmer (T. dicoccoides). Wild emmer is itself the result of a hybridization between two diploid wild grasses, T. urartu and a wild goatgrass such as Aegilops searsii or Ae. speltoides. The hybridization that formed wild emmer (having genome AABB) occurred in the wild, long before domestication, and was driven by natural selection.

As used herein, the term hexaploid wheat refers to wheat species that have six homologous copies of each chromosome, such as bread wheat. Either domesticated emmer or durum wheat hybridized with another wild diploid grass (Aegilops tauschii, having genome DD) to make the hexaploid wheat (having genome AABBDD).

As used herein, SSIIa-Aa refers to both wild type “aa” alleles being present but SSIIa-Ab refers to both “bb” alleles being present. SSIIa and SSIIb would be two different forms of the same enzyme.

As used herein, the term “gelatinization temperature” refers to the temperature at which starch is dissolved in water during heating. Gelatinization temperature is related to amylose content with increased amylose content associated with increased gelatinization temperature.

As used herein, the term “starch retrogradation” refers to the firmness of starch water gels with increased amylose associated with increased starch retrogradation and firmer starch based gels.

As used herein, the term “flour swelling power” or FSP refers to the weight of flour or starch based gel relative to the weight of the original sample after heating in the presence of excess water. Increased amylose is associated with decreased FSP.

As used herein, the term “grain hardness” refers to the pressure required to fracture grains and is related to particle size after milling, milling yield, and some end product quality traits. Increased grain hardness is associated with increased flour particle size, increased starch damage and decreased break flour yield.

As used herein, the term “semolina” refers to the coarse, purified wheat middlings of durum wheat.

As used herein, the term “resistant amylose” refers to amylose which resists digestion and thus serves a purpose in the manufacturing of reduced glycemic index food products.

As used herein, the term “resistant starch” refers to starch that resists digestion and behaves like dietary fiber. Increased amylose is believed to be associated with increased resistant starch.

As used herein, the term “allele” refers to any of several alternative forms of a gene.

As used herein, the term “wild type functional allele” refers to an allele that exhibits normal gene function. For example, in some embodiments, the wild type functional allele exhibits normal gene function comparable to that of the corresponding allele in a wild species. For example, in some embodiments, a wild type functional SSII allele would exhibit similar levels of SSII protein accumulation in an SDS PAGE gel than a wild type SSII allele (e.g., SSII-A, SSII-B, or SSII, D).

In some embodiments, the present invention teaches the use of “null” alleles, which are alleles that lack that gene's normal function (e.g., trace, or no gene function). In some embodiments, null alleles can be caused by one or more genetic mutations. For example, in some embodiments, the mutation producing the null allele is located on the coding portions of the gene. In some embodiments, a leaky allele can comprise one or more i) missense mutations, ii) nonsense mutations, iii) silent mutations (e.g., rare codon usage), iv) splice junction mutations (e.g. affecting transcript processing), v) insertions/or deletions, vi) promoter and or UTR mutations (e.g., affecting transcript expression or half life), or a combination thereof.

As used herein, the term “leaky alleles” refers to alleles that confer an intermediate phenotype between that of wild-type alleles and null alleles of the same gene. For example, leaky alleles can encode gene products that exhibit activities lower than wild-type alleles, but higher activity than “null” alleles. Thus in the case of a gene coding for an enzyme, a leaky allele-encoded enzyme would consume substrate and/or generate products at lower rates/levels than the corresponding wild type allele-encoded enzyme, but at higher rates/levels than completely null alleles of the same gene. In some embodiments, leaky alleles can be caused by one or more genetic mutations. For example, in some embodiments, the mutation producing the leaky allele is located on the coding portions of the gene. In some embodiments, a leaky allele can comprise one or more i) missense mutations, ii) nonsense mutations, iii) silent mutations (e.g., rare codon usage), iv) splice junction mutations (e.g. affecting transcript processing), v) promoter and or UTR mutations (e.g., affecting transcript expression or half life), or a combination thereof.

As used herein, the term SSII leaky wheat refers to a wheat plant comprising one or more starch synthase II leaky alleles. In some embodiments, the SSII leaky wheat does not comprise any SSII wild type alleles. For example, SSII “leaky allele” wheat plants can produce seed of an intermediate size, which is measurably larger than the seed size of null SSSII alleles but no larger than the wild-type allele (normal seed size).

As used herein, “starch” refers to starch in its natural or native form as well as also referring to starch modified by physical, chemical, enzymatic and biological processes.

As used herein, “amylose” refers to a starch polymer that is an essentially linear assemblage of D-anhydroglucose units which are linked by alpha 1,6-D-glucosidic bonds.

As used herein, “amylose content” refers to the percentage of the amylose type polymer in relation to other starch polymers such as amylopectin.

As used herein, the term “grain” refers to mature wheat kernels produced by commercial growers for purposes other than growing or reproducing the species.

As used herein, the term “kernel” refers to the wheat caryopsis comprising a mature embryo and endosperm which are products of double fertilization.

As used herein, the term “line” is used broadly to include, but is not limited to, a group of plants vegetatively propagated from a single parent plant, via tissue culture techniques or a group of inbred plants which are genetically very similar due to descent from a common parent(s). A plant is said to “belong” to a particular line if it (a) is a primary transformant (T0) plant regenerated from material of that line; (b) has a pedigree comprised of a T0 plant of that line; or (c) is genetically very similar due to common ancestry (e.g., via inbreeding or selfing). In this context, the term “pedigree” denotes the lineage of a plant, e.g. in terms of the sexual crosses effected such that a gene or a combination of genes, in heterozygous (hemizygous) or homozygous condition, imparts a desired trait to the plant.

As used herein, the term “locus” (plural: “loci”) refers to any site that has been defined genetically. A locus may be a gene, or part of a gene, or a DNA sequence that has some regulatory role, and may be occupied by the same or different sequences.

The invention provides methods for obtaining plants or plant cells through transformation. As used herein, the term “transformation” refers to the transfer of nucleic acid (i.e., a nucleotide polymer) into a cell. As used herein, the term “genetic transformation” refers to the transfer and incorporation of DNA, especially recombinant DNA, into a cell.

The invention provides plant and plant cell transformants. As used herein, the term “transformant” refers to a cell, tissue or organism that has undergone transformation. The original transformant is designated as “T0” or “T₀.” Selfing the T0 produces a first transformed generation designated as “T1” or “T₁.”

The invention provides plant transgenes. As used herein, the term “transgene” refers to a nucleic acid that is inserted into an organism, host cell or vector in a manner that ensures its function.

The invention provides plant transgenic plants, plant parts, and plant cells. As used herein, the term “transgenic” refers to cells, cell cultures, organisms (e.g., plants), and progeny which have received a foreign or modified gene by one of the various methods of transformation, wherein the foreign or modified gene is from the same or different species than the species of the organism receiving the foreign or modified gene.

The invention provides plant transposition events. As used herein, the term “transposition event” refers to the movement of a transposon from a donor site to a target site.

The invention provides plant varieties. As used herein, the term “variety” refers to a subdivision of a species, consisting of a group of individuals within the species that are distinct in form or function from other similar arrays of individuals.

The invention provides plant vectors, plasmids, or constructs. As used herein, the term “vector”, “plasmid”, or “construct” refers broadly to any plasmid or virus encoding an exogenous nucleic acid. The term should also be construed to include non-plasmid and non-viral compounds which facilitate transfer of nucleic acid into virions or cells, such as, for example, polylysine compounds and the like. The vector may be a viral vector that is suitable as a delivery vehicle for delivery of the nucleic acid, or mutant thereof, to a cell, or the vector may be a non-viral vector which is suitable for the same purpose. Examples of viral and non-viral vectors for delivery of DNA to cells and tissues are well known in the art and are described, for example, in Ma et al. (1997, Proc. Natl. Acad. Sci. U.S.A. 94:12744-12746).

The invention provides isolated, chimeric, recombinant or synthetic polynucleotide sequences. As used herein, the term “polynucleotide”, “polynucleotide sequence”, or “nucleic acid” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like. The terms “nucleic acid” and “nucleotide sequence” are used interchangeably. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof. Nucleotides (usually found in their 5′-monophosphate form) are referred to by a single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

The invention provides isolated, chimeric, recombinant or polypeptide sequences. As used herein, the terms “polypeptide,” “peptide,” and “protein” are used interchangeably herein to refer to polymers of amino acids of any length. These terms also include proteins that are post-translationally modified through reactions that include glycosylation, acetylation and phosphorylation.

The invention provides homologous and orthologous polynucleotides and polypeptides. As used herein, the term “homologous” or “homologue” or “ortholog” is known in the art and refers to related sequences that share a common ancestor or family member and are determined based on the degree of sequence identity. The terms “homology”, “homologous”, “substantially similar” and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant invention such as deletion or insertion of one or more nucleotides that do not substantially alter the functional properties of the resulting nucleic acid fragment relative to the initial, unmodified fragment. It is therefore understood, as those skilled in the art will appreciate, that the invention encompasses more than the specific exemplary sequences. These terms describe the relationship between a gene found in one species, subspecies, variety, cultivar or strain and the corresponding or equivalent gene in another species, subspecies, variety, cultivar or strain. For purposes of this invention homologous sequences are compared. “Homologous sequences” or “homologues” or “orthologs” are thought, believed, or known to be functionally related. A functional relationship may be indicated in any one of a number of ways, including, but not limited to: (a) degree of sequence identity and/or (b) the same or similar biological function. Preferably, both (a) and (b) are indicated. The degree of sequence identity may vary, but in one embodiment, is at least 50% (when using standard sequence alignment programs known in the art), at least 60%, at least 65%, at least 70%, at least 75%, at least 80%, at least 85%, at least 90%, at least about 91%, at least about 92%, at least about 93%, at least about 94%, at least about 95%, at least about 96%, at least about 97%, at least about 98%, or at least 98.5%, or at least about 99%, or at least 99.5%, or at least 99.8%, or at least 99.9%. Homology can be determined using software programs readily available in the art, such as those discussed in Current Protocols in Molecular Biology (F. M. Ausubel et al., eds., 1987) Supplement 30, section 7.718, Table 7.71. Some alignment programs are MacVector (Oxford Molecular Ltd, Oxford, U.K.), ALIGN Plus (Scientific and Educational Software, Pennsylvania) and AlignX (Vector NTI, Invitrogen, Carlsbad, Calif.). Another alignment program is Sequencher (Gene Codes, Ann Arbor, Mich.), using default parameters. In some embodiments, the sequence alignments and sequence identities of the present invention are calculated using standard settings of the ClustalOmega tool found in (http://www.ebi.ac.uk/Tools/msa/clustalo/).

The invention provides polynucleotides with nucleotide change when compared to a wild-type reference sequence. As used herein, the term “nucleotide change” refers to, e.g., nucleotide substitution, deletion, and/or insertion, as is well understood in the art. For example, mutations contain alterations that produce silent substitutions, additions, or deletions, but do not alter the properties or activities of the encoded protein or how the proteins are made.

The invention provides polypeptides with protein modification when compared to a wild-type reference sequence. As used herein, the term “protein modification” refers to, e.g., amino acid substitution, amino acid modification, deletion, and/or insertion, as is well understood in the art.

The invention provides polynucleotides and polypeptides derived from wild-type reference sequences. As used herein, the term “derived from” refers to the origin or source, and may include naturally occurring, recombinant, unpurified, or purified molecules, and may also include cells whose origin is a plant or plant part. A nucleic acid or an amino acid derived from an origin or source may have all kinds of nucleotide changes or protein modification as defined elsewhere herein.

The invention provides portions or fragments of the nucleic acid sequences and polypeptide sequences of the present invention. As used herein, the term “at least a portion” or “fragment” of a nucleic acid or polypeptide means a portion having the minimal size characteristics of such sequences, or any larger fragment of the full length molecule, up to and including the full length molecule. For example, a portion of a nucleic acid may be 12 nucleotides, 13 nucleotides, 14 nucleotides, 15 nucleotides, 16 nucleotides, 17 nucleotides, 18 nucleotides, 19 nucleotides, 20 nucleotides, 22 nucleotides, 24 nucleotides, 26 nucleotides, 28 nucleotides, 30 nucleotides, 32 nucleotides, 34 nucleotides, 36 nucleotides, 38 nucleotides, 40 nucleotides, 45 nucleotides, 50 nucleotides, 55 nucleotides, and so on, going up to the full length nucleic acid. Similarly, a portion of a polypeptide may be 4 amino acids, 5 amino acids, 6 amino acids, 7 amino acids, and so on, going up to the full length polypeptide. The length of the portion to be used will depend on the particular application. A portion of a nucleic acid useful as hybridization probe may be as short as 12 nucleotides; in one embodiment, it is 20 nucleotides. A portion of a polypeptide useful as an epitope may be as short as 4 amino acids. A portion of a polypeptide that performs the function of the full-length polypeptide would generally be longer than 4 amino acids.

The invention provides sequences having high similarity or identity to the nucleic acid sequences and polypeptide sequences of the present invention. As used herein, “sequence identity” or “identity” in the context of two nucleic acid or polypeptide sequences includes reference to the residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988).

The invention provides sequences substantially complementary to the nucleic acid sequences of the present invention. As used herein, the term “substantially complementary” means that two nucleic acid sequences have at least about 65%, preferably about 70% or 75%, more preferably about 80% or 85%, even more preferably 90% or 95%, and most preferably about 98% or 99%, sequence complementarities to each other. This means that primers and probes must exhibit sufficient complementarity to their template and target nucleic acid, respectively, to hybridize under stringent conditions. Therefore, the primer and probe sequences need not reflect the exact complementary sequence of the binding region on the template and degenerate primers can be used. For example, a non-complementary nucleotide fragment may be attached to the 5′-end of the primer, with the remainder of the primer sequence being complementary to the strand. Alternatively, non-complementary bases or longer sequences can be interspersed into the primer, provided that the primer has sufficient complementarity with the sequence of one of the strands to be amplified to hybridize therewith, and to thereby form a duplex structure which can be extended by polymerizing means. The non-complementary nucleotide sequences of the primers may include restriction enzyme sites. Appending a restriction enzyme site to the end(s) of the target sequence would be particularly helpful for cloning of the target sequence. A substantially complementary primer sequence is one that has sufficient sequence complementarity to the amplification template to result in primer binding and second-strand synthesis. The skilled person is familiar with the requirements of primers to have sufficient sequence complementarity to the amplification template.

The invention provides biologically active variants or functional variants of the nucleic acid sequences and polypeptide sequences of the present invention. As used herein, the phrase “a biologically active variant” or “functional variant” with respect to a protein refers to an amino acid sequence that is altered by one or more amino acids with respect to a reference sequence, while still maintains substantial biological activity of the reference sequence. The variant can have “conservative” changes, wherein a substituted amino acid has similar structural or chemical properties, e.g., replacement of leucine with isoleucine. Alternatively, a variant can have “nonconservative” changes, e.g., replacement of a glycine with a tryptophan. Analogous minor variations can also include amino acid deletion or insertion, or both. Guidance in determining which amino acid residues can be substituted, inserted, or deleted without eliminating biological or immunological activity can be found using computer programs well known in the art, for example, DNASTAR software. For polynucleotides, a variant comprises a polynucleotide having deletions (i.e., truncations) at the 5′ and/or 3′ end; deletion and/or addition of one or more nucleotides at one or more internal sites in the reference polynucleotide; and/or substitution of one or more nucleotides at one or more sites in the reference polynucleotide. As used herein, a “reference” polynucleotide comprises a nucleotide sequence produced by the methods disclosed herein. Variant polynucleotides also include synthetically derived polynucleotides, such as those generated, for example, by using site directed mutagenesis but which still comprise genetic regulatory element activity. Generally, variants of a particular polynucleotide or nucleic acid molecule of the invention will have at least about 60%, 65%, 70%, 75%, 80%, 85%, 90%, 91%, 91.5%, 92%, 92.5%, 93%, 93.5%, 94%, 94.5%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or more sequence identity to that particular polynucleotide as determined by sequence alignment programs and parameters as described elsewhere herein.

Variant polynucleotides also encompass sequences derived from a mutagenic and recombinogenic procedure such as DNA shuffling. Strategies for such DNA shuffling are known in the art. See, for example, Stemmer (1994) PNAS 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) PNAS 94:4504-4509; Crameri et al. (1998) Nature 391:288-291; and U.S. Pat. Nos. 5,605,793 and 5,837,458. For PCR amplifications of the polynucleotides disclosed herein, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in Sambrook et al. (1989) Molecular Cloning: A Laboratory Manual (2nd ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

The invention provides primers that are derived from the nucleic acid sequences and polypeptide sequences of the present invention. The term “primer” as used herein refers to an oligonucleotide which is capable of annealing to the amplification target allowing a DNA polymerase to attach, thereby serving as a point of initiation of DNA synthesis when placed under conditions in which synthesis of primer extension product is induced, i.e., in the presence of nucleotides and an agent for polymerization such as DNA polymerase and at a suitable temperature and pH. The (amplification) primer is preferably single stranded for maximum efficiency in amplification. Preferably, the primer is an oligodeoxyribonucleotide. The primer must be sufficiently long to prime the synthesis of extension products in the presence of the agent for polymerization. The exact lengths of the primers will depend on many factors, including temperature and composition (A/T vs. G/C content) of primer. A pair of bi-directional primers consists of one forward and one reverse primer as commonly used in the art of DNA amplification such as in PCR amplification.

The invention provides polynucleotide sequences that can hybridize with the nucleic acid sequences of the present invention. The terms “stringency” or “stringent hybridization conditions” refer to hybridization conditions that affect the stability of hybrids, e.g., temperature, salt concentration, pH, formamide concentration and the like. These conditions are empirically optimized to maximize specific binding and minimize non-specific binding of primer or probe to its target nucleic acid sequence. The terms as used include reference to conditions under which a probe or primer will hybridize to its target sequence, to a detectably greater degree than other sequences (e.g. at least 2-fold over background). Stringent conditions are sequence dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength and pH) at which 50% of a complementary target sequence hybridizes to a perfectly matched probe or primer. Typically, stringent conditions will be those in which the salt concentration is less than about 1.0 M Na⁺ ion, typically about 0.01 to 1.0 M Na+ ion concentration (or other salts) at pH 7.0 to 8.3 and the temperature is at least about 30° C. for short probes or primers (e.g. 10 to 50 nucleotides) and at least about 60° C. for long probes or primers (e.g. greater than 50 nucleotides). Stringent conditions may also be achieved with the addition of destabilizing agents such as formamide. Exemplary low stringent conditions or “conditions of reduced stringency” include hybridization with a buffer solution of 30% formamide, 1 M NaCl, 1% SDS at 37° C. and a wash in 2×SSC at 40° C. Exemplary high stringency conditions include hybridization in 50% formamide, 1M NaCl, 1% SDS at 37° C., and a wash in 0.1×SSC at 60° C. Hybridization procedures are well known in the art and are described by e.g. Ausubel et al., 1998 and Sambrook et al., 2001.

The invention provides coding sequences. As used herein, “coding sequence” refers to a DNA sequence that codes for a specific amino acid sequence.

The invention provides regulatory sequences. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence.

The invention provides promoter sequences. As used herein, “promoter” refers to a DNA sequence capable of controlling the expression of a coding sequence or functional RNA. The promoter sequence consists of proximal and more distal upstream elements, the latter elements often referred to as enhancers. Accordingly, an “enhancer” is a DNA sequence that can stimulate promoter activity, and may be an innate element of the promoter or a heterologous element inserted to enhance the level or tissue specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity.

In some embodiments, the invention provides plant promoters. As used herein, a “plant promoter” is a promoter capable of initiating transcription in plant cells whether or not its origin is a plant cell, e.g. it is well known that Agrobacterium promoters are functional in plant cells. Thus, plant promoters include promoter DNA obtained from plants, plant viruses and bacteria such as Agrobacterium and Bradyrhizobium bacteria. A plant promoter can be a constitutive promoter or a non-constitutive promoter.

The invention provides recombinant genes comprising 3′ non-coding sequences or 3′ untranslated regions. As used herein, the “3′ non-coding sequences” or “3′ untranslated regions” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht, I. L., et al. (1989) Plant Cell 1:671-680.

The invention provides RNA transcripts. As used herein, “RNA transcript” refers to the product resulting from RNA polymerase-catalyzed transcription of a DNA sequence. When the RNA transcript is a perfect complementary copy of the DNA sequence, it is referred to as the primary transcript. An RNA transcript is referred to as the mature RNA when it is an RNA sequence derived from post-transcriptional processing of the primary transcript. “Messenger RNA (mRNA)” refers to the RNA that is without introns and that can be translated into protein by the cell. “cDNA” refers to a DNA that is complementary to and synthesized from an mRNA template using the enzyme reverse transcriptase. The cDNA can be single-stranded or converted into the double-stranded form using the Klenow fragment of DNA polymerase I. “Sense” RNA refers to RNA transcript that includes the mRNA and can be translated into protein within a cell or in vitro. “Antisense RNA” refers to an RNA transcript that is complementary to all or part of a target primary transcript or mRNA, and that blocks the expression of a target gene (U.S. Pat. No. 5,107,065). The complementarity of an antisense RNA may be with any part of the specific gene transcript, i.e., at the 5′ non-coding sequence, 3′ non-coding sequence, introns, or the coding sequence. “Functional RNA” refers to antisense RNA, ribozyme RNA, or other RNA that may not be translated but yet has an effect on cellular processes. The terms “complement” and “reverse complement” are used interchangeably herein with respect to mRNA transcripts, and are meant to define the antisense RNA of the message.

The invention provides recombinant genes in which a gene of interest is operably linked to a promoter sequence. As used herein, the term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. In another example, the complementary RNA regions of the invention can be operably linked, either directly or indirectly, 5′ to the target mRNA, or 3′ to the target mRNA, or within the target mRNA, or a first complementary region is 5′ and its complement is 3′ to the target mRNA.

The invention provides recombinant expression cassettes and recombinant constructs. As used herein, the term “recombinant” refers to an artificial combination of two otherwise separated segments of sequence, e.g., by chemical synthesis or by the manipulation of isolated segments of nucleic acids by genetic engineering techniques. As used herein, the phrases “recombinant construct”, “expression construct”, “chimeric construct”, “construct”, and “recombinant DNA construct” are used interchangeably herein. A recombinant construct comprises an artificial combination of nucleic acid fragments, e.g., regulatory and coding sequences that are not found together in nature. For example, a chimeric construct may comprise regulatory sequences and coding sequences that are derived from different sources, or regulatory sequences and coding sequences derived from the same source, but arranged in a manner different than that found in nature. Such construct may be used by itself or may be used in conjunction with a vector. If a vector is used then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells comprising any of the isolated nucleic acid fragments of the invention. The skilled artisan will also recognize that different independent transformation events will result in different levels and patterns of expression (Jones et al., (1985) EMBO J. 4:2411-2418; De Almeida et al., (1989) Mol. Gen. Genetics 218:78-86), and thus that multiple events must be screened in order to obtain lines displaying the desired expression level and pattern. Such screening may be accomplished by Southern analysis of DNA, Northern analysis of mRNA expression, immunoblotting analysis of protein expression, or phenotypic analysis, among others. Vectors can be plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating.

In yet another embodiment, the present invention provides a tissue culture of regenerable cells of a wheat plant obtained from the wheat lines of the present invention (e.g., bread wheat), wherein the tissue regenerates plants having all or substantially all of the morphological and physiological characteristics of the wheat plants provided by the present invention. In one such embodiment, the tissue culture is derived from a plant part selected from the group consisting of leaves, roots, root tips, root hairs, anthers, pistils, stamens, pollen, ovules, flowers, seeds, embryos, stems, buds, cotyledons, hypocotyls, cells and protoplasts. In another such embodiment, the present invention includes a wheat plant regenerated from the above described tissue culture.

This invention provides the cells, cell culture, tissues, tissue culture, seed, whole plant and plant parts of bread wheat germplasm designated leaky parent ‘122’ or ‘624’ or derived from leaky parent ‘122’ or ‘624’, or any of its offspring.

This invention provides the cells, cell culture, tissues, tissue culture, seed, whole plant and plant parts of durum wheat germplasm designated leaky parent ‘213’ or ‘217’ or derived from leaky parent ‘213’ or ‘217’ or any of its offspring.

This invention provides the cells, cell culture, tissues, tissue culture, seed, whole plant and plant parts of durum wheat germplasm designated leaky parent ‘1174’, ‘1513’, ‘134’, or ‘1704’ or derived from leaky parent ‘1174’, ‘1513’, ‘134’, ‘1704’ or any of its offspring.

For example methods of wheat tissue culture please see (Altpeter et al., 1996; Smidansky et al., 2002)

### Wheat

Wheat is a plant species belonging to the genus of Triticum. Non-limiting examples of wheat species include, T. aestivum (a.k.a., common wheat, or bread wheat, hexaploid), T. aethiopicum, T. araraticum, T. boeoticum, T. carthlicum, T. compactum, T. dicoccoides, T. dicoccum (a.k.a., emmer wheat, tetraploid), T. durum (a.k.a., durum wheat, tetraploid), T. ispahanicum, T. karamyschevii, T. macha, T. militinae, T. monococcum (Einkorn wheat, diploid), T. polonicum, T. spelta (a.k.a. spelt, hexaploid), T. sphaerococcum, T. timopheevii, T. turanicum, T. turgidum, T. urartu, T. vavilovii, T. zhukovskyi, and any hybridization thereof.

Some wheat species are diploid, with two sets of chromosomes, but many are stable polyploids, with four sets (tetraploid) or six sets (hexaploid) of chromosomes.

Einkorn wheat (T. monococcum) is diploid (AA, two complements of seven chromosomes, 2n=14). Most tetraploid wheat (e.g. emmer and durum wheat) are derived from wild emmer, T. dicoccoides. Wild emmer is itself the result of a hybridization between two diploid wild grasses, T. urartu and a wild goatgrass such as Aegilops searsii or Aegilops speltoides. The hybridization that formed wild emmer (AABB) occurred in the wild, long before domestication, and was driven by natural selection (Hancock, James F. (2004) Plant Evolution and the Origin of Crop Species. CABI Publishing. ISBN 0-85199-685-X). Hexaploid wheat (AABBDD) evolved in farmers' fields. Either domesticated emmer or durum wheat hybridized with yet another wild diploid grass (Aegilops tauschii) to make the hexaploid wheat, spelt wheat and bread wheat. These have three sets of paired chromosomes.

Therefore, in hexaploid wheat, most genes exist in triplicated homologous sets, one from each genome (i.e., the A genome, the B genome, or the D genome), while in tetraploid wheat, most genes exist in doubled homologous sets, one from each genome (i.e., the A genome or the B genome). Due to random mutations that occur along genomes, the alleles isolated from different genomes are not necessarily identical.

The presence of certain alleles of wheat genes is important for crop phenotypes. Some alleles encode functional polypeptides with equal or substantially equal activity of a reference allele. Some alleles encode polypeptides having increased activity when compared to a reference allele. Some alleles are in disrupted versions which do not encode functional polypeptides, or only encode polypeptides having less activity compared to a reference allele. Each of the different alleles can be utilized depending on the specific goals of a breeding program.

### Wheat Starch Synthesis Genes

Starch is the major reserve carbohydrate in plants. It is present in practically every type of tissue: leaf, fruit, root, shoot, stem, pollen, and seed. In cereal grains, starch is the primary source of stored energy. The amount of starch contained in cereal grains varies depending on species, and developmental stages.

Two types of starch granules are found in the wheat endosperm. The large (A-type) starch granules of wheat are disk-like or lenticular in shape, with an average diameter of 10-35 μm, whereas the small (B-type) starch granules are roughly spherical or polygonal in shape, ranging from 1 to 10 μm in diameter.

Bread wheat (Triticum aestivum L.) starch normally consists of roughly 25% amylose and 75% amylopectin (reviewed in Hannah and James, 2008). Amylose is a linear chain of glucose molecules linked by α-1,4 linkages. Amylopectin consists of glucose residues linked by α-1,4 linkages with α-1,6 branch points.

Starch synthesis is catalyzed by starch synthases. Amylose and amylopectin are synthesized by two pathways having a common substrate, ADP-glucose. AGPase catalyzes the initial step in starch synthesis in plants. Waxy proteins granule bound starch synthase I (GBSSI) is encoded by Wx genes which are responsible for amylose synthesis. Soluble starch synthase, such as starch synthase I (SSI or SI), II (SSII or SII), and III (SSIII or SIM, starch branching enzymes (e.g., SBEI, SBEIIa and SBEIIb), and starch debranching enzymes of isoamylase- and limit dextrinase-type (ISA and LD) are believed to play key roles in amylopectin synthesis.

SSI of wheat is partitioned between the granule and the soluble fraction (Li et al., 1999, Peng et al., 2001). Wheat SSII is predominantly granule-bound with only a small amount present in the soluble fraction (Gao and Chibbar, 2000). SSIII is exclusively found in the soluble fraction of wheat endosperm (Li et al., 2000).

In some embodiments, the present disclosure will refer to a SSII allele with a specific amino acid or nucleotide sequence mutation. For example, in some embodiments, the present disclosure teaches SSII-D-E656K. This notation is refers to the gene-genome-substitution of the allele in question. Thus, SSII-D-E656K refers to the starch synthase II gene in the D genome of a hexaploid wheat, wherein the sequence comprises a mutation causing the SSII protein to exhibit an amino acid change of E at position 365 to K.

SBEs can be separated into two major groups. SBE type I (or class B) comprises SBEI from maize (Baba et al, 1991), wheat (Morell et al, 1997, Repellin et al, 1997, Baga et al, 1999b), potato (Kossman et al, 1991), rice (Kawasaki et al, 1993), and cassava (Salehuzzaman et al., 1992), and SBEII from pea (Burton et aL, 1995). The other group, SBE type II (or class A), comprises SBEII from maize (Gao et al, 1997), wheat (Nair et al, 1997), potato (Larsson et al, 1996), and Arabidopsis (Fisher et aL, 1996), SBEIII from rice (Mizuno et al, 1993), and SBEI from pea (Bhattacharyya et al, 1990). SBEI and SBEII are generally immunologically unrelated but have distinct catalytic activities. SBEI transfers long glucan chains and prefers amylose as a substrate, while SBEII acts primarily on amylopectin (Guan and Preiss, 1993). SBEII is further sub classified into SBElla and SBEllb, each of which differs slightly in catalytic properties. The two SBEII forms are encoded by different genes and expressed in a tissue-specific manner (Gao et al., 1997, Fisher et al., 1996). Expression patterns of SBElla and SBEllb in a particular tissue are specific to plant species. For example, the endosperm-specific SBEII in rice is SBElla (Yamanouchi and Nakamura, 1992), while that in barley is SBEllb (Sun et al., 1998).

SBE can be either alpha-1,4-targeting enzymes, such as amylases, starch phosphorylase (EC 2.4.1.1), disproportionating enzyme (EC 2.4.1.25), or alpha-1,6-targeting enzymes, such as direct debranching enzymes (e.g., limit dextrinase, EC 3.2.1.41, or isoamylase, EC 3.2.2.68), indirect debranching enzymes (e.g., alpha-1,4- and alpha-4,6-targeting enzymes). Several starch biosynthetic proteins can be found bound to the interior of starch granules. A subset of these proteins has been designated the starch granule proteins (SGPs). Bread wheat starch granule proteins (SGPs) at least include SGP-1, SGP-2 and SGP-3 all with molecular masses>80 kd and the waxy protein (GBSS). Using SDS-PAGE, Yamamori and Endo (1996) separated the SGPs from bread wheat starch into SGP-1, SGP-2, SGP-3 and WX. The SGP-1 fraction was further resolved into SGP-A1, SGP-B1, and SGP-D1 and the associated genes localized to the homologous group 7 chromosomes (Yamamori and Endo, 1996). SGP-1 proteins are isoforms of SSII encoded by the genes SSII-A, SSIIa-B, SSII-D on the short arms of group 7 chromosomes (Li et al., 1999).

In some embodiments, this specification will refer to SSII alleles leading to SGP-1 mutations as SGP1, or SGP-1 mutations.

Increased Amylose is observed by about 8% in the SGP-1 null line compared to the wild type inferring that SGP-1 is involved in amylopectin synthesis (Yamamori et al. (2000). The SGP-1 null line also shows deformed starch granules, lower overall starch content, altered amylopectin content, and reduced binding of SGP-2 and SGP-3 to starch granules. SGP-1 proteins are starch synthase class II enzymes and genes encoding these enzymes are designated SSII-A1, SSII-B1, and SSII-D1 (Li et al., 1999).

Durum wheat (Triticum turgidum L. var. durum) being tetraploid lacks the D genome of bread wheat but homoalleles for genes encoding the SGP-1 proteins are present on the A and B genomes (Lafiandra et al., 2010).

SGP-1 mutations are thought to alter the interactions of other granule bound enzymes by reducing their entrapment in starch granules. Similarly, barley SSIIa sex6 locus mutations have seeds with decreased starch content, increased amylose content (+45%) (70.3% for two SGP-1 mutants vs. 25.4% wild-type), deformed starch granules, and decreased binding of other SGPs (Morell et al. 2003). These barley ssIIa mutants had normal expression of SSI, SBEIIa, and SBEIIb based on western blot analysis of the soluble protein fraction demonstrating that there was not a global down regulation of starch synthesis genes. In SGP-1 triple mutant in bread wheat, SSI, SBEIIa, and SBEIIb proteins were stably expressed in developing seeds even though they are not present in the starch granule fraction (Kosar-Hashemi et al. 2007). Similar results relating the loss of SSII and increased amylose have been observed in both maize (Zhang et al. 2004) and pea (Craig et al. 1998).

Elimination of another important gene for amylopectin synthesis, SbeIIa, in durum wheat through RNA interference resulted in amylose increases ranging from +8% to +50% (24% wild-type vs. 31-75% SbeIIa RNAi lines), although protein content was found to be similar or, in some cases, lower than wild type. (Sestili et al. 2010b). It was determined through qRT-PCR that the silencing of SbeIIa resulted in elevated expression of the Waxy genes, SSIII, limit dextrinase (Ld1), and isoamylase-1 (Iso1). The very high amylose results observed by Sestili et al. (2010b) in some of their transgenic lines may not have been due solely to reduction of SbeIIa expression since SbeIIa mutagenesis resulted in amylose levels increases more similar to those of SSIIa mutations (28% sbeIIa double mutant versus 23% wild-type) (Hazard et al. 2012). To date a detailed expression profile of starch synthesis genes in a SGP-1 null background has not been reported. RNA-Seq is an emerging method that employs next-generation sequencing technologies that allow for gene expression analysis at the transcript level. RNA-Seq offers single-nucleotide resolution that is highly reproducible (Marioni et al. 2008) and compared to other methods has a greater sequencing sensitivity, a large dynamic range, and the ability to distinguish between differing alleles or isoforms of an expressed gene. RNA-Seq is therefore an ideal method to use to determine the effect a null SGP-1 genotype has on expression of other starch synthesis genes.

Cereals with high amylose content are desirable because they have more resistant starch. Resistant starch is starch that resists break down in the intestines of humans and animals and thus acts more like dietary fiber while promoting microbial fermentation (reviewed in Nugent 2005). Products that have high resistant starch levels are viewed as healthy as they increase overall colon health and decrease sugar release during food digestion. Rats fed whole seed meal from SbeIIa RNAi silenced bread wheat with an amylose content of 80% showed significant improvements in bowel health indices and increases in short-chained fatty acids (SCFAs), the end products of microbial fermentation (Regina et al. 2006). Similarly, when null ssIIa barley was fed to humans there was significant improvement in several bowel health indices and increases in SCFAs (Bird et al. 2008). An extruded cereal made from the ssIIa null barley also resulted in a lower glycemic index and lower plasma insulin response when fed to humans (King et al. 2008). The Yamamori et al. (2000) SGP-1 single mutants were crossed and backcrossed to an Italian breeding line then interbred to produce a triple null line from which whole grain bread was prepared. The resultant bread with the addition of lactic acid had increased resistant starch and a decreased glycemic index, but did not impact insulin levels (Hallstrom et al. 2011). Recently a high amylose corn was shown to alter insulin sensitivity in overweight men making them less likely to have insulin resistance, the pathophysiologic feature of diabetes (Maki et al. 2012).

In addition to the positive impact of increased amylose upon glycemic index, higher amylose can result in enhanced wheat product quality. Pasta that is firmer when cooked is preferred as it resists overcooking and it is expected that high amylose should result in increased noodle firmness. In some embodiments, resistance to overcooking is positively correlated with pasta firmness. Current high amylose wheat based foods are prepared using standard amylose content wheat flour with the addition of high amylose maize starch (Thompson, 2000). To test the impact of high amylose upon durum quality Soh et al. (2006) varied durum flour amylose content by reconstituting durum flour with the addition of high amylose maize starch and wheat gluten. The increased amylose flours had weaker less extensible dough but resulted in firmer pasta. Pastas are a popular food item globally and are primarily made from durum semolina which is also utilized in a host of other culturally important foods.

In some embodiments, the present invention develops a high-amylose wheat line through the creation or identification of leaky mutations in SSII In some embodiments, the present invention teaches DNA or RNA sequencing to examine the effect of an SSII leaky genotype on the expression of other genes involved in starch synthesis. These lines are tested for their end product quality and potential health benefits.

The ratio of amylose to amylopectin can be changed by selecting for alternate forms of the Wx loci or other starch synthase loci. Bread wheat carrying the null allele at all three Wx loci (Nakamura, et al., 1995) and durum wheat (Lafiandra et al., 2010 and Vignaux et at., 2004) with null alleles at both Wx loci are nearly devoid of amylose. On the other hand, bread wheat lines null at the three SGP-1 loci had 37.5% amylose compared to 24.9% amylose for the wild type genotype, determined by differential scanning calorimetry (Morita et al., 2005). Durum wheat lines with null alleles for both SGP-1 loci had 43.6% amylose compared to 23.0% for the wild type genotype (Lafiandra et al., 2010). Genotypes with a null allele at only one of the Wx loci (partial waxy) show only small reductions in amylose content. For example, Martin et al. (2004) showed a 2.4% difference in amylose between the wild type and null alleles in a recombinant inbred population segregating for Wx-B1. Vignaux et at., (2004) showed partial waxy durum genotypes reduced amylose by 1% but that difference was not significant.

High Fiber and Amylose Flour and Resulting Products

In some embodiments, the SSII leaky wheat plants of the present invention have higher fiber content. In Europe and in North America, pasta is traditionally prepared using 100% durum flour (Fuad and Prabhasanker 2010). In fact, the properties inherent in durum wheat flour make it ideally suited for pasta production since it imparts excellent color due to relatively high yellow pigments levels and good mixing properties inherent in native glutenin proteins (Dexter and Matson 1979; Fuad and Prabhasanker 2010). Recently, there has been a movement towards the production of flour products with improved nutritional properties including increased fiber and amylose content, as well as flour products having increased protein content.

Flour with increased dietary fiber is associated with better gastrointestinal health, and lower risk of diabetes and heart disease. Flour with high amylose content is also desirable as it has a higher content of resistant starch that is not absorbed during digestion and thus produces health benefits similar to those of dietary fiber. The increased amylose content of flour also influences the gelatinization and pasting properties of starch. Peak viscosity, final viscosity, break down, set back and peak time measured by Rapid Visco Analyzer (RVA) all declined with increasing amylose content for durum wheat (Lafiandra et al., 2010). The altered starch properties translate into changes in end product properties such as increased firmness and resistance to overcooking.

Increasing the dietary fiber, amylose, and/or protein content of wheat flour products can be achieved by incorporating various protein or dietary fiber enriched fractions such as pea flour, cereal-soluble or insoluble fiber. These types of mixed enriched flour blends however can lead to consumer acceptance issues. For example, blending barley flour into durum wheat to increase dietary fiber in pasta led to a dark colored product (Casiraghi et al., 2013). Fortification of pasta with pea flour deteriorated dough handling characteristics, and increased pasta cooking losses and led to lower tolerance to overcooking (Nielsen et al., 1980). Modifying durum wheat to increase amylose, protein, and dietary fiber is preferable to durum flour additives since it would result in a pasta having the improved nutrition while also retaining many of the desirable properties of durum flour. The final product then would match the North American and European preference for 100% durum pasta. Durum wheat flour with increased amylose, protein, and dietary fiber used in the preparation of pasta would likely be preferable even to that of standard whole grain durum pasta which is much darker in appearance and has reduced cooked firmness leading to reduced consumer acceptability (Manthey and Schorno 2002).

In some embodiments, the SSII leaky wheat varieties of the present invention contain starch with higher amylose content. There has been recent interest in flours with higher amylose for food products. The main reason being that starch high in amylose has a higher fraction of resistant starch. Resistant starch is that fraction not absorbed in the small intestine during digestion (reviewed in Nugent 2005). Resistant starch is believed to provide health benefits similar to dietary fiber. Commercial high amylose food products have traditionally been developed using high amylose maize starch (Thompson, 2000). The development of high amylose bread wheat genotypes has made it possible to test the impact of high amylose wheat starch on end product quality. High amylose wheat flour produced harder textured dough and more viscous, and bread loaves that were smaller than normal flour (Morita et al., 2002). Substituting up to 50% high amylose wheat flour with the remainder being normal wheat flour gave bread quality that was not significantly different from the 100% normal wheat flour control (Hung et al., 2005). Durum and bread wheat flours varying in amylose content can be made by reconstituting them with high amylose maize starch (Soh et al., 2006). The high amylose durum wheat flours had dough that was weaker and less extensible. The pasta produced from these flours tended to be firmer with more cooking loss with increasing amylose content.

Even small, incremental increases in amylose may impact end product quality. Consumers prefer pasta that is firm and is tolerant to over cooking. Reduced amylose produces noodles that are softer in texture (Oda et al 1980; Miura and Tanii 1994; Zhao et al 1998). The impact of small increases in amylose content on durum product quality is not known. For example, attention has been devoted to Asian noodle quality from partial waxy flours. Partial waxy soft wheat cultivars, due to a mutation at one of the Wx loci, are preferred for udon noodles as they confer softer texture to the noodles (Oda et al 1980; Miura and Tanii 1994; Zhao et al 1998). Partial waxy genotype did not differ from wild type for white salted noodle firmness in a hard wheat recombinant inbred population (Martin et al., 2004). However, partial waxy genotype conferred greater loaf volume and bread was softer textured than that from the wild type.

### Identification and Creation of Mutant Starch Synthesis Genes in Wheat

Wheat with one or more mutant alleles of one or more starch synthesis genes can be created and identified. In some embodiments, such mutant alleles happen naturally during evolution. In some embodiments, such mutant alleles are created by artificial methods, such as mutagenesis (e.g., chemical mutagenesis, radiation mutagenesis, transposon mutagenesis, insertional mutagenesis, signature tagged mutagenesis, site-directed mutagenesis, and natural mutagenesis), antisense, knock-outs, and/or RNA interference. In some embodiments, the mutant alleles of the present invention are null alleles in which little to no gene function remains. In other embodiments, the mutant alleles of the present invention are leaky alleles, where partial gene function remains to create intermediate phenotypes.

Various types of mutagenesis can be used to produce and/or isolate variant nucleic acids that encode for protein molecules and/or to further modify/mutate the proteins of a starch synthesis gene. They include but are not limited to site-directed, random point mutagenesis, homologous recombination (DNA shuffling), mutagenesis using uracil containing templates, oligonucleotide-directed mutagenesis, phosphorothioate-modified DNA mutagenesis, mutagenesis using gapped duplex DNA or the like. Additional suitable methods include point mismatch repair, mutagenesis using repair-deficient host strains, restriction-selection and restriction-purification, deletion mutagenesis, mutagenesis by total gene synthesis, double-strand break repair, and the like. Mutagenesis, e.g., involving chimeric constructs, is also included in the present invention. In one embodiment, mutagenesis can be guided by known information of the naturally occurring molecule or altered or mutated naturally occurring molecule, e.g., sequence, sequence comparisons, physical properties, crystal structure or the like. For more information of mutagenesis in plants, such as agents, protocols, see Acquaah et al. (Principles of plant genetics and breeding, Wiley-Blackwell, 2007, ISBN 1405136464, 9781405136464, which is herein incorporated by reference in its entity). Methods of disrupting plant genes using RNA interference is described later in the specification.

Gene function can also be interrupted and/or altered by RNA interference (RNAi). RNAi is the process of sequence-specific, post-transcriptional gene silencing or transcriptional gene silencing in animals and plants, initiated by double-stranded RNA (dsRNA) that is homologous in sequence to the silenced gene. The preferred RNA effector molecules useful in this invention must be sufficiently distinct in sequence from any host polynucleotide sequences for which function is intended to be undisturbed after any of the methods of this invention are performed. Computer algorithms may be used to define the essential lack of homology between the RNA molecule polynucleotide sequence and host, essential, normal sequences.

The term “dsRNA” or “dsRNA molecule” or “double-strand RNA effector molecule” refers to an at least partially double-strand ribonucleic acid molecule containing a region of at least about 19 or more nucleotides that are in a double-strand conformation. The double-stranded RNA effector molecule may be a duplex double-stranded RNA formed from two separate RNA strands or it may be a single RNA strand with regions of self-complementarity capable of assuming an at least partially double-stranded hairpin conformation (i.e., a hairpin dsRNA or stem-loop dsRNA). In various embodiments, the dsRNA consists entirely of ribonucleotides or consists of a mixture of ribonucleotides and deoxynucleotides, such as RNA/DNA hybrids. The dsRNA may be a single molecule with regions of self-complementarity such that nucleotides in one segment of the molecule base pair with nucleotides in another segment of the molecule. In one aspect, the regions of self-complementarity are linked by a region of at least about 3-4 nucleotides, or about 5, 6, 7, 9 to 15 nucleotides or more, which lacks complementarity to another part of the molecule and thus remains single-stranded (i.e., the “loop region”). Such a molecule will assume a partially double-stranded stem-loop structure, optionally, with short single stranded 5′ and/or 3′ ends. In one aspect the regions of self-complementarity of the hairpin dsRNA or the double-stranded region of a duplex dsRNA will comprise an Effector Sequence and an Effector Complement (e.g., linked by a single-stranded loop region in a hairpin dsRNA). The Effector Sequence or Effector Strand is that strand of the double-stranded region or duplex which is incorporated in or associates with RISC. In one aspect the double-stranded RNA effector molecule will comprise an at least 19 contiguous nucleotide effector sequence, preferably 19 to 29, 19 to 27, or 19 to 21 nucleotides, which is a reverse complement to a starch synthesis gene.

In some embodiments, the dsRNA effector molecule of the invention is a “hairpin dsRNA”, a “dsRNA hairpin”, “short-hairpin RNA” or “shRNA”, i.e., an RNA molecule of less than approximately 400 to 500 nucleotides (nt), or less than 100 to 200 nt, in which at least one stretch of at least 15 to 100 nucleotides (e.g., 17 to 50 nt, 19 to 29 nt) is based paired with a complementary sequence located on the same RNA molecule (single RNA strand), and where said sequence and complementary sequence are separated by an unpaired region of at least about 4 to 7 nucleotides (or about 9 to about 15 nt, about 15 to about 100 nt, about 100 to about 1000 nt) which forms a single-stranded loop above the stem structure created by the two regions of base complementarity. The shRNA molecules comprise at least one stem-loop structure comprising a double-stranded stem region of about 17 to about 500 bp; about 17 to about 50 bp; about 40 to about 100 bp; about 18 to about 40 bp; or from about 19 to about 29 bp; homologous and complementary to a target sequence to be inhibited; and an unpaired loop region of at least about 4 to 7 nucleotides, or about 9 to about 15 nucleotides, about 15 to about 100 nt, about 250-500 bp, about 100 to about 1000 nt, which forms a single-stranded loop above the stem structure created by the two regions of base complementarity. It will be recognized, however, that it is not strictly necessary to include a “loop region” or “loop sequence” because an RNA molecule comprising a sequence followed immediately by its reverse complement will tend to assume a stem-loop conformation even when not separated by an irrelevant “stuffer” sequence.

The expression construct of the present invention comprising DNA sequence which can be transcribed into one or more double-stranded RNA effector molecules can be transformed into a wheat plant, wherein the transformed plant produces different starch compositions than the untransformed plant. The target sequence to be inhibited by the dsRNA effector molecule include, but are not limited to, coding region, 5′ UTR region, 3′ UTR region of fatty acids synthesis genes.

The effects of RNAi can be both systemic and heritable in plants. In plants, RNAi is thought to propagate by the transfer of siRNAs between cells through plasmodesmata. The heritability comes from methylation of promoters targeted by RNAi; the new methylation pattern is copied in each new generation of the cell. A broad general distinction between plants and animals lies in the targeting of endogenously produced miRNAs; in plants, miRNAs are usually perfectly or nearly perfectly complementary to their target genes and induce direct mRNA cleavage by RISC, while animals' miRNAs tend to be more divergent in sequence and induce translational repression. Detailed methods for RNAi in plants are described in David Allis et al (Epigenetics, CSHL Press, 2007, ISBN 0879697245, 9780879697242), Sohail et al (Gene silencing by RNA interference: technology and application, CRC Press, 2005, ISBN 0849321417, 9780849321412), Engelke et al. (RAN Interference, Academic Press, 2005, ISBN 0121827976, 9780121827977), and Doran et al. (RNA Interference: Methods for Plants and Animals, CABI, 2009, ISBN 1845934105, 9781845934101), which are all herein incorporated by reference in their entireties for all purposes.

### Gene Editing Technologies

In some embodiments, the wheat varieties of the present invention comprise one or more gene modifications produced via gene editing technologies. In some embodiments, the SGP mutant alleles of the present invention are created via gene editing technologies. In some embodiments, the wheat plants of the present disclosure comprise one or more mutant genes that have been modified using any genome editing tool, including, but not limited to tools such as: ZFNs, TALENS, CRISPR, and Mega nuclease technologies. In some embodiments, persons having skill in the art will recognize SGP mutant alleles of the present invention can be created with many other gene editing technologies.

In some embodiments, the gene editing tools of the present disclosure comprise proteins or polynucleotides which have been custom designed to target and cut at specific deoxyribonucleic acid (DNA) sequences. In some embodiments, gene editing proteins are capable of directly recognizing and binding to selected DNA sequences. In other embodiments, the gene editing tools of the present disclosure form complexes, wherein nuclease components rely on nucleic acid molecules for binding and recruiting the complex to the target DNA sequence.

In some embodiments, the single component gene editing tools comprise a binding domain capable of recognizing specific DNA sequences in the genome of the plant and a nuclease that cuts double-stranded DNA. The rationale for the development of gene editing technology for plant breeding is the creation of a tool that allows the introduction of site-specific mutations in the plant genome or the site-specific integration of genes.

Many methods are available for delivering genes into plant cells, e.g. transfection, electroporation, viral vectors and Agrobacterium mediated transfer. Genes can be expressed transiently from a plasmid vector. Once expressed, the genes generate the targeted mutation that will be stably inherited, even after the degradation of the plasmid containing the gene.

In some embodiments, the SGP mutant alleles of the present invention been modified through Zinc Finger Nucleases. Three variants of the ZFN technology are recognized in plant breeding (with applications ranging from producing single mutations or short deletions/insertions in the case of ZFN-1 and -2 techniques up to targeted introduction of new genes in the case of the ZFN-3 technique):

ZFN-1:

Genes encoding ZFNs are delivered to plant cells without a repair template. The ZFNs bind to the plant DNA and generate site specific double-strand breaks (DSBs). The natural DNA-repair process (which occurs through nonhomologous end-joining, NHEJ) leads to site specific mutations, in one or only a few base pairs, or to short deletions or insertions.

ZFN-2:

Genes encoding ZFNs are delivered to plant cells along with a repair template homologous to the targeted area, spanning a few kilo base pairs. The ZFNs bind to the plant DNA and generate site-specific DSBs. Natural gene repair mechanisms generate site-specific point mutations e.g. changes to one or a few base pairs through homologous recombination and the copying of the repair template.

ZFN-3:

Genes encoding ZFNs are delivered to plant cells along with a stretch of DNA which can be several kilo base pairs long and the ends of which are homologous to the DNA sequences flanking the cleavage site. As a result, the DNA stretch is inserted into the plant genome in a site specific manner.

In some embodiments, the SGP mutant alleles of the present disclosure are compatible with plants that have been modified through Transcription activator-like (TAL) effector nucleases (TALENs). TALENS are polypeptides with repeat polypeptide arms capable of recognizing and binding to specific nucleic acid regions. By engineering, the polypeptide arms to recognize selected target sequences, the TAL nucleases can be use to direct double stranded DNA breaks to specific genomic regions. These breaks can then be repaired via recombination to edit, delete, insert, or otherwise modify the DNA of a host organism. In some embodiments, TALENSs are used alone for gene editing (e.g., for the deletion or disruption of a gene). In other embodiments, TALs are used in conjunction with donor sequences and/or other recombination factor proteins that will assist in the Non-homologous end joining (MD) process to replace the targeted DNA region. For more information on the TAIL-mediated gene editing compositions and methods of the present disclosure, see U.S. Pat. Nos. 8,440,432; 8,440,432; U.S. Pat. No. 8,450,471; U.S. Pat. No. 8,586,526; U.S. Pat. No. 8,586,363; U.S. Pat. No. 8,592,645; U.S. Pat. Nos. 8,697,853; 8,704,041; 8,921,112; and 8,912,138, each of which is hereby incorporated in its entirety for all purposes.

In some embodiments, the SGP mutant alleles of the present disclosure are produced through Clustered Regularly Interspaced Short Palindromic Repeats (CRISPR) or CRISPR-associated (Cas) gene editing tools. CRISPR proteins were originally discovered as bacterial adaptive immunity systems which protected bacteria against viral and plasmid invasion.

There are at least three main CRISPR system types (Type I, II, and III) and at least 10 distinct subtypes (Makarova, K. S., et al., Nat Rev Microbiol. 2011 May 9; 9(6):467-477). Type I and III systems use Cas protein complexes and short guide polynucleotide sequences to target selected DNA regions. Type II systems rely on a single protein (e.g. Cas9) and the targeting guide polynucleotide, where a portion of the 5′ end of a guide sequence is complementary to a target nucleic acid. For more information on the CRISPR gene editing compositions and methods of the present disclosure, see U.S. Pat. Nos. 8,697,359; 8,889,418; 8,771,945; and 8,871,445, each of which is hereby incorporated in its entirety for all purposes. The present invention is also compatible with CRISPR-Cpfl sytems as described in (Zetsche, B. et al. Cell. 2015 163, 759-771).

In some embodiments, the SGP mutant alleles of the present disclosure have been modified through meganucleases. In some embodiments, meganucleases are engineered endonucleases capable of targeting selected DNA sequences and inducing DNA breaks. In some embodiments, new meganucleases targeting specific regions are developed through recombinant techniques which combine the DNA binding motifs from various other identified nucleases. In other embodiments, new meganucleases are created through semi-rational mutational analysis, which attempts to modify the structure of existing binding domains to obtain specificity for additional sequences. For more information on the use of meganucleases for genome editing, see Silva et al., 2011 Current Gene Therapy 11 pg. 11-27; and Stoddard et al., 2014 Mobile DNA 5 pg. 7, each of which is hereby incorporated in its entirety for all purposes.

In some embodiments, mutant starch synthesis genes in wheat can be identified by screening wheat populations based on one or more phenotypes.

In some embodiments, the phenotype is changes in flour swelling power.

In some embodiments, mutant starch synthesis genes in wheat can be identified by screening wheat populations based on PCR amplification and sequencing of one or more starch synthesis genes in wheat.

In some embodiments, the present invention teaches starch synthesis leaky alleles in bread wheat and/or durum wheat.

In some embodiments, mutant starch synthesis genes in wheat can be identified by TILLING®. Detailed description on methods and compositions on TILLING® can be found in U.S. Pat. No. 5,994,075, US 2004/0053236 A1, WO 2005/055704, and WO 2005/048692, each of which is hereby incorporated by reference for all purposes.

TILLING® (Targeting Induced Local Lesions in Genomes) is a method in molecular biology that allows directed identification of mutations in a specific gene. TILLING® was introduced in 2000, using the model plant Arabidopsis thaliana. TILLING® has since been used as a reverse genetics method in other organisms such as zebrafish, corn, wheat, rice, soybean, tomato and lettuce. The method combines a standard and efficient technique of mutagenesis with a chemical mutagen (e.g., Ethyl methanesulfonate (EMS)) with a sensitive DNA screening-technique that identifies single base mutations (also called point mutations) in a target gene. EcoTILLING is a method that uses TILLING® techniques to look for natural mutations in individuals, usually for population genetics analysis. See Comai, et al., 2003, Efficient discovery of DNA polymorphisms in natural populations by EcoTILLING. The Plant Journal 37, 778-786. Gilchrist et al. 2006. Use of EcoTILLING as an efficient SNP discovery tool to survey genetic variation in wild populations of Populus trichocarpa. Mol. Ecol. 15, 1367-1378. Mejlhede et al. 2006. EcoTILLING for the identification of allelic variation within the powdery mildew resistance genes mlo and Mla of barley. Plant Breeding 125, 461-467. Nieto et al. 2007, EcoTILLING for the identification of allelic variants of melon eIF4E, a factor that controls virus susceptibility. BMC Plant Biology 7, 34-42, each of which is incorporated by reference hereby for all purposes. DEcoTILLING is a modification of TILLING® and EcoTILLING which uses an inexpensive method to identify fragments (Garvin et al., 2007, DEco-TILLING: An inexpensive method for SNP discovery that reduces ascertainment bias. Molecular Ecology Notes 7, 735-746).

The invention also encompasses mutants of a starch synthesis gene. In some embodiments, the starch synthesis gene is selected from the group consisting of genes encoding GBSS, waxy proteins, SBE I and II, starch de-branching enzymes, and SSI, SSII, SSIII, and SSIV. In some embodiments, the starch synthesis gene is SSII. The mutant may contain alterations in the amino acid sequences of the constituent proteins. The term “mutant” with respect to a polypeptide refers to an amino acid sequence that is altered by one or more amino acids with respect to a reference sequence. The mutant can have “conservative” changes, or “nonconservative” changes, e.g., analogous minor variations can also include amino acid deletions or insertions, or both.

The mutations in a starch synthesis gene can be in the coding region or the non-coding region of the starch synthesis genes. The mutations can either lead to, or not lead to amino acid changes in the encoded starch synthesis gene. In some embodiments, the mutations can be missense, severe missense, silent, nonsense mutations. For example, the mutation can be nucleotide substitution, insertion, deletion, or genome re-arrangement, which in turn may lead to reading frame shift, amino acid substitution, insertion, deletion, and/or polypeptides truncation. As a result, the mutant starch synthesis gene encodes a starch synthesis polypeptide having modified activity on compared to a polypeptide encoded by a reference allele.

As used herein, a nonsense mutation is a point mutation, e.g., a single-nucleotide polymorphism (SNP), in a sequence of DNA that results in a premature stop codon, or a nonsense codon in the transcribed mRNA, and in a truncated, incomplete, and usually nonfunctional protein product. A missense mutation (a type of nonsynonymous mutation) is a point mutation in which a single nucleotide is changed, resulting in a codon that codes for a different amino acid (mutations that change an amino acid to a stop codon are considered nonsense mutations, rather than missense mutations). This can render the resulting protein nonfunctional. Silent mutations are DNA mutations that do not result in a change to the amino acid sequence of a protein. They may occur in a non-coding region (outside of a gene or within an intron), or they may occur within an exon in a manner that does not alter the final amino acid sequence. A severe missense mutation changes the amino acid, which lead to dramatic changes in conformation, charge status etc.

The mutations can be located at any portion of a starch synthesis gene, for example, at the 5′, the middle, or the 3′ of a starch synthesis gene, resulting in mutations in any portions of the encoded starch synthesis protein. In other embodiments, mutations of the present invention can be located on the promoter region of the starch synthesis gene leading to altered expression of the gene. For example, in some embodiments, the present invention teaches a wheat plant with reduced starch synthase activity due to a mutation in one or more of the promoters of the starch synthase genes. In some embodiments, the present invention may have different mutations in each of the starch synthase alleles. In other embodiments, the starch synthase alleles can have the same mutation.

For example, in some embodiments, the present invention teaches a wheat plant with one or more mutations in the starch synthase gene transcribed region, and one or more mutations in the starch synthase promoters.

In other embodiments, the present invention teaches a wheat plant with one or more mutations in the non-coding region of the starch synthase allele (e.g., 5′UTR, 3′UTR, introns, splice junctions).

Mutant starch synthesis protein of the present invention can have one or more modifications to the reference allele, or biologically active variant, or fragment thereof. Particularly suitable modifications include amino acid substitutions, insertions, deletions, or truncations. In some embodiments, at least one non-conservative amino acid substitution, insertion, or deletion in the protein is made to disrupt or modify the protein activity. The substitutions may be single, where only one amino acid in the molecule has been substituted, or they may be multiple, where two or more amino acids have been substituted in the same molecule. Insertional mutants are those with one or more amino acids inserted immediately adjacent to an amino acid at a particular position in the reference protein molecule, biologically active variant, or fragment thereof. The insertion can be one or more amino acids. The insertion can consist, e.g., of one or two conservative amino acids. Amino acids similar in charge and/or structure to the amino acids adjacent to the site of insertion are defined as conservative. Alternatively, mutant starch synthesis protein includes the insertion of an amino acid with a charge and/or structure that is substantially different from the amino acids adjacent to the site of insertion. In some other embodiments, the mutant starch synthesis protein is a truncated protein losing one or more domains compared to a reference protein.

In some examples, mutants can have at least 1, 2, 3, 4, 5, 10, 15, 20, 25, 30, 40, 50, or 100 amino acid changes. In some embodiments, at least one amino acid change is a conserved substitution. In some embodiments, at least one amino acid change is a non-conserved substitution. In some embodiments, the mutant protein has a modified enzymatic activity when compared to a wild type allele. In some embodiments, the mutant protein has a decreased or increased enzymatic activity when compared to a wild type allele. In some embodiments, the decreased or increased enzymatic activity when compared to a wild type allele leads to amylose content change in the wheat.

Conservative amino acid substitutions are those substitutions that, when made, least interfere with the properties of the original protein, that is, the structure and especially the function of the protein is conserved and not significantly changed by such substitutions. Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain. Further information about conservative substitutions can be found, for instance, in Ben Bassat et al. (J. Bacteriol., 169:751-757, 1987), O'Regan et al. (Gene, 77:237-251, 1989), Sahin-Toth et al. (Protein Sci., 3:240-247, 1994), Hochuli et al. (Bio/Technology, 6:1321-1325, 1988) and in widely used textbooks of genetics and molecular biology. The Blosum matrices are commonly used for determining the relatedness of polypeptide sequences. The Blosum matrices were created using a large database of trusted alignments (the BLOCKS database), in which pairwise sequence alignments related by less than some threshold percentage identity were counted (Henikoff et al., Proc. Natl. Acad. Sci. USA, 89:10915-10919, 1992). A threshold of 90% identity was used for the highly conserved target frequencies of the BLOSUM90 matrix. A threshold of 65% identity was used for the BLOSUM65 matrix. Scores of zero and above in the Blosum matrices are considered “conservative substitutions” at the percentage identity selected. The following table shows exemplary conservative amino acid substitutions.

TABLE 1
Amino Acid Substitution Chart

| | | Highly Conserved | Conserved |
|---|---|---|---|
| | Very Highly - | Substitutions | Substitutions |
| Original | Conserved | (from the | (from the |
| Residue | Substitutions | Blosum90 Matrix) | Blosum65 Matrix) |
| Ala | Ser | Gly, Ser, Thr | Cys, Gly, Ser, Thr, Val |
| Arg | Lys | Gln, His, Lys | Asn, Gln, Glu, His, Lys |
| Asn | Gln; His | Asp, Gln, His, Lys, | Arg, Asp, Gln, Glu, His, |
| | | Ser, Thr | Lys, Ser, Thr |
| Asp | Glu | Asn, Glu | Asn, Gln, Glu, Ser |
| Cys | Ser | None | Ala |
| Gln | Asn | Arg, Asn, Glu, His, | Arg, Asn, Asp, Glu, His, |
| | | Lys, Met | Lys, Met, Ser |
| Glu | Asp | Asp, Gln, Lys | Arg, Asn, Asp, Gln, His, |
| | | | Lys, Ser |
| Gly | Pro | Ala | Ala, Ser |
| His | Asn; Gln | Arg, Asn, Gln, Tyr | Arg, Asn, Gln, Glu, Tyr |
| Ile | Leu; Val | Leu, Met, Val | Leu, Met, Phe, Val |
| Leu | Ile; Val | Ile, Met, Phe, Val | Ile, Met, Phe, Val |
| Lys | Arg; Gln; Glu | Arg, Asn, Gln, Glu | Arg, Asn, Gln, Glu, Ser, |
| Met | Leu; Ile | Gln, Ile, Leu, Val | Gln, Ile, Leu, Phe, Val |
| Phe | Met; Leu; Tyr | Leu, Trp, Tyr | Ile, Leu, Met, Trp, Tyr |
| Ser | Thr | Ala, Asn, Thr | Ala, Asn, Asp, Gln, Glu, |
| | | | Gly, Lys, Thr |
| Thr | Ser | Ala, Asn, Ser | Ala, Asn, Ser, Val |
| Trp | Tyr | Phe, Tyr | Phe, Tyr |
| Tyr | Trp; Phe | His, Phe, Trp | His, Phe, Trp |
| Val | Ile; Leu | Ile, Leu, Met | Ala, Ile, Leu, Met, Thr |

In some embodiments, the mutant durum wheat comprises mutations associated with a starch synthesis gene of the same genome that can be traced back to one common ancestor, such as the “A” type genome of durum wheat or the “B” type genome of durum wheat. For example, a mutant durum wheat having a mutated SSII-A or a mutated SSII-B is included. In some embodiments, one or both alleles of the starch synthesis gene within a given type of genome are mutated.

In some embodiments, the mutant durum wheat comprise mutations associated with the same starch synthesis gene of different genomes that can be traced back to two common ancestors, such as the “A” type genome and the “B” type genome of durum wheat. For example, a mutant durum wheat having a mutated SSII-A and a mutated SSII-B is included. In some embodiments, one or both alleles of the starch synthesis gene within the two types of genomes are mutated.

In some embodiments, the mutant bread wheat comprises mutations associated with a starch synthesis gene of the same genome that can be traced back to one common ancestor, such as the “A” type genome of bread wheat or the “B” type genome of bread wheat, or the “D” type genome of bread wheat. For example, a mutant bread wheat having a mutated SSII-A, a mutated SSII-B, or a mutated SSII-D is included. In some embodiments, one or more alleles of the starch synthesis gene within a given type of genome are mutated.

In some embodiments, the mutant bread wheat comprise mutations associated with the same starch synthesis gene of different genomes that can be traced back to two or three common ancestors, such as the “A” type genome, the “B” type genome, and the “D” type genome of bread wheat. For example, a mutant bread wheat having a mutated SSII-A, a mutated SSII-B, and a mutated SSII-D is included. In some embodiments, one or more alleles of the starch synthesis gene within the two types of genomes are mutated.

In some embodiments, the present invention teaches one or more of the mutant SSII alleles are leaky. In some embodiments, two of the SSII alleles are null, and one is leaky. In some embodiments, one of the SSII alleles is null and two are leaky. In yet another embodiment, all SSII alleles are leaky

In some embodiments, one SSII alleles is null, and one is leaky. In some embodiments, both SSII alleles are leaky.

### Wheat Grain Milling

Useful examples of processes for preparing a milled wheat material will be understood by the skilled artisan to include steps of milling and separating, along with related process steps, as are presently known or developed in the future. According to exemplary such methods, mill-quality wheat grain can be processed by milling steps that may include one or more of bran removal such as pearling, pearling to remove germ, other forms of abrading, grinding, sizing, tempering, etc.

In traditional milling methods the wheat is gathered, cleaned and tempered and then ground in order to form refined wheat flour and millfeed (coarse fraction). The first step in this process, cleaning the wheat, includes removing various impurities such as weed seeds, stones, mud-balls, and metal parts, from the wheat. The cleaning of the wheat typically begins by using a separator in which vibrating screens are used to removes bits of wood and straw and anything else that is too big or too small to be wheat. Next, an aspirator is used, which relies on air currents to remove dust and lighter impurities. Then a destoner is used to separate the heavy contaminants such as stones that are the same size as wheat. Air is drawn though a bed of wheat on an oscillating deck that is covered with a woven wire cloth. A separation is made based on the difference in specific gravity and surface friction. The wheat then passes through a series of disc or cylinder separators which separate based on shape and length, rejecting contaminates that are longer, shorter, rounder or more angular than a typical wheat kernel. Finally, a scourer removes a portion of the bran layer, crease dirt, and other smaller impurities.

Once the wheat is cleaned, it is tempered in order to be conditioned for milling. Moisture is added to the wheat kernel in order to toughen the bran layers while mellowing the endosperm. Thus, the parts of the wheat kernel are easier to separate and tend to separate more easily. Prior to milling, the tempered wheat is stored for a period of eight to twenty-four hours to allow the moisture to fully absorb into the wheat kernel. The milling process is basically a gradual reduction of the wheat kernels. The grinding process produces a mixture of granulites containing bran and endosperm, which is sized by using sifters and purifiers. The coarse particles of endosperm are then ground into flour by a series of rollermills. When milling wheat, the wheat kernel typically yields 75% refined wheat flour (fine fraction) and 25% coarse fraction. The coarse fraction is that portion of the wheat kernel which is not processed into refined wheat flour, typically including the bran, germ, and small amounts of residual endosperm.

The recovered coarse fraction can then be ground through a grinder, preferably a gap mill, to form an ultrafine-milled coarse fraction having a particle size distribution less than or equal to about 150 μm. The gap mill tip speed normally operates between 115 m/s to 130 m/s. Additionally, after sifting, any ground coarse fraction having a particle size greater than 150 μm can be returned to the process for further milling.

After the fine fraction (refined wheat flour) and the coarse fraction (coarse product) have been separated, the coarse fraction is divided and each portion of the coarse fraction is sent through a separate grinder for further downstream process.

Traditional wheat milling can yield up to three separate products. The first product is refined wheat flour, comprised of the fine fraction, which contains the endosperm of the wheat kernel. The second product is the ultrafine-milled coarse fraction, and the third product is an ultrafine-milled whole-grain wheat flour.

Persons having skill in the art will recognize that the wheat varieties of the present disclosure are compatible with any wheat milling process. The description of an exemplary traditional wheat milling process is provided for illustrative purposes, but should in no way be construed as limiting the milling steps of the present disclosure.

### Methods of Modifying Wheat Phenotypes

The present invention further provides methods of modifying/altering/improving wheat phenotypes. As used herein, the term “modifying” or “altering” refers to any change of phenotypes when compared to a reference variety, e.g., changes associated with starch properties, and or seed weight properties. The term “improving” refers to any change that makes the wheat better in one or more qualities for industrial or nutritional applications. Such improvement includes, but is not limited to, improved quality as meal, improved quality as raw material to produce a wide range of end products.

In some embodiments, the modified/altered/improved phenotypes are related to starch. Starch is the most common carbohydrate in the human diet and is contained in many foods. The major sources of starch intake worldwide are the cereals (rice, wheat, and maize) and the root vegetables (potatoes and cassava). Widely used prepared foods containing starch are bread, pancakes, cereals, noodles, pasta, porridge and tortilla. The starch industry extracts and refines starches from seeds, roots and tubers, by wet grinding, washing, sieving and drying. Today, the main commercial refined starches are corn, tapioca, wheat and potato starch.

Starch can be hydrolyzed into simpler carbohydrates by acids, various enzymes, or a combination of the two. The resulting fragments are known as dextrins. The extent of conversion is typically quantified by dextrose equivalent (DE), which is roughly the fraction of the glycosidic bonds in starch that have been broken.

Some starch sugars are by far the most common starch based food ingredient and are used as sweetener in many drinks and foods. They include, but are not limited to, maltodextrin, various glucose syrup, dextrose, high fructose syrup, and sugar alcohols.

A modified starch is a starch that has been chemically modified to allow the starch to function properly under conditions frequently encountered during processing or storage, such as high heat, high shear, low pH, freeze/thaw and cooling. Typical modified starches for technical applications are cationic starches, hydroxyethyl starch and carboxymethylated starches.

As an additive for food processing, food starches are typically used as thickeners and stabilizers in foods such as puddings, custards, soups, sauces, gravies, pie fillings, and salad dressings, and to make noodles and pastas.

In the pharmaceutical industry, starch is also used as an excipient, as tablet disintegrant or as binder.

Starch can also be used for industrial applications, such as papermaking, corrugated board adhesives, clothing starch, construction industry, manufacture of various adhesives or glues for book-binding, wallpaper adhesives, paper sack production, tube winding, gummed paper, envelope adhesives, school glues and bottle labeling. Starch derivatives, such as yellow dextrins, can be modified by addition of some chemicals to form a hard glue for paper work; some of those forms use borax or soda ash, which are mixed with the starch solution at 50-70° C. to create a very good adhesive.

Starch is also used to make some packing peanuts, and some drop ceiling tiles. Textile chemicals from starch are used to reduce breaking of yarns during weaving; the warp yarns are sized. Starch is mainly used to size cotton based yarns. Modified starch is also used as textile printing thickener. In the printing industry, food grade starch is used in the manufacture of anti-set-off spray powder used to separate printed sheets of paper to avoid wet ink being set off. Starch is used to produce various bioplastics, synthetic polymers that are biodegradable. An example is polylactic acid. For body powder, powdered starch is used as a substitute for talcum powder, and similarly in other health and beauty products. In oil exploration, starch is used to adjust the viscosity of drilling fluid, which is used to lubricate the drill head and suspend the grinding residue in petroleum extraction. Glucose from starch can be further fermented to biofuel corn ethanol using the so called wet milling process. Today most bioethanol production plants use the dry milling process to ferment corn or other feedstock directly to ethanol. Hydrogen production can use starch as the raw material, using enzymes.

Resistant starch is starch that escapes digestion in the small intestine of healthy individuals. High amylose starch from corn has a higher gelatinization temperature than other types of starch and retains its resistant starch content through baking, mild extrusion and other food processing techniques. It is used as an insoluble dietary fiber in processed foods such as bread, pasta, cookies, crackers, pretzels and other low moisture foods. It is also utilized as a dietary supplement for its health benefits. Published studies have shown that Type 2 resistant corn helps to improve insulin sensitivity, increases satiety and improves markers of colonic function. It has been suggested that resistant starch contributes to the health benefits of intact whole grains.

Resistant starch can be produced from the wheat plants of the present invention. The resistant starch may have one or more the following features:

1) Fiber fortification: the resistant starch is a good or excellent fiber source. The United States Department of Agriculture and the health organizations of other foreign countries set the standards for what constitutes a good or excellent source of dietary fiber.

2) Low caloric contribution: the starch may contain less than about 10 kcal/g, 5 kcal/g, 1 kcal/g, or 0.5 kcal/g, which results in about 90% calorie reduction compared to typical starch.

3) Low glycemic/insulin response

4) Good flour replacement, because it is (1) easy to be incorporated into formulations with minimum or no formulation changes necessary, (2) natural fit for wheat-based products, and (3) potential to reduce retrogradation and staling. Staling is a chemical and physical process in bread and other foods that reduces their palatability.

5) Low water binding capacity: the starch possesses lower water holding capacity than most other fiber sources, including other types of resistant starches. It reduces water in the formula, ideal for targeting crispiness, and improves shelf life regarding micro-activity and retrogradation.

6) Process tolerant: the starch is stable against energy intensive procedures, such as extrusion, pressure cooking, etc.

7) Sensory attributes: such as smooth, non-gritty texture, white, “invisible” fiber source, and neutral in flavor.

Therefore, flour or starch produced from the wheat of the present invention can be used to replace bread wheat flour or starch, to produce wheat bread, muffins, buns, pasta, noodles, tortillas, pizza dough, breakfast cereals, cookies, waffles, bagels, biscuits, snack foods, brownies, pretzels, rolls, cakes, and crackers, wherein the food products may have one or more desired features.

In some embodiments, the leaky allele wheat of the present invention has one or more distinguishing phenotypes when compared to a wild-type wheat of the same species, which includes, but are not limited to, modified gelatinization temperature (e.g., a modified amylopectin gelatinization peaks, and/or a modified enthalpy), modified amylose content, modified resistant amylose content, modified starch quality, modified flour swelling power, modified protein content (e.g., higher protein content), modified kernel weight, modified kernel hardness, and modified semolina yield. In some embodiments, the mutant wheat with leaky SSII (i.e., SGP-1) alleles of the present invention also has increased seed weight or seed size when compared against a corresponding plant with an SSII-null (SGP-null) allele variant. In particular embodiments, the leaky allele wheat of the present invention provides both (i) increased seed weight or size and (ii) one or more of the foregoing distinguishing phenotypes.

In some embodiments, the methods relate to modifying gelatinization temperature of wheat, such as modifying amylopectin gelatinization peaks and/or modifying enthalpy. Modified gelatinization temperature results in altered temperatures required for cooking starch based products. Different degrees of starch gelatinization impact the level of resistant starch. For example, endothermic peaks I and II of FIG. 5 are due to the resolved gelatinization and the melting of the fat/amylose complex, respectively. In some embodiments, the amylopectin gelatinization profile of the wheat of the present invention is changed compared to reference wheat, such as a wild-type wheat. In some embodiments, the amylopectin gelatinization temperature of the wheat of the present invention is significantly lower than that of a wild-type control. For example, the amylopectin gelatinization temperature of the wheat of the present invention is about 1° C., 2° C., 3° C., 4° C., 5° C., 6° C., 7° C., 8° C., 9° C., 10° C., 15° C., 20° C., 25° C. or more lower than that of a wild-type control based on peak height on a Differential Scanning calorimetry (DSC) thermogram, under the same heating rate. Starches having reduced gelatinization are associated with those starches having increased amylose and reduced glycemic index. They are also associated with having firmer starch based gels upon retrogradation as in cooked and cooled pasta.

In some embodiments, the change in enthalpy of the wheat starch of the present invention is dramatically smaller compared to that of a wild type control. For example, as measured by DSC thermogram, the heat flow transfer in the wheat starch of the present invention is only about ½, ⅓, or ¼ of that of a wild-type control.

Starch gelatinization is a process that breaks down the intermolecular bonds of starch molecules in the presence of water and heat, allowing the hydrogen bonding sites (the hydroxyl hydrogen and oxygen) to engage more water. This irreversibly dissolves the starch granule. Penetration of water increases randomness in the general starch granule structure and decreases the number and size of crystalline regions. Crystalline regions do not allow water entry. Heat causes such regions to become diffuse, so that the chains begin to separate into an amorphous form. Under the microscope in polarized light starch loses its birefringence and its extinction cross. This process is used in cooking to make roux sauce. The gelatinization temperature of starch depends upon plant type and the amount of water present, pH, types and concentration of salt, sugar, fat and protein in the recipe, as well as derivatisation technology used. The gelatinization temperature depends on the degree of cross-linking of the amylopectin, and can be modified by genetic manipulation of starch synthase genes.

In one embodiment, the methods relate to modifying amylose content of wheat, such as resistant amylose content. Flour with increased resistant amylose content can be used to make firmer pasta with greater resistance to overcooking as well as reduced glycemic index and increased dietary fiber and resistant starch. In some embodiments, the amylose content and/or the resistant amylose content of the wheat of the present invention and the products produced from said wheat, is modified (e.g., increased) by about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 79%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, 300%, 400%, 500%, 600%, 700%, 800%, 900%, 1000% or more compared to that of a wild-type wheat, or a check wheat variety with all wild type SSII alleles.

In some embodiments, the amylose content and/or resistant amylose content of the wheat of the present invention and products produced from said wheat is about 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 79%, 79%, 80%, 81%, 82%, 83%, 84%, 85%, 86%, 87%, 88%, 89%, 90%, 91%, 92%, 93%, 94%, 95%, 96%, 97%, 98%, or 99%. Thus, wheat with all wild type SSII alleles analyzed by exemplary methods described herein, was found to have an amylose content of about 30% as compared to a high amylose wheat of the invention which was found to have significantly more than 30% amylose content including, e.g., about 42.4% amylose.

In some embodiments, the amylose content and/or resistant amylose content of the wheat of the present invention and products produced from said wheat is greater than about 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 79%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some embodiments, the methods relate to modifying starch quality of wheat.

In some embodiments, the methods relate to modifying flour swelling power (FSP) of wheat. Reduced FSP should result in reduced weight of the noodles and increased firmness. In some embodiments, based on the methods described in Mukasa et al. (Comparison of flour swelling power and water-soluble protein content between self-pollinating and cross-pollinating buckwheat, Fagopyrum 22:45-50 (2005), the FSP of the wheat of the present invention is modified (e.g., decreased) by about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 79%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, 300%, 400%, 500%, 600%, 700%, 800%, 900%, 1000% or more compared to that of a wild-type wheat, or a check wheat variety with all wild type SSII alleles.

In some embodiments, the FSP of the wheat of the present invention and products produced from said wheat is 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 (g/g).

In some embodiments, the FSP of the wheat of the present invention and products produced from said wheat is lower than 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 (g/g).

In some embodiments, the methods relate to modifying amylopectin content of wheat. In some embodiments, amylose and amylopectin are interrelated so decreasing amylopectin is the same benefit as increased amylose. In some embodiments, decreasing amylose (and/or increasing amylopectin) is associated with increased FSP, reduced retrogradation and softer baked products and noodles. In some embodiments, increasing amylopectin is also associated with reduced rate of staling. In some embodiments, the amylopectin content of the wheat of the present invention is modified (e.g., decreased) by about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 79%, 79%, 80%, 81%, 82%, 83%, 84%, 85%, 86%, 87%, 88%, 89%, 90%, 91%, 92%, 93%, 94%, 95%, 96%, 97%, 98%, 99%, 100% or more compared to that of a wild-type wheat, or a check wheat variety with all wild type SSII alleles.

In some embodiments, the amylopectin content of the wheat of the present invention and products produced from said wheat is about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 79%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some embodiments, the amylopectin content of the wheat of the present invention and products produced from said wheat is lower than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 79%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some embodiments, the methods relate to modifying protein content of wheat. In some embodiments, the protein content of the wheat of the present invention and the products produced from said wheat, is modified (e.g., increased) by about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 79%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, 300%, 400%, 500%, 600%, 700%, 800%, 900%, 1000% or more compared to that of a wild-type wheat, or a check wheat variety with all wild type SSII alleles.

In some embodiments, the protein content of the wheat of the present invention and products produced from said wheat is about 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 79%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some embodiments, the protein content of the wheat of the present invention and products produced from said wheat is greater than about 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 79%, 79%, 80%, 81%, 82%, 83%, 84%, 85%, 86%, 87%, 88%, 89%, 90%, 91%, 92%, 93%, 94%, 95%, 96%, 97%, 98%, or 99%.

Increased protein content means greater nutritional value (reduced glycemic index) as well as greater functionality. In terms of pasta quality, increased protein content would be associated with reduced FSP and increased pasta firmness.

In some embodiments, the methods relate to modifying dietary fiber content in the wheat grain. In some embodiments, the dietary fiber content in the wheat grain of the present invention and the products produced from said wheat, is modified (e.g., increased) by about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 79%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, 300%, 400%, 500%, 600%, 700%, 800%, 900%, 1000% or more compared to that of a wild-type wheat, or a check wheat variety with all wild type SSII alleles.

In some embodiments, the dietary fiber content of the wheat of the present invention and products produced from said wheat is about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 79%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some embodiments, the dietary fiber content of the wheat of the present invention and products produced from said wheat is greater than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 79%, 79%, 80%, 81%, 82%, 83%, 84%, 85%, 86%, 87%, 88%, 89%, 90%, 91%, 92%, 93%, 94%, 95%, 96%, 97%, 98%, or 99%.

Advantages of consuming products made from grain with increased dietary fiber include, but are not limited to the production of healthful compounds during the fermentation of the fiber, and increased bulk, softened stool, and shortened transit time through the intestinal tract.

In some embodiments, the methods relate to modifying fat content in the wheat grain. In some embodiments, the fat content in the wheat grain of the present invention is modified (e.g., increased) by about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 79%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, 300%, 400%, 500%, 600%, 700%, 800%, 900%, 1000% or more compared to that of a wild-type wheat, or a check wheat variety with all wild type SSII alleles.

In some embodiments, the fat content of the wheat of the present invention and products produced from said wheat is about 0%, 0.1%, 0.2%, 0.3%, 0.4%, 0.5%, 0.6%, 0.7%, 0.8%, 0.9%, 1%, 1.2%, 1.3%, 1.4%, 1.5%, 1.6%, 1.7%, 1.8%, 1.9%, 2%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40%.

In some embodiments, the fat content of the wheat of the present invention and products produced from said wheat is greater than about 0%, 0.1%, 0.2%, 0.3%, 0.4%, 0.5%, 0.6%, 0.7%, 0.8%, 0.9%, 1%, 1.2%, 1.3%, 1.4%, 1.5%, 1.6%, 1.7%, 1.8%, 1.9%, 2%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40%.

In some embodiments, the methods relate to modifying resistant starch content in the wheat grain. In some embodiments, the resistant starch content in the wheat grain of the present invention is modified (e.g., increased) by about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 79%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, 300%, 400%, 500%, 600%, 700%, 800%, 900%, 1000% or more compared to that of a wild-type wheat, or a check wheat variety with all wild type SSII alleles.

In some embodiments, the resistant starch content of the wheat of the present invention and products produced from said wheat is about 0.1%, 0.2%, 0.3%, 0.4%, 0.5%, 0.6%, 0.7%, 0.8%, 0.9%, 1%, 1.2%, 1.3%, 1.4%, 1.5%, 1.6%, 1.7%, 1.8%, 1.9%, 2%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 79%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some embodiments, the resistant starch content of the wheat of the present invention and products produced from said wheat is greater than about 0.1%, 0.2%, 0.3%, 0.4%, 0.5%, 0.6%, 0.7%, 0.8%, 0.9%, 1%, 1.2%, 1.3%, 1.4%, 1.5%, 1.6%, 1.7%, 1.8%, 1.9%, 2%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 79%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some embodiments, the methods relate to modifying ash content in the wheat grain. In some embodiments, the ash content in the wheat grain of the present invention is modified (e.g., increased) by about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 79%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, 300%, 400%, 500%, 600%, 700%, 800%, 900%, 1000% or more compared to that of a wild-type wheat, or a check wheat variety with all wild type SSII alleles.

In some embodiments, the ash content of the wheat of the present invention and products produced from said wheat is about 0.1%, 0.2%, 0.3%, 0.4%, 0.5%, 0.6%, 0.7%, 0.8%, 0.9%, 1%, 1.2%, 1.3%, 1.4%, 1.5%, 1.6%, 1.7%, 1.8%, 1.9%, 2%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40%.

In some embodiments, the ash content of the wheat of the present invention and products produced from said wheat is greater than about 0.1%, 0.2%, 0.3%, 0.4%, 0.5%, 0.6%, 0.7%, 0.8%, 0.9%, 1%, 1.2%, 1.3%, 1.4%, 1.5%, 1.6%, 1.7%, 1.8%, 1.9%, 2%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.1%, 5.2%, 5.3%, 5.4%, 5.5%, 5.6%, 5.7%, 5.8%, 5.9%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40%.

In some embodiments, the methods relate to modifying kernel weight of wheat. In some embodiments, the kernel weight of the wheat of the present invention is modified (e.g., decreased) by about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 79%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, 300%, 400%, 500%, 600%, 700%, 800%, 900%, 1000% or more compared to that of a wild-type wheat, or a wheat with all wild type SSII alleles.

In some embodiments, the kernel weight of the wheat of the present invention is modified (e.g., increased) by about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 79%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, 300%, 400%, 500%, 600%, 700%, 800%, 900%, 1000% or more compared to that of a full SSII null mutant wheat plant (e.g., SSII-A and SSII-B null durum, or SSII-A, SSII-B, and SSII-D null bread wheat).

For example, in some embodiments, the SGP1 leaky wheat of the present invention may have increased kernel weight compared to an SGP-null segregant or other appropriate check line. Increased seed weight without impacting seed number leads to increased yield and generally increased starch content.

In some embodiments, the kernel weight of the wheat grain of the present invention is about 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, or 50 mg.

In some embodiments, the kernel weight of the wheat grain of the present invention is greater than about 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, or 50 mg.

Thus, SSII triple null allele wheat analyzed by exemplary methods described herein, was found to have a kernel weight of about 25 mg as compared to a high amylose SSII leaky and two SSII null allele wheat product of the invention which was found to have significantly more than 25 mg kernel weight, including, e.g., about 28 mg.

In some embodiments, the methods relate to modifying kernel hardness of wheat. In some embodiments, the kernel hardness of the wheat of the present invention is modified (e.g., increased or decreased) for about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 79%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, 300%, 400%, 500%, 600%, 700%, 800%, 900%, 1000% or more compared to that of a wild-type durum wheat, or a wheat with all wild type SSII alleles.

In some embodiments, the kernel hardness of the wheat grain of the present invention is about 50, 51, 52, 53, 54, 55, 56, 57, 58, 59, 60, 61, 62, 63, 64, 65, 66, 67, 68, 69, 70, 71, 72, 73, 74, 75, 76, 77, 79, 79, 80, 81, 82, 83, 84, 85, 86, 87, 88, 89, 90, 91, 92, 93, 94, 95, 96, 97, 98, 99, or 100.

In some embodiments, the kernel hardness is measured by the methods described in Osborne, B. G., Z. Kotwal, et al. (1997). “Application of the Single-Kernel Characterization System to Wheat Receiving Testing and Quality Prediction.” Cereal Chemistry Journal 74(4): 467-470, which is incorporated herein by reference in its entirety. Kernel hardness impacts milling properties of wheat. For example, in some embodiments, the SGP1 leaky wheat of the present invention may have reduced kernel hardness compared to wild-type. In some embodiments, reducing kernel hardness is associated with increased break flour yield and reduced flour ash and starch damage. In some embodiments, milling energy would also be reduced. In some embodiments, increased kernel hardness is associated with increased milling energy, increased starch damage after milling and increased flour particle size.

In some embodiments, mutations in one or more copies of one or more SSII leaky alleles are integrated together to create mutant plants with double, triple, quadruple etc. mutations. In some embodiments, SSII leaky alleles located in the A genome and/or the B genome of a durum wheat, or one or more of the A, B, and D genomes of hexaploid bread wheat. Such mutants can be created using transgenic technology, by classic breeding methods, or using both techniques.

In some embodiments, mutations described herein can be integrated into wheat species by classic breeding methods, with or without the help of marker-facilitated gene transfer methods, such as T. aestivum, T. aethiopicum, T. araraticum, T. boeoticum, T. carthlicum, T. compactum, T. dicoccoides, T. dicoccum, T. ispahanicum, T. karamyschevii, T. macha, T. militinae, T. monococcum, T. polonicum, T. spelta, T. sphaerococcum, T. timopheevii, T. turanicum, T. turgidum, T. urartu, T. vavilovii, and T. zhukovskyi.

In one embodiment, mutants of a starch synthesis gene having mutations in evolutionarily conserved regions or sites can be used to produce wheat plants with improved or altered phenotypes. In one embodiment, mutants due to nonsense mutations (premature stop codon), can be used to produce wheat plants with improved or altered phenotypes. In one embodiment, mutants not in evolutionarily conserved regions or sites, can also be used to produce wheat plants with improved or altered phenotypes.

In some other embodiments, SSII leaky alleles can be integrated with other mutant genes and/or transgenes. Based on the teaching of the present invention, one skilled in the art will be able to pick preferred target genes and decide when disruption or overexpression is needed to achieve certain goals, such as mutants and/or transgenes which can generally improve plant health, plant biomass, plant resistance to biotic and abiotic factors, plant yields, wherein the final preferred fatty acid production is increased. Such mutants and/or transgenes include, but are not limited to pathogen resistance genes and genes controlling plant traits related to seed yield.

Additional genes encoding polypeptides that can ultimately affect starch synthesis can be modulated to achieve a desired starch production. Such polypeptides include but are not limited to, soluble starch synthases (SSS), Granule bound starch synthases (GBSS), such as GBSSI, GBSSII, ADP-glucose pyrophosphorylases (AGPases), starch branching enzymes (a.k.a., SBE, such as SBE I and SBE II), starch de-branching enzymes (a.k.a., SDBE), and starch synthases I, II, III, and IV.

The modulation can be achieved through breeding methods which integrate desired alleles into a single wheat plant. The desired alleles can be either naturally occurring ones or created through mutagenesis. In some embodiments, the desired alleles result in increased activity of the encoded polypeptide in a plant cell when compared to a reference allele. For example, the desired alleles can lead to increased polypeptide concentration in a plant cell, and/or polypeptides having increased enzymatic activity and/or increased stability compared to a reference allele. In some embodiments, the desired alleles result in decreased activity of the encoded polypeptide in a plant cell when compared to a reference allele. For example, the desired alleles can be either null-mutation, or encode polypeptides having decreased activity, decreased stability, and/or being wrongfully targeted in a plant cell compared to a reference allele.

The modulation can also be achieved through introducing a transgene into a wheat variety, wherein the transgene can either overexpress a gene of interest or negatively regulate a gene of interest.

In some embodiments, an SSII leaky allele of the present invention is combined with one or more alleles which result in increased amylose synthesis are introduced to a wheat plant, such as alleles resulting in modified soluble starch synthase activity or modified granule-bound starch synthase activity. In some embodiments, said alleles locate in the A genome and/or the B genome of a durum wheat, or one or more of the A, B, and D genomes of hexaploid bread wheat.

In some embodiments, an SSII leaky allele of the present invention is combined with one or more alleles which result in decreased amylose synthesis are introduced to a wheat plant, such as alleles resulting in modified soluble starch synthase activity or modified granule-bound starch synthase activity. In some embodiments, said alleles locate in the A genome and/or the B genome of a durum wheat, or one or more of the A, B, and D genomes of hexaploid bread wheat.

In some embodiments, an SSII leaky allele of the present invention is combined with one or more alleles which result in increased amylopectin synthesis are introduced to a wheat plant, such as alleles resulting in modified SSI, and/or SSIII activity, modified starch branching enzyme (e.g., SBEI, SBEIIa and SBEIIb) activity, or modified starch debranching enzyme activity. In some embodiments, said alleles locate in the A genome and/or the B genome of a durum wheat, or one or more of the A, B, and D genomes of hexaploid bread wheat.

In some embodiments, an SSII leaky allele of the present invention is combined with one or more alleles which result in decreased amylopectin synthesis are introduced to a wheat plant, such as alleles resulting in modified SSI, and/or SSIII activity, modified starch branching enzyme (e.g., SBEI, SBEIIa and SBEIIb) activity, or modified starch debranching enzyme activity. In some embodiments, said alleles locate in the A genome and/or the B genome of a durum wheat, or one or more of the A, B, and D genomes of hexaploid bread wheat.

Methods of disrupting and/or altering a target gene have been known to one skilled in the art. These methods include, but are not limited to, mutagenesis (e.g., chemical mutagenesis, radiation mutagenesis, transposon mutagenesis, insertional mutagenesis, signature tagged mutagenesis, site-directed mutagenesis, and natural mutagenesis), knock-outs/knock-ins, antisense, RNA interference, and gene editing, and other tools described in this application.

The present invention also provides methods of breeding wheat species producing altered levels of fatty acids in the seed oil and/or meal. In one embodiment, such methods comprise

i) making a cross between the SSII leaky allele wheat of the present invention to a second wheat species to make F1 plants;

ii) backcrossing said F1 plants to said second wheat species;

iii) repeating backcrossing step until said leaky allele mutations are integrated into the genome of said second wheat species. Optionally, such method can be facilitated by molecular markers.

The present invention provides methods of breeding species close to wheat, wherein said species produces altered/improved starch. In one embodiment, such methods comprise

i) making a cross between the SSII leaky allele wheat of the present invention to a species close to wheat to make F1 plants;

ii) backcrossing said F1 plants to said species that is close to wheat;

iii) repeating backcrossing step until said leaky allele mutations are integrated into the genome of said species that is close to wheat. Special techniques (e.g., somatic hybridization) may be necessary in order to successfully transfer a gene from wheat to another species and/or genus. Optionally, such method can be facilitated by molecular markers.

The present invention also provides unique starch compositions.

In some embodiments, provided are wheat starch compositions having modified starch quality compared to the starch compositions derived from a reference wheat species, such as a wild-type wheat species. In particular embodiments, the wheat starch compositions having modified starch compositions are made from grain comprising one or more SSII leaky allele. The wheat starch composition can be made, for example, from grain comprising no SSII wild-type alleles, at least one SSII leaky alleles, and optionally one or more SSII null alleles in accordance with the invention.

In some embodiments, provided are wheat starch compositions having modified gelatinization temperature compared to the starch compositions derived from a reference wheat species, such as a wild-type wheat species. In some embodiments, the wheat starch compositions of the present invention has modified amylopectin gelatinization peaks and/or modified enthalpy. In some embodiments, the amylopectin gelatinization temperature of the wheat starch of the present invention is about 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C. or higher or lower than that of a wild-type control based on peak height on a Differential Scanning Calorimetry (DSC) thermogram, under the same heat rate, or based on a Rapid Visco Analyzer test. In some embodiments, increased amylose would result in increased gelatinization temperature, the temperature of amylopectin gelatinization.

Using the methods of the present application, wheat grains with beneficial features can be produced. Such features include but are not limited to, modified dietary fiber content, modified protein content, modified fat content, modified resistant starch content, modified ash content; and modified amylose content. In some embodiments, wheat grains with one or more of the following features compared to the grain made from a control wheat plant are created: (1) increased dietary fiber content; (2) increased protein content; (3) increased fat content; (4) increased resistance starch content; (5) increased ash content; and (6) increased amylose content. The wheat grain with said beneficial features can be used to produce food products, such as noodle and pasta.

### Plant Transformation

The present invention provides transgenic wheat plants with one or more SSII leaky alleles. The modification can be either disruption or overexpression.

Binary vector suitable for wheat transformation includes, but are not limited to the vectors described by Zhang et al., 2000 (An efficient wheat transformation procedure: transformed calli with long-term morphogenic potential for plant regeneration, Plant Cell Reports (2000) 19: 241-250), Cheng et al., 1997 (Genetic Transformation of Wheat Mediated by Agrobacterium tumefaciens, Plant Physiol. (1997) 115: 971-980), Abdul et al., (Genetic Transformation of Wheat (Triticum aestivum L): A Review, TGG 2010, Vol. 1, No. 2, pp 1-7), Pastori et al., 2000 (Age dependent transformation frequency in elite wheat varieties, J. Exp. Bot. (2001) 52 (357): 857-863), Jones 2005 (Wheat transformation: current technology and applications to grain development and composition, Journal of Cereal Science Volume 41, Issue 2, March 2005, Pages 137-147), Galovic et al., 2010 (MATURE EMBRYO-DERIVED WHEAT TRANSFORMATION WITH MAJOR STRESS MODULATED ANTIOXIDANT TARGET GENE, Arch. Biol. Sci., Belgrade, 62 (3), 539-546), or similar ones. Wheat plants are transformed by using any method described in the above references.

To construct the transformation vector, the region between the left and right T-DNA borders of a backbone vector is replaced with an expression cassette consisting of a constitutively expressed selection marker gene (e.g., the NptII kanamycin resistance gene) followed by one or more of the expression elements operably linked to a reporter gene (e.g., GUS or GFP). The final constructs are transferred to Agrobacterium for transformation into wheat plants by any of the methods described in Zhang et al., 2000, Cheng et al., 1997, Abdul et al., Pastori et al., 2000, Jones 2005, Galovic et al., 2010, U.S. Pat. No. 7,197,9964 or similar ones to generate polynucleotide::GFP fusions in transgenic plants.

For efficient plant transformation, a selection method must be employed such that whole plants are regenerated from a single transformed cell and every cell of the transformed plant carries the DNA of interest. These methods can employ positive selection, whereby a foreign gene is supplied to a plant cell that allows it to utilize a substrate present in the medium that it otherwise could not use, such as mannose or xylose (for example, refer U.S. Pat. No. 5,767,378; U.S. Pat. No. 5,994,629). More typically, however, negative selection is used because it is more efficient, utilizing selective agents such as herbicides or antibiotics that either kill or inhibit the growth of nontransformed plant cells and reducing the possibility of chimeras. Resistance genes that are effective against negative selective agents are provided on the introduced foreign DNA used for the plant transformation. For example, one of the most popular selective agents used is the antibiotic kanamycin, together with the resistance gene neomycin phosphotransferase (nptII), which confers resistance to kanamycin and related antibiotics (see, for example, Messing & Vierra, Gene 19: 259-268 (1982); Bevan et al., Nature 304:184-187 (1983)). However, many different antibiotics and antibiotic resistance genes can be used for transformation purposes (refer U.S. Pat. No. 5,034,322, U.S. Pat. No. 6,174,724 and U.S. Pat. No. 6,255,560). In addition, several herbicides and herbicide resistance genes have been used for transformation purposes, including the bar gene, which confers resistance to the herbicide phosphinothricin (White et al., Nucl Acids Res 18: 1062 (1990), Spencer et al., Theor Appl Genet 79: 625-631(1990), U.S. Pat. No. 4,795,855, U.S. Pat. No. 5,378,824 and U.S. Pat. No. 6,107,549). In addition, the dhfr gene, which confers resistance to the anticancer agent methotrexate, has been used for selection (Bourouis et al., EMBO J. 2(7): 1099-1104 (1983).

The expression control elements used to regulate the expression of a given protein can either be the expression control element that is normally found associated with the coding sequence (homologous expression element) or can be a heterologous expression control element. A variety of homologous and heterologous expression control elements are known in the art and can readily be used to make expression units for use in the present invention. Transcription initiation regions, for example, can include any of the various opine initiation regions, such as octopine, mannopine, nopaline and the like that are found in the Ti plasmids of Agrobacterium tumefaciens. Alternatively, plant viral promoters can also be used, such as the cauliflower mosaic virus 19S and 35S promoters (CaMV 19S and CaMV 35S promoters, respectively) to control gene expression in a plant (U.S. Pat. Nos. 5,352,605; 5,530,196 and 5,858,742 for example). Enhancer sequences derived from the CaMV can also be utilized (U.S. Pat. Nos. 5,164,316; 5,196,525; 5,322,938; 5,530,196; 5,352,605; 5,359,142; and 5,858,742 for example). Lastly, plant promoters such as prolifera promoter, fruit specific promoters, Ap3 promoter, heat shock promoters, seed specific promoters, etc. can also be used.

Methods of producing transgenic plants are well known to those of ordinary skill in the art. Transgenic plants can now be produced by a variety of different transformation methods including, but not limited to, electroporation; microinjection; microprojectile bombardment, also known as particle acceleration or biolistic bombardment; viral-mediated transformation; and Agrobacterium-mediated transformation. See, for example, U.S. Pat. Nos. 5,405,765; 5,472,869; 5,538,877; 5,538,880; 5,550,318; 5,641,664; 5,736,369 and 5,736369; International Patent Application Publication Nos. WO2002/038779 and WO/2009/117555; Lu et al., (Plant Cell Reports, 2008, 27:273-278); Watson et al., Recombinant DNA, Scientific American Books (1992); Hinchee et al., Bio/Tech. 6:915-922 (1988); McCabe et al., Bio/Tech. 6:923-926 (1988); Toriyama et al., Bio/Tech. 6:1072-1074 (1988); Fromm et al., Bio/Tech. 8:833-839 (1990); Mullins et al., Bio/Tech. 8:833-839 (1990); Hiei et al., Plant Molecular Biology 35:205-218 (1997); Ishida et al., Nature Biotechnology 14:745-750 (1996); Zhang et al., Molecular Biotechnology 8:223-231 (1997); Ku et al., Nature Biotechnology 17:76-80 (1999); and, Raineri et al., Bio/Tech. 8:33-38 (1990)), each of which is expressly incorporated herein by reference in their entirety.

### Breeding Methods

Classic breeding methods can be included in the present invention to introduce one or more SSII leaky allele mutations of the present invention into other plant varieties, or other close-related species that are compatible to be crossed with the transgenic plant of the present invention.

Open-Pollinated Populations.

The improvement of open-pollinated populations of such crops as rye, many maizes and sugar beets, herbage grasses, legumes such as alfalfa and clover, and tropical tree crops such as cacao, coconuts, oil palm and some rubber, depends essentially upon changing gene-frequencies towards fixation of favorable alleles while maintaining a high (but far from maximal) degree of heterozygosity. Uniformity in such populations is impossible and trueness-to-type in an open-pollinated variety is a statistical feature of the population as a whole, not a characteristic of individual plants. Thus, the heterogeneity of open-pollinated populations contrasts with the homogeneity (or virtually so) of inbred lines, clones and hybrids.

Population improvement methods fall naturally into two groups, those based on purely phenotypic selection, normally called mass selection, and those based on selection with progeny testing. Interpopulation improvement utilizes the concept of open breeding populations; allowing genes to flow from one population to another. Plants in one population (cultivar, strain, ecotype, or any germplasm source) are crossed either naturally (e.g., by wind) or by hand or by bees (commonly Apis mellifera L. or Megachile rotundata F.) with plants from other populations. Selection is applied to improve one (or sometimes both) population(s) by isolating plants with desirable traits from both sources.

There are basically two primary methods of open-pollinated population improvement. First, there is the situation in which a population is changed en masse by a chosen selection procedure. The outcome is an improved population that is indefinitely propagable by random-mating within itself in isolation. Second, the synthetic variety attains the same end result as population improvement but is not itself propagable as such; it has to be reconstructed from parental lines or clones. These plant breeding procedures for improving open-pollinated populations are well known to those skilled in the art and comprehensive reviews of breeding procedures routinely used for improving cross-pollinated plants are provided in numerous texts and articles, including: Allard, Principles of Plant Breeding, John Wiley & Sons, Inc. (1960); Simmonds, Principles of Crop Improvement, Longman Group Limited (1979); Hallauer and Miranda, Quantitative Genetics in Maize Breeding, Iowa State University Press (1981); and, Jensen, Plant Breeding Methodology, John Wiley & Sons, Inc. (1988).

Mass Selection.

In mass selection, desirable individual plants are chosen, harvested, and the seed composited without progeny testing to produce the following generation. Since selection is based on the maternal parent only, and there is no control over pollination, mass selection amounts to a form of random mating with selection. As stated herein, the purpose of mass selection is to increase the proportion of superior genotypes in the population.

Synthetics.

A synthetic variety is produced by crossing inter se a number of genotypes selected for good combining ability in all possible hybrid combinations, with subsequent maintenance of the variety by open pollination. Whether parents are (more or less inbred) seed-propagated lines, as in some sugar beet and beans (Vicia) or clones, as in herbage grasses, clovers and alfalfa, makes no difference in principle. Parents are selected on general combining ability, sometimes by test crosses or topcrosses, more generally by polycrosses. Parental seed lines may be deliberately inbred (e.g. by selfing or sib crossing). However, even if the parents are not deliberately inbred, selection within lines during line maintenance will ensure that some inbreeding occurs. Clonal parents will, of course, remain unchanged and highly heterozygous.

Whether a synthetic can go straight from the parental seed production plot to the farmer or must first undergo one or two cycles of multiplication depends on seed production and the scale of demand for seed. In practice, grasses and clovers are generally multiplied once or twice and are thus considerably removed from the original synthetic.

While mass selection is sometimes used, progeny testing is generally preferred for polycrosses, because of their operational simplicity and obvious relevance to the objective, namely exploitation of general combining ability in a synthetic.

The number of parental lines or clones that enter a synthetic varies widely. In practice, numbers of parental lines range from 10 to several hundred, with 100-200 being the average. Broad based synthetics formed from 100 or more clones would be expected to be more stable during seed multiplication than narrow based synthetics.

Pedigreed Varieties.

A pedigreed variety is a superior genotype developed from selection of individual plants out of a segregating population followed by propagation and seed increase of self pollinated offspring and careful testing of the genotype over several generations. This is an open pollinated method that works well with naturally self pollinating species. This method can be used in combination with mass selection in variety development. Variations in pedigree and mass selection in combination are the most common methods for generating varieties in self pollinated crops.

Hybrids.

A hybrid is an individual plant resulting from a cross between parents of differing genotypes. Commercial hybrids are now used extensively in many crops, including corn (maize), sorghum, sugarbeet, sunflower and broccoli. Hybrids can be formed in a number of different ways, including by crossing two parents directly (single cross hybrids), by crossing a single cross hybrid with another parent (three-way or triple cross hybrids), or by crossing two different hybrids (four-way or double cross hybrids).

Strictly speaking, most individuals in an out breeding (i.e., open-pollinated) population are hybrids, but the term is usually reserved for cases in which the parents are individuals whose genomes are sufficiently distinct for them to be recognized as different species or subspecies. Hybrids may be fertile or sterile depending on qualitative and/or quantitative differences in the genomes of the two parents. Heterosis, or hybrid vigor, is usually associated with increased heterozygosity that results in increased vigor of growth, survival, and fertility of hybrids as compared with the parental lines that were used to form the hybrid. Maximum heterosis is usually achieved by crossing two genetically different, highly inbred lines.

The production of hybrids is a well-developed industry, involving the isolated production of both the parental lines and the hybrids which result from crossing those lines. For a detailed discussion of the hybrid production process, see, e.g., Wright, Commercial Hybrid Seed Production 8:161-176, In Hybridization of Crop Plants.

### Differential Scanning Calorimetry

Differential scanning calorimetry or DSC is a thermoanalytical technique in which the difference in the amount of heat required to increase the temperature of a sample and reference is measured as a function of temperature. Both the sample and reference are maintained at nearly the same temperature throughout the experiment. Generally, the temperature program for a DSC analysis is designed such that the sample holder temperature increases linearly as a function of time. The reference sample should have a well-defined heat capacity over the range of temperatures to be scanned. DSC can be used to analyze Thermal Phase Change, Thermal Glass Transition Temperature (Tg), Crystalline Melt Temperature, Endothermic Effects, Exothermic Effects, Thermal Stability, Thermal Formulation Stability, Oxidative Stability Studies, Transition Phenomena, Solid State Structure, and Diverse Range of Materials. The DSC thermogram can be used to determine Tg Glass Transition Temperature, Tm Melting point, A Hm Energy Absorbed (joules/gram), Tc Crystallization Point, and AHc Energy Released (joules/gram).

DSC can be used to measure the gelatinization of starch. See Application Brief, TA No. 6, SII Nanotechnology Inc., “Measurements of gelatinization of starch by DSC”, 1980; Donovan 1979 Phase transitions of the starch-water system. Bio-polymers, 18, 263-275.; Donovan, J. W., & Mapes, C. J. (1980). Multiple phase transitions of starches and Nageli arnylodextrins. Starch, 32, 190-193. Eliasson, A.-C. (1980). Effect of water content on the gelatinization of wheat starch. Starch, 32, 270-272. Lund, D. B. (1984). Influence of time, temperature, moisture, ingredients and processing conditions on starch gelatinization. CRC Critical Reviews in Food Science and Nutrition, 20 (4), 249-257. Shogren, R. L. (1992). Effect of moisture content on the melting and subsequent physical aging of cornstarch. Carbohydrate Polymers, 19, 83-90. Stevens, D. J., & Elton, G. A. H. (1971). Thermal properties of the starch water system. Staerke, 23, 8-11. Wootton, M., & Bamunuarachchi, A. (1980). Application of differential scanning calorimetry to starch gelatinization. Starch, 32, 126-129. Zobel, H. F., & Gelation, X. (1984). Gelation. Gelatinization of starch and mechanical properties of starch pastes. In R. Whistler, J. N. Bemiller & E. F. Paschall, Starch: chemistry and technology (pp. 285-309). Orlando, Fla.: Academic Press. Gelatinization profile is dependent on heating rates and water contents. Unless specifically defined, the comparison in DSC between the starch from the wheat of the present application and the starch from a wild-type reference, or other reference wheat is under the same heating rates and/or same water content. In some embodiments, the present application provides starch compositions having modified gelatinization temperature as measured by DSC.

DSC can be used to measure the glass transition temperature of starch. See Chinachoti, P. (1996). Characterization of thermomechanical properties in starch and cereal products. Journal of Thermal Analysis, 47, 195-213. Maurice et al. 1985 Polysaccharide-water interactions—thermal behavior of rice starch. In D. Simatos & S. L. Multon, Properties of water in foods

(pp. 211-227). Dordrecht: Nilhoff; Slade, L., & Levine, H. (1987). Recent advances in starch retrogradation. In S. S. Stivala, V. Crescenzi & I. C. M. Dea, Industrial polysaccharides (pp. 387-430). New York: Gordon and Breach. Stepto, R. F. T., & Tomka, I. (1987). Chimia, 41 (3), 76-81. Zeleznak, K. L., & Hoseney, R. C. (1997). The glass transition in starch. Cereal Chemistry, 64 (2), 121-124. In some embodiments, the present application provides starch compositions having modified glass transition temperature as measured by DSC.

DSC can be used to measure the crystallization of starch. See Biliaderis, C. G., Page, C. M., Slade, L., & Sirett, R. R. (1985). Thermal behavior of amylose-lipid complexes. Carbohydrate Polymers, 5, 367-389. Ring, S. G., Colinna, P., I'Anson, K. J., Kalichevsky, M. T., Miles, M. J., Morris, V. J., & Orford, P. D. (1987). Carbohydrate Research, 162, 277-293. In some embodiments, the present application provides starch compositions having modified crystallization temperature as measured by DSC.

DSC can also be used to calculate the heat capacity change between the starch made from the wheat plants of the present application and a wild-type wheat plant. The heat capacity of a sample is calculated from the shift in the baseline at the starting transient:

Cp=dH/dt×dt/dT

wherein dH/dt is the shift in the baseline of the thermogram and dt/dT is the inverse of the heating rate. The unit of the heat flow is mW or mcal/second, and the unit of heating rate can be ° C./min or ° C./second. In some embodiments, at the heating rate of 10° C./min, the heat capacity of the starch made from the wheat of the present application as measured by DSC is modified (e.g., increased or decreased) for about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 79%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, 300%, 400%, 500%, 600%, 700%, 800%, 900%, 1000% or more compared to that of the starch made from a wild-type wheat, or a wheat with two wild type SSII alleles and only one SSII leaky allele.

This invention is further illustrated by the following examples which should not be construed as limiting. The contents of all references, patents and published patent applications cited throughout this application, as well as the Figures and the Sequence Listing, are incorporated herein by reference.

### EXAMPLES

### Example 1

### Identification of SSII Leaky Mutants and Creation of New SGP Hexaploid Wheat Mutant Varieties

The following example demonstrates the creation and identification of SSII leaky allele mutant hexaploid bread wheat plants with improved properties, including both elevated amylose (relative to null alleles) and near normal seed weight, by screening and selecting for SSII mutant alleles with reduced SSII protein abundance in purified starch.

### PCR Screening for EMS Mutations in SSII-A and SSII-B and SSII-D.

Leaf tissue from Alpowa RJ mutant plant populations suspected of having leaky mutant alleles was collected at Feekes growth stage 1.3, stored at −80° C. and DNA was extracted following Riede and Anderson (1996). Coding regions of SSII-A and SSII-B and SSII-D were amplified from duplicate DNA samples using previously described primers and PCR conditions (Chibbar et al. 2005, Shimbata et al. 2005, Sestili et al. 2010a). Amplicons were sequenced and resultant DNA sequences were analyzed for single nucleotide polymorphisms using Seqman Pro in the Lasergene 10.1 Suite (DNASTAR, Madison, Wis.). Table 2 provides a non-exclusive list of the SSII mutants identified in this example.

TABLE 2
Starch synthase II (SGP-1) mutations in EMS derived Alpowa RJ hexaploid wheat population.

| RJ | PCR | DNA | DNA | Original | New | AA | Original | New | | SDS |
|---|---|---|---|---|---|---|---|---|---|---|
| ID^{1,2} | Fragment | Mutation | location² | Codon | Codon | # | AA | AA | Location | PAGE³ |
| 302 | A4 | C to T | 2120 | GCC | GTC | 707 | A | V | exon | null |
| 493 | A4A | C to T | 758 | CCT | CTT | 253 | P | L | exon | null |
| 253 | 7A-F4a | G to A | 1289 | TGC | TAC | 430 | C | Y | exon | null |
| 42 | A\grave{ }4A | C to T | 956 | CCC | CTC | 319 | P | L | exon | partial |
| 435 | B2 | G to A | 1943 | GGC | GAC | 648 | G | D | exon | null |
| 597 | B2 | G to A | 1787 | TGC | TAC | 596 | C | Y | exon | null |
| 269 | B2 | G to A | 1685 | GGC | GAC | 562 | G | D | exon | null |
| 521 | B2 | G to A | 1685 | GGC | GAC | 562 | G | D | exon | null |
| 63 | B2 | G to A | 1685 | GGC | GAC | 562 | G | D | exon | null |
| 102 | B1 | C to T | 751 | CCG | TCG | 251 | P | S | exon | partial |
| 416 | B1 | G to A | 957 | TGG | TGA | 319 | W | stop | exon | null |
| 514 | B2 | C to T | 1816 | CTG | TTG | 606 | L | L | exon | partial |
| 183 | D4 | G to A | 2441 | na | na | na | na | na | splice jct | null |
| 597 | D4 | G to A | 2159 | GGC | GAC | 720 | G | D | exon | null |
| 647 | D4 | C to T | 1978 | CAG | TAG | 660 | Q | Stop | exon | null |
| 624 | D4 | G to A | 1966 | GAG | AAG | 656 | E | K | exon | partial |
| 414 | D4 | C to T | 2354 | GCC | GTC | 785 | A | V | exon | partial |
| 122 | 7D-F3 | C to T | 1262 | GCT | GTT | 421 | A | V | exon | partial |

¹RJ Lines marked by underlining were chosen for crossing to create triple nulls where three unique combinations of SGP1 mutants are targeted to ensure best possible SGP-1 null yield and seed size. Note that RJ 597 contains mutations in both SGP-D1 and SGP-B1.
²Indicates the location of the nucleic acid mutation based on the SSII protein coding gene sequence of the corresponding genome, the count beginning from the first nucleotide of the start codon.
³Deleterious mutations were confirmed via SDS PAGE. Null indicates the lack of the corresponding protein while partial denotes reduced level.
⁴ Splice junction mutation location based on start of published genomic region as described in SEQ ID No. 34.

### Starch Extraction

In order to measure SGP-1 protein abundance, starch was first extracted by grinding seeds in a Braun coffee mill (Proctor Gamble, Cincinnati, Ohio) for 10 s and then placed in a 2 ml microcentrifuge tube along with two 6.5 mm zirconia balls and agitated for 30 s in a Mini-beadbeater-96. The zirconia balls were removed from the microcentrifuge tubes and 1.0 ml of 0.1 M NaCl was added to the whole grain flour which was then left to steep for 30 min. at room temperature. After 30 min., a dough ball was made by mixing the wet flour using a plastic Kontes Pellet Pestle (Kimble Chase, Vineland, N.J.) and the gluten ball was removed from the samples after pressing out the starch. The liquid starch suspension was then transferred to a new pre-weighed 2.0 ml tube and 0.5 ml ddH₂O was added to the remnant starch pellet in the first tube. The first tube was vortexed, left to settle for 1 min. and the liquid starch suspension transferred to the second tube. The starch suspension containing tubes were centrifuged at 5,000 g and the liquid was aspirated off. Next, 0.5 ml of SDS extraction buffer (55 mM Tris-Cl pH 6.8, 2.3% SDS, 5% BME, 10% glycerol) was added, the samples were vortexed till suspended, and then centrifuged at 5,000 g. The SDS buffer was aspirated off and the SDS buffer extraction was repeated once more. Then, 0.5 ml of 80% CsCl was added to the starch pellets, samples were vortexed till suspended, and centrifuged at 7,500 g. The CsCl was aspirated off and the starch pellets were washed twice with 0.5 ml ddH₂O, and once in acetone with centrifugation speeds of 10,000 g. After supernatant aspiration the starch pellets were left to dry overnight in a fume hood.

### SDS-PAGE of Starch Granule Proteins

In order to measure SGP-1 protein abundance, 7.5 μl of SDS loading buffer (SDS extraction buffer plus bromophenol blue) was added per mg of starch. Samples were heated for 15 min. at 70° C., centrifuged for 1 min at 10,000 g, and then 40 μl of sample was loaded on a 10% (w/v) acrylamide gel prepared using a 30% acrylamide/0.8% piperazine diacrylamide w/v stock solution. The gel had a standard 4% w/v acrylamide stacking gel prepared using a 30% acrylamide/0.8% piperazine diacrylamide w/v stock solution. Gels were run (25 mA/gel for 45 min. and then 35 mA/gel for three hrs), silver stained following standard procedures, and photographed on a light box with a digital camera.

Flour swelling power (FSP) was also determined for the Alpowa population described in this example. Varieties exhibiting a starch synthase mutation, with reduced SGP-1 protein abundance, and reduced flour swelling power were selected to be used in the breeding methods described in this application. Varieties with these criteria were hypothesized to comprise leaky alleles which retain small amounts of SGP-1 starch synthase activity (either A, B, or D). Selected parents from this screen are depicted below in Table 3.

TABLE 3
Non-Exclusive list of SSII Leaky Alleles for SGP-1.

1. RJ-414 (partial D, =A785V, FSP = 8.51)
2. RJ-624 (partial D, =E656K, FSP = 7.27)
3. RJ-514 (partial A, =L606L, FSP = 8.36)
4. RJ-102 (partial B, =P251S, FSP = 7.47)
5. RJ-122 (partial D, =A421V, FSP = 6.53)
6. RJ-42 (partial A, =P319L, FSP = 7.46)
7. Control Alpowa FSP = 8.8

The varieties of Table 3 were crossed with crossed to RJ-597/302 SSII triple null #72 variety in order to develop plants with 2 null mutant alleles and at least one leaky allele. The resulting F₂ populations (6,000+ plants) were grown in the field and genotyped at the 3-4 leaf stage from field grown plants using the markers developed for the leaky plants from Table 3, and SSII null mutations of RJ-597/302. Three key allelic groups were harvested: (i) homozygous for all three of the SSII null mutations, (ii) homozygous for two of the SSII null mutations with one leaky allele, and (iii) a leaky allele and homozygous for two SSII wild-type alleles. Because excess reduced seed size F₂ seeds were planted, more SSII triple mutants were obtained than would be expected by chance (Table 4). 1023 individual F₂ plants for each population were sampled in the field in Bozeman and genotyped at 3-4 leaf stage from field grown plants. The expected frequency of each homozygous class was 1/64 (1.56%) or -16 homozygotes for each group. All homozygotes were harvested for each of the three homozygous classes shown in the table. SSII nulls are overrepresented due to phenotyping of F₂ seeds for reduced seed size (Table 4).

TABLE 4
Leaky allele F₂ population segregation data.

| Leaky | SSII | SSII Double | SSII double |
|---|---|---|---|
| Parent | null | Null + 1 leaky | WT and 1 leaky |
| 42 | 25 | 16 | 22 |
| 102 | 49 | 11 | 16 |
| 122 | 6 | 12 | 16 |
| 414 | 33 | 20 | 26 |
| 514 | 27 | 14 | 13 |
| 624 | 35 | 9 | 14 |

### Example 2

### Amylose Content of SSII Leaky Varieties

Starch was prepared from each of the three homozygous classes for each of the six populations described in Table 4.

### Starch Extraction

Seeds from each genotype and line were ground in a Braun coffee mill (Proctor Gamble, Cincinnati, Ohio) for 10 s and then placed in a 2 ml microcentrifuge tube along with two 6.5 mm yttria stabilized zirconia ceramic balls (Stanford Materials, Irvine, Calif.) which were then agitated for 30 s in a Mini-beadbeater-96 (Biospec Products, Bartlesville, Okla.) with an oscillation distance of 3.2 cm and a shaking speed of 36 oscillations/s. The zirconia balls were removed from the tubes and 1.0 ml of 0.1 M NaCl was added to the whole grain flour which was then left to steep for 30 min. at room temperature. After 30 min., a dough ball was made by mixing the wet flour using a plastic Kontes Pellet Pestle (Kimble Chase, Vineland, N.J.) and the gluten ball was removed from the samples after pressing out the starch. The liquid starch suspension was then transferred to a new pre-weighed 2.0 ml tube and 0.5 ml ddH₂O was added to the remnant starch pellet in the first tube. The first tube was vortexed, left to settle for 1 min. and the liquid starch suspension transferred to the second tube. The starch suspension containing tubes were centrifuged at 5,000 g and the liquid was aspirated off. To the starch pellets, 0.5 ml of SDS extraction buffer (55 mM Tris-Cl pH 6.8, 2.3% SDS, 5% BME, 10% glycerol) was added, the samples were vortexed until suspended, and then centrifuged at 5,000 g. The SDS buffer was aspirated off and the SDS buffer extraction was repeated once more. Next, 0.5 ml of 80% CsCl was added to the starch pellets, samples were vortexed until suspended, and then centrifuged at 7,500 g. The CsCl was aspirated off and the starch pellets were washed twice with 0.5 ml ddH₂O, and once in acetone with centrifugation speeds of 10,000 g. After aspirating off the acetone the pellets were left to dry overnight in a fume hood.

Amylose content was determined using differential scanning calorimeter (DSC) with a Pyris 7 Diamond DSC (Perkin Elmer, Norwalk Conn., USA) following the methods described in Hansen et al. (2010). Amylose results were averaged for each group and p values were calculated comparing WT and Leaky amylose values (Table 5).

TABLE 5
The effect of the six leaky alleles on starch amylose content.

| | Amylose (%) | | | |
|---|---|---|---|---|
| Leaky | SSII | SSII double | SSII double | Leaky vs Wt |
| Parent | null | null + 1 leaky | WT and 1 leaky | P value |
| 42 | 53.4 | 28.5 | 30.0 | 0.27 |
| 102 | 56.7 | 30.1 | 26.6 | 0.16 |
| 122 | 47.2 | 42.4 | 30.6 | 0.00 |
| 414 | 49.5 | 30.8 | 28.2 | 0.11 |
| 514 | 51.2 | 31.4 | 31.0 | 0.45 |
| 624 | 51.2 | 40.3 | 29.7 | 0.01 |
| Average | 51.5 | 33.9 | 29.4 | 0.2 |
| 122, 624 | 49.2 | 41.4 | 30.2 | 0.00 |

The results suggest that four of the six initially identified “leaky” alleles were likely too leaky since they accumulated wild-type levels of amylose content (leaky parents 42, 102, 414, and 514 in Table 5). The two remaining “leaky” alleles (122 and 624) exhibited increased amylose content over the wild type, but below the SSII null group.

### Example 3

### Seed Size of SSII Leaky Varieties

In order to determine the effect of the SSII leaky alleles on seed weight, homozygous seed from ‘624’, ‘122’, and ‘414’ wheat lines were individually weighed. Seed size was determined on 200 seeds per line. Average weights in milligrams and percent differences comparisons to WT segregants (SSII double WT+1 leaky) are summarized in Table 6.

TABLE 6
The effect of the six leaky alleles upon F₂ seed size

| | Leaky Line | | | | | |
|---|---|---|---|---|---|---|
| | 624 | | 122 | | 414 | |
| Starch | Seed | Seed | Seed | Seed | Seed | Seed |
| Synthase II | weight | size vs | weight | size vs | weight | size vs |
| Genotype | (mg) | WT (%) | (mg) | WT (%) | (mg) | WT (%) |
| SSII null | 25 a | −21.9 | 22 a | −42.1 | 27 a | −34.1 |
| SSII double | 28 b | −12.5 | 28 b | −26.3 | 39 b | −4.9 |
| null + 1 leaky | | | | | | |
| SSII double | 32 c | | 38 c | | 41 b | |
| WT + 1 leaky | | | | | | |

The impact of the leaky allele upon individual seed size was consistent with the amylose data such that plants with the leaky allele class (SSII double null and 1 leaky) had seed weight intermediate between SSII null and WT (Table 6).

### Example 4

### Reduced Seed Weight of SSII Null Durum Varieties

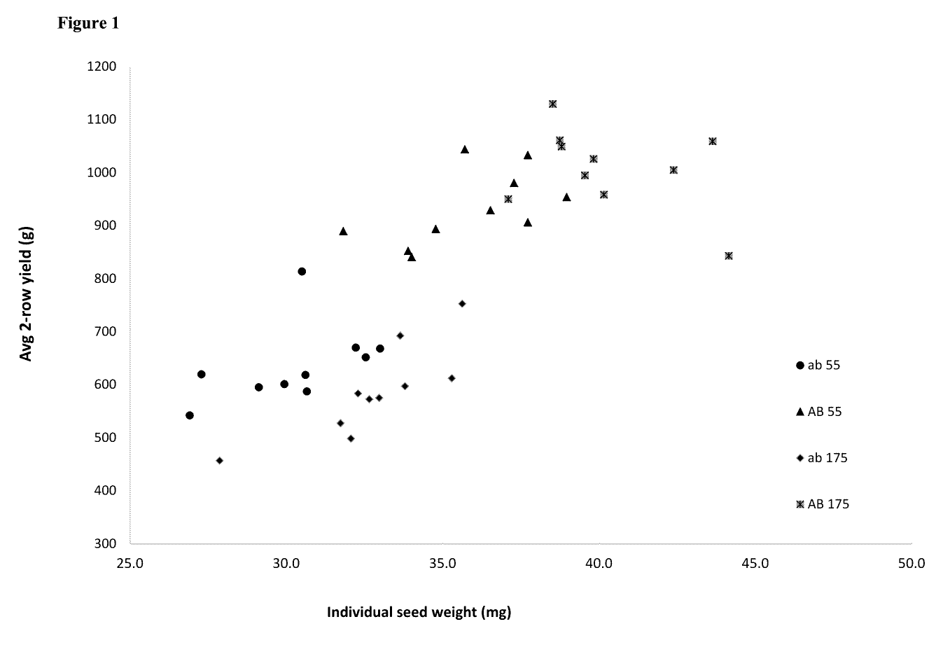

Field data summaries demonstrated that the high amylose content achieved in SSII null tetraploid durum wheat varieties, such as M175 and M55 is linked to significant reductions in seed size and row-yield (Table 7 and FIG. 1). M147 and M55 SSII null mutants were grown in fields together with Wild Type check lines.

Three total separate trials were conducted in Bozeman Mont. (BZ) during Year 1 and Year 2, and Arizona (AZ) in Year 2. The resulting wheat grains were harvested and analyzed for total yield, seed weight, and nutritional composition.

TABLE 7
High dietary fiber durum yield trials. ‘AB’ indicates
wildtype, and ‘ab’ indicates null SSII genes in the
A and B genomes. Comparisons between mutant lines
and wild-type sister lines for the three field trials conducted
in populations created by crossing SSII nulls 175 or 55 with
Mountrail, as indicated below.

| | Year 1 BZ | | Year 2 AZ | | Year 2 BZ | |
|---|---|---|---|---|---|---|
| | Yield Decrease (%) | | | | | |
| SSII Null Parent | 175 | 55 | 175 | 55 | 175 | 55 |
| ab vs AB | −34.1 | −25.4 | −42.6 | −30.7 | −41.5 | −31.8 |
| | Individual Seed weight Decrease (%) | | | | | |
| SSII Null Parent | 175 | 55 | 175 | 55 | 175 | 55 |
| ab vs AB | −24.3 | −16.8 | −21.6 | −18.5 | −18.6 | −15.5 |
| | Protein increase (%) | | | | | |
| SSII Null Parent | 175 | 55 | 175 | 55 | 175 | 55 |
| ab vs AB | 17.0 | 15.3 | 29.0 | 18.8 | 18.5 | 16.8 |

*Each SSII genotypic group has an n = 10 and averages are based off four replicates. For the Bozeman locations the dryland and irrigated environments were averaged together and plots were two-rows. In Arizona there was only one environment and plots were single rows.

Grain harvested from M175 and M55 (ab) lines exhibited higher amylose contents than their check line counterparts (AB), data not shown. However, SSII null varieties M175 and M55 consistently exhibited reduced yields and reduced seed weight compared to their AB check line counterparts.

### Example 5

### Identification of SSII Leaky Mutants and Creation of New SGP Mutant Durum Wheat Varieties

### Creation and Screening of a Mutagenized Durum Wheat Population

Durum wheat accessions obtained from the USDA National Small Grains Collection (NSGC, Aberdeen, Id.) and ICARDA were screened for those that were null for SGP-A1 and/or SGP-Bl using SDS-PAGE of starch granule bound proteins. From the 200 NSGC Triticum durum core collection accessions screened, one line, PI-330546, lacked SGP-A1 and none lacked SGP-Bl. From the 55 ICARDA Triticum durum accessions screened, one line, IG-86304, lacked SGP-A1 and none lacked SGP-Bl. These two lines were crossed independently with the cultivar “Mountrail” (PVP 9900266) (Elias and Miller, 2000) and advanced via single seed decent to the F₅ generation. Lines homozygous for the SGP-A1 null trait that had seed and plant characteristics similar to Mountrail from each cross were then treated with ethyl methane sulfonate (EMS) as described in Feiz et al. (2009) with the exception that 0.5% EMS was used.

### PCR Screening for EMS Mutations in SSII-B.

Leaf tissue from Mountrail SSII-A mutant plant populations named Mountrail/M123, and Mountrail/MS42 suspected of having leaky SSII-B mutant alleles was collected PCR screened for leaky mutations in the SSII-B gene regions as described in Example 1.

Between these two populations three segments of the SSII-B gene were screened from over 500 lines and five missense mutants were identified (Table 8)

TABLE 8
Potential Leaky Mutations in SSIIB in Mountrail/M123
and Mountrail/MS42 EMS populations.

| | | | Nucleotide | Amino Acid |
|---|---|---|---|---|
| EMS Source | ID | Gene | Change^{a} | Change^{b} |
| M123-1-5-22 | 213 | ssII-B | C998T | P333L |
| M123-1-5-39 | 217 | ssII-B | C997T | P333S |
| M123-3-6-280 | 4 | ssII-B | G224A | R75K |
| MS42-35-326 | 275 | ssII-B | G853A | D285N |
| MS42-38-462 | 224 | ssII-B | G989A | G330D |

^{a}Nucleotide changes are numbered relative to the starting methionine of each coding sequence. Notation represents original base, position within coding sequence and altered base.
^{b}Amino acid changes are numbered relative to the starting methionine in each of the proteins. Notation represents original base, position within peptide and altered base.

### Example 6

### Characterization of New SSII Leaky Durum Wheat Mutants

### Seed Size and Amylose Content

Identified heterozygous M₁ mutants from Example 5 were advanced in the greenhouse one generation and M₂ plants were genotyped. Seed harvested from M₂ homozygous leaky mutant lines was compared to seed from sister wild-type lines for individual seed size and apparent amylose content via iodine staining as described in Examples 2 and 3, and known to those having skill in the art. Results of these comparisons are shown in Table 9 below. Line MS42-38-462-224 had no comparison group because it was homozygous in the M₁ generation. Also, line M123-3-6-280-4 was discovered and planted later than the other four leaky mutants. Currently, M₂ plants from this line are being genotyped to identify homozygous sister mutant and wild-type lines which can then be measured for amylose content.

TABLE 9
Apparent Amylose of Potential Leaky Durum Mutants from
Mountrail/M123 and Mountrail/MS42 EMS populations.

| | | Amy- | Amy- | | |
|---|---|---|---|---|---|
| | SSII-B | lose | lose | IKW | IKW |
| ID | Genotype | (%)^{a} | (%) n^{b} | (mg)^{c} | n |
| M123-1-5-22-213 | Wild-type | 25.6 | 5 | 34.0 | 15 |
| M123-1-5-22-213 | Leaky Mutant | 38.8 | 5 | 35.4 | 9 |
| Sudent t-test P | | 0.00 | | 0.50 | |
| M123-1-5-39-217 | Wild-type | 28.0 | 5 | 36.7 | 11 |
| M123-1-5-39-217 | Leaky Mutant | 31.6 | 5 | 37.2 | 12 |
| Sudent t-test P | | 0.04 | | 0.85 | |
| MS42-35-326-275 | Wild-type | 24.5 | 5 | 28.4 | 13 |
| MS42-35-326-275 | Leaky Mutant | 25.5 | 5 | 28.4 | 11 |
| Sudent t-test P | | 0.41 | | 0.74 | |
| MS42-38-462-224 | Leaky Mutant | 30.2 | 5 | 39.2 | 8 |
| Divide | Wild-type | 27.6 | 3 | 52.0 | 5 |
| M175 | Double Mutant | 53.3 | 3 | 32.8 | 5 |

^{a}Amylose %-apparent amylose content was determined via iodine staining.
^{b}Amylose % n-seed from two individual plants was bulked to create one rep (n). If there was not 10 individuals for 5 bulked reps, single plants were used for reps (n).
^{c}IKW—individual kernel weight

Out of the four lines tested, one line (M123-1-5-22-213) displayed the most desirable phenotype, with seeds that were significantly higher in amylose content (39% vs 26%) but did not have a significant decrease in individual kernel weight (Table 9). Two other lines (M123-1-5-39-217 and MS42-38-462-224) had a moderate change in amylose content (˜30%) and line MS42-35-326-275 showed no change in amylose content (Table 9).

### Example 7

### Further Field Characterization of SSII Leaky Durum Wheat Mutants

Since greenhouse grown plants are sometimes not ideal for measuring the effect of a mutation, the Mountrail SGP mutant lines from Example 6 were field-tested in Arizona as single rows to increase the seed and validate their field performance.

Seed for this trial has been harvested and is currently being characterized for seed traits (Single Kernal Characterization and Near-Infrared Reflectance Spectroscopy protein) and apparent amylose content. Additionally, to confirm these findings the most promising lines will be crossed to an elite durum cultivar(s) and advanced to the F₂ generation where amylose content comparison can be made between appropriate haplotypes.

It is expected that M123-1-5-22-213 will exhibit significantly increased amylose content, while also maintaining similar kernel weight to its wild type check line counterparts.

### Example 8

### Identification of Additional SSII Leaky Alleles

In order to identify additional alleles that have the desired amount of leaky function, a new EMS population was created in a ‘Divide’ background. Divide carries wild-type functional alleles of both SSII-A and SSII-B. Single M₁ plants containing mutations in SSII-A and SSII-B were identified by screening two segments of each gene from over 1,000 M₁ lines as described in Example 1 of this application. A total of 9 SSII-A and 9 SSII-B missense alleles were selected for advancement and were planted in the greenhouse for further genotyping and crossing (Table 10). Only missense alleles with a SIFT value indicating negative impact upon protein function are shown.

Starch granule proteins were then extracted from each line by using SDS-PAGE as described in Example 1. Results from these SSII protein analyses are summarized in Table 10 below. Lines exhibiting no difference in SSII protein accumulation from their Wild-Type counterparts are labeled “WT.” Lines exhibiting reduced accumulations of SSII proteins in the SDS-PAGE gels are labeled as “Partial.”

TABLE 10
Potential leaky mutations found in EMS Divide population.

| | | | Nucleotide | Amino acid | SDS |
|---|---|---|---|---|---|
| EMS Source | ID | Gene | change^{a} | change^{b} | PAGE^{c} |
| EMS Divide | 1631 | ssII-A | C356T | A119V | WT |
| EMS Divide | 1214 | ssII-A | C364T | P122S | WT |
| EMS Divide | 81 | ssII-A | C466T | P156S | WT |
| EMS Divide | 904 | ssII-A | C554T | P185L | WT |
| EMS Divide | 280 | ssII-A | G1825A | V609I | WT |
| EMS Divide | 674 | ssII-A | G1936A | V646I | WT |
| EMS Divide | 1174 | ssII-A | G1987A | E663K | Partial |
| EMS Divide | 1513 | ssII-A | G2041A | A681T | Partial |
| EMS Divide | 134 | ssII-A | G2162A | G721E | Partial |
| EMS Divide | 90 | ssII-B | G1567A | D523N | WT |
| EMS Divide | 145 | ssII-B | G1868A | G623E | WT |
| EMS Divide | 1664 | ssII-B | C1892T | A631V | WT |
| EMS Divide | 93 | ssII-B | G1921A | D641N | WT |
| EMS Divide | 1237 | ssII-B | C1975T | R659W | WT |
| EMS Divide | 57 | ssII-B | G2017A | V673M | WT |
| EMS Divide | 1704 | ssII-B | C2077T | P693S | Partial |
| EMS Divide | 47 | ssII-B | C2254T | L752F | WT |
| EMS Divide | 887 | ssII-B | C2269T | R757C | WT |

^{a}Nucleotide changes are numbered relative to the starting methionine of each coding sequence. Notation represents original base, position within coding sequence and altered base.
^{b}Amino acid changes are numbered relative to the starting methionine in each of the proteins. Notation represents original base, position within peptide and altered base.
^{c}Deleterious mutations were confirmed via SDS PAGE. Partial denotes reduced level of the corresponding SSII protein. WT (wild-type) denotes a level of the SSII protein comparable to that extracted from starch of the non-mutated parent line.

Four lines (#134, 1174, 1513, and 1704; 3 SSI-A and 1 SSII-B) were identified as exhibiting “partial” reductions in the abundance of the corresponding SSII protein. These lines are therefore more likely to exhibit the desired amount of leaky function to confer plants with intermediate amylose levels when paired with an SSII null allele. All other lines were identified as wild-type.

### Example 9

### Creation of Durum Wheat Plants with ssii Leaky Mutants in an SSII Null Background

Since the ‘Divide’ mutants from Example 8 still have at least one wild-type copy of both SSII-A and SSII-B genes, these potentially leaky mutants must be crossed to a SSII double null line to determine the impact of the leaky allele. All 18 potentially leaky mutations were successfully crossed to the SSII double-null line #127 from a previous Divide//Mountrail/175 population. The F₁'s from these crosses will be confirmed and advanced in the greenhouse.

Resulting F₂'s will then be genotyped to identify those lines that carry the appropriate SSII allelic combinations from which seed can be tested for amylose and size. The resulting seed from the F2 Divide trials will be tested for amylose content, protein content, seed size, and total yield as described in the preceding examples.

It is expected that one or more of the SSII A and/or SSII B alleles identified in Table 10 will exhibit increased amylose content compared to wild type control plants, but with substantially similar or greater kernel weight than SSII double null plants.

### Example 10

### Wheat Breeding Program Using the Wheat Plants Having Leaky SSII Expression

Non-limiting methods for wheat breeding and agriculturally important traits (e.g., improving wheat yield, biotic stress tolerance, and abiotic stress tolerance etc.) are described in Slafer and Araus, 2007, (“Physiological traits for improving wheat yield under a wide range of conditions”, Scale and Complexity in Plant Systems Research: Gene-Plant-Crop Relations, 147-156); Reynolds (“Physiological approaches to wheat breeding”, Agriculture and Consumer Protection. Food and Agriculture Organization of the United Nations); Richard et al., (“Physiological Traits to Improve the Yield of Rainfed Wheat: Can Molecular Genetics Help”, published by International Maize and Wheat Improvement Center.); Reynolds et al. (“Evaluating Potential Genetic Gains in Wheat Associated with Stress-Adaptive Trait Expression in Elite Genetic Resources under Drought and Heat Stress Crop science”, Crop Science 2007 47: Supplement_3: S-172-S-189); Setter et al., (Review of wheat improvement for waterlogging tolerance in Australia and India: the importance of anaerobiosis and element toxicities associated with different soils. Annals of Botany, Volume 103(2): 221-235); Foulkes et al., (Major Genetic Changes in Wheat with Potential to Affect Disease Tolerance. Phytopathology, July, Volume 96, Number 7, Pages 680-688 (doi: 10.1094/PHYTO-96-0680); Rosyara et al., 2006 (Yield and yield components response to defoliation of spring wheat genotypes with different level of resistance to Helminthosporium leaf blight. Journal of Institute of Agriculture and Animal Science 27. 42-48.); U.S. Pat. Nos. 7,652,204, 6,197,518, 7,034,208, 7,528,297, 6,407,311; U.S. Published Patent Application Nos. 20080040826, 20090300783, 20060223707, 20110027233, 20080028480, 20090320152, 20090320151; WO/2001/029237A2; WO/2008/025097A1; and WO/2003/057848A2.

A wheat plant comprising modified starch with certain leaky SSII allele(s) of the present invention can be self-crossed to produce offspring comprising the same phenotypes.

A wheat plant comprising modified starch or certain allele(s) of starch synthesis genes of the present invention (“donor plant”) can also crossed with another plant (“recipient plant”) to produce a F1 hybrid plant. Some of the F1 hybrid plants can be back-crossed to the recipient plant for 1, 2, 3, 4, 5, 6, 7, or more times. After each backcross, seeds are harvested and planted to select plants that comprise modified starch, and preferred traits inherited from the recipient plant. Such selected plants can be used as either a male or female plant to backcross with the recipient plant.

### Example 11

### Further Characterizations

### Starch Content

The starch content of the SSII leaky lines and a wild-type control wheat line can be measured by one or more methods as described herein, or those described in Moreels et al. (Measurement of Starch Content of Commercial Starches, Starch 39(12):414-416, 1987) or Chiang et al. (Measurement of Total and Gelatinized Starch by Glucoamylase and o-toluidine reagent, Cereal Chem. 54(3):429-435), each of which is incorporated by reference in its entirety. Starch content in the SSII leaky lines is expected to be slightly reduced compared to that of the wild-type control wheat line.

### Glycemic Index

The glycemic index of the SSII leaky lines and a wild-type control wheat line can be measured by one or more methods as described herein, or those described in Brouns et al. (Glycemic index methodology, Nutrition Research Reviews, 18(1):145-171, 2005), Wolever et al. (The glycemic index: methodology and clinical implications, Am. J. Clin. Nutr. 54(5):846-54, 1991), or Goni et al., A starch hydrolysis procedure to estimate glycemic index, Human Study, 17(3):427-437, 1997), each of which is incorporated by reference in its entirety.

The glycemic index, glycemic index, or GI is the measurement of glucose (blood sugar) level increase from carbohydrate consumption. Glucose has a glycemic index of 100, by definition, and other foods have a lower glycemic index. The glycemic index of wheat pasta or bread can be measured by calculating the incremental area under the two-hour blood glucose response curve (AUC) following a 12-hour fast and ingestion of 50 g of available carbohydrates of DHA175 or wild-type pasta. The AUC of the test food is divided by the AUC of the standard (either glucose or white bread, giving two different definitions) and multiplied by 100. The average GI value is calculated from data collected in 5 human subjects. Both the standard and test food must contain an equal amount of available carbohydrate.

### Pasta Quality

Quality of pasta made by the flour of the SSII leaky lines and a wild-type control wheat line can be tested by one or more methods as described herein, or those described in Landi (Durum wheat, semolina and pasta quality characteristics for an Italian food company, Cheam-Options Mediterraneennes, pages 33-42) or Cole (Prediction and measurement of pasta quality, International Journal of Food Science and Technology, 26(2):133-151, 1991), each of which is incorporated by reference in its entirety.

Pasta firmness and resistance to overcooking can be measured. Pasta firmness is expected to be dramatically increased and overcooking reduced in the SSII leaky lines compared to that of the wild-type control wheat line.

Other qualitative factors of pasta can also be considered in evaluating pasta quality, including but not limited to the following: (1) the type of place of origin of the wheat from which the flour is produced; (2) the characteristics of the flour; (3) the manufacturing processes of kneading, drawing and drying; (4) possible added ingredients; and (5) the hygiene of preservation.

### Rapid Visco Analyzer (RVA)

Starch of the SSII leaky lines and a wild-type control wheat line can be tested in a Rapid Visco Analyzer (RVA) by one or more methods as described herein, or those described in Newport Scientific Method ST-00 Revision 3 (General Method for Testing Starch in Rapid Visco Analyzer, 1998), Ross (Amylose, amylopectin, and amylase: Wheat in the RVA, Oregon State University, 55^{th} Conference Presentation, 2008), Bao et al., (Starch RVA profile parameters of rice are mainly controlled by Wx gene, Chinese Science Bulletin, 44(22):2047-2051, 1999), Ravi et al., (Use of Rapid Visco Analyzer (RVA) for measuring the pasting characteristics of wheat flour as influenced by additives, Journal of the Science of Food and Agriculture, 79(12):1571-1576, 1999), or Gamel et al. (Application of the Rapid Visco Analyzer (RVA) as an Effective Rheological Tool for Measurement of β-Glucan Viscosity, 89(1):52-58, 2012), each of which is incorporated by reference in its entirety.

The SSII leaky lines are expected to have reduced peak viscosity compared to that of the wild-type control wheat line.

### Resistant Starch

Resistant starch content of the SSII leaky lines and a wild-type control wheat line can be tested by one or more methods as described herein, or those described in McCleary et al., (Measurement of resistant starch, J. AOAC Int. 2002, 85(3):665-675), Muir and O'Dea (Measurement of resistant starch: factors affecting the amount of starch escaping digestion in vitro, Am. J. Clin. Nutr. 56:123-127, 1992), Berry (Resistant starch: Formation and measurement of starch that survives exhaustive digestion with amylolytic enzymes during the determination of dietary fibre, Journal of Cereal Science, 4(4):301-314, 1986), Englyst et al., (Measurement of resistant starch in vitro and in vivo, British Journal of Nutrition, 75(5):749-755, 1996), each of which is incorporated by reference in its entirety.

The SSII leaky lines are expected to have increased resistant starch compared to the wild-type control wheat line in both dry and cooked pasta trials.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the non-limiting exemplary methods and materials are described herein.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. Nothing herein is to be construed as an admission that the present invention is not entitled to antedate such publication by virtue of prior invention.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

### REFERENCES

Altpeter F, V Vasil, V Srivastava, E Stoger, I K Vasil. Accelerated production of transgenic wheat (Triticum aestivum L.) plants. Plant Cell Rep 16:12-17, 1996.
Bird A. R., M. S. Vuaran, R. A. King, M. Noakes, J. Keogh, M. K. Morell, and D. L. Topping. 2008. Wholegrain foods made from a novel high-amylose barley variety (Himalaya 292) improve indices of bowel health in human subjects. Brit. J. Nutr. 99:1032-1040.
Casiraghi, M.C., Pagani, M. A., Erba, D., Marti, A., Cecchini, C., Grazia D'Egidio, M. G. Quality and nutritional properties of pasta products enriched with immature wheat grain. August 2013 Vol 64:5 544-550.
Ceoloni C, Biagetti M, Ciaffi M, Forte P, Pasquini M (1996) Wheat chromosome engineering at the 4× level: the potential of different alien gene transfers into durum wheat. Euphytica 89:87-97
Chibbar, R. N., M. Baga, S. Ganeshan, P. J. Hucl, A. Limin, C. Perron, I. Ratnayaka, A. Kapoor, V. Verma, and D. B. Fowler 2005. Carbohydrate modification to add value to wheat grain. In: Chung O. K., and Lookhart G. L. (eds.) Third international wheat quality conference, Manhattan, Kans., USA, pp 75-84.
Craig, J., J. R. Lloyd, K. Tomlinson, L. Barber, A. Edwards, T. L. Wang, C. Martin, C. L. Hedley, and A. M. Smith. 1998. Mutations in the gene encoding starch synthase II profoundly alter amylopectin structure in pea embryos. Plant Cell 10:413-426.
Denyer K., C. M. Hylton, C. F. Jenner, and A. M. Smith. 1995. Identification of Multiple Isoforms of Soluble and Granule-Bound Starch Synthase in Developing Wheat Endosperm. Planta 196:256-265.
Dexter J E, Matson, R R (1979). Changes in spaghetti protein solubility during cooking. Cereal Chem. 56: 394-397.
Feiz, L., J. M. Martin, and M. J. Giroux. 2009. Creation and functional analysis of new Puroindoline alleles in Triticum aestivum. Theor. and Appl. Genet. 118:247-257.
Foschia M, Peressini D, Sensidoni, A, Brennan C S (2013). The effects of dietary fibre addition on the quality of common cereal products. Journal of Cereal Science 58(2): 216-227.
Fuad T, Prabhasankar P (2010). “Role of Ingredients in Pasta Product Quality: A Review on Recent Developments.” Critical Reviews in Food Science and Nutrition 50(8): 787-798.
Gilbert J, Procunier J D, Aung T (2000) Influence of the D genome in conferring resistance to fusarium head blight in spring wheat. Euphytica 114:181-186
Hallstrom, E., F. Sestili, D. Lafiandra, I. Bjorck, and E. Ostman. 2011. A novel wheat variety with elevated content of amylose increases resistant starch formation and may beneficially influence glycaemia in healthy subjects. Food & Nutr. Res. 55:7074.
Hazard, B., X. Zhang, P. Colasuonno, C. Uauy, D. M. Beckles, and J. Dubcovsky. 2012. Induced mutations in the starch branching enzyme II (SBEII) genes increase amylose and resistant starch content in durum wheat. Crop Sci. 52:1754-1766.
King R. A., M. Noakes, A. R. Bird, and M. K. Morell, and D. L. Topping. 2008. An extruded breakfast cereal made from a high amylose barley cultivar has a low glycemic index and lower plasma insulin response than one made from a standard barley. J. Cereal Sci. 48:526-530.
Konik-Rose C., J. Thistleton, H. Chanvrier, I. Tan, P. Halley, M. Gidley, B. Kosar-Hashemi, H. Wang, O. Larroque, J. Ikea, S. McMaugh, A. Regina, S. Rahman, M. Morell, and Z. Y. Li. 2007. Effects of starch synthase IIa gene dosage on grain, protein and starch in endosperm of wheat. Theor. Appl. Genet. 115:1053-1065.
Kosar-Hashemi, B., Z. Y. Li, O. Larroque, A. Regina, Y. M. Yamamori, M. K. Morell, and S. Rahman. 2007. Multiple effects of the starch synthase II mutation in developing wheat endosperm. Funct. Plant Biol. 34:431-438.
Lafiandra, D., F. Sestili, R. D'Ovidio, M. Janni, E. Botticella, G. Ferrazzano, M. Silvestri, R. Ranieri, and E. DeAmbrogio. 2010. Approaches for Modification of Starch Composition in Durum Wheat. Cereal Chem. 87:28-34.
Lanning S P, Blake N K, Sherman J D, Talbert L E (2008) Variable production of tetraploid and hexaploid progeny lines from spring wheat by durum wheat crosses Crop Sci 48.199-202
Li, Z., X. Chu, G. Mouille, L. Yan, B. Kosar-Hashemi, S. Hey, J. Napier, P. Shewry, B. Clarke, R. Appels, M. K. Morell, and S. Rahman. 1999. The localization and expression of the class II starch synthases of wheat. Plant Physiol. 120:1147-1156.
Maki, K.C., C. L. Pelkman, E. T. Finocchiaro, K. M. Kelley, A. L. Lawless, A. L. Schild, and T. M. Rains. 2012. Resistant starch from high-amylose maize increases insulin sensitivity in overweight and obese men. J. Nutrit. 142:717-723.
Manthey F A, and Schorno A (2002) Physical and cooking quality of spaghetti made from whole wheat durum. Cereal Chem. 79:504-510.
Marioni, J.C., C. E. Mason, S. M. Mane, M. Stephens, and Y. Gilad. 2008. RNA-seq: an assessment of technical reproducibility and comparison with gene expression arrays. Genome Res. 18:1509-1517.
Martin A, Simpfendorfer S, Hare R A, Eberhard F S, Sutherland M W (2011) Retention of D genome chromosomes in pentaploid wheat crosses. Heredity 107:315-319
Miura, H., S. Tanii, T. Nakamura, and N. Watanabe. 1994. Genetic control of amylose content in wheat endosperm starch and differential effects of 3 Wx genes. Theoret. Appl. Genet. 89:276-280.
Morell, M. K., B. Kosar-Hashemi, M. Cmiel, M. S. Samuel, P. Chandler, S. Rahman, A. Buleon, I. L. Batey, and Z. Y. Li. 2003. Barley sex6 mutants lack starch synthase Ha activity and contain a starch with novel properties. Plant J. 34:172-184.
Nielsen, M. A., Sumner, A. K., and Whalley, L. L., Fortification of Pasta with Pea Flour and Air-Classified Pea Protein Concentrate. Cereal Chem. 57(3):203-206 1980 Nugent, A. P. 2005. Health properties of resistant starch. Nutrition Bulletin 30(1): 27-54.
Polaske, N. W., A. L. Wood, M. R. Campbell, M.C. Nagan, and L. M. Pollak. 2005. Amylose determination of native high-amylose corn starches by differential scanning calorimetry. Starch/Starke 57:118-123.
Regina, A., A. Bird, D. Topping, S. Bowden, J. Freeman, T. Barsby, B. Kosar-Hashemi, Z. Y. Li, S. Rahman, and M. Morell. 2006. High-amylose wheat generated by RNA interference improves indices of large-bowel health in rats. Proc. Natl. Acad. Sci. USA 103:3546-3551.
Sestili, F., E. Botticella, Z. Bedo, A. Phillips, and D. Lafiandra. 2010a. Production of novel allelic variation for genes involved in starch biosynthesis through mutagenesis. Mol. Breeding 25:145-154.
Sestili, F., M. Janni, A. Doherty, E. Botticella, R. D'Ovidio, S. Masci, J. D. Jones, and D. Lafiandra. 2010b. Increasing the amylose content of durum wheat through silencing of the SBEIIa genes. BMC Plant Biol. 10:144.
Shimbata, T., T. Nakamura, P. Vrinten, M. Saito, J. Yonemaru, Y. Seto, and H. Yasuda. 2005. Mutations in wheat starch synthase II genes and PCR-based selection of a SGP-1 null line. Theoret. Appl. Genet. 111:1072-1079.
Smidansky E D, Clancy M, Meyer F D, Lanning S P, Blake N K, Talbert L E, Giroux M J (2002) Enhanced ADP-glucose pyrophosphorylase activity in wheat endosperm increase seed yield. Proc Natl Acad Sci USA 99:1724-1729
Soh, H. N., M. J. Sissons, and M. A. Turner. 2006. Effect of starch granule size distribution and elevated amylose content on durum dough rheology and spaghetti cooking quality. Cereal Chem. 83:513-519.
Tetlow, I. J., K. G. Beisel, S. Cameron, A. Makhmoudova, F. Liu, N. S. Bresolin, R. Wait, M. K. Morell, and M. J. Emes. 2008. Analysis of protein complexes in wheat amyloplasts reveals functional interactions among starch biosynthetic enzymes. Plant Physiol. 146:1878-1891.
Tetlow, I. J. 2006. Understanding storage starch biosynthesis in plants: a means to quality improvement. Can. J. Bot. 84:1167-1185.
Tetlow I. J., M. K. Morell, and M. J. Emes. 2004a. Recent developments in understanding the regulation of starch metabolism in higher plants. J. Exp. Bot. 55:2131-2145.
Tetlow, I. J., R. Wait, Z. X. Lu, R. Akkasaeng, C. G. Bowsher, S. Esposito, B. Kosar-Hashemi, M. K. Morell, and M. J. Emes. 2004b. Protein phosphorylation in amyloplasts regulates starch branching enzyme activity and protein-protein interactions. Plant Cell 16:694-708.
Thompson, D. B. 2000. Strategies for the manufacture of resistant starch. Trends in Food Sci. Tech. 11:245-253.
Yamamori, M., T. Nakamura, T. R. Endo, and T. Nagamine. 1994. Waxy protein deficiency and chromosomal location of coding genes in common wheat. Theoret. Appl. Genet. 89:179-184.
Yamamori, M., and T. R. Endo. 1996. Variation of starch granule proteins and chromosome mapping of their coding genes in common wheat. Theoret. Appl. Genet. 93:275-281.
Yamamori, M., S. Fujita, K. Hayakawa, J. Matsuki, and T. Yasui. 2000. Genetic elimination of a starch granule protein, SGP-1, of wheat generates an altered starch with apparent high amylose. Theoret. Appl. Genet. 101:21-29.
Yamamori M., M. Kato, M. Yui, and M. Kawasaki M. 2006. Resistant starch and starch pasting properties of a starch synthase IIa-deficient wheat with apparent high amylose. Aust. J. Agr. Res. 57:531-535.
Zhang, X. L., C. Colleoni, V. Ratushna, M. Sirghle-Colleoni, M. G. James, and A. M. Myers. 2004. Molecular characterization demonstrates that the Zea mays gene sugary2 codes for the starch synthase isoform SSIIa. Plant Molecular Biology 54:865-879.
Crosbie, G. B., Lambe, W. J., Tsutsui, H., and Gilmour, R. F. 1992. Further evaluation of the flour swelling volume test for identifying wheats potentially suitable for Japanese noodles. J. Cereal Sci. 15:271-280.
Elias E. M. and Miller. J. D. 2000. Registration of Mountrail durum wheat. Crop Sci. 40:1499.
Epstein, J., Morris, C. F., Huber, K. C., 2002. Instrumental texture of white salted noodles prepared from recombinant inbred lines of wheat differing in the three granule bound starch synthase (Waxy) genes. J. Cereal Sci. 35, 51-63.
Hannah, L. C. and James, M. 2008. The complexities of starch biosynthesis in cereal endosperms. Current Opinions in Biotechnology 19:160-165.
Hatcher, D. W., Dexter, J. E., Bellido, G. G., Clarke, J. M., and Anderson, M. J. 2009. Impact of genotype and environment on the quality of amber durum wheat alkaline noodles. Cereal Chem. 86:452-462.
Hung, P. V., Yamamori, M., and Morita, N. 2005. Formation of enzyme resistant starch in bread as affected by high amylose wheat flour substitutions. Cereal Chem. 82:690-694.
Lafiandra, D., Sestili, F., D'Ovidio, R., Janni, M., Botticella, E., Ferrazzano, G., Silvestri, M., Ranieri, R., and DeAmbrogio, E. 2010. Approaches for modification of starch composition in durum wheat. Cereal Chem. 87:28-34.
Li, Z., Chu, X., Mouille, G., Yan, L., Kosar-Hashemi, B., Hey, S., Napier, J., Shewry, P., Clarke, B., Appels, R., Matthew K. Morell, M. K., and Rahman, S. 1999. The localization and expression of the class II starch synthases of wheat. Plant Physiology 120:1147-1155.
Littell. R.C., Stroup, W. W., and Fruend, R. J. 2002. SAS for linear models. 4m ed. SAS Institute Inc, Cary, N.C.
Miura, H., and Tanii, S. 1994. Endosperm starch properties in several wheat cultivars preferred for Japanese noodles. Euphytica 72:171-175.
Martin, J. M., Berg, J. E., Hofer, P., Kephart, K. D., Nash, D. and Bruckner, P. L. 2010. Divergent selection for polyphenol oxidase and grain protein and their impacts on white salted noodle, bread and agronomic traits in wheat. Crop Sci. 50:1298-1309.
Martin, J. M., Talbert, L. E., Habernicht, D. K., Lanning, S. P., Sherman, J. D., Carlson, G., and Giroux, M. J. 2004. Reduced amylose effects on bread and white salted noodle quality. Cereal Chem. 81:188-193.
Morita, N., Maeda, T., Miyazaki, M. Yamamori, M., Miura, H., and Ohtsuka, I. 2002. Dough and baking properties of high-amylose and waxy wheat flours. Cereal Chem. 79:491-495.
Nakamura, T., Yamamori, M., Hirano, H., Hidaka, S., and Nagamine, T., 1995. Production of waxy (amylose-free) wheats. Mol. Gen. Genet. 248:253-259.
Nugent, A. P. 2005. Health properties of resistant starch. Nutrition Bulletin 30(1): 27-54.
Oda, M., Yasuda, Y., Okazaki, S., Yamauchi, Y., and Yokoyama, Y. 1980. A method of flour quality assessment for Japanese noodles. Cereal Chem. 57; 253-254.
SAS Institute. 2011. SAS 9.3 for Windows. SAS Inst., Cary, N.C.
Soh, H. N., Sissons, M. J., and Turner, M. A. 2006. Effect of starch granule size distribution and elevated amylose content on durum dough rheology and spaghetti cooking quality. Cereal Chem. 83:513-519.
Tester, R. F., and Morrison, W. R. 1990. Swelling and gelatinisation of cereal starches. I. Effects of amylopectin, amylose and lipids. Cereal Chemistry, 67, 551-557.
Thompson, D. B. 2000. Strategies for the manufacture of resistant starch. Trends Food Sci. Technol. 11:245-253.
Uauy et al. 2009 (Uauy C, Paraiso F, Colasuonno P, Tran R K, Tsai H, Berardi S, Comai L, Dubcovsky J. A modified TILLING approach to detect induced mutations in tetraploid and hexaploid wheat. BMC Plant Biol. 2009; 9:115.) (cites SBEIIa: GenBank AF338431, SBEIIb: GenBank AY740398].
Vignaux, N., Doehlert, D.C., Elias, E. M. McMullen, M. S., Grant. L. A., and Kianian1, S. F. 2005. Quality of spaghetti made from full and partial waxy durum wheat. Cereal Chem. 82:93-100.
Vignaux, N., Doehlert, D.C., Hegstad, J., Elias, E. M. McMullen, M. S., Grant. L. A., and Kianian1, S. F. 2004. Grain quality characteristics and milling performance of full and partial waxy durum lines. Cereal Chem. 81:377-383.
Yamamori, M. and Endo, T. R. 1996. Variation of starch granule proteins and chromosome mapping of their coding genes in common wheat. Theor. Appl. Genet. 93:275-281.
Yamamori, M., Fujita, S. Hayakawa, K., Matsuki, J., and Yasui, T. 2000. Genetic elimination of a starch granule protein, SGP-1, of wheat generates an altered starch with apparent high amylose. Theor. Appl. Genet. 101:21-29.
Zhao, X.C., Batey, I. L., Sharp, P. J., Crosbie, G., Barclay, I., Wilson, R., Morell, M. K., and Appels, R. 1998. A single genetic locus associated with starch granule properties and noodle quality in wheat. J. Cereal Sc. 27: 7-13.

1. A high amylose grain produced from a wheat plant comprising:
a) at least one SSII leaky allele; and
b) no SSII wild type functional alleles;
wherein the high amylose grain has an increased proportion of starch amylose compared to the proportion of starch amylose of a control grain from an appropriate wild type wheat check variety grown under similar field conditions, and wherein the high amylose grain has higher seed weight compared to a grain from an appropriate null wheat check variety grown under similar field conditions, wherein the null wheat check variety comprises only SSII null alleles.

2. The high amylose grain of claim 1 wherein the proportion of starch amylose of said grain is at least 25% higher compared to the starch amylose of a control grain from an appropriate wild type wheat check variety grown under similar field conditions.

3. The high amylose grain of claim 1 wherein said grain has at least a 10% higher seed weight than the grain from an appropriate null wheat check variety grown under similar field conditions.

4. The high amylose grain of claim 1, wherein the wheat plant is a hexaploid wheat comprising a first, second, and third genome.

5. The high amylose grain of claim 4, wherein the hexaploid wheat comprises homozygous SSII null alleles in the first and second genomes, and the SSII leaky allele in the third genome.

6. The high amylose grain of claim 5, wherein the SSII leaky allele is homozygous in the third genome.

7. The high amylose grain of claim 5, wherein the SSII leaky allele comprises a missense mutation encoding for a protein with an SSII-D-E656K and/or SSII-D-A421V amino acid substitution.

8. The high amylose grain of claim 5, wherein the SSII leaky allele encodes the protein of SEQ ID No. 40 or SEQ ID No. 44.

9. The high amylose grain of claim 1, wherein the wheat plant is a tetraploid wheat comprising a first and second genome.

10. The high amylose grain of claim 9, wherein the tetraploid wheat comprises homozygous SSII null alleles in the first genome, and the SSII leaky allele in the second genome.

11. The high amylose grain of claim 10, wherein the SSII leaky allele is homozygous in the second genome.

12. The high amylose grain of claim 9, wherein the SSII leaky allele comprises a missense mutation encoding for a protein with an SSII-B-P333L and/or SSII-B-P333S amino acid substitution.

13. The high amylose grain of claim 9, wherein the SSII leaky allele encodes the protein of SEQ ID No. 46 or SEQ ID No. 48.

14. The high amylose grain of claim 1, wherein the at least one of the SSII leaky alleles comprises a missense mutation encoding for an SSII protein with an amino acid substitution selected from the group consisting of: SSII-D-E656K, SSII-D-A421V, SSII-D-A785V, SSII-B-P251S, SSII-A-P319L, SSII-B-P333L, SSII-B-P333S, SSII-A-E663K, SSII-A-A681T, SSII-A-G721E, and SSII-A-P693S.

15. A method for producing a wheat plant with one or more wheat starch synthase (SSII) leaky allele(s) and no SSII wild type functional alleles, said method comprising:
a. mutagenizing a wheat grain to form a mutagenized population of grain;
b. growing one or more wheat plants from said mutagenized wheat grain;
c. screening the resulting plants from step (b) to identify wheat plants with an SSII leaky mutant allele;
d. crossing a SSII leaky wheat plant derived from step (c) with a second wheat plant comprising at least one SSII null allele, or at least one SSII leaky allele;
e. harvesting the resulting grain from step (d);
f. growing the harvested grain into a plant; and
g. selecting for a wheat plant comprising one or more SSII leaky allele(s) and no wild type functional SSII alleles.
wherein the resulting plant comprises one or more SSII leaky allele(s) and no SSII wild type functional alleles.

16. A method for producing a wheat plant with one or more wheat starch synthase (SSII) leaky alleles and no wild type functional SSII alleles, said method comprising:
a. crossing a wheat plant comprising at least one SSII leaky alleles with a second wheat plant in which all the SSII alleles are selected from the group consisting of null genes, leaky alleles, and combinations thereof;
b. harvesting the resulting grain;
c. growing the harvested grain into a plant; and,
d. selecting for a wheat plant comprising one or more SSII leaky alleles and no wild type functional SSII alleles.
wherein the resulting plant comprises one or more SSII leaky allele(s) and no SSII wild type functional alleles.

17. The method of claim 15, wherein the at least one of the SSII leaky alleles comprises a missense mutation encoding for an SGP-1 protein with an amino acid substitution selected from the group consisting of: SSII-D-E656K, SSII-D-A421V, SSII-D-A785V, SSII-B-P251S, SSII-A-P319L, SSII-B-P333L, and SSII-B-P333S.

18. The method of claim 16, wherein the at least one of the SSII leaky alleles comprises a missense mutation encoding for a protein with a SSII-D-E656K and/or SSII-D-A421V amino acid substitution.

19. The method of claim 16, wherein the at least one of the SSII leaky alleles encodes the protein of SEQ ID No. 40 or SEQ ID No. 44.

20. The method of claim 16, wherein the at least one of the SSII leaky alleles comprise a missense mutation encoding for a protein with an SSII-B-P333L and/or SSII-B-P333S amino acid substitution.


SEQUENCE LISTING (90 sequences)

SEQ ID NO: 1 - DNA, 6898 bp, Triticum aestivum
  misc_feature (1)..(6898): Starch Synthase II Genome A Genomic Wild Type
gggggccgttcgtacgtacccgcccctcgtgtaaagccgccgccgtcgtcgccgtcccccgctcgcggccatttccccggcctgaccccgtgcgtttaccccacagagcacactccagtccagtccagcccactgccgccgcgctactccccactcccgctgccaccacctccgcctgcgccgcgctctgggcggaggaccaacccgcgcatcgtaccatcgcccgccccgatcccggccgccgccatgtcgtcggcggtcgcgtccgccgcgtccttcctcgcgctcgcctccgcctcccccgggagatcacgcaggcgggcgagggtgagcgcgccgccaccccacgccggggccggcaggctgcactggccgccgtggccgccgcagcgcacggctcgcgacggaggtgtggccgcgcgcgccgccgggaagaaggacgcgagggtcgacgacgacgccgcgtccgcgaggcagccccgcgcacgccgcggtggcgccgccaccaaggtagttggttcgttatgacttgctgtatggcgcgtgcgcctcgagatcagctcacgaattgtttctacaaaacgcacgcgctcgtgtgcaggtcgcggagcggagggatcccgtcaagacgctcgatcgcgacgccgcggaaggtggcgcgccggcaccgccggcaccgaggcaggacgccgcccgtccaccgagtatgaacggcacgccggtgaacggtgagaacaaatctaccggcggcggcggcgcgaccaaagacagcgggctgcccgcacccgcacgcgcgccccatccgtcgacccagaacagagtaccagtgaacggtgaaaacaaagctaacgtcgcctcgccgccgacgagcatagccgaggtcgtggctccggattccgcagctaccatttccatcagtgacaaggcgccggagtccgttgtcccagccgagaagccgccgccgtcgtccggctcaaatttcgtggtctcggcttctgctcccaggctggacattgacagcgatgttgaacctgaactgaagaagggtgcggtcatcgtcgaagaagctccaaacccaaaggctctttcgccgcctgcagcccccgctgtacaagaagacctttgggacttcaagaaatacattggcttcgaggagcccgtggaggccaaggatgatggctgggctgttgcagatgatgcgggctcctttgaacatcaccagaaccatgattccggacctttggcaggggagaacgtcatgaacgtggtcgtcgtggctgctgaatgttctccctggtgcaaaacaggcatggacattacctcttcagtctctcttcccgttgttcataaaactttgctcgaatcactcataagaacaaacattgtgttgcataggtggtcttggagatgttgcgggtgctctgcccaaggctttggcaaagagaggacatcgtgttatggtactgcaggctttcacttaactctgttgagtccatatgttcgaataatatcagtgattggcataatgttattaagtgcaagacatgaaagtgttcttctgttagagtatttcatagccaaccctggaggttaggttgttggggcctactgggtgcgggagggggtttgcaaaaagtggtggttagcagtcggatttcacaaataaggaggctgataaccacgccatcagtgaagggaatgagtgtcgggtacccgatcgaccgttttgcccgacgtcaggtttacctgccctgtagatccgaataagtagttcctatcctcagttaagtaccaaatatcgccagcacccgtgtgtgtatttatagtactggatgatcaatttatcaacatttccggttaatggttgctatcatattcactgtaattgttagtaaacagtggatgtttgtaatgtagatgatggctaaatgtatgttgtcaagctttcattttaaagaaaattttattgggagctagttttcgggtttggttagagccaccaaaaccccagaatttttgggagttggcttgtgacagagggttttggggagttaactttcgggattcagttagagacgctcttactagttccagtaaagagtaaactattttctgcagtcatcccaattgttctgtagaaattaaaagtagaaaatagttgtggtatcatataaaccatatattattcaaaatctagaatcatggacttggctagactttgatgatctgaaattttaaatttgatgataattgagaaatgatcctttctatcttaggttgtggtaccaaggtatggggactatgaggaagcctacgatgtcggagtccgaaaatactacaaggctgctggacaggtaagcgaaaatgcaatcaaaggggagctgaaatttcaatgcttactatcataataaatcaattttaagtaaaaaaatttgtcctgcaggatatggaagtgaattatttccatgcttatatcgatggagttgattttgtgttcattgacgctcctatcttccgacaccgtcaggaagacatttatgggggcagcagacaggttaattttctatatgttggtgtttgattgcactgataaactgagaataagccaaggcctactgactggcatatgattacacattttattttttcaggaaattatgaagcgcatgattttgttctgcaaggccgctgtcgaggtatcctctccaactcaattgacaacctattaccactatacaattatgtgtatgcatgtatttcaacagatacgtaatctcccttgtgaagtgtatatatactaataacatttcaatacctcacatgcacatttggtcaagcgttatgatttaacttctgataatctattgcactgatgaacaataatattgatgatccttgttacttcatcgttatgtttatgttctcttcaccggcgcattgattttggaaatagcatttccacctgccacaaacaataatatatactcctactttcatccaatgtagatattttcgcacttggcatatcatcccattaaatattattggtccatcatttttattcctctataatttgcaggttccttggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgcgcaccaggttccttttctcctaatcttgttttttctctagtctctactattcactccacattgtttgaggaaactaaaggggttgcaaaattatgatggcttatgaaagttatggaggtaaatgcatcagtggtgcttgaacttgtcacgcatgttcactttggtgcttacagttgtagactacggaaaactggtgcaaaaacttggctattgtgtgcaatacggtgtattttccgtatgtagggtcaaatgttgcctatgtggcattgtattcccgtctatagatgttagaccgtgcctacatcgccattgggcccacacactccctattacatgtgggacccacttgtcagcctatgacataaataaaatggaaatttataataaaaatgatggcctggggtcttgaaaatgggacctcgcaggtatgccgctagccagcacgccctaatcattaatcccctatgcacttcagtatgtgtgtgtctgtgtgtggagtcggggggggggggggtatgtattcttatatcctttgctctaaggctatcattggcgtgctagcaccgccgggtctccatcaacaccgacatcatccacgaccccatccagctcttcctcaacaagcccacctccagcctcaactcgggcagcaccttcatggtggcctaggtgctctgcaccatcgctcgaagtggcaacgtcgttgtcatgaccatccaccaacccaacacgcaaaatcctcaacatcatttgacagtgagcatgcccctcttgtcatttccccctcatacccaaacctgtctcgataacccttggagctgcacaagttgtgaccatcgcctgcgtcgctgcacaacgcctgacctagccggaccattatagaagcctgccttgggagcccatacctccctgcacatcctcctctttccccatagaccgtgccgccatcgcaaatcgacttctcctctcctccttctcctgctctggccgttttccccgccgcgaagctgcaatccatggcgagttggccatggccctattccccaattgctcgcactaggaggtcctccttgaagcctagcacctttccccctcactaattgcaagttggggagcccctcacgagctccctacattggccgtagtcgcctgccgcctcaactctggtccagacctcgttcccgtggcctcgacgacatctcctcgacctcccattccacacgcggcctggagaggatcaccgcatgttcatccatccgacccgaatcatcatagaaccaacgccagagaggtcatcccgacgacgtcgcactgttcctctatttcccccaagctgtgtcgcgtcataatataagacgggcttgtttgtatctctaggggtcatcgggttcaatggctagctcatgcatggacctgactttaggtcccaggttcgaacccccgcgtgcacataatttatttgctatttatttctcctatctactaataagtgggacccacacatcatactaagccctttttgtctcttgcctgctgataagtgggacccacacgcaatacttagccagagagagaacatgagcttgttggtgccgcgttggcaagccacgccagcagtcttaacggctacaaacagaggatatggtgtcacatcaacgtgcagagcgtttacgaatggaaagtgtactatatgcacacaagagccagagccaggtttttgcaccagttttttgtattctacaactgcgagcaccaaagtgtacatgccgaaccaaagtgaacacggcgagtccattcttttctggtacgatgggtggctcaaagacaccccaatagaagctatcacctcggacattgccaattgggtgccgaactacattaaagtggcaaggtcagttgattgcgctatgtgttggatcagggacataaacgattccataaatattgcaatgttcattcaaattcttaacatttgcgaggcgcttcatgatttccatctccctcatatcagagacatttggtcgtgtacactaaatttctcaggtcacttctcgtctaaatccgcatatgtagctcacttcaatgacttgactttggtccagctaacgccatttggcgctcttgggcccctttgcgtagcaattttttcatatggctcgctccgcgcaataggatttggatcacgggcagacgcgctagatgaggtcttccacacaatgaacattgcgttctttgctctgctcttccagaagacacttgtgattttattacgagttgtgccatagatgccggtgggtttcagctaggaacagggtgtcaccttcggacaagaagaagttgcatagtttggtcgtcttaactgcttggtcgatttggaaggagcacaacaacagtctttgaaggcaaagctaattccctcgatcaagttattagacggatcaaatgtggtacagtgccatggctagttgcttggagtcacttttaagctaggtcgcttgccatcacgctttgtgttaagcgcttggggtcgcttttgctcaatttgtattttgttgttatgtgtttttagttatgtagcctgaactttctggacttagttttttcctctataatgatcacatgctttggtcagttattcctttcttgggtactccgttgggctaattctttctcttgattgatgttgtatatgcagggccgtggcccagtagatgaattcccgttcaccgagttgcctgagcactacctggaacacttcagactgtacgaccccgtgggtggtgagcacgccaactacttcgccgccggcctgaagatggcggaccaggttgtcgtggtgagccccgggtacctgtgggagctcaagacggtggagggcggctgggggcttcacgacatcatacggcagaacgactggaagacccgcggcatcgtcaacggcatcgacaacatggagtggaaccccgaggtggacgtccacctccagtcggacggctacaccaacttctccctgagcacgctggactccggcaagcggcagtgcaaggaggccctgcagcgcgagctgggcctgcaggtccgcgccgacgtgccgctgctcggcttcatcggccgcctggacgggcagaagggcgtggagatcatcgcggacgccatgccctggatcgtgagccaggacgtgcagctggtcatgctgggcaccggccgccacgacctggagagcatgctgcggcacttcgagcgggagcaccacgacaaggtgcgcgggtgggtggggttctccgtgcgcctggcgcaccggatcacggcgggcgccgacgcgctcctcatgccctcccggttcgagccgtgcgggctgaaccagctctacgccatggcctacggcaccgtccccgtcgtgcacgccgtcggcgggctgagggacaccgtgccgccgttcgaccccttcaaccactccggcctcgggtggacgttcgaccgcgccgaggcgcacaagctgatcgaggcgctcgggcactgcctccgcacctaccgggactacaaggagagctggaggggcctccaggagcgcggcatgtcgcaggacttcagctgggagcatgccgccaagctctacgaggacgtcctcctcaaggccaagtaccagtggtga

SEQ ID NO: 2 - DNA, 2400 bp, Triticum aestivum
  misc_feature (1)..(2400): Starch Synthase II Genome A CDS Wild Type
atgtcgtcggcggtcgcgtccgccgcgtccttcctcgcgctcgcctccgcctcccccgggagatcacgcaggcgggcgagggtgagcgcgccgccaccccacgccggggccggcaggctgcactggccgccgtggccgccgcagcgcacggctcgcgacggaggtgtggccgcgcgcgccgccgggaagaaggacgcgagggtcgacgacgacgccgcgtccgcgaggcagccccgcgcacgccgcggtggcgccgccaccaaggtcgcggagcggagggatcccgtcaagacgctcgatcgcgacgccgcggaaggtggcgcgccggcaccgccggcaccgaggcaggacgccgcccgtccaccgagtatgaacggcacgccggtgaacggtgagaacaaatctaccggcggcggcggcgcgaccaaagacagcgggctgcccgcacccgcacgcgcgccccatccgtcgacccagaacagagtaccagtgaacggtgaaaacaaagctaacgtcgcctcgccgccgacgagcatagccgaggtcgtggctccggattccgcagctaccatttccatcagtgacaaggcgccggagtccgttgtcccagccgagaagccgccgccgtcgtccggctcaaatttcgtggtctcggcttctgctcccaggctggacattgacagcgatgttgaacctgaactgaagaagggtgcggtcatcgtcgaagaagctccaaacccaaaggctctttcgccgcctgcagcccccgctgtacaagaagacctttgggacttcaagaaatacattggcttcgaggagcccgtggaggccaaggatgatggctgggctgttgcagatgatgcgggctcctttgaacatcaccagaaccatgattccggacctttggcaggggagaacgtcatgaacgtggtcgtcgtggctgctgaatgttctccctggtgcaaaacaggtggtcttggagatgttgcgggtgctctgcccaaggctttggcaaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctatcttccgacaccgtcaggaagacatttatgggggcagcagacaggaaattatgaagcgcatgattttgttctgcaaggccgctgtcgaggttccttggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgcgcaccagggccgtggcccagtagatgaattcccgttcaccgagttgcctgagcactacctggaacacttcagactgtacgaccccgtgggtggtgagcacgccaactacttcgccgccggcctgaagatggcggaccaggttgtcgtggtgagccccgggtacctgtgggagctcaagacggtggagggcggctgggggcttcacgacatcatacggcagaacgactggaagacccgcggcatcgtcaacggcatcgacaacatggagtggaaccccgaggtggacgtccacctccagtcggacggctacaccaacttctccctgagcacgctggactccggcaagcggcagtgcaaggaggccctgcagcgcgagctgggcctgcaggtccgcgccgacgtgccgctgctcggcttcatcggccgcctggacgggcagaagggcgtggagatcatcgcggacgccatgccctggatcgtgagccaggacgtgcagctggtcatgctgggcaccggccgccacgacctggagagcatgctgcggcacttcgagcgggagcaccacgacaaggtgcgcgggtgggtggggttctccgtgcgcctggcgcaccggatcacggcgggcgccgacgcgctcctcatgccctcccggttcgagccgtgcgggctgaaccagctctacgccatggcctacggcaccgtccccgtcgtgcacgccgtcggcgggctgagggacaccgtgccgccgttcgaccccttcaaccactccggcctcgggtggacgttcgaccgcgccgaggcgcacaagctgatcgaggcgctcgggcactgcctccgcacctaccgggactacaaggagagctggaggggcctccaggagcgcggcatgtcgcaggacttcagctgggagcatgccgccaagctctacgaggacgtcctcctcaaggccaagtaccagtggtga

SEQ ID NO: 3 - protein, 798 aa, Triticum aestivum
  MISC_FEATURE (1)..(798): Starch Synthase II Genome A Protein Wild Type
MSSAVASAASFLALASASPGRSRRRARVSAPPPHAGAGRLHWPPWPPQRTARDGGVAARAAGKKDARVDDDAASARQPRARRGGAATKVAERRDPVKTLDRDAAEGGAPAPPAPRQDAARPPSMNGTPVNGENKSTGGGGATKDSGLPAPARAPHPSTQNRVPVNGENKANVASPPTSIAEVVAPDSAATISISDKAPESVVPAEKPPPSSGSNFVVSSAPRLDIDSDVEPELKKGAVIVEEAPNPKALSPPAAPAVQEDLWDFKKYIGFEEPVEAKDDGWAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPIFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDVHLQSDGYTNFSLSTLDSGKRQCKEALQRELGLQVRADVPLLGFIGRLDGQKGVEIIADAMPWIVSQDVQLVMLGTGRHDLESMLRHFEREHHDKVRGWVGFSVRLAHRITAGADALLMPSRFEPCGLNQLYAMAYGTVPVVHAVGGLRDTVPPFDPFNHSGLGWTFDRAEAHKLIEALGHCLRTYRDYKESWRGLQERGMSQDFSWEHAAKLYEDVLLKAKYQW

SEQ ID NO: 4 - DNA, 2400 bp, Artificial Sequence
  RJ 302 Mutant Starch Synthase II Genome A CDS
atgtcgtcggcggtcgcgtccgccgcgtccttcctcgcgctcgcctccgcctcccccgggagatcacgcaggcgggcgagggtgagcgcgccgccaccccacgccggggccggcaggctgcactggccgccgtggccgccgcagcgcacggctcgcgacggaggtgtggccgcgcgcgccgccgggaagaaggacgcgagggtcgacgacgacgccgcgtccgcgaggcagccccgcgcacgccgcggtggcgccgccaccaaggtcgcggagcggagggatcccgtcaagacgctcgatcgcgacgccgcggaaggtggcgcgccggcaccgccggcaccgaggcaggacgccgcccgtccaccgagtatgaacggcacgccggtgaacggtgagaacaaatctaccggcggcggcggcgcgaccaaagacagcgggctgcccgcacccgcacgcgcgccccatccgtcgacccagaacagagtaccagtgaacggtgaaaacaaagctaacgtcgcctcgccgccgacgagcatagccgaggtcgtggctccggattccgcagctaccatttccatcagtgacaaggcgccggagtccgttgtcccagccgagaagccgccgccgtcgtccggctcaaatttcgtggtctcggcttctgctcccaggctggacattgacagcgatgttgaacctgaactgaagaagggtgcggtcatcgtcgaagaagctccaaacccaaaggctctttcgccgcctgcagcccccgctgtacaagaagacctttgggacttcaagaaatacattggcttcgaggagcccgtggaggccaaggatgatggctgggctgttgcagatgatgcgggctcctttgaacatcaccagaaccatgattccggacctttggcaggggagaacgtcatgaacgtggtcgtcgtggctgctgaatgttctccctggtgcaaaacaggtggtcttggagatgttgcgggtgctctgcccaaggctttggcaaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctatcttccgacaccgtcaggaagacatttatgggggcagcagacaggaaattatgaagcgcatgattttgttctgcaaggccgctgtcgaggttccttggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgcgcaccagggccgtggcccagtagatgaattcccgttcaccgagttgcctgagcactacctggaacacttcagactgtacgaccccgtgggtggtgagcacgccaactacttcgccgccggcctgaagatggcggaccaggttgtcgtggtgagccccgggtacctgtgggagctcaagacggtggagggcggctgggggcttcacgacatcatacggcagaacgactggaagacccgcggcatcgtcaacggcatcgacaacatggagtggaaccccgaggtggacgtccacctccagtcggacggctacaccaacttctccctgagcacgctggactccggcaagcggcagtgcaaggaggccctgcagcgcgagctgggcctgcaggtccgcgccgacgtgccgctgctcggcttcatcggccgcctggacgggcagaagggcgtggagatcatcgcggacgccatgccctggatcgtgagccaggacgtgcagctggtcatgctgggcaccggccgccacgacctggagagcatgctgcggcacttcgagcgggagcaccacgacaaggtgcgcgggtgggtggggttctccgtgcgcctggcgcaccggatcacggcgggcgccgacgcgctcctcatgccctcccggttcgagccgtgcgggctgaaccagctctacgtcatggcctacggcaccgtccccgtcgtgcacgccgtcggcgggctgagggacaccgtgccgccgttcgaccccttcaaccactccggcctcgggtggacgttcgaccgcgccgaggcgcacaagctgatcgaggcgctcgggcactgcctccgcacctaccgggactacaaggagagctggaggggcctccaggagcgcggcatgtcgcaggacttcagctgggagcatgccgccaagctctacgaggacgtcctcctcaaggccaagtaccagtggtga

SEQ ID NO: 5 - protein, 799 aa, Artificial Sequence
  RJ 302 Mutant Starch Synthase II Genome A Protein
MSSAVASAASFLALASASPGRSRRRARVSAPPPHAGAGRLHWPPWPPQRTARDGGVAARAAGKKDARVDDDAASARQPRARRGGAATKVAERRDPVKTLDRDAAEGGAPAPPAPRQDAARPPSMNGTPVNGENKSTGGGGATKDSGLPAPARAPHPSTQNRVPVNGENKANVASPPTSIAEVVAPDSAATISISDKAPESVVPAEKPPPSSGSNFVVSASAPRLDIDSDVEPELKKGAVIVEEAPNPKALSPPAAPAVQEDLWDFKKYIGFEEPVEAKDDGWAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPIFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDVHLQSDGYTNFSLSTLDSGKRQCKEALQRELGLQVRADVPLLGFIGRLDGQKGVEIIADAMPWIVSQDVQLVMLGTGRHDLESMLRHFEREHHDKVRGWVGFSVRLAHRITAGADALLMPSRFEPCGLNQLYVMAYGTVPVVHAVGGLRDTVPPFDPFNHSGLGWTFDRAEAHKLIEALGHCLRTYRDYKESWRGLQERGMSQDFSWEHAAKLYEDVLLKAKYQW

SEQ ID NO: 6 - DNA, 2400 bp, Artificial Sequence
  RJ 493 Mutant Starch Synthase II Genome A CDS
atgtcgtcggcggtcgcgtccgccgcgtccttcctcgcgctcgcctccgcctcccccgggagatcacgcaggcgggcgagggtgagcgcgccgccaccccacgccggggccggcaggctgcactggccgccgtggccgccgcagcgcacggctcgcgacggaggtgtggccgcgcgcgccgccgggaagaaggacgcgagggtcgacgacgacgccgcgtccgcgaggcagccccgcgcacgccgcggtggcgccgccaccaaggtcgcggagcggagggatcccgtcaagacgctcgatcgcgacgccgcggaaggtggcgcgccggcaccgccggcaccgaggcaggacgccgcccgtccaccgagtatgaacggcacgccggtgaacggtgagaacaaatctaccggcggcggcggcgcgaccaaagacagcgggctgcccgcacccgcacgcgcgccccatccgtcgacccagaacagagtaccagtgaacggtgaaaacaaagctaacgtcgcctcgccgccgacgagcatagccgaggtcgtggctccggattccgcagctaccatttccatcagtgacaaggcgccggagtccgttgtcccagccgagaagccgccgccgtcgtccggctcaaatttcgtggtctcggcttctgctcccaggctggacattgacagcgatgttgaacctgaactgaagaagggtgcggtcatcgtcgaagaagctccaaacccaaaggctctttcgccgcttgcagcccccgctgtacaagaagacctttgggacttcaagaaatacattggcttcgaggagcccgtggaggccaaggatgatggctgggctgttgcagatgatgcgggctcctttgaacatcaccagaaccatgattccggacctttggcaggggagaacgtcatgaacgtggtcgtcgtggctgctgaatgttctccctggtgcaaaacaggtggtcttggagatgttgcgggtgctctgcccaaggctttggcaaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctatcttccgacaccgtcaggaagacatttatgggggcagcagacaggaaattatgaagcgcatgattttgttctgcaaggccgctgtcgaggttccttggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgcgcaccagggccgtggcccagtagatgaattcccgttcaccgagttgcctgagcactacctggaacacttcagactgtacgaccccgtgggtggtgagcacgccaactacttcgccgccggcctgaagatggcggaccaggttgtcgtggtgagccccgggtacctgtgggagctcaagacggtggagggcggctgggggcttcacgacatcatacggcagaacgactggaagacccgcggcatcgtcaacggcatcgacaacatggagtggaaccccgaggtggacgtccacctccagtcggacggctacaccaacttctccctgagcacgctggactccggcaagcggcagtgcaaggaggccctgcagcgcgagctgggcctgcaggtccgcgccgacgtgccgctgctcggcttcatcggccgcctggacgggcagaagggcgtggagatcatcgcggacgccatgccctggatcgtgagccaggacgtgcagctggtcatgctgggcaccggccgccacgacctggagagcatgctgcggcacttcgagcgggagcaccacgacaaggtgcgcgggtgggtggggttctccgtgcgcctggcgcaccggatcacggcgggcgccgacgcgctcctcatgccctcccggttcgagccgtgcgggctgaaccagctctacgccatggcctacggcaccgtccccgtcgtgcacgccgtcggcgggctgagggacaccgtgccgccgttcgaccccttcaaccactccggcctcgggtggacgttcgaccgcgccgaggcgcacaagctgatcgaggcgctcgggcactgcctccgcacctaccgggactacaaggagagctggaggggcctccaggagcgcggcatgtcgcaggacttcagctgggagcatgccgccaagctctacgaggacgtcctcctcaaggccaagtaccagtggtga

SEQ ID NO: 7 - protein, 799 aa, Artificial Sequence
  RJ 493 Mutant Starch Synthase II Genome A Protein
MSSAVASAASFLALASASPGRSRRRARVSAPPPHAGAGRLHWPPWPPQRTARDGGVAARAAGKKDARVDDDAASARQPRARRGGAATKVAERRDPVKTLDRDAAEGGAPAPPAPRQDAARPPSMNGTPVNGENKSTGGGGATKDSGLPAPARAPHPSTQNRVPVNGENKANVASPPTSIAEVVAPDSAATISISDKAPESVVPAEKPPPSSGSNFVVSASAPRLDIDSDVEPELKKGAVIVEEAPNPKALSPLAAPAVQEDLWDFKKYIGFEEPVEAKDDGWAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPIFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDVHLQSDGYTNFSLSTLDSGKRQCKEALQRELGLQVRADVPLLGFIGRLDGQKGVEIIADAMPWIVSQDVQLVMLGTGRHDLESMLRHFEREHHDKVRGWVGFSVRLAHRITAGADALLMPSRFEPCGLNQLYAMAYGTVPVVHAVGGLRDTVPPFDPFNHSGLGWTFDRAEAHKLIEALGHCLRTYRDYKESWRGLQERGMSQDFSWEHAAKLYEDVLLKAKYQW

SEQ ID NO: 8 - DNA, 2400 bp, Artificial Sequence
  RJ 253 Mutant Starch Synthase II Genome A CDS
atgtcgtcggcggtcgcgtccgccgcgtccttcctcgcgctcgcctccgcctcccccgggagatcacgcaggcgggcgagggtgagcgcgccgccaccccacgccggggccggcaggctgcactggccgccgtggccgccgcagcgcacggctcgcgacggaggtgtggccgcgcgcgccgccgggaagaaggacgcgagggtcgacgacgacgccgcgtccgcgaggcagccccgcgcacgccgcggtggcgccgccaccaaggtcgcggagcggagggatcccgtcaagacgctcgatcgcgacgccgcggaaggtggcgcgccggcaccgccggcaccgaggcaggacgccgcccgtccaccgagtatgaacggcacgccggtgaacggtgagaacaaatctaccggcggcggcggcgcgaccaaagacagcgggctgcccgcacccgcacgcgcgccccatccgtcgacccagaacagagtaccagtgaacggtgaaaacaaagctaacgtcgcctcgccgccgacgagcatagccgaggtcgtggctccggattccgcagctaccatttccatcagtgacaaggcgccggagtccgttgtcccagccgagaagccgccgccgtcgtccggctcaaatttcgtggtctcggcttctgctcccaggctggacattgacagcgatgttgaacctgaactgaagaagggtgcggtcatcgtcgaagaagctccaaacccaaaggctctttcgccgcctgcagcccccgctgtacaagaagacctttgggacttcaagaaatacattggcttcgaggagcccgtggaggccaaggatgatggctgggctgttgcagatgatgcgggctcctttgaacatcaccagaaccatgattccggacctttggcaggggagaacgtcatgaacgtggtcgtcgtggctgctgaatgttctccctggtgcaaaacaggtggtcttggagatgttgcgggtgctctgcccaaggctttggcaaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctatcttccgacaccgtcaggaagacatttatgggggcagcagacaggaaattatgaagcgcatgattttgttctgcaaggccgctgtcgaggttccttggcacgttccatacggcggtgtcccttatggggatggaaatctggtgtttattgcaaatgattggcacacggcactcctgcctgtctatctgaaagcatattacagggaccatggtttgatgcagtacactcggtccattatggtgatacataacatcgcgcaccagggccgtggcccagtagatgaattcccgttcaccgagttgcctgagcactacctggaacacttcagactgtacgaccccgtgggtggtgagcacgccaactacttcgccgccggcctgaagatggcggaccaggttgtcgtggtgagccccgggtacctgtgggagctcaagacggtggagggcggctgggggcttcacgacatcatacggcagaacgactggaagacccgcggcatcgtcaacggcatcgacaacatggagtggaaccccgaggtggacgtccacctccagtcggacggctacaccaacttctccctgagcacgctggactccggcaagcggcagtgcaaggaggccctgcagcgcgagctgggcctgcaggtccgcgccgacgtgccgctgctcggcttcatcggccgcctggacgggcagaagggcgtggagatcatcgcggacgccatgccctggatcgtgagccaggacgtgcagctggtcatgctgggcaccggccgccacgacctggagagcatgctgcggcacttcgagcgggagcaccacgacaaggtgcgcgggtgggtggggttctccgtgcgcctggcgcaccggatcacggcgggcgccgacgcgctcctcatgccctcccggttcgagccgtgcgggctgaaccagctctacgccatggcctacggcaccgtccccgtcgtgcacgccgtcggcgggctgagggacaccgtgccgccgttcgaccccttcaaccactccggcctcgggtggacgttcgaccgcgccgaggcgcacaagctgatcgaggcgctcgggcactgcctccgcacctaccgggactacaaggagagctggaggggcctccaggagcgcggcatgtcgcaggacttcagctgggagcatgccgccaagctctacgaggacgtcctcctcaaggccaagtaccagtggtga

SEQ ID NO: 9 - protein, 799 aa, Artificial Sequence
  RJ 253 Mutant Starch Synthase II Genome A Protein
MSSAVASAASFLALASASPGRSRRRARVSAPPPHAGAGRLHWPPWPPQRTARDGGVAARAAGKKDARVDDDAASARQPRARRGGAATKVAERRDPVKTLDRDAAEGGAPAPPAPRQDAARPPSMNGTPVNGENKSTGGGGATKDSGLPAPARAPHPSTQNRVPVNGENKANVASPPTSIAEVVAPDSAATISISDKAPESVVPAEKPPPSSGSNFVVSASAPRLDIDSDVEPELKKGAVIVEEAPNPKALSPPAAPAVQEDLWDFKKYIGFEEPVEAKDDGWAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPIFRHRQEDIYGGSRQEIMKRMILFCKAAVEVPWHVPYGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDVHLQSDGYTNFSLSTLDSGKRQCKEALQRELGLQVRADVPLLGFIGRLDGQKGVEIIADAMPWIVSQDVQLVMLGTGRHDLESMLRHFEREHHDKVRGWVGFSVRLAHRITAGADALLMPSRFEPCGLNQLYAMAYGTVPVVHAVGGLRDTVPPFDPFNHSGLGWTFDRAEAHKLIEALGHCLRTYRDYKESWRGLQERGMSQDFSWEHAAKLYEDVLLKAKYQW

SEQ ID NO: 10 - DNA, 2400 bp, Artificial Sequence
  RJ 42 Mutant Starch Synthase II Genome A CDS
atgtcgtcggcggtcgcgtccgccgcgtccttcctcgcgctcgcctccgcctcccccgggagatcacgcaggcgggcgagggtgagcgcgccgccaccccacgccggggccggcaggctgcactggccgccgtggccgccgcagcgcacggctcgcgacggaggtgtggccgcgcgcgccgccgggaagaaggacgcgagggtcgacgacgacgccgcgtccgcgaggcagccccgcgcacgccgcggtggcgccgccaccaaggtcgcggagcggagggatcccgtcaagacgctcgatcgcgacgccgcggaaggtggcgcgccggcaccgccggcaccgaggcaggacgccgcccgtccaccgagtatgaacggcacgccggtgaacggtgagaacaaatctaccggcggcggcggcgcgaccaaagacagcgggctgcccgcacccgcacgcgcgccccatccgtcgacccagaacagagtaccagtgaacggtgaaaacaaagctaacgtcgcctcgccgccgacgagcatagccgaggtcgtggctccggattccgcagctaccatttccatcagtgacaaggcgccggagtccgttgtcccagccgagaagccgccgccgtcgtccggctcaaatttcgtggtctcggcttctgctcccaggctggacattgacagcgatgttgaacctgaactgaagaagggtgcggtcatcgtcgaagaagctccaaacccaaaggctctttcgccgcctgcagcccccgctgtacaagaagacctttgggacttcaagaaatacattggcttcgaggagcccgtggaggccaaggatgatggctgggctgttgcagatgatgcgggctcctttgaacatcaccagaaccatgattccggacctttggcaggggagaacgtcatgaacgtggtcgtcgtggctgctgaatgttctctctggtgcaaaacaggtggtcttggagatgttgcgggtgctctgcccaaggctttggcaaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctatcttccgacaccgtcaggaagacatttatgggggcagcagacaggaaattatgaagcgcatgattttgttctgcaaggccgctgtcgaggttccttggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgcgcaccagggccgtggcccagtagatgaattcccgttcaccgagttgcctgagcactacctggaacacttcagactgtacgaccccgtgggtggtgagcacgccaactacttcgccgccggcctgaagatggcggaccaggttgtcgtggtgagccccgggtacctgtgggagctcaagacggtggagggcggctgggggcttcacgacatcatacggcagaacgactggaagacccgcggcatcgtcaacggcatcgacaacatggagtggaaccccgaggtggacgtccacctccagtcggacggctacaccaacttctccctgagcacgctggactccggcaagcggcagtgcaaggaggccctgcagcgcgagctgggcctgcaggtccgcgccgacgtgccgctgctcggcttcatcggccgcctggacgggcagaagggcgtggagatcatcgcggacgccatgccctggatcgtgagccaggacgtgcagctggtcatgctgggcaccggccgccacgacctggagagcatgctgcggcacttcgagcgggagcaccacgacaaggtgcgcgggtgggtggggttctccgtgcgcctggcgcaccggatcacggcgggcgccgacgcgctcctcatgccctcccggttcgagccgtgcgggctgaaccagctctacgccatggcctacggcaccgtccccgtcgtgcacgccgtcggcgggctgagggacaccgtgccgccgttcgaccccttcaaccactccggcctcgggtggacgttcgaccgcgccgaggcgcacaagctgatcgaggcgctcgggcactgcctccgcacctaccgggactacaaggagagctggaggggcctccaggagcgcggcatgtcgcaggacttcagctgggagcatgccgccaagctctacgaggacgtcctcctcaaggccaagtaccagtggtga

SEQ ID NO: 11 - protein, 799 aa, Artificial Sequence
  RJ 42 Mutant Starch Synthase II Genome A Protein
MSSAVASAASFLALASASPGRSRRRARVSAPPPHAGAGRLHWPPWPPQRTARDGGVAARAAGKKDARVDDDAASARQPRARRGGAATKVAERRDPVKTLDRDAAEGGAPAPPAPRQDAARPPSMNGTPVNGENKSTGGGGATKDSGLPAPARAPHPSTQNRVPVNGENKANVASPPTSIAEVVAPDSAATISISDKAPESVVPAEKPPPSSGSNFVVSASAPRLDIDSDVEPELKKGAVIVEEAPNPKALSPPAAPAVQEDLWDFKKYIGFEEPVEAKDDGWAVADDAGSFEHHQNHDSGPLAGENVMNVVVVAAECSLWCKTGGLGDVAGALPKALAKRGHRVMVVVPRYGDYEEAYDVGVRKYYKAAGQDMEVNYFHAYIDGVDFVFIDAPIFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDVHLQSDGYTNFSLSTLDSGKRQCKEALQRELGLQVRADVPLLGFIGRLDGQKGVEIIADAMPWIVSQDVQLVMLGTGRHDLESMLRHFEREHHDKVRGWVGFSVRLAHRITAGADALLMPSRFEPCGLNQLYAMAYGTVPVVHAVGGLRDTVPPFDPFNHSGLGWTFDRAEAHKLIEALGHCLRTYRDYKESWRGLQERGMSQDFSWEHAAKLYEDVLLKAKYQW

SEQ ID NO: 12 - DNA, 6811 bp, Triticum aestivum
  misc_feature (1)..(6811): Starch Synthase II Genome B Genomic Wild Type
gggggccgttcgtacgtacccacccctcgtgtaaagccgccgccgtcgtcgccgtcccccgctcgcggccatttcctcggcctgaccccgtgcgtttaccccacacagagcacactccagtccagtccagcccactgccgcgctactccccactcccactgccaccacctccgcctgcgccgcgctctgggcggaccaacccgcgcatcgtatcacgatcacccaccccgatcccggccgccgccatgtcgtcggcggtcgcgtccgccgcgtccttcctcgcgctcgcgtccgcctcccccgggagatcacggaggaggacgagggtgagcgcgtcgccaccccacaccggggctggcaggttgcactggccgccgtcgccgccgcagcgcacggctcgcgacggagcagtggccgcgcgcgccgccgggaagaaggacgcggggatcgacgacgccgcgcccgcgaggcagccccgcgcactccgcggtggcgccgccaccaaggtagttagttatgaccaagttatgacgcgtgcgcgcgccttgagatcatcgtcgtctcgctgacaaattgtttatacaaaacgcacgcgcgcgcgtgtgtgcaggttgcggagcggagggatcccgtcaagacgctcgatcgcgacgccgcggaaggtggcgcgccgtccccgccggcaccgaggcaggaggacgcccgtctgccgagcatgaacggcatgccggtgaacggtgaaaacaaatctaccggcggcggcggcgcgactaaagacagcgggctgcccgcacccgcacgcgcgccccagccgtcgagccagaacagagtaccggtgaatggtgaaaacaaagctaacgtcgcctcgccgccgacgagcatagccgaggtcgcggctccggatcccgcagctaccatttccatcagtgacaaggcgccagagtccgttgtcccagccgagaaggcgccgccgtcgtccggctcaaatttcgtgccctcggcttctgctcccgggtctgacactgtcagcgacgtggaacttgaactgaagaagggtgcggtcattgtcaaagaagctccaaacccaaaggctctttcgccgcctgcagcacccgctgtacaacaagacctttgggacttcaagaaatacattggtttcgaggagcccgtggaggccaaggatgatggccgggctgttgcagatgatgcgggctccttcgaacaccaccagaatcacgattccgggcctttggcaggggagaacgtcatgaacgtggtcgtcgtggctgctgaatgttctccctggtgcaaaacaggcatggacattacctcttcagtgtcttttttctctctgttcataaaacctggcttgaattactcataagaacaaacgttgtgttgcataggtggtcttggagatgttgccggtgctttgcccaaggctttggcgaagagaggacatcgtgttatggtaccacatgctttcatttaactctgttgaatccatatgttcgaataatatcagtgagcagtataatgttattaagtgcaagacatgaaagtgttcttctgttatagagtatttcatagccaaccctggaggttaggttgttggggcctactgggtgtgggagggggtttgaaaaaagtgttggttagcagtcggatttcacaaagaacgctgataaccacgccatcagtgaagggaatgaatgtcgggtacccgatcgaccgttttgcccgacgtcaggtttacccgccctgtaggtccgaataagtagttcctatcctcagttaagtaccaaatatcggcagcgcccgtgtgtgtatttatagtactggatgatcaatttatcaacattttcagttaatggttgctatcatattcactgtaattgttagtaaacagtggatgtttgtaatgtagatgatggctaaatgtatgttgtcaagctttcatttcaatgcaatttttattgggagctagttttggggttcggttagagccaccaaaaccccagaatttctgggagttggcttgtgagagagggttttggcgagttgactttcgggattcagttagagacgctcttactagttccagtaaagaagtaaactattttctgcagacatcccaattattctgtagaaattagaagtagaaaatagttatggtatcatataaccatatattattcaaaatctagaatcatggacttggctagactttgatgatctgaaattttaaatttgatgataattgagaaatgatcctttctatcttaggttgtggtaccaaggtatggggactatgaggaagcctacgatgtcggagtccgaaaatactacaaggctgctggacaggtaagcgaaaatgcaatcaaaggggagctgaaatttcaatgcttactatcataataaatcaattttaagtgtttttttttgtcctgcaggatatggaagtgaattatttccatgcttatatcgatggagttgattttgtgttcattgacgctcctctcttccgacaccgccaggaagacatttatgggggcagcagacaggttaatcttctatatgttggtgtttgattgcactgataaactgagaacaagccaaggcctactgaccggcatatgattacacattttattttttcaggaaattatgaagcgcatgattttgttctgcaaggccgctgtcgaggtatcctctccaactcaattgacaacctattacaactatacaattatgtgtatgcatgtatttcaacagatacgtaatctcttgtgaagtgcatatatactaacaacatttcaataccttacatgcacatttggtcaagcgttatgatttaacttctaataatctattgcactgatgaacaattatcttgatgatccttggtacttcatcgttatgtttccatgttctcttcaccgattgatttggaaatagcatttccacctgccacaaacaataatatacactcctactttcatccaatgtagatattttcgcacttggcatatcatcccattaaatattattggtccatcatttttattcctctataatttgcaggttccatggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgctcaccaggttccttttctccaaatcttgatttttctctagtctctactatttactccacattgtttgaggaaactaaacgggttgcaaaattatgatggcttatgaaagttatagtcttatataggtaaatgcaccagtggtgcttgcacttgtcacgcgtgttcactttggtgcttacagttgtagacaataaaaaactagtgcaaaaacttggctgttgtgtgcaatacggtgcattttccgtatgtaggagtcaaatattgcctgtgtggcattgtattcccgtctatagctgttagaccatgcctacgtcgccattgggcccacacaccctctattacatgtgggccccacttgtcagcctatgacataaataaatggaaatttataatgaaaatgatggcctggggtcttgaaaatgggccgtcgcaggtatgctggtagccagcatgccctaatcattaatccccctatgcacttcatgtcttgtgtatgtgtgtgtgggaaagggggggggtatgtatgcttatgcttatatcctttgctccaaggctgccatcctcaacaagcccacctccagcttcaacacggccagcgccttcatgatggcccaggtgctccgcaccatcgatcgaagcggcaacgtcgtcgtcacgaccatccaccaacccaacgcacaaaatcctcaggctccgttcggtacggaggaagagaaaatgcaggaaaaaacaacttcatgggaacaaggtttggtgaacagtaaaaacatgtggaatctgaaaatgtaggtaccagaaaaaccggcctgttcggtttgcaggaaaaagcatacttgcagcagcactgtttggccctttccagtgtaaggctaaccatagtgggagtaacataactaatattatgtacttggaactcacaaacatgcttatgtggcaggcaattaaagaagagagagagagtcatagtaacatagttagataccgtatcataataaatattatgttactatgtgtcatgcatgacaataaatgagaccatctgtgatactacgttatgatattatgcactatagatgtagtatcatacactagtatcatatgcatgatactagtgtatgttactccccactatgaccagcctaacgaggggatgggcatcagtagtgattaccagtttttattattttttattcggctggacgccctactcttgtggtgcgtagcggaaaaaggcagtgctagcttcgggactccgcgagcacatgaggttctaccggattttagatttcctataaaaagtacaggctcacgcaacttttcaatggaacagaccatcaagattcctttgaaccgaacgcactgcatgcaagaattccaatgaaaaacgagccgtcaaatgttcctgcgaatttcctctataccgaacagaccctcaacatcctcaaatagtgagcatgcccctcttgtcctttccccctcgtacccaaacgccatttggcgctcttggtgttggatcatttgcctcgggcatttgccaatttggcgccgaaccatattaaagctatgactacttggtgttggatcagggacgcaaagaatcccataaatattgcaacgttcattcaaattcttaacatttgcgaggcgcttcatgatttccatctccgtcaggtctgagacatttggtcgtgtacactaaatttctcaggtcacttctcgtctaaatccgcatatgtagctcacttcaatgacttgcctttggtctagctaacgccatttggcgctcttgggcccccttgcttagcaaatttttcatatggctcgcactgcgcaagaggatttagatcacgggcagacgcgctagacgaggtcttccgcacaatgaacattgcgttctttgctctgctcttcccggagacacttgtgatcttattacgagttgtgccatttcaaacatctgtctctccatggtcgctccagccatagatgccttgttctctgaatggtgggtttcagctaggaacagggtgccaccttcgacaagaagttgcatagtttggtcgtcttgactgcttggtcgatttggaaggaacgcaacataagagtctttgaaggcaaagctaatttctttgatcaagttattagccagatcaaatgtgatgtattctactggtacaaggccggggctatttgctttgagtcactttttagctaaggccgcttgggctaagcgcttggggtcgtttttgctcaactgtagcctgaactttctggactttgtaattttttttatcctctataatgatcacatacagctctcctgcatggttcgaaaaggaaaaatgtgaacatgtgtggcaagtttaagcacaacccgtgcatttacctcaaagttatacaacactgacatgctgaattacattttttttgaggatctgacatgccgaattacatgctttggacagttattcatttcttcggtacaccattggctaattatttctcttgacagttgctgaattagtacatgctttggtcgcagttattcctttgttcggtactctgttgggctaattatttctcttgattgatgttgcatgcagggccgtggcccagtagatgagttcccgttcaccgagttgcctgagcactacctggaacacttcagactgtacgaccccgtgggtggtgaacacgccaactacttcgccgccggcctgaagatggcggaccaggttgtcgtcgtgagcccggggtacctgtgggagctgaagacggtggagggcggctgggggcttcacgacatcatacggcagaacgactggaagacccgcggcatcgtgaacggcatcgacaacatggagtggaaccccgaggtggacgtccacctcaagtcggacggctacaccaacttctccctggggacgctggactccggcaagcggcagtgcaaggaggccctgcagcgggagctgggcctgcaggtccgcggcgacgtgccgctgctcggcttcatcgggcgcctggacgggcagaagggcgtggagatcatcgcggacgcgatgccctggatcgtgagccaggacgtgcagctggtcatgctgggcaccgggcgccacgacctggagggcatgctgcggcacttcgagcgggagcaccacgacaaggtgcgcgggtgggtggggttctccgtgcggctggcgcaccggatcacggccggcgccgacgcgctcctcatgccctcccggttcgagccgtgcggactgaaccagctctacgccatggcctacggcaccgtccccgtcgtgcatgccgtcggcggcctgagggacaccgtgccgccgttcgaccccttcaaccactccgggctcgggtggacgttcgaccgcgcagaggcgcagaagctgatcgaggcgctcgggcactgcctccgcacctaccgggactacaaggagagctggagggggctccaggagcgcggcatgtcgcaggacttcagctgggagcatgccgccaagctctacgaggacgtcctcgtcaaggccaagtaccagtggtga

SEQ ID NO: 13 - DNA, 2397 bp, Triticum aestivum
  misc_feature (1)..(2397): Starch Synthase II Genome B CDS Wild Type
atgtcgtcggcggtcgcgtccgccgcgtccttcctcgcgctcgcgtccgcctcccccgggagatcacggaggaggacgagggtgagcgcgtcgccaccccacaccggggctggcaggttgcactggccgccgtcgccgccgcagcgcacggctcgcgacggagcagtggccgcgcgcgccgccgggaagaaggacgcggggatcgacgacgccgcgcccgcgaggcagccccgcgcactccgcggtggcgccgccaccaaggttgcggagcggagggatcccgtcaagacgctcgatcgcgacgccgcggaaggtggcgcgccgtccccgccggcaccgaggcaggaggacgcccgtctgccgagcatgaacggcatgccggtgaacggtgaaaacaaatctaccggcggcggcggcgcgactaaagacagcgggctgcccgcacccgcacgcgcgccccagccgtcgagccagaacagagtaccggtgaatggtgaaaacaaagctaacgtcgcctcgccgccgacgagcatagccgaggtcgcggctccggatcccgcagctaccatttccatcagtgacaaggcgccagagtccgttgtcccagccgagaaggcgccgccgtcgtccggctcaaatttcgtgccctcggcttctgctcccgggtctgacactgtcagcgacgtggaacttgaactgaagaagggtgcggtcattgtcaaagaagctccaaacccaaaggctctttcgccgcctgcagcacccgctgtacaacaagacctttgggacttcaagaaatacattggtttcgaggagcccgtggaggccaaggatgatggccgggctgttgcagatgatgcgggctccttcgaacaccaccagaatcacgattccgggcctttggcaggggagaacgtcatgaacgtggtcgtcgtggctgctgaatgttctccctggtgcaaaacaggtggtcttggagatgttgccggtgctttgcccaaggctttggcgaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctctcttccgacaccgccaggaagacatttatgggggcagcagacaggaaattatgaagcgcatgattttgttctgcaaggccgctgtcgaggttccatggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgctcaccagggccgtggcccagtagatgagttcccgttcaccgagttgcctgagcactacctggaacacttcagactgtacgaccccgtgggtggtgaacacgccaactacttcgccgccggcctgaagatggcggaccaggttgtcgtcgtgagcccggggtacctgtgggagctgaagacggtggagggcggctgggggcttcacgacatcatacggcagaacgactggaagacccgcggcatcgtgaacggcatcgacaacatggagtggaaccccgaggtggacgtccacctcaagtcggacggctacaccaacttctccctggggacgctggactccggcaagcggcagtgcaaggaggccctgcagcgggagctgggcctgcaggtccgcggcgacgtgccgctgctcggcttcatcgggcgcctggacgggcagaagggcgtggagatcatcgcggacgcgatgccctggatcgtgagccaggacgtgcagctggtcatgctgggcaccgggcgccacgacctggagggcatgctgcggcacttcgagcgggagcaccacgacaaggtgcgcgggtgggtggggttctccgtgcggctggcgcaccggatcacggccggcgccgacgcgctcctcatgccctcccggttcgagccgtgcggactgaaccagctctacgccatggcctacggcaccgtccccgtcgtgcatgccgtcggcggcctgagggacaccgtgccgccgttcgaccccttcaaccactccgggctcgggtggacgttcgaccgcgcagaggcgcagaagctgatcgaggcgctcgggcactgcctccgcacctaccgggactacaaggagagctggagggggctccaggagcgcggcatgtcgcaggacttcagctgggagcatgccgccaagctctacgaggacgtcctcgtcaaggccaagtaccagtggtga

SEQ ID NO: 14 - protein, 798 aa, Triticum aestivum
  MISC_FEATURE (1)..(798): Starch Synthase II Genome B Protein Wild Type
MSSAVASAASFLALASASPGRSRRRTRVSASPPHTGAGRLHWPPSPPQRTARDGAVAARAAGKKDAGIDDAAPARQPRALRGGAATKVAERRDPVKTLDRDAAEGGAPSPPAPRQEDARLPSMNGMPVNGENKSTGGGGATKDSGLPAPARAPQPSSQNRVPVNGENKANVASPPTSIAEVAAPDPAATISISDKAPESVVPAEKAPPSSGSNFVPSASAPGSDTVSDVELELKKGAVIVKEAPNPKALSPPAAPAVQQDLWDFKKYIGFEEPVEAKDDGRAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPLFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDVHLKSDGYTNFSLGTLDSGKRQCKEALQRELGLQVRGDVPLLGFIGRLDGQKGVEIIADAMPWIVSQDVQLVMLGTGRHDLEGMLRHFEREHHDKVRGWVGFSVRLAHRITAGADALLMPSRFEPCGLNQLYAMAYGTVPVVHAVGGLRDTVPPFDPFNHSGLGWTFDRAEAQKLIEALGHCLRTYRDYKESWRGLQERGMSQDFSWEHAAKLYEDVLVKAKYQW

SEQ ID NO: 15 - DNA, 2397 bp, Artificial Sequence
  RJ 435 Mutant Starch Synthase II Genome B CDS
atgtcgtcggcggtcgcgtccgccgcgtccttcctcgcgctcgcgtccgcctcccccgggagatcacggaggaggacgagggtgagcgcgtcgccaccccacaccggggctggcaggttgcactggccgccgtcgccgccgcagcgcacggctcgcgacggagcagtggccgcgcgcgccgccgggaagaaggacgcggggatcgacgacgccgcgcccgcgaggcagccccgcgcactccgcggtggcgccgccaccaaggttgcggagcggagggatcccgtcaagacgctcgatcgcgacgccgcggaaggtggcgcgccgtccccgccggcaccgaggcaggaggacgcccgtctgccgagcatgaacggcatgccggtgaacggtgaaaacaaatctaccggcggcggcggcgcgactaaagacagcgggctgcccgcacccgcacgcgcgccccagccgtcgagccagaacagagtaccggtgaatggtgaaaacaaagctaacgtcgcctcgccgccgacgagcatagccgaggtcgcggctccggatcccgcagctaccatttccatcagtgacaaggcgccagagtccgttgtcccagccgagaaggcgccgccgtcgtccggctcaaatttcgtgccctcggcttctgctcccgggtctgacactgtcagcgacgtggaacttgaactgaagaagggtgcggtcattgtcaaagaagctccaaacccaaaggctctttcgccgcctgcagcacccgctgtacaacaagacctttgggacttcaagaaatacattggtttcgaggagcccgtggaggccaaggatgatggccgggctgttgcagatgatgcgggctccttcgaacaccaccagaatcacgattccgggcctttggcaggggagaacgtcatgaacgtggtcgtcgtggctgctgaatgttctccctggtgcaaaacaggtggtcttggagatgttgccggtgctttgcccaaggctttggcgaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctctcttccgacaccgccaggaagacatttatgggggcagcagacaggaaattatgaagcgcatgattttgttctgcaaggccgctgtcgaggttccatggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgctcaccagggccgtggcccagtagatgagttcccgttcaccgagttgcctgagcactacctggaacacttcagactgtacgaccccgtgggtggtgaacacgccaactacttcgccgccggcctgaagatggcggaccaggttgtcgtcgtgagcccggggtacctgtgggagctgaagacggtggagggcggctgggggcttcacgacatcatacggcagaacgactggaagacccgcggcatcgtgaacggcatcgacaacatggagtggaaccccgaggtggacgtccacctcaagtcggacggctacaccaacttctccctggggacgctggactccggcaagcggcagtgcaaggaggccctgcagcgggagctgggcctgcaggtccgcggcgacgtgccgctgctcggcttcatcgggcgcctggacgggcagaagggcgtggagatcatcgcggacgcgatgccctggatcgtgagccaggacgtgcagctggtcatgctggacaccgggcgccacgacctggagggcatgctgcggcacttcgagcgggagcaccacgacaaggtgcgcgggtgggtggggttctccgtgcggctggcgcaccggatcacggccggcgccgacgcgctcctcatgccctcccggttcgagccgtgcggactgaaccagctctacgccatggcctacggcaccgtccccgtcgtgcatgccgtcggcggcctgagggacaccgtgccgccgttcgaccccttcaaccactccgggctcgggtggacgttcgaccgcgcagaggcgcagaagctgatcgaggcgctcgggcactgcctccgcacctaccgggactacaaggagagctggagggggctccaggagcgcggcatgtcgcaggacttcagctgggagcatgccgccaagctctacgaggacgtcctcgtcaaggccaagtaccagtggtga

SEQ ID NO: 16 - protein, 798 aa, Artificial Sequence
  RJ 435 Mutant Starch Synthase II Genome B Protein
MSSAVASAASFLALASASPGRSRRRTRVSASPPHTGAGRLHWPPSPPQRTARDGAVAARAAGKKDAGIDDAAPARQPRALRGGAATKVAERRDPVKTLDRDAAEGGAPSPPAPRQEDARLPSMNGMPVNGENKSTGGGGATKDSGLPAPARAPQPSSQNRVPVNGENKANVASPPTSIAEVAAPDPAATISISDKAPESVVPAEKAPPSSGSNFVPSASAPGSDTVSDVELELKKGAVIVKEAPNPKALSPPAAPAVQQDLWDFKKYIGFEEPVEAKDDGRAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPLFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDVHLKSDGYTNFSLGTLDSGKRQCKEALQRELGLQVRGDVPLLGFIGRLDGQKGVEIIADAMPWIVSQDVQLVMLDTGRHDLEGMLRHFEREHHDKVRGWVGFSVRLAHRITAGADALLMPSRFEPCGLNQLYAMAYGTVPVVHAVGGLRDTVPPFDPFNHSGLGWTFDRAEAQKLIEALGHCLRTYRDYKESWRGLQERGMSQDFSWEHAAKLYEDVLVKAKYQW

SEQ ID NO: 17 - DNA, 2397 bp, Artificial Sequence
  RJ 597 Mutant Starch Synthase II Genome B CDS
atgtcgtcggcggtcgcgtccgccgcgtccttcctcgcgctcgcgtccgcctcccccgggagatcacggaggaggacgagggtgagcgcgtcgccaccccacaccggggctggcaggttgcactggccgccgtcgccgccgcagcgcacggctcgcgacggagcagtggccgcgcgcgccgccgggaagaaggacgcggggatcgacgacgccgcgcccgcgaggcagccccgcgcactccgcggtggcgccgccaccaaggttgcggagcggagggatcccgtcaagacgctcgatcgcgacgccgcggaaggtggcgcgccgtccccgccggcaccgaggcaggaggacgcccgtctgccgagcatgaacggcatgccggtgaacggtgaaaacaaatctaccggcggcggcggcgcgactaaagacagcgggctgcccgcacccgcacgcgcgccccagccgtcgagccagaacagagtaccggtgaatggtgaaaacaaagctaacgtcgcctcgccgccgacgagcatagccgaggtcgcggctccggatcccgcagctaccatttccatcagtgacaaggcgccagagtccgttgtcccagccgagaaggcgccgccgtcgtccggctcaaatttcgtgccctcggcttctgctcccgggtctgacactgtcagcgacgtggaacttgaactgaagaagggtgcggtcattgtcaaagaagctccaaacccaaaggctctttcgccgcctgcagcacccgctgtacaacaagacctttgggacttcaagaaatacattggtttcgaggagcccgtggaggccaaggatgatggccgggctgttgcagatgatgcgggctccttcgaacaccaccagaatcacgattccgggcctttggcaggggagaacgtcatgaacgtggtcgtcgtggctgctgaatgttctccctggtgcaaaacaggtggtcttggagatgttgccggtgctttgcccaaggctttggcgaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctctcttccgacaccgccaggaagacatttatgggggcagcagacaggaaattatgaagcgcatgattttgttctgcaaggccgctgtcgaggttccatggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgctcaccagggccgtggcccagtagatgagttcccgttcaccgagttgcctgagcactacctggaacacttcagactgtacgaccccgtgggtggtgaacacgccaactacttcgccgccggcctgaagatggcggaccaggttgtcgtcgtgagcccggggtacctgtgggagctgaagacggtggagggcggctgggggcttcacgacatcatacggcagaacgactggaagacccgcggcatcgtgaacggcatcgacaacatggagtggaaccccgaggtggacgtccacctcaagtcggacggctacaccaacttctccctggggacgctggactccggcaagcggcagtacaaggaggccctgcagcgggagctgggcctgcaggtccgcggcgacgtgccgctgctcggcttcatcgggcgcctggacgggcagaagggcgtggagatcatcgcggacgcgatgccctggatcgtgagccaggacgtgcagctggtcatgctgggcaccgggcgccacgacctggagggcatgctgcggcacttcgagcgggagcaccacgacaaggtgcgcgggtgggtggggttctccgtgcggctggcgcaccggatcacggccggcgccgacgcgctcctcatgccctcccggttcgagccgtgcggactgaaccagctctacgccatggcctacggcaccgtccccgtcgtgcatgccgtcggcggcctgagggacaccgtgccgccgttcgaccccttcaaccactccgggctcgggtggacgttcgaccgcgcagaggcgcagaagctgatcgaggcgctcgggcactgcctccgcacctaccgggactacaaggagagctggagggggctccaggagcgcggcatgtcgcaggacttcagctgggagcatgccgccaagctctacgaggacgtcctcgtcaaggccaagtaccagtggtga

SEQ ID NO: 18 - protein, 798 aa, Artificial Sequence
  RJ 597 Mutant Starch Synthase II Genome B Protein
MSSAVASAASFLALASASPGRSRRRTRVSASPPHTGAGRLHWPPSPPQRTARDGAVAARAAGKKDAGIDDAAPARQPRALRGGAATKVAERRDPVKTLDRDAAEGGAPSPPAPRQEDARLPSMNGMPVNGENKSTGGGGATKDSGLPAPARAPQPSSQNRVPVNGENKANVASPPTSIAEVAAPDPAATISISDKAPESVVPAEKAPPSSGSNFVPSASAPGSDTVSDVELELKKGAVIVKEAPNPKALSPPAAPAVQQDLWDFKKYIGFEEPVEAKDDGRAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPLFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDVHLKSDGYTNFSLGTLDSGKRQYKEALQRELGLQVRGDVPLLGFIGRLDGQKGVEIIADAMPWIVSQDVQLVMLGTGRHDLEGMLRHFEREHHDKVRGWVGFSVRLAHRITAGADALLMPSRFEPCGLNQLYAMAYGTVPVVHAVGGLRDTVPPFDPFNHSGLGWTFDRAEAQKLIEALGHCLRTYRDYKESWRGLQERGMSQDFSWEHAAKLYEDVLVKAKYQW

SEQ ID NO: 19 - DNA, 2397 bp, Artificial Sequence
  RJ 269 Mutant Starch Synthase II Genome B CDS
atgtcgtcggcggtcgcgtccgccgcgtccttcctcgcgctcgcgtccgcctcccccgggagatcacggaggaggacgagggtgagcgcgtcgccaccccacaccggggctggcaggttgcactggccgccgtcgccgccgcagcgcacggctcgcgacggagcagtggccgcgcgcgccgccgggaagaaggacgcggggatcgacgacgccgcgcccgcgaggcagccccgcgcactccgcggtggcgccgccaccaaggttgcggagcggagggatcccgtcaagacgctcgatcgcgacgccgcggaaggtggcgcgccgtccccgccggcaccgaggcaggaggacgcccgtctgccgagcatgaacggcatgccggtgaacggtgaaaacaaatctaccggcggcggcggcgcgactaaagacagcgggctgcccgcacccgcacgcgcgccccagccgtcgagccagaacagagtaccggtgaatggtgaaaacaaagctaacgtcgcctcgccgccgacgagcatagccgaggtcgcggctccggatcccgcagctaccatttccatcagtgacaaggcgccagagtccgttgtcccagccgagaaggcgccgccgtcgtccggctcaaatttcgtgccctcggcttctgctcccgggtctgacactgtcagcgacgtggaacttgaactgaagaagggtgcggtcattgtcaaagaagctccaaacccaaaggctctttcgccgcctgcagcacccgctgtacaacaagacctttgggacttcaagaaatacattggtttcgaggagcccgtggaggccaaggatgatggccgggctgttgcagatgatgcgggctccttcgaacaccaccagaatcacgattccgggcctttggcaggggagaacgtcatgaacgtggtcgtcgtggctgctgaatgttctccctggtgcaaaacaggtggtcttggagatgttgccggtgctttgcccaaggctttggcgaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctctcttccgacaccgccaggaagacatttatgggggcagcagacaggaaattatgaagcgcatgattttgttctgcaaggccgctgtcgaggttccatggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgctcaccagggccgtggcccagtagatgagttcccgttcaccgagttgcctgagcactacctggaacacttcagactgtacgaccccgtgggtggtgaacacgccaactacttcgccgccggcctgaagatggcggaccaggttgtcgtcgtgagcccggggtacctgtgggagctgaagacggtggagggcggctgggggcttcacgacatcatacggcagaacgactggaagacccgcggcatcgtgaacgacatcgacaacatggagtggaaccccgaggtggacgtccacctcaagtcggacggctacaccaacttctccctggggacgctggactccggcaagcggcagtgcaaggaggccctgcagcgggagctgggcctgcaggtccgcggcgacgtgccgctgctcggcttcatcgggcgcctggacgggcagaagggcgtggagatcatcgcggacgcgatgccctggatcgtgagccaggacgtgcagctggtcatgctgggcaccgggcgccacgacctggagggcatgctgcggcacttcgagcgggagcaccacgacaaggtgcgcgggtgggtggggttctccgtgcggctggcgcaccggatcacggccggcgccgacgcgctcctcatgccctcccggttcgagccgtgcggactgaaccagctctacgccatggcctacggcaccgtccccgtcgtgcatgccgtcggcggcctgagggacaccgtgccgccgttcgaccccttcaaccactccgggctcgggtggacgttcgaccgcgcagaggcgcagaagctgatcgaggcgctcgggcactgcctccgcacctaccgggactacaaggagagctggagggggctccaggagcgcggcatgtcgcaggacttcagctgggagcatgccgccaagctctacgaggacgtcctcgtcaaggccaagtaccagtggtga

SEQ ID NO: 20 - protein, 798 aa, Artificial Sequence
  RJ 269 Mutant Starch Synthase II Genome B Protein
MSSAVASAASFLALASASPGRSRRRTRVSASPPHTGAGRLHWPPSPPQRTARDGAVAARAAGKKDAGIDDAAPARQPRALRGGAATKVAERRDPVKTLDRDAAEGGAPSPPAPRQEDARLPSMNGMPVNGENKSTGGGGATKDSGLPAPARAPQPSSQNRVPVNGENKANVASPPTSIAEVAAPDPAATISISDKAPESVVPAEKAPPSSGSNFVPSASAPGSDTVSDVELELKKGAVIVKEAPNPKALSPPAAPAVQQDLWDFKKYIGFEEPVEAKDDGRAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPLFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNDIDNMEWNPEVDVHLKSDGYTNFSLGTLDSGKRQCKEALQRELGLQVRGDVPLLGFIGRLDGQKGVEIIADAMPWIVSQDVQLVMLGTGRHDLEGMLRHFEREHHDKVRGWVGFSVRLAHRITAGADALLMPSRFEPCGLNQLYAMAYGTVPVVHAVGGLRDTVPPFDPFNHSGLGWTFDRAEAQKLIEALGHCLRTYRDYKESWRGLQERGMSQDFSWEHAAKLYEDVLVKAKYQW

SEQ ID NO: 21 - DNA, 2397 bp, Artificial Sequence; identical to SEQ ID NO: 19
  RJ 521 Mutant Starch Synthase II Genome B CDS

SEQ ID NO: 22 - protein, 798 aa, Artificial Sequence; identical to SEQ ID NO: 20
  RJ 521 Mutant Starch Synthase II Genome B Protein

SEQ ID NO: 23 - DNA, 2397 bp, Artificial Sequence; identical to SEQ ID NO: 19
  RJ 63 Mutant Starch Synthase II Genome B CDS

SEQ ID NO: 24 - protein, 798 aa, Artificial Sequence; identical to SEQ ID NO: 20
  RJ 63 Mutant Starch Synthase II Genome B Protein

SEQ ID NO: 25 - DNA, 2397 bp, Artificial Sequence
  RJ 102 Mutant Starch Synthase II Genome B CDS
atgtcgtcggcggtcgcgtccgccgcgtccttcctcgcgctcgcgtccgcctcccccgggagatcacggaggaggacgagggtgagcgcgtcgccaccccacaccggggctggcaggttgcactggccgccgtcgccgccgcagcgcacggctcgcgacggagcagtggccgcgcgcgccgccgggaagaaggacgcggggatcgacgacgccgcgcccgcgaggcagccccgcgcactccgcggtggcgccgccaccaaggttgcggagcggagggatcccgtcaagacgctcgatcgcgacgccgcggaaggtggcgcgccgtccccgccggcaccgaggcaggaggacgcccgtctgccgagcatgaacggcatgccggtgaacggtgaaaacaaatctaccggcggcggcggcgcgactaaagacagcgggctgcccgcacccgcacgcgcgccccagccgtcgagccagaacagagtaccggtgaatggtgaaaacaaagctaacgtcgcctcgccgccgacgagcatagccgaggtcgcggctccggatcccgcagctaccatttccatcagtgacaaggcgccagagtccgttgtcccagccgagaaggcgccgccgtcgtccggctcaaatttcgtgccctcggcttctgctcccgggtctgacactgtcagcgacgtggaacttgaactgaagaagggtgcggtcattgtcaaagaagctccaaacccaaaggctctttcgtcgcctgcagcacccgctgtacaacaagacctttgggacttcaagaaatacattggtttcgaggagcccgtggaggccaaggatgatggccgggctgttgcagatgatgcgggctccttcgaacaccaccagaatcacgattccgggcctttggcaggggagaacgtcatgaacgtggtcgtcgtggctgctgaatgttctccctggtgcaaaacaggtggtcttggagatgttgccggtgctttgcccaaggctttggcgaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctctcttccgacaccgccaggaagacatttatgggggcagcagacaggaaattatgaagcgcatgattttgttctgcaaggccgctgtcgaggttccatggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgctcaccagggccgtggcccagtagatgagttcccgttcaccgagttgcctgagcactacctggaacacttcagactgtacgaccccgtgggtggtgaacacgccaactacttcgccgccggcctgaagatggcggaccaggttgtcgtcgtgagcccggggtacctgtgggagctgaagacggtggagggcggctgggggcttcacgacatcatacggcagaacgactggaagacccgcggcatcgtgaacggcatcgacaacatggagtggaaccccgaggtggacgtccacctcaagtcggacggctacaccaacttctccctggggacgctggactccggcaagcggcagtgcaaggaggccctgcagcgggagctgggcctgcaggtccgcggcgacgtgccgctgctcggcttcatcgggcgcctggacgggcagaagggcgtggagatcatcgcggacgcgatgccctggatcgtgagccaggacgtgcagctggtcatgctgggcaccgggcgccacgacctggagggcatgctgcggcacttcgagcgggagcaccacgacaaggtgcgcgggtgggtggggttctccgtgcggctggcgcaccggatcacggccggcgccgacgcgctcctcatgccctcccggttcgagccgtgcggactgaaccagctctacgccatggcctacggcaccgtccccgtcgtgcatgccgtcggcggcctgagggacaccgtgccgccgttcgaccccttcaaccactccgggctcgggtggacgttcgaccgcgcagaggcgcagaagctgatcgaggcgctcgggcactgcctccgcacctaccgggactacaaggagagctggagggggctccaggagcgcggcatgtcgcaggacttcagctgggagcatgccgccaagctctacgaggacgtcctcgtcaaggccaagtaccagtggtga

SEQ ID NO: 26 - protein, 798 aa, Artificial Sequence
  RJ 102 Mutant Starch Synthase II Genome B Protein
MSSAVASAASFLALASASPGRSRRRTRVSASPPHTGAGRLHWPPSPPQRTARDGAVAARAAGKKDAGIDDAAPARQPRALRGGAATKVAERRDPVKTLDRDAAEGGAPSPPAPRQEDARLPSMNGMPVNGENKSTGGGGATKDSGLPAPARAPQPSSQNRVPVNGENKANVASPPTSIAEVAAPDPAATISISDKAPESVVPAEKAPPSSGSNFVPSASAPGSDTVSDVELELKKGAVIVKEAPNPKALSSPAAPAVQQDLWDFKKYIGFEEPVEAKDDGRAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPLFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDVHLKSDGYTNFSLGTLDSGKRQCKEALQRELGLQVRGDVPLLGFIGRLDGQKGVEIIADAMPWIVSQDVQLVMLGTGRHDLEGMLRHFEREHHDKVRGWVGFSVRLAHRITAGADALLMPSRFEPCGLNQLYAMAYGTVPVVHAVGGLRDTVPPFDPFNHSGLGWTFDRAEAQKLIEALGHCLRTYRDYKESWRGLQERGMSQDFSWEHAAKLYEDVLVKAKYQW

SEQ ID NO: 27 - DNA, 2397 bp, Artificial Sequence
  RJ 416 Mutant Starch Synthase II Genome B CDS
atgtcgtcggcggtcgcgtccgccgcgtccttcctcgcgctcgcgtccgcctcccccgggagatcacggaggaggacgagggtgagcgcgtcgccaccccacaccggggctggcaggttgcactggccgccgtcgccgccgcagcgcacggctcgcgacggagcagtggccgcgcgcgccgccgggaagaaggacgcggggatcgacgacgccgcgcccgcgaggcagccccgcgcactccgcggtggcgccgccaccaaggttgcggagcggagggatcccgtcaagacgctcgatcgcgacgccgcggaaggtggcgcgccgtccccgccggcaccgaggcaggaggacgcccgtctgccgagcatgaacggcatgccggtgaacggtgaaaacaaatctaccggcggcggcggcgcgactaaagacagcgggctgcccgcacccgcacgcgcgccccagccgtcgagccagaacagagtaccggtgaatggtgaaaacaaagctaacgtcgcctcgccgccgacgagcatagccgaggtcgcggctccggatcccgcagctaccatttccatcagtgacaaggcgccagagtccgttgtcccagccgagaaggcgccgccgtcgtccggctcaaatttcgtgccctcggcttctgctcccgggtctgacactgtcagcgacgtggaacttgaactgaagaagggtgcggtcattgtcaaagaagctccaaacccaaaggctctttcgccgcctgcagcacccgctgtacaacaagacctttgggacttcaagaaatacattggtttcgaggagcccgtggaggccaaggatgatggccgggctgttgcagatgatgcgggctccttcgaacaccaccagaatcacgattccgggcctttggcaggggagaacgtcatgaacgtggtcgtcgtggctgctgaatgttctccctgatgcaaaacaggtggtcttggagatgttgccggtgctttgcccaaggctttggcgaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctctcttccgacaccgccaggaagacatttatgggggcagcagacaggaaattatgaagcgcatgattttgttctgcaaggccgctgtcgaggttccatggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgctcaccagggccgtggcccagtagatgagttcccgttcaccgagttgcctgagcactacctggaacacttcagactgtacgaccccgtgggtggtgaacacgccaactacttcgccgccggcctgaagatggcggaccaggttgtcgtcgtgagcccggggtacctgtgggagctgaagacggtggagggcggctgggggcttcacgacatcatacggcagaacgactggaagacccgcggcatcgtgaacggcatcgacaacatggagtggaaccccgaggtggacgtccacctcaagtcggacggctacaccaacttctccctggggacgctggactccggcaagcggcagtgcaaggaggccctgcagcgggagctgggcctgcaggtccgcggcgacgtgccgctgctcggcttcatcgggcgcctggacgggcagaagggcgtggagatcatcgcggacgcgatgccctggatcgtgagccaggacgtgcagctggtcatgctgggcaccgggcgccacgacctggagggcatgctgcggcacttcgagcgggagcaccacgacaaggtgcgcgggtgggtggggttctccgtgcggctggcgcaccggatcacggccggcgccgacgcgctcctcatgccctcccggttcgagccgtgcggactgaaccagctctacgccatggcctacggcaccgtccccgtcgtgcatgccgtcggcggcctgagggacaccgtgccgccgttcgaccccttcaaccactccgggctcgggtggacgttcgaccgcgcagaggcgcagaagctgatcgaggcgctcgggcactgcctccgcacctaccgggactacaaggagagctggagggggctccaggagcgcggcatgtcgcaggacttcagctgggagcatgccgccaagctctacgaggacgtcctcgtcaaggccaagtaccagtggtga

SEQ ID NO: 28 - protein, 797 aa, Artificial Sequence
  RJ 416 Mutant Starch Synthase II Genome B Protein
MSSAVASAASFLALASASPGRSRRRTRVSASPPHTGAGRLHWPPSPPQRTARDGAVAARAAGKKDAGIDDAAPARQPRALRGGAATKVAERRDPVKTLDRDAAEGGAPSPPAPRQEDARLPSMNGMPVNGENKSTGGGGATKDSGLPAPARAPQPSSQNRVPVNGENKANVASPPTSIAEVAAPDPAATISISDKAPESVVPAEKAPPSSGSNFVPSASAPGSDTVSDVELELKKGAVIVKEAPNPKALSPPAAPAVQQDLWDFKKYIGFEEPVEAKDDGRAVADDAGSFEHHQNHDSGPLAGENVMNVVVVAAECSPCKTGGLGDVAGALPKALAKRGHRVMVVVPRYGDYEEAYDVGVRKYYKAAGQDMEVNYFHAYIDGVDFVFIDAPLFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDVHLKSDGYTNFSLGTLDSGKRQCKEALQRELGLQVRGDVPLLGFIGRLDGQKGVEIIADAMPWIVSQDVQLVMLGTGRHDLEGMLRHFEREHHDKVRGWVGFSVRLAHRITAGADALLMPSRFEPCGLNQLYAMAYGTVPVVHAVGGLRDTVPPFDPFNHSGLGWTFDRAEAQKLIEALGHCLRTYRDYKESWRGLQERGMSQDFSWEHAAKLYEDVLVKAKYQW

SEQ ID NO: 29 - DNA, 2397 bp, Artificial Sequence
  RJ 514 Mutant Starch Synthase II Genome B CDS
atgtcgtcggcggtcgcgtccgccgcgtccttcctcgcgctcgcgtccgcctcccccgggagatcacggaggaggacgagggtgagcgcgtcgccaccccacaccggggctggcaggttgcactggccgccgtcgccgccgcagcgcacggctcgcgacggagcagtggccgcgcgcgccgccgggaagaaggacgcggggatcgacgacgccgcgcccgcgaggcagccccgcgcactccgcggtggcgccgccaccaaggttgcggagcggagggatcccgtcaagacgctcgatcgcgacgccgcggaaggtggcgcgccgtccccgccggcaccgaggcaggaggacgcccgtctgccgagcatgaacggcatgccggtgaacggtgaaaacaaatctaccggcggcggcggcgcgactaaagacagcgggctgcccgcacccgcacgcgcgccccagccgtcgagccagaacagagtaccggtgaatggtgaaaacaaagctaacgtcgcctcgccgccgacgagcatagccgaggtcgcggctccggatcccgcagctaccatttccatcagtgacaaggcgccagagtccgttgtcccagccgagaaggcgccgccgtcgtccggctcaaatttcgtgccctcggcttctgctcccgggtctgacactgtcagcgacgtggaacttgaactgaagaagggtgcggtcattgtcaaagaagctccaaacccaaaggctctttcgccgcctgcagcacccgctgtacaacaagacctttgggacttcaagaaatacattggtttcgaggagcccgtggaggccaaggatgatggccgggctgttgcagatgatgcgggctccttcgaacaccaccagaatcacgattccgggcctttggcaggggagaacgtcatgaacgtggtcgtcgtggctgctgaatgttctccctggtgcaaaacaggtggtcttggagatgttgccggtgctttgcccaaggctttggcgaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctctcttccgacaccgccaggaagacatttatgggggcagcagacaggaaattatgaagcgcatgattttgttctgcaaggccgctgtcgaggttccatggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgctcaccagggccgtggcccagtagatgagttcccgttcaccgagttgcctgagcactacctggaacacttcagactgtacgaccccgtgggtggtgaacacgccaactacttcgccgccggcctgaagatggcggaccaggttgtcgtcgtgagcccggggtacctgtgggagctgaagacggtggagggcggctgggggcttcacgacatcatacggcagaacgactggaagacccgcggcatcgtgaacggcatcgacaacatggagtggaaccccgaggtggacgtccacctcaagtcggacggctacaccaacttctccctggggacgctggactccggcaagcggcagtgcaaggaggccctgcagcgggagctgggcttgcaggtccgcggcgacgtgccgctgctcggcttcatcgggcgcctggacgggcagaagggcgtggagatcatcgcggacgcgatgccctggatcgtgagccaggacgtgcagctggtcatgctgggcaccgggcgccacgacctggagggcatgctgcggcacttcgagcgggagcaccacgacaaggtgcgcgggtgggtggggttctccgtgcggctggcgcaccggatcacggccggcgccgacgcgctcctcatgccctcccggttcgagccgtgcggactgaaccagctctacgccatggcctacggcaccgtccccgtcgtgcatgccgtcggcggcctgagggacaccgtgccgccgttcgaccccttcaaccactccgggctcgggtggacgttcgaccgcgcagaggcgcagaagctgatcgaggcgctcgggcactgcctccgcacctaccgggactacaaggagagctggagggggctccaggagcgcggcatgtcgcaggacttcagctgggagcatgccgccaagctctacgaggacgtcctcgtcaaggccaagtaccagtggtga

SEQ ID NO: 30 - protein, 798 aa, Artificial Sequence; identical to SEQ ID NO: 14
  RJ 514 Mutant Starch Synthase II Genome B Protein

SEQ ID NO: 31 - DNA, 7010 bp, Triticum aestivum
  misc_feature (1)..(7010): Starch Synthase II Genome D Genomic Wild Type
gggggccgttcgtacgtacccgcccctcgtgtaaagccgccgccgtcgtcgccgtcccccgctcgcggccatttcttcggcctgaccccgttcgtttacccccacacagagcacactccagtccagtccagcccactgccaccgcgctactctccactcccactgccaccacctccgcctgcgccgcgctctgggcggaccaacccgcgaaccgtaccatctcccgccccgatccatgtcgtcggcggtcgcgtccgccgcatccttcctcgcgctcgcgtcagcctcccccgggagatcacgcaggcgggcgagggtgagcgcgcagccaccccacgccggggccggcaggttgcactggccgccgtggccgccgcagcgcacggctcgcgacggagctgtggcggcgctcgccgccgggaagaaggacgcggggatcgacgacgccgccgcgtccgtgaggcagccccgcgcactccgcggtggcgccgccaccaaggtagttagttatgaccaagttatgacgcgtgcgcgcgcctcgagatcatcgtcgtctcgctcacgaattgtttatttatacaaaacgcacgcccgcgtgtgcaggtcgcggagcgaagggatcccgtcaagacgctcgaccgcgacgccgcggaaggcggcgggccgtccccgccggcagcgaggcaggacgccgcccgtccgccgagtatgaacggcatgccggtgaacggcgagaacaaatctaccggcggcggcggcgcgactaaagacagcgggctgcccacgcccgcacgcgcgccccatccgtcgacccagaacagagcaccggtgaacggtgaaaacaaagctaacgtcgcctcgccgccgacgagcatagccgaggccgcggcttcggattccgcagctaccatttccatcagcgacaaggcgccggagtccgttgtcccagctgagaagacgccgccgtcgtccggctcaaatttcgagtcctcggcctctgctcccgggtctgacactgtcagcgacgtggaacaagaactgaagaagggtgcggtcgttgtcgaagaagctccaaagccaaaggctctttcgccgcctgcagcccccgctgtacaagaagacctttgggatttcaagaaatacattggtttcgaggagcccgtggaggccaaggatgatggccgggctgtcgcagatgatgcgggctcctttgaacaccaccagaatcacgactccggacctttggcaggggagaatgtcatgaacgtggtcgtcgtggctgctgagtgttctccctggtgcaaaacaggcatggacattacctcttcagtctctcttcctgttgttcataaaactttgctcgaattactcataagaacaaacattgtgttgcataggtggtctgggagatgttgcgggtgctctgcccaaggctttggcaaagagaggacatcgtgttatggtactacaagctttcatttaactctgttgggtccatatgttcgaataatatcagtgagtagtataatgttattaagtgcaagacatgaaagtgttcttttgtcatactccctccgtaaattaatataagagcgtttagattactactttagtgatctaaacgctcttatagtagtttacagacggagtagagtatttcatagccaaccctggaggttaggttgctgaggcctactgggtgggggagggggtttgaaacaagtggtggttagcagccagatttcacaaagaaggaggctgataaccacaccatcagtgaaggaatgaatgtcgggtacccgatcgaccgttttgcccaacgtcgggtttacccgccctatagatccgaataagtagttcctatcttcaattaggtaccaaatatcgccagcgcccgtgtgtgtatttatactactggatgatcaatttatcaacatttccggttaatggtttctatcatattcactgtaattgttagtaaacagtagatgtttgtaatgtagatgatggataaatgtatgttgtcgagctttcatttcaatgcaattttgattgggagctagtttcgcggttcggttagagccatcaaaaccccagaatttttgggagttggcttgtgagagagggttttggggagttaactttcgggattcagttagagacgctcttactagttccagtaaagagtaaactattttctgcaggcatcccaattattctgtagaaattagaagtggaaaatagttatggtatcatataaaccatatattattcaaaatctagaatcatggacttggctagactttgataatctgaaattttaaatttgatgataattgagaaatgatcctttctatcttaggttgtggtaccaaggtatggggactatgaagaagcctacgatgtcggagtccgaaaatactacaaggctgctggacaggtaagcaaaaatgcaatcgaaggggagctgaaattttattgcttattgtcataataaatcaatttttaagtgttttttttgtcctgcaggatatggaagtgaattatttccatgcttatatcgatggagttgattttgtgttcattgacgctcctctcttccgacaccgtcaggaagacatttatgggggcagcagacaggttaatcttctatatgttggtgtttgattgcactgataaactgagaacaagccaaggcctactgactggcatatgattacacattttattttttcaggaaattatgaagcgcatgattttgttctgcaaggccgctgttgaggtatctctccaactcaattgacaacctattaccactatacaattatgtgtatgcatgtatttcaacagatacataatctcttgtgaagtgcatatatactaataacatttcaataccttacatgcacatttggtcaagcgttatgatttaacttctgataatctattgcactgatgaacaattatcttgatgatccttgttacttcatcgttatgtttccatgttctcttcaccgcgaattgatttggaaatagcatttccacctgccacaaacaataatatacactcctactttcatccaatttagatattttcgtacttggcatatcatcccattaaatattattggtccatcatttttattcctctataatttgcaggttccatggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgctcaccaggttccttttctcctaatcttgatttttctctagtctctactatttactccacattgtttgaggaaactaaacgggttgcaaaattatgatggcttatgaaagttatagtcttatagaggtaaatgcaccagtggtgcttgaacttgtcacgcgtgttcactttggtgcttacagttgtagactatgaaaaacgggtgcaaaaacttgctgttgtgtgccatacggtgcattttccgtatgtaggagtcaaacgttgcctatgtgggcattgtattcccgtctatagctgttagaccgtgcctacgtcgccattgggcccacacactctctatttacatgtgggccccacttgtcaacctatgacataaataaatggaaatttataataaaaatgatggcctggggtcttgaaaatgggacctcgcaggtatgctggtagccagcacgccctaaacattaatcccctatgcacttcatgtcttgtgtatgtgtgtgtctgtgtggggagggggggggtatgtatgcttatatcctttgctccaaggctaccatcctcaacaagcccacctccgcttcaacacggccagcgccttcatgatggcccaggtgctccgcaccatcgctcaaagcggcaacgtcgttgtcatgaccatccaccaacccaacacacaaaatcctcaacatccgcaaatagtgagcatgcccctcttgtcctttcccctcgtacccaaacatgtcttgataacccttggagctgcacaagttgtgaccatcgcctgcgtcgcctcatagagcccgacctagccggaccgttatagaagcctacttgggagcccatacctccctgcacatcctcctctttccccatagatcgtgccgccatcgcaaaccaacttctcctctccttctcccactctggccgtttcccccgccgcgaagctgcaatacatgccgagttggccatggccctattccccaattgctcgcactaggaggtcctcctctaagcctagcaccttttcccctcaccaattgcaagttggggagcccctcgcgagctccctacgtcggctgcagttgcctgccgcctcaactctgatccagacctcgttcccgtggcctcggcgacatctcctcgacctcccattccacacgtggcctggcgaggatcaccgcatgttcatccatgtgaaccgaatcatcatagaactaacaccggagaggtcatcccgacggcgtcgcactgttcctctattccccccaagccgtgtcgcgtcataatataagacggacttatttgtatcccttgggtcatcggttcaatggctatttctttctcctgtctactgataagtgggacccacacgccacactaagccctttctttctcctacccgttgataagtgggacccacacacagtacttagccagagagagaacatgagcttgttggtgccacgtcggcaagccatgtcagcagtcttaacggctacaaacaacggatatggtgtcacgtgagcgtttacgaatggaaagtgcatcatactgcatgcgagagccagagccaggtttttgcaccagttttctgtattttacaactgcgagcatcaaagtgtacatatgccgaaccaaagtgaacatggtgagtccattcttttctggtgcggtgggtggctcaaagacaccccaatagaagctattgcctccgacattgccaattcggtgccgaaccatattgaagtggtgaggtcagttgcttgtgctatgactactaggtattggatgagggacataaaggatctcataaatattgcaatgttcattcaaattcttaacatttgcgaagcgcttcatgatttccatctcccctagatcagagacacttggtcgtgtacactgaatttctcaggtcgcttctcgtctaaatccgcatatgtagctcacttcaatgacttgcctttggtccagctaacgccatttgcgtagcaaatttttcatatggctcgctctgcgcaagaggatttggatcacgggcagacgcgctagacaaggtcttccgcacaatgaacattgagttttttgatccgctcttcccgaagacacttgtgatcttattacgagttgtgccatttcaaacatctgtctctccatggtcgccccagccatagatgccttgttctctgaatggtgggtttcagctaggaacagggtgccaccttcggacaagaagttgcgtagtttggtcgtcttaactgcttggttgatttggaaggaacacaacaacagtctttgaaggcaaagctaattccttcgatcaagttattagacggatcaagtgtgatgaatcctactggtacaatgccgttgctagttgcttggagtcactatttggctaggtcgcttgccatcccgctctgtgctaagcgcttggggtcgcttttgctcaatttgtattttgttgttatgtgtttttagtaatgtaacctgaactttctggactaagtagaaaaaaattctcctccataatgatcacatacagttctcctgcatggttcgaaaaaaaaatgagaacatccgtggcaagtttaagcaccaccggtgcatttttacctcaaagttatatacaacactgacatgccgaattacatgctttggtcagttattccattcttcggtactccgttgggctaattctttctcttcatgttgcatgcagggccgtggccctgtagatgaattcccgttcaccgagttgcctgagcactacctggaacacttcagactgtacgaccccgtgggtggtgaacacgccaactacttcgccgccggcctgaagatggcggaccaggttgtcgtggtgagccccgggtacctgtgggagctgaagacggtggagggcggctgggggcttcacgacatcatacggcagaacgactggaagacccgcggcatcgtcaacggcatcgacaacatggagtggaaccccgaggtggacgcccacctcaagtcggacggctacaccaacttctccctgaggacgctggactccggcaagcggcagtgcaaggaggccctgcagcgcgagctgggcctgcaggtccgcgccgacgtgccgctgctcggcttcatcggccgcctggacgggcagaagggcgtggagatcatcgcggacgccatgccctggatcgtgagccaggacgtgcagctggtgatgctgggcaccgggcgccacgacctggagagcatgctgcagcacttcgagcgggagcaccacgacaaggtgcgcgggtgggtggggttctccgtgcgcctggcgcaccggatcacggcgggggcggacgcgctcctcatgccctcccggttcgagccgtgcgggctgaaccagctctacgccatggcctacggcaccgtccccgtcgtgcacgccgtcggcggcctcagggacaccgtgccgccgttcgaccccttcaaccactccgggctcgggtggacgttcgaccgcgccgaggcgcacaagctgatcgaggcgctcgggcactgcctccgcacctaccgagacttcaaggagagctggagggccctccaggagcgcggcatgtcgcaggacttcagctgggagcacgccgccaagctctacgaggacgtcctcgtcaaggccaagtaccagtggtga

SEQ ID NO: 32 - DNA, 2400 bp, Triticum aestivum
  misc_feature (1)..(2400): Starch Synthase II Genome D CDS Wild Type
atgtcgtcggcggtcgcgtccgccgcatccttcctcgcgctcgcgtcagcctcccccgggagatcacgcaggcgggcgagggtgagcgcgcagccaccccacgccggggccggcaggttgcactggccgccgtggccgccgcagcgcacggctcgcgacggagctgtggcggcgctcgccgccgggaagaaggacgcggggatcgacgacgccgccgcgtccgtgaggcagccccgcgcactccgcggtggcgccgccaccaaggtcgcggagcgaagggatcccgtcaagacgctcgaccgcgacgccgcggaaggcggcgggccgtccccgccggcagcgaggcaggacgccgcccgtccgccgagtatgaacggcatgccggtgaacggcgagaacaaatctaccggcggcggcggcgcgactaaagacagcgggctgcccacgcccgcacgcgcgccccatccgtcgacccagaacagagcaccggtgaacggtgaaaacaaagctaacgtcgcctcgccgccgacgagcatagccgaggccgcggcttcggattccgcagctaccatttccatcagcgacaaggcgccggagtccgttgtcccagctgagaagacgccgccgtcgtccggctcaaatttcgagtcctcggcctctgctcccgggtctgacactgtcagcgacgtggaacaagaactgaagaagggtgcggtcgttgtcgaagaagctccaaagccaaaggctctttcgccgcctgcagcccccgctgtacaagaagacctttgggatttcaagaaatacattggtttcgaggagcccgtggaggccaaggatgatggccgggctgtcgcagatgatgcgggctcctttgaacaccaccagaatcacgactccggacctttggcaggggagaatgtcatgaacgtggtcgtcgtggctgctgagtgttctccctggtgcaaaacaggtggtctgggagatgttgcgggtgctctgcccaaggctttggcaaagagaggacatcgtgttatggttgtggtaccaaggtatggggactatgaagaagcctacgatgtcggagtccgaaaatactacaaggctgctggacaggatatggaagtgaattatttccatgcttatatcgatggagttgattttgtgttcattgacgctcctctcttccgacaccgtcaggaagacatttatgggggcagcagacaggaaattatgaagcgcatgattttgttctgcaaggccgctgttgaggttccatggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgctcaccagggccgtggccctgtagatgaattcccgttcaccgagttgcctgagcactacctggaacacttcagactgtacgaccccgtgggtggtgaacacgccaactacttcgccgccggcctgaagatggcggaccaggttgtcgtggtgagccccgggtacctgtgggagctgaagacggtggagggcggctgggggcttcacgacatcatacggcagaacgactggaagacccgcggcatcgtcaacggcatcgacaacatggagtggaaccccgaggtggacgcccacctcaagtcggacggctacaccaacttctccctgaggacgctggactccggcaagcggcagtgcaaggaggccctgcagcgcgagctgggcctgcaggtccgcgccgacgtgccgctgctcggcttcatcggccgcctggacgggcagaagggcgtggagatcatcgcggacgccatgccctggatcgtgagccaggacgtgcagctggtgatgctgggcaccgggcgccacgacctggagagcatgctgcagcacttcgagcgggagcaccacgacaaggtgcgcgggtgggtggggttctccgtgcgcctggcgcaccggatcacggcgggggcggacgcgctcctcatgccctcccggttcgagccgtgcgggctgaaccagctctacgccatggcctacggcaccgtccccgtcgtgcacgccgtcggcggcctcagggacaccgtgccgccgttcgaccccttcaaccactccgggctcgggtggacgttcgaccgcgccgaggcgcacaagctgatcgaggcgctcgggcactgcctccgcacctaccgagacttcaaggagagctggagggccctccaggagcgcggcatgtcgcaggacttcagctgggagcacgccgccaagctctacgaggacgtcctcgtcaaggccaagtaccagtggtga

SEQ ID NO: 33 - protein, 799 aa, Triticum aestivum
  MISC_FEATURE (1)..(799): Starch Synthase II Genome D Protein Wild Type
MSSAVASAASFLALASASPGRSRRRARVSAQPPHAGAGRLHWPPWPPQRTARDGAVAALAAGKKDAGIDDAAASVRQPRALRGGAATKVAERRDPVKTLDRDAAEGGGPSPPAARQDAARPPSMNGMPVNGENKSTGGGGATKDSGLPTPARAPHPSTQNRAPVNGENKANVASPPTSIAEAAASDSAATISISDKAPESVVPAEKTPPSSGSNFESSASAPGSDTVSDVEQELKKGAVVVEEAPKPKALSPPAAPAVQEDLWDFKKYIGFEEPVEAKDDGRAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPLFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDAHLKSDGYTNFSLRTLDSGKRQCKEALQRELGLQVRADVPLLGFIGRLDGQKGVEIIADAMPWIVSQDVQLVMLGTGRHDLESMLQHFEREHHDKVRGWVGFSVRLAHRITAGADALLMPSRFEPCGLNQLYAMAYGTVPVVHAVGGLRDTVPPFDPFNHSGLGWTFDRAEAHKLIEALGHCLRTYRDFKESWRALQERGMSQDFSWEHAAKLYEDVLVKAKYQW

SEQ ID NO: 34 - DNA, 7010 bp, Artificial Sequence
  RJ 183 Mutant Starch Synthase II Genome D Genomic
gggggccgttcgtacgtacccgcccctcgtgtaaagccgccgccgtcgtcgccgtcccccgctcgcggccatttcttcggcctgaccccgttcgtttacccccacacagagcacactccagtccagtccagcccactgccaccgcgctactctccactcccactgccaccacctccgcctgcgccgcgctctgggcggaccaacccgcgaaccgtaccatctcccgccccgatccatgtcgtcggcggtcgcgtccgccgcatccttcctcgcgctcgcgtcagcctcccccgggagatcacgcaggcgggcgagggtgagcgcgcagccaccccacgccggggccggcaggttgcactggccgccgtggccgccgcagcgcacggctcgcgacggagctgtggcggcgctcgccgccgggaagaaggacgcggggatcgacgacgccgccgcgtccgtgaggcagccccgcgcactccgcggtggcgccgccaccaaggtagttagttatgaccaagttatgacgcgtgcgcgcgcctcgagatcatcgtcgtctcgctcacgaattgtttatttatacaaaacgcacgcccgcgtgtgcaggtcgcggagcgaagggatcccgtcaagacgctcgaccgcgacgccgcggaaggcggcgggccgtccccgccggcagcgaggcaggacgccgcccgtccgccgagtatgaacggcatgccggtgaacggcgagaacaaatctaccggcggcggcggcgcgactaaagacagcgggctgcccacgcccgcacgcgcgccccatccgtcgacccagaacagagcaccggtgaacggtgaaaacaaagctaacgtcgcctcgccgccgacgagcatagccgaggccgcggcttcggattccgcagctaccatttccatcagcgacaaggcgccggagtccgttgtcccagctgagaagacgccgccgtcgtccggctcaaatttcgagtcctcggcctctgctcccgggtctgacactgtcagcgacgtggaacaagaactgaagaagggtgcggtcgttgtcgaagaagctccaaagccaaaggctctttcgccgcctgcagcccccgctgtacaagaagacctttgggatttcaagaaatacattggtttcgaggagcccgtggaggccaaggatgatggccgggctgtcgcagatgatgcgggctcctttgaacaccaccagaatcacgactccggacctttggcaggggagaatgtcatgaacgtggtcgtcgtggctgctgagtgttctccctggtgcaaaacaggcatggacattacctcttcagtctctcttcctgttgttcataaaactttgctcgaattactcataagaacaaacattgtgttgcataggtggtctgggagatgttgcgggtgctctgcccaaggctttggcaaagagaggacatcgtgttatggtactacaagctttcatttaactctgttgggtccatatgttcgaataatatcagtgagtagtataatgttattaagtgcaagacatgaaagtgttcttttgtcatactccctccgtaaattaatataagagcgtttagattactactttagtgatctaaacgctcttatagtagtttacagacggagtagagtatttcatagccaaccctggaggttaggttgctgaggcctactgggtgggggagggggtttgaaacaagtggtggttagcagccagatttcacaaagaaggaggctgataaccacaccatcagtgaaggaatgaatgtcgggtacccgatcgaccgttttgcccaacgtcgggtttacccgccctatagatccgaataagtagttcctatcttcaattaggtaccaaatatcgccagcgcccgtgtgtgtatttatactactggatgatcaatttatcaacatttccggttaatggtttctatcatattcactgtaattgttagtaaacagtagatgtttgtaatgtagatgatggataaatgtatgttgtcgagctttcatttcaatgcaattttgattgggagctagtttcgcggttcggttagagccatcaaaaccccagaatttttgggagttggcttgtgagagagggttttggggagttaactttcgggattcagttagagacgctcttactagttccagtaaagagtaaactattttctgcaggcatcccaattattctgtagaaattagaagtggaaaatagttatggtatcatataaaccatatattattcaaaatctagaatcatggacttggctagactttgataatctgaaattttaaatttgatgataattgagaaatgatcctttctatcttaggttgtggtaccaaggtatggggactatgaagaagcctacgatgtcggagtccgaaaatactacaaggctgctggacagataagcaaaaatgcaatcgaaggggagctgaaattttattgcttattgtcataataaatcaatttttaagtgttttttttgtcctgcaggatatggaagtgaattatttccatgcttatatcgatggagttgattttgtgttcattgacgctcctctcttccgacaccgtcaggaagacatttatgggggcagcagacaggttaatcttctatatgttggtgtttgattgcactgataaactgagaacaagccaaggcctactgactggcatatgattacacattttattttttcaggaaattatgaagcgcatgattttgttctgcaaggccgctgttgaggtatctctccaactcaattgacaacctattaccactatacaattatgtgtatgcatgtatttcaacagatacataatctcttgtgaagtgcatatatactaataacatttcaataccttacatgcacatttggtcaagcgttatgatttaacttctgataatctattgcactgatgaacaattatcttgatgatccttgttacttcatcgttatgtttccatgttctcttcaccgcgaattgatttggaaatagcatttccacctgccacaaacaataatatacactcctactttcatccaatttagatattttcgtacttggcatatcatcccattaaatattattggtccatcatttttattcctctataatttgcaggttccatggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgctcaccaggttccttttctcctaatcttgatttttctctagtctctactatttactccacattgtttgaggaaactaaacgggttgcaaaattatgatggcttatgaaagttatagtcttatagaggtaaatgcaccagtggtgcttgaacttgtcacgcgtgttcactttggtgcttacagttgtagactatgaaaaacgggtgcaaaaacttgctgttgtgtgccatacggtgcattttccgtatgtaggagtcaaacgttgcctatgtgggcattgtattcccgtctatagctgttagaccgtgcctacgtcgccattgggcccacacactctctatttacatgtgggccccacttgtcaacctatgacataaataaatggaaatttataataaaaatgatggcctggggtcttgaaaatgggacctcgcaggtatgctggtagccagcacgccctaaacattaatcccctatgcacttcatgtcttgtgtatgtgtgtgtctgtgtggggagggggggggtatgtatgcttatatcctttgctccaaggctaccatcctcaacaagcccacctccgcttcaacacggccagcgccttcatgatggcccaggtgctccgcaccatcgctcaaagcggcaacgtcgttgtcatgaccatccaccaacccaacacacaaaatcctcaacatccgcaaatagtgagcatgcccctcttgtcctttcccctcgtacccaaacatgtcttgataacccttggagctgcacaagttgtgaccatcgcctgcgtcgcctcatagagcccgacctagccggaccgttatagaagcctacttgggagcccatacctccctgcacatcctcctctttccccatagatcgtgccgccatcgcaaaccaacttctcctctccttctcccactctggccgtttcccccgccgcgaagctgcaatacatgccgagttggccatggccctattccccaattgctcgcactaggaggtcctcctctaagcctagcaccttttcccctcaccaattgcaagttggggagcccctcgcgagctccctacgtcggctgcagttgcctgccgcctcaactctgatccagacctcgttcccgtggcctcggcgacatctcctcgacctcccattccacacgtggcctggcgaggatcaccgcatgttcatccatgtgaaccgaatcatcatagaactaacaccggagaggtcatcccgacggcgtcgcactgttcctctattccccccaagccgtgtcgcgtcataatataagacggacttatttgtatcccttgggtcatcggttcaatggctatttctttctcctgtctactgataagtgggacccacacgccacactaagccctttctttctcctacccgttgataagtgggacccacacacagtacttagccagagagagaacatgagcttgttggtgccacgtcggcaagccatgtcagcagtcttaacggctacaaacaacggatatggtgtcacgtgagcgtttacgaatggaaagtgcatcatactgcatgcgagagccagagccaggtttttgcaccagttttctgtattttacaactgcgagcatcaaagtgtacatatgccgaaccaaagtgaacatggtgagtccattcttttctggtgcggtgggtggctcaaagacaccccaatagaagctattgcctccgacattgccaattcggtgccgaaccatattgaagtggtgaggtcagttgcttgtgctatgactactaggtattggatgagggacataaaggatctcataaatattgcaatgttcattcaaattcttaacatttgcgaagcgcttcatgatttccatctcccctagatcagagacacttggtcgtgtacactgaatttctcaggtcgcttctcgtctaaatccgcatatgtagctcacttcaatgacttgcctttggtccagctaacgccatttgcgtagcaaatttttcatatggctcgctctgcgcaagaggatttggatcacgggcagacgcgctagacaaggtcttccgcacaatgaacattgagttttttgatccgctcttcccgaagacacttgtgatcttattacgagttgtgccatttcaaacatctgtctctccatggtcgccccagccatagatgccttgttctctgaatggtgggtttcagctaggaacagggtgccaccttcggacaagaagttgcgtagtttggtcgtcttaactgcttggttgatttggaaggaacacaacaacagtctttgaaggcaaagctaattccttcgatcaagttattagacggatcaagtgtgatgaatcctactggtacaatgccgttgctagttgcttggagtcactatttggctaggtcgcttgccatcccgctctgtgctaagcgcttggggtcgcttttgctcaatttgtattttgttgttatgtgtttttagtaatgtaacctgaactttctggactaagtagaaaaaaattctcctccataatgatcacatacagttctcctgcatggttcgaaaaaaaaatgagaacatccgtggcaagtttaagcaccaccggtgcatttttacctcaaagttatatacaacactgacatgccgaattacatgctttggtcagttattccattcttcggtactccgttgggctaattctttctcttcatgttgcatgcagggccgtggccctgtagatgaattcccgttcaccgagttgcctgagcactacctggaacacttcagactgtacgaccccgtgggtggtgaacacgccaactacttcgccgccggcctgaagatggcggaccaggttgtcgtggtgagccccgggtacctgtgggagctgaagacggtggagggcggctgggggcttcacgacatcatacggcagaacgactggaagacccgcggcatcgtcaacggcatcgacaacatggagtggaaccccgaggtggacgcccacctcaagtcggacggctacaccaacttctccctgaggacgctggactccggcaagcggcagtgcaaggaggccctgcagcgcgagctgggcctgcaggtccgcgccgacgtgccgctgctcggcttcatcggccgcctggacgggcagaagggcgtggagatcatcgcggacgccatgccctggatcgtgagccaggacgtgcagctggtgatgctgggcaccgggcgccacgacctggagagcatgctgcagcacttcgagcgggagcaccacgacaaggtgcgcgggtgggtggggttctccgtgcgcctggcgcaccggatcacggcgggggcggacgcgctcctcatgccctcccggttcgagccgtgcgggctgaaccagctctacgccatggcctacggcaccgtccccgtcgtgcacgccgtcggcggcctcagggacaccgtgccgccgttcgaccccttcaaccactccgggctcgggtggacgttcgaccgcgccgaggcgcacaagctgatcgaggcgctcgggcactgcctccgcacctaccgagacttcaaggagagctggagggccctccaggagcgcggcatgtcgcaggacttcagctgggagcacgccgccaagctctacgaggacgtcctcgtcaaggccaagtaccagtggtga

SEQ ID NO: 35 - DNA, 2400 bp, Artificial Sequence
  RJ 597 Mutant Starch Synthase II Genome D CDS
atgtcgtcggcggtcgcgtccgccgcatccttcctcgcgctcgcgtcagcctcccccgggagatcacgcaggcgggcgagggtgagcgcgcagccaccccacgccggggccggcaggttgcactggccgccgtggccgccgcagcgcacggctcgcgacggagctgtggcggcgctcgccgccgggaagaaggacgcggggatcgacgacgccgccgcgtccgtgaggcagccccgcgcactccgcggtggcgccgccaccaaggtcgcggagcgaagggatcccgtcaagacgctcgaccgcgacgccgcggaaggcggcgggccgtccccgccggcagcgaggcaggacgccgcccgtccgccgagtatgaacggcatgccggtgaacggcgagaacaaatctaccggcggcggcggcgcgactaaagacagcgggctgcccacgcccgcacgcgcgccccatccgtcgacccagaacagagcaccggtgaacggtgaaaacaaagctaacgtcgcctcgccgccgacgagcatagccgaggccgcggcttcggattccgcagctaccatttccatcagcgacaaggcgccggagtccgttgtcccagctgagaagacgccgccgtcgtccggctcaaatttcgagtcctcggcctctgctcccgggtctgacactgtcagcgacgtggaacaagaactgaagaagggtgcggtcgttgtcgaagaagctccaaagccaaaggctctttcgccgcctgcagcccccgctgtacaagaagacctttgggatttcaagaaatacattggtttcgaggagcccgtggaggccaaggatgatggccgggctgtcgcagatgatgcgggctcctttgaacaccaccagaatcacgactccggacctttggcaggggagaatgtcatgaacgtggtcgtcgtggctgctgagtgttctccctggtgcaaaacaggtggtctgggagatgttgcgggtgctctgcccaaggctttggcaaagagaggacatcgtgttatggttgtggtaccaaggtatggggactatgaagaagcctacgatgtcggagtccgaaaatactacaaggctgctggacaggatatggaagtgaattatttccatgcttatatcgatggagttgattttgtgttcattgacgctcctctcttccgacaccgtcaggaagacatttatgggggcagcagacaggaaattatgaagcgcatgattttgttctgcaaggccgctgttgaggttccatggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgctcaccagggccgtggccctgtagatgaattcccgttcaccgagttgcctgagcactacctggaacacttcagactgtacgaccccgtgggtggtgaacacgccaactacttcgccgccggcctgaagatggcggaccaggttgtcgtggtgagccccgggtacctgtgggagctgaagacggtggagggcggctgggggcttcacgacatcatacggcagaacgactggaagacccgcggcatcgtcaacggcatcgacaacatggagtggaaccccgaggtggacgcccacctcaagtcggacggctacaccaacttctccctgaggacgctggactccggcaagcggcagtgcaaggaggccctgcagcgcgagctgggcctgcaggtccgcgccgacgtgccgctgctcggcttcatcggccgcctggacgggcagaagggcgtggagatcatcgcggacgccatgccctggatcgtgagccaggacgtgcagctggtgatgctgggcaccgggcgccacgacctggagagcatgctgcagcacttcgagcgggagcaccacgacaaggtgcgcgggtgggtggggttctccgtgcgcctggcgcaccggatcacggcgggggcggacgcgctcctcatgccctcccggttcgagccgtgcgggctgaaccagctctacgccatggcctacggcaccgtccccgtcgtgcacgccgtcgacggcctcagggacaccgtgccgccgttcgaccccttcaaccactccgggctcgggtggacgttcgaccgcgccgaggcgcacaagctgatcgaggcgctcgggcactgcctccgcacctaccgagacttcaaggagagctggagggccctccaggagcgcggcatgtcgcaggacttcagctgggagcacgccgccaagctctacgaggacgtcctcgtcaaggccaagtaccagtggtga

SEQ ID NO: 36 - protein, 799 aa, Artificial Sequence
  RJ 597 Mutant Starch Synthase II Genome D Protein
MSSAVASAASFLALASASPGRSRRRARVSAQPPHAGAGRLHWPPWPPQRTARDGAVAALAAGKKDAGIDDAAASVRQPRALRGGAATKVAERRDPVKTLDRDAAEGGGPSPPAARQDAARPPSMNGMPVNGENKSTGGGGATKDSGLPTPARAPHPSTQNRAPVNGENKANVASPPTSIAEAAASDSAATISISDKAPESVVPAEKTPPSSGSNFESSASAPGSDTVSDVEQELKKGAVVVEEAPKPKALSPPAAPAVQEDLWDFKKYIGFEEPVEAKDDGRAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPLFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDAHLKSDGYTNFSLRTLDSGKRQCKEALQRELGLQVRADVPLLGFIGRLDGQKGVEIIADAMPWIVSQDVQLVMLGTGRHDLESMLQHFEREHHDKVRGWVGFSVRLAHRITAGADALLMPSRFEPCGLNQLYAMAYGTVPVVHAVDGLRDTVPPFDPFNHSGLGWTFDRAEAHKLIEALGHCLRTYRDFKESWRALQERGMSQDFSWEHAAKLYEDVLVKAKYQW

SEQ ID NO: 37 - DNA, 2400 bp, Artificial Sequence
  RJ 647 Mutant Starch Synthase II Genome D CDS
atgtcgtcggcggtcgcgtccgccgcatccttcctcgcgctcgcgtcagcctcccccgggagatcacgcaggcgggcgagggtgagcgcgcagccaccccacgccggggccggcaggttgcactggccgccgtggccgccgcagcgcacggctcgcgacggagctgtggcggcgctcgccgccgggaagaaggacgcggggatcgacgacgccgccgcgtccgtgaggcagccccgcgcactccgcggtggcgccgccaccaaggtcgcggagcgaagggatcccgtcaagacgctcgaccgcgacgccgcggaaggcggcgggccgtccccgccggcagcgaggcaggacgccgcccgtccgccgagtatgaacggcatgccggtgaacggcgagaacaaatctaccggcggcggcggcgcgactaaagacagcgggctgcccacgcccgcacgcgcgccccatccgtcgacccagaacagagcaccggtgaacggtgaaaacaaagctaacgtcgcctcgccgccgacgagcatagccgaggccgcggcttcggattccgcagctaccatttccatcagcgacaaggcgccggagtccgttgtcccagctgagaagacgccgccgtcgtccggctcaaatttcgagtcctcggcctctgctcccgggtctgacactgtcagcgacgtggaacaagaactgaagaagggtgcggtcgttgtcgaagaagctccaaagccaaaggctctttcgccgcctgcagcccccgctgtacaagaagacctttgggatttcaagaaatacattggtttcgaggagcccgtggaggccaaggatgatggccgggctgtcgcagatgatgcgggctcctttgaacaccaccagaatcacgactccggacctttggcaggggagaatgtcatgaacgtggtcgtcgtggctgctgagtgttctccctggtgcaaaacaggtggtctgggagatgttgcgggtgctctgcccaaggctttggcaaagagaggacatcgtgttatggttgtggtaccaaggtatggggactatgaagaagcctacgatgtcggagtccgaaaatactacaaggctgctggacaggatatggaagtgaattatttccatgcttatatcgatggagttgattttgtgttcattgacgctcctctcttccgacaccgtcaggaagacatttatgggggcagcagacaggaaattatgaagcgcatgattttgttctgcaaggccgctgttgaggttccatggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgctcaccagggccgtggccctgtagatgaattcccgttcaccgagttgcctgagcactacctggaacacttcagactgtacgaccccgtgggtggtgaacacgccaactacttcgccgccggcctgaagatggcggaccaggttgtcgtggtgagccccgggtacctgtgggagctgaagacggtggagggcggctgggggcttcacgacatcatacggcagaacgactggaagacccgcggcatcgtcaacggcatcgacaacatggagtggaaccccgaggtggacgcccacctcaagtcggacggctacaccaacttctccctgaggacgctggactccggcaagcggcagtgcaaggaggccctgcagcgcgagctgggcctgcaggtccgcgccgacgtgccgctgctcggcttcatcggccgcctggacgggcagaagggcgtggagatcatcgcggacgccatgccctggatcgtgagccaggacgtgcagctggtgatgctgggcaccgggcgccacgacctggagagcatgctgtagcacttcgagcgggagcaccacgacaaggtgcgcgggtgggtggggttctccgtgcgcctggcgcaccggatcacggcgggggcggacgcgctcctcatgccctcccggttcgagccgtgcgggctgaaccagctctacgccatggcctacggcaccgtccccgtcgtgcacgccgtcggcggcctcagggacaccgtgccgccgttcgaccccttcaaccactccgggctcgggtggacgttcgaccgcgccgaggcgcacaagctgatcgaggcgctcgggcactgcctccgcacctaccgagacttcaaggagagctggagggccctccaggagcgcggcatgtcgcaggacttcagctgggagcacgccgccaagctctacgaggacgtcctcgtcaaggccaagtaccagtggtga

SEQ ID NO: 38 - protein, 659 aa, Artificial Sequence
  RJ 647 Mutant Starch Synthase II Genome D Protein
MSSAVASAASFLALASASPGRSRRRARVSAQPPHAGAGRLHWPPWPPQRTARDGAVAALAAGKKDAGIDDAAASVRQPRALRGGAATKVAERRDPVKTLDRDAAEGGGPSPPAARQDAARPPSMNGMPVNGENKSTGGGGATKDSGLPTPARAPHPSTQNRAPVNGENKANVASPPTSIAEAAASDSAATISISDKAPESVVPAEKTPPSSGSNFESSASAPGSDTVSDVEQELKKGAVVVEEAPKPKALSPPAAPAVQEDLWDFKKYIGFEEPVEAKDDGRAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPLFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDAHLKSDGYTNFSLRTLDSGKRQCKEALQRELGLQVRADVPLLGFIGRLDGQKGVEIIADAMPWIVSQDVQLVMLGTGRHDLESML

SEQ ID NO: 39 - DNA, 2400 bp, Artificial Sequence
  RJ 624 Mutant Starch Synthase II Genome D CDS
atgtcgtcggcggtcgcgtccgccgcatccttcctcgcgctcgcgtcagcctcccccgggagatcacgcaggcgggcgagggtgagcgcgcagccaccccacgccggggccggcaggttgcactggccgccgtggccgccgcagcgcacggctcgcgacggagctgtggcggcgctcgccgccgggaagaaggacgcggggatcgacgacgccgccgcgtccgtgaggcagccccgcgcactccgcggtggcgccgccaccaaggtcgcggagcgaagggatcccgtcaagacgctcgaccgcgacgccgcggaaggcggcgggccgtccccgccggcagcgaggcaggacgccgcccgtccgccgagtatgaacggcatgccggtgaacggcgagaacaaatctaccggcggcggcggcgcgactaaagacagcgggctgcccacgcccgcacgcgcgccccatccgtcgacccagaacagagcaccggtgaacggtgaaaacaaagctaacgtcgcctcgccgccgacgagcatagccgaggccgcggcttcggattccgcagctaccatttccatcagcgacaaggcgccggagtccgttgtcccagctgagaagacgccgccgtcgtccggctcaaatttcgagtcctcggcctctgctcccgggtctgacactgtcagcgacgtggaacaagaactgaagaagggtgcggtcgttgtcgaagaagctccaaagccaaaggctctttcgccgcctgcagcccccgctgtacaagaagacctttgggatttcaagaaatacattggtttcgaggagcccgtggaggccaaggatgatggccgggctgtcgcagatgatgcgggctcctttgaacaccaccagaatcacgactccggacctttggcaggggagaatgtcatgaacgtggtcgtcgtggctgctgagtgttctccctggtgcaaaacaggtggtctgggagatgttgcgggtgctctgcccaaggctttggcaaagagaggacatcgtgttatggttgtggtaccaaggtatggggactatgaagaagcctacgatgtcggagtccgaaaatactacaaggctgctggacaggatatggaagtgaattatttccatgcttatatcgatggagttgattttgtgttcattgacgctcctctcttccgacaccgtcaggaagacatttatgggggcagcagacaggaaattatgaagcgcatgattttgttctgcaaggccgctgttgaggttccatggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgctcaccagggccgtggccctgtagatgaattcccgttcaccgagttgcctgagcactacctggaacacttcagactgtacgaccccgtgggtggtgaacacgccaactacttcgccgccggcctgaagatggcggaccaggttgtcgtggtgagccccgggtacctgtgggagctgaagacggtggagggcggctgggggcttcacgacatcatacggcagaacgactggaagacccgcggcatcgtcaacggcatcgacaacatggagtggaaccccgaggtggacgcccacctcaagtcggacggctacaccaacttctccctgaggacgctggactccggcaagcggcagtgcaaggaggccctgcagcgcgagctgggcctgcaggtccgcgccgacgtgccgctgctcggcttcatcggccgcctggacgggcagaagggcgtggagatcatcgcggacgccatgccctggatcgtgagccaggacgtgcagctggtgatgctgggcaccgggcgccacgacctgaagagcatgctgcagcacttcgagcgggagcaccacgacaaggtgcgcgggtgggtggggttctccgtgcgcctggcgcaccggatcacggcgggggcggacgcgctcctcatgccctcccggttcgagccgtgcgggctgaaccagctctacgccatggcctacggcaccgtccccgtcgtgcacgccgtcggcggcctcagggacaccgtgccgccgttcgaccccttcaaccactccgggctcgggtggacgttcgaccgcgccgaggcgcacaagctgatcgaggcgctcgggcactgcctccgcacctaccgagacttcaaggagagctggagggccctccaggagcgcggcatgtcgcaggacttcagctgggagcacgccgccaagctctacgaggacgtcctcgtcaaggccaagtaccagtggtga

SEQ ID NO: 40 - protein, 799 aa, Artificial Sequence
  RJ 624 Mutant Starch Synthase II Genome D Protein
MSSAVASAASFLALASASPGRSRRRARVSAQPPHAGAGRLHWPPWPPQRTARDGAVAALAAGKKDAGIDDAAASVRQPRALRGGAATKVAERRDPVKTLDRDAAEGGGPSPPAARQDAARPPSMNGMPVNGENKSTGGGGATKDSGLPTPARAPHPSTQNRAPVNGENKANVASPPTSIAEAAASDSAATISISDKAPESVVPAEKTPPSSGSNFESSASAPGSDTVSDVEQELKKGAVVVEEAPKPKALSPPAAPAVQEDLWDFKKYIGFEEPVEAKDDGRAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPLFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDAHLKSDGYTNFSLRTLDSGKRQCKEALQRELGLQVRADVPLLGFIGRLDGQKGVEIIADAMPWIVSQDVQLVMLGTGRHDLKSMLQHFEREHHDKVRGWVGFSVRLAHRITAGADALLMPSRFEPCGLNQLYAMAYGTVPVVHAVGGLRDTVPPFDPFNHSGLGWTFDRAEAHKLIEALGHCLRTYRDFKESWRALQERGMSQDFSWEHAAKLYEDVLVKAKYQW

SEQ ID NO: 41 - DNA, 2400 bp, Artificial Sequence
  RJ 414 Mutant Starch Synthase II Genome D CDS
atgtcgtcggcggtcgcgtccgccgcatccttcctcgcgctcgcgtcagcctcccccgggagatcacgcaggcgggcgagggtgagcgcgcagccaccccacgccggggccggcaggttgcactggccgccgtggccgccgcagcgcacggctcgcgacggagctgtggcggcgctcgccgccgggaagaaggacgcggggatcgacgacgccgccgcgtccgtgaggcagccccgcgcactccgcggtggcgccgccaccaaggtcgcggagcgaagggatcccgtcaagacgctcgaccgcgacgccgcggaaggcggcgggccgtccccgccggcagcgaggcaggacgccgcccgtccgccgagtatgaacggcatgccggtgaacggcgagaacaaatctaccggcggcggcggcgcgactaaagacagcgggctgcccacgcccgcacgcgcgccccatccgtcgacccagaacagagcaccggtgaacggtgaaaacaaagctaacgtcgcctcgccgccgacgagcatagccgaggccgcggcttcggattccgcagctaccatttccatcagcgacaaggcgccggagtccgttgtcccagctgagaagacgccgccgtcgtccggctcaaatttcgagtcctcggcctctgctcccgggtctgacactgtcagcgacgtggaacaagaactgaagaagggtgcggtcgttgtcgaagaagctccaaagccaaaggctctttcgccgcctgcagcccccgctgtacaagaagacctttgggatttcaagaaatacattggtttcgaggagcccgtggaggccaaggatgatggccgggctgtcgcagatgatgcgggctcctttgaacaccaccagaatcacgactccggacctttggcaggggagaatgtcatgaacgtggtcgtcgtggctgctgagtgttctccctggtgcaaaacaggtggtctgggagatgttgcgggtgctctgcccaaggctttggcaaagagaggacatcgtgttatggttgtggtaccaaggtatggggactatgaagaagcctacgatgtcggagtccgaaaatactacaaggctgctggacaggatatggaagtgaattatttccatgcttatatcgatggagttgattttgtgttcattgacgctcctctcttccgacaccgtcaggaagacatttatgggggcagcagacaggaaattatgaagcgcatgattttgttctgcaaggccgctgttgaggttccatggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgctcaccagggccgtggccctgtagatgaattcccgttcaccgagttgcctgagcactacctggaacacttcagactgtacgaccccgtgggtggtgaacacgccaactacttcgccgccggcctgaagatggcggaccaggttgtcgtggtgagccccgggtacctgtgggagctgaagacggtggagggcggctgggggcttcacgacatcatacggcagaacgactggaagacccgcggcatcgtcaacggcatcgacaacatggagtggaaccccgaggtggacgcccacctcaagtcggacggctacaccaacttctccctgaggacgctggactccggcaagcggcagtgcaaggaggccctgcagcgcgagctgggcctgcaggtccgcgccgacgtgccgctgctcggcttcatcggccgcctggacgggcagaagggcgtggagatcatcgcggacgccatgccctggatcgtgagccaggacgtgcagctggtgatgctgggcaccgggcgccacgacctggagagcatgctgcagcacttcgagcgggagcaccacgacaaggtgcgcgggtgggtggggttctccgtgcgcctggcgcaccggatcacggcgggggcggacgcgctcctcatgccctcccggttcgagccgtgcgggctgaaccagctctacgccatggcctacggcaccgtccccgtcgtgcacgccgtcggcggcctcagggacaccgtgccgccgttcgaccccttcaaccactccgggctcgggtggacgttcgaccgcgccgaggcgcacaagctgatcgaggcgctcgggcactgcctccgcacctaccgagacttcaaggagagctggagggccctccaggagcgcggcatgtcgcaggacttcagctgggagcacgccgtcaagctctacgaggacgtcctcgtcaaggccaagtaccagtggtga

SEQ ID NO: 42 - protein, 799 aa, Artificial Sequence
  RJ 414 Mutant Starch Synthase II Genome D Protein
MSSAVASAASFLALASASPGRSRRRARVSAQPPHAGAGRLHWPPWPPQRTARDGAVAALAAGKKDAGIDDAAASVRQPRALRGGAATKVAERRDPVKTLDRDAAEGGGPSPPAARQDAARPPSMNGMPVNGENKSTGGGGATKDSGLPTPARAPHPSTQNRAPVNGENKANVASPPTSIAEAAASDSAATISISDKAPESVVPAEKTPPSSGSNFESSASAPGSDTVSDVEQELKKGAVVVEEAPKPKALSPPAAPAVQEDLWDFKKYIGFEEPVEAKDDGRAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPLFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDAHLKSDGYTNFSLRTLDSGKRQCKEALQRELGLQVRADVPLLGFIGRLDGQKGVEIIADAMPWIVSQDVQLVMLGTGRHDLESMLQHFEREHHDKVRGWVGFSVRLAHRITAGADALLMPSRFEPCGLNQLYAMAYGTVPVVHAVGGLRDTVPPFDPFNHSGLGWTFDRAEAHKLIEALGHCLRTYRDFKESWRALQERGMSQDFSWEHAVKLYEDVLVKAKYQW

SEQ ID NO: 43 - DNA, 2400 bp, Artificial Sequence
  RJ 122 Mutant Starch Synthase II Genome D CDS
atgtcgtcggcggtcgcgtccgccgcatccttcctcgcgctcgcgtcagcctcccccgggagatcacgcaggcgggcgagggtgagcgcgcagccaccccacgccggggccggcaggttgcactggccgccgtggccgccgcagcgcacggctcgcgacggagctgtggcggcgctcgccgccgggaagaaggacgcggggatcgacgacgccgccgcgtccgtgaggcagccccgcgcactccgcggtggcgccgccaccaaggtcgcggagcgaagggatcccgtcaagacgctcgaccgcgacgccgcggaaggcggcgggccgtccccgccggcagcgaggcaggacgccgcccgtccgccgagtatgaacggcatgccggtgaacggcgagaacaaatctaccggcggcggcggcgcgactaaagacagcgggctgcccacgcccgcacgcgcgccccatccgtcgacccagaacagagcaccggtgaacggtgaaaacaaagctaacgtcgcctcgccgccgacgagcatagccgaggccgcggcttcggattccgcagctaccatttccatcagcgacaaggcgccggagtccgttgtcccagctgagaagacgccgccgtcgtccggctcaaatttcgagtcctcggcctctgctcccgggtctgacactgtcagcgacgtggaacaagaactgaagaagggtgcggtcgttgtcgaagaagctccaaagccaaaggctctttcgccgcctgcagcccccgctgtacaagaagacctttgggatttcaagaaatacattggtttcgaggagcccgtggaggccaaggatgatggccgggctgtcgcagatgatgcgggctcctttgaacaccaccagaatcacgactccggacctttggcaggggagaatgtcatgaacgtggtcgtcgtggctgctgagtgttctccctggtgcaaaacaggtggtctgggagatgttgcgggtgctctgcccaaggctttggcaaagagaggacatcgtgttatggttgtggtaccaaggtatggggactatgaagaagcctacgatgtcggagtccgaaaatactacaaggctgctggacaggatatggaagtgaattatttccatgcttatatcgatggagttgattttgtgttcattgacgctcctctcttccgacaccgtcaggaagacatttatgggggcagcagacaggaaattatgaagcgcatgattttgttctgcaaggccgttgttgaggttccatggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgctcaccagggccgtggccctgtagatgaattcccgttcaccgagttgcctgagcactacctggaacacttcagactgtacgaccccgtgggtggtgaacacgccaactacttcgccgccggcctgaagatggcggaccaggttgtcgtggtgagccccgggtacctgtgggagctgaagacggtggagggcggctgggggcttcacgacatcatacggcagaacgactggaagacccgcggcatcgtcaacggcatcgacaacatggagtggaaccccgaggtggacgcccacctcaagtcggacggctacaccaacttctccctgaggacgctggactccggcaagcggcagtgcaaggaggccctgcagcgcgagctgggcctgcaggtccgcgccgacgtgccgctgctcggcttcatcggccgcctggacgggcagaagggcgtggagatcatcgcggacgccatgccctggatcgtgagccaggacgtgcagctggtgatgctgggcaccgggcgccacgacctggagagcatgctgcagcacttcgagcgggagcaccacgacaaggtgcgcgggtgggtggggttctccgtgcgcctggcgcaccggatcacggcgggggcggacgcgctcctcatgccctcccggttcgagccgtgcgggctgaaccagctctacgccatggcctacggcaccgtccccgtcgtgcacgccgtcggcggcctcagggacaccgtgccgccgttcgaccccttcaaccactccgggctcgggtggacgttcgaccgcgccgaggcgcacaagctgatcgaggcgctcgggcactgcctccgcacctaccgagacttcaaggagagctggagggccctccaggagcgcggcatgtcgcaggacttcagctgggagcacgccgccaagctctacgaggacgtcctcgtcaaggccaagtaccagtggtga

SEQ ID NO: 44 - protein, 799 aa, Artificial Sequence
  RJ 122 Mutant Starch Synthase II Genome D Protein
MSSAVASAASFLALASASPGRSRRRARVSAQPPHAGAGRLHWPPWPPQRTARDGAVAALAAGKKDAGIDDAAASVRQPRALRGGAATKVAERRDPVKTLDRDAAEGGGPSPPAARQDAARPPSMNGMPVNGENKSTGGGGATKDSGLPTPARAPHPSTQNRAPVNGENKANVASPPTSIAEAAASDSAATISISDKAPESVVPAEKTPPSSGSNFESSASAPGSDTVSDVEQELKKGAVVVEEAPKPKALSPPAAPAVQEDLWDFKKYIGFEEPVEAKDDGRAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPLFRHRQEDIYGGSRQEIMKRMILFCKAVVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDAHLKSDGYTNFSLRTLDSGKRQCKEALQRELGLQVRADVPLLGFIGRLDGQKGVEIIADAMPWIVSQDVQLVMLGTGRHDLESMLQHFEREHHDKVRGWVGFSVRLAHRITAGADALLMPSRFEPCGLNQLYAMAYGTVPVVHAVGGLRDTVPPFDPFNHSGLGWTFDRAEAHKLIEALGHCLRTYRDFKESWRALQERGMSQDFSWEHAAKLYEDVLVKAKYQW

SEQ ID NO: 45 - DNA, 2397 bp, Artificial Sequence
  M123 ID 213 Mutant Starch Synthase II Genome B CDS
atgtcgtcggcggtcgcgtccgccgcgtccttcctcgcgctcgcgtccgcctcccccgggagatcacggaggaggacgagggtgagcgcgtcgccaccccacaccggggctggcaggttgcactggccgccgtcgccgccgcagcgcacggctcgcgacggagcagtggccgcgcgcgccgccgggaagaaggacgcggggatcgacgacgccgcgcccgcgaggcagccccgcgcactccgcggtggcgccgccaccaaggttgcggagcggagggatcccgtcaagacgctcgatcgcgacgccgcggaaggtggcgcgccgtccccgccggcaccgaggcaggaggacgcccgtctgccgagcatgaacggcatgccggtgaacggtgaaaacaaatctaccggcggcggcggcgcgactaaagacagcgggctgcccgcacccgcacgcgcgccccagccgtcgagccagaacagagtaccggtgaatggtgaaaacaaagctaacgtcgcctcgccgccgacgagcatagccgaggtcgcggctccggatcccgcagctaccatttccatcagtgacaaggcgccagagtccgttgtcccagccgagaaggcgccgccgtcgtccggctcaaatttcgtgccctcggcttctgctcccgggtctgacactgtcagcgacgtggaacttgaactgaagaagggtgcggtcattgtcaaagaagctccaaacccaaaggctctttcgccgcctgcagcacccgctgtacaacaagacctttgggacttcaagaaatacattggtttcgaggagcccgtggaggccaaggatgatggccgggctgttgcagatgatgcgggctccttcgaacaccaccagaatcacgattccgggcctttggcaggggagaacgtcatgaacgtggtcgtcgtggctgctgaatgttctccctggtgcaaaacaggtggtcttggagatgttgccggtgctttgctcaaggctttggcgaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctctcttccgacaccgccaggaagacatttatgggggcagcagacaggaaattatgaagcgcatgattttgttctgcaaggccgctgtcgaggttccatggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgctcaccagggccgtggcccagtagatgagttcccgttcaccgagttgcctgagcactacctggaacacttcagactgtacgaccccgtgggtggtgaacacgccaactacttcgccgccggcctgaagatggcggaccaggttgtcgtcgtgagcccggggtacctgtgggagctgaagacggtggagggcggctgggggcttcacgacatcatacggcagaacgactggaagacccgcggcatcgtgaacggcatcgacaacatggagtggaaccccgaggtggacgtccacctcaagtcggacggctacaccaacttctccctggggacgctggactccggcaagcggcagtgcaaggaggccctgcagcgggagctgggcctgcaggtccgcggcgacgtgccgctgctcggcttcatcgggcgcctggacgggcagaagggcgtggagatcatcgcggacgcgatgccctggatcgtgagccaggacgtgcagctggtcatgctgggcaccgggcgccacgacctggagggcatgctgcggcacttcgagcgggagcaccacgacaaggtgcgcgggtgggtggggttctccgtgcggctggcgcaccggatcacggccggcgccgacgcgctcctcatgccctcccggttcgagccgtgcggactgaaccagctctacgccatggcctacggcaccgtccccgtcgtgcatgccgtcggcggcctgagggacaccgtgccgccgttcgaccccttcaaccactccgggctcgggtggacgttcgaccgcgcagaggcgcagaagctgatcgaggcgctcgggcactgcctccgcacctaccgggactacaaggagagctggagggggctccaggagcgcggcatgtcgcaggacttcagctgggagcatgccgccaagctctacgaggacgtcctcgtcaaggccaagtaccagtggtga

SEQ ID NO: 46 - protein, 798 aa, Artificial Sequence
  M123 ID 213 Mutant Starch Synthase II Genome B Protein
MSSAVASAASFLALASASPGRSRRRTRVSASPPHTGAGRLHWPPSPPQRTARDGAVAARAAGKKDAGIDDAAPARQPRALRGGAATKVAERRDPVKTLDRDAAEGGAPSPPAPRQEDARLPSMNGMPVNGENKSTGGGGATKDSGLPAPARAPQPSSQNRVPVNGENKANVASPPTSIAEVAAPDPAATISISDKAPESVVPAEKAPPSSGSNFVPSASAPGSDTVSDVELELKKGAVIVKEAPNPKALSPPAAPAVQQDLWDFKKYIGFEEPVEAKDDGRAVADDAGSFEHHQNHDSGPLAGENVMNVVVVAAECSPWCKTGGLGDVAGALLKALAKRGHRVMVVVPRYGDYEEAYDVGVRKYYKAAGQDMEVNYFHAYIDGVDFVFIDAPLFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDVHLKSDGYTNFSLGTLDSGKRQCKEALQRELGLQVRGDVPLLGFIGRLDGQKGVEIIADAMPWIVSQDVQLVMLGTGRHDLEGMLRHFEREHHDKVRGWVGFSVRLAHRITAGADALLMPSRFEPCGLNQLYAMAYGTVPVVHAVGGLRDTVPPFDPFNHSGLGWTFDRAEAQKLIEALGHCLRTYRDYKESWRGLQERGMSQDFSWEHAAKLYEDVLVKAKYQW

SEQ ID NO: 47 - DNA, 2397 bp, Artificial Sequence
  M123 ID 217 Mutant Starch Synthase II Genome B CDS
atgtcgtcggcggtcgcgtccgccgcgtccttcctcgcgctcgcgtccgcctcccccgggagatcacggaggaggacgagggtgagcgcgtcgccaccccacaccggggctggcaggttgcactggccgccgtcgccgccgcagcgcacggctcgcgacggagcagtggccgcgcgcgccgccgggaagaaggacgcggggatcgacgacgccgcgcccgcgaggcagccccgcgcactccgcggtggcgccgccaccaaggttgcggagcggagggatcccgtcaagacgctcgatcgcgacgccgcggaaggtggcgcgccgtccccgccggcaccgaggcaggaggacgcccgtctgccgagcatgaacggcatgccggtgaacggtgaaaacaaatctaccggcggcggcggcgcgactaaagacagcgggctgcccgcacccgcacgcgcgccccagccgtcgagccagaacagagtaccggtgaatggtgaaaacaaagctaacgtcgcctcgccgccgacgagcatagccgaggtcgcggctccggatcccgcagctaccatttccatcagtgacaaggcgccagagtccgttgtcccagccgagaaggcgccgccgtcgtccggctcaaatttcgtgccctcggcttctgctcccgggtctgacactgtcagcgacgtggaacttgaactgaagaagggtgcggtcattgtcaaagaagctccaaacccaaaggctctttcgccgcctgcagcacccgctgtacaacaagacctttgggacttcaagaaatacattggtttcgaggagcccgtggaggccaaggatgatggccgggctgttgcagatgatgcgggctccttcgaacaccaccagaatcacgattccgggcctttggcaggggagaacgtcatgaacgtggtcgtcgtggctgctgaatgttctccctggtgcaaaacaggtggtcttggagatgttgccggtgctttgtccaaggctttggcgaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctctcttccgacaccgccaggaagacatttatgggggcagcagacaggaaattatgaagcgcatgattttgttctgcaaggccgctgtcgaggttccatggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgctcaccagggccgtggcccagtagatgagttcccgttcaccgagttgcctgagcactacctggaacacttcagactgtacgaccccgtgggtggtgaacacgccaactacttcgccgccggcctgaagatggcggaccaggttgtcgtcgtgagcccggggtacctgtgggagctgaagacggtggagggcggctgggggcttcacgacatcatacggcagaacgactggaagacccgcggcatcgtgaacggcatcgacaacatggagtggaaccccgaggtggacgtccacctcaagtcggacggctacaccaacttctccctggggacgctggactccggcaagcggcagtgcaaggaggccctgcagcgggagctgggcctgcaggtccgcggcgacgtgccgctgctcggcttcatcgggcgcctggacgggcagaagggcgtggagatcatcgcggacgcgatgccctggatcgtgagccaggacgtgcagctggtcatgctgggcaccgggcgccacgacctggagggcatgctgcggcacttcgagcgggagcaccacgacaaggtgcgcgggtgggtggggttctccgtgcggctggcgcaccggatcacggccggcgccgacgcgctcctcatgccctcccggttcgagccgtgcggactgaaccagctctacgccatggcctacggcaccgtccccgtcgtgcatgccgtcggcggcctgagggacaccgtgccgccgttcgaccccttcaaccactccgggctcgggtggacgttcgaccgcgcagaggcgcagaagctgatcgaggcgctcgggcactgcctccgcacctaccgggactacaaggagagctggagggggctccaggagcgcggcatgtcgcaggacttcagctgggagcatgccgccaagctctacgaggacgtcctcgtcaaggccaagtaccagtggtga

SEQ ID NO: 48 - protein, 798 aa, Artificial Sequence
  M123 ID 217 Mutant Starch Synthase II Genome B Protein
MSSAVASAASFLALASASPGRSRRRTRVSASPPHTGAGRLHWPPSPPQRTARDGAVAARAAGKKDAGIDDAAPARQPRALRGGAATKVAERRDPVKTLDRDAAEGGAPSPPAPRQEDARLPSMNGMPVNGENKSTGGGGATKDSGLPAPARAPQPSSQNRVPVNGENKANVASPPTSIAEVAAPDPAATISISDKAPESVVPAEKAPPSSGSNFVPSASAPGSDTVSDVELELKKGAVIVKEAPNPKALSPPAAPAVQQDLWDFKKYIGFEEPVEAKDDGRAVADDAGSFEHHQNHDSGPLAGENVMNVVVVAAECSPWCKTGGLGDVAGALSKALAKRGHRVMVVVPRYGDYEEAYDVGVRKYYKAAGQDMEVNYFHAYIDGVDFVFIDAPLFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDVHLKSDGYTNFSLGTLDSGKRQCKEALQRELGLQVRGDVPLLGFIGRLDGQKGVEIIADAMPWIVSQDVQLVMLGTGRHDLEGMLRHFEREHHDKVRGWVGFSVRLAHRITAGADALLMPSRFEPCGLNQLYAMAYGTVPVVHAVGGLRDTVPPFDPFNHSGLGWTFDRAEAQKLIEALGHCLRTYRDYKESWRGLQERGMSQDFSWEHAAKLYEDVLVKAKYQW

SEQ ID NO: 49 - DNA, 2397 bp, Artificial Sequence
  M123 ID 4 Mutant Starch Synthase II Genome B CDS
atgtcgtcggcggtcgcgtccgccgcgtccttcctcgcgctcgcgtccgcctcccccgggagatcacggaggaggacgagggtgagcgcgtcgccaccccacaccggggctggcaggttgcactggccgccgtcgccgccgcagcgcacggctcgcgacggagcagtggccgcgcgcgccgccgggaagaaggacgcggggatcgacgacgccgcgcccgcgaagcagccccgcgcactccgcggtggcgccgccaccaaggttgcggagcggagggatcccgtcaagacgctcgatcgcgacgccgcggaaggtggcgcgccgtccccgccggcaccgaggcaggaggacgcccgtctgccgagcatgaacggcatgccggtgaacggtgaaaacaaatctaccggcggcggcggcgcgactaaagacagcgggctgcccgcacccgcacgcgcgccccagccgtcgagccagaacagagtaccggtgaatggtgaaaacaaagctaacgtcgcctcgccgccgacgagcatagccgaggtcgcggctccggatcccgcagctaccatttccatcagtgacaaggcgccagagtccgttgtcccagccgagaaggcgccgccgtcgtccggctcaaatttcgtgccctcggcttctgctcccgggtctgacactgtcagcgacgtggaacttgaactgaagaagggtgcggtcattgtcaaagaagctccaaacccaaaggctctttcgccgcctgcagcacccgctgtacaacaagacctttgggacttcaagaaatacattggtttcgaggagcccgtggaggccaaggatgatggccgggctgttgcagatgatgcgggctccttcgaacaccaccagaatcacgattccgggcctttggcaggggagaacgtcatgaacgtggtcgtcgtggctgctgaatgttctccctggtgcaaaacaggtggtcttggagatgttgccggtgctttgcccaaggctttggcgaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctctcttccgacaccgccaggaagacatttatgggggcagcagacaggaaattatgaagcgcatgattttgttctgcaaggccgctgtcgaggttccatggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgctcaccagggccgtggcccagtagatgagttcccgttcaccgagttgcctgagcactacctggaacacttcagactgtacgaccccgtgggtggtgaacacgccaactacttcgccgccggcctgaagatggcggaccaggttgtcgtcgtgagcccggggtacctgtgggagctgaagacggtggagggcggctgggggcttcacgacatcatacggcagaacgactggaagacccgcggcatcgtgaacggcatcgacaacatggagtggaaccccgaggtggacgtccacctcaagtcggacggctacaccaacttctccctggggacgctggactccggcaagcggcagtgcaaggaggccctgcagcgggagctgggcctgcaggtccgcggcgacgtgccgctgctcggcttcatcgggcgcctggacgggcagaagggcgtggagatcatcgcggacgcgatgccctggatcgtgagccaggacgtgcagctggtcatgctgggcaccgggcgccacgacctggagggcatgctgcggcacttcgagcgggagcaccacgacaaggtgcgcgggtgggtggggttctccgtgcggctggcgcaccggatcacggccggcgccgacgcgctcctcatgccctcccggttcgagccgtgcggactgaaccagctctacgccatggcctacggcaccgtccccgtcgtgcatgccgtcggcggcctgagggacaccgtgccgccgttcgaccccttcaaccactccgggctcgggtggacgttcgaccgcgcagaggcgcagaagctgatcgaggcgctcgggcactgcctccgcacctaccgggactacaaggagagctggagggggctccaggagcgcggcatgtcgcaggacttcagctgggagcatgccgccaagctctacgaggacgtcctcgtcaaggccaagtaccagtggtga

SEQ ID NO: 50 - protein, 798 aa, Artificial Sequence
  M123 ID 4 Mutant Starch Synthase II Genome B Protein
MSSAVASAASFLALASASPGRSRRRTRVSASPPHTGAGRLHWPPSPPQRTARDGAVAARAAGKKDAGIDDAAPAKQPRALRGGAATKVAERRDPVKTLDRDAAEGGAPSPPAPRQEDARLPSMNGMPVNGENKSTGGGGATKDSGLPAPARAPQPSSQNRVPVNGENKANVASPPTSIAEVAAPDPAATISISDKAPESVVPAEKAPPSSGSNFVPSASAPGSDTVSDVELELKKGAVIVKEAPNPKALSPPAAPAVQQDLWDFKKYIGFEEPVEAKDDGRAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPLFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDVHLKSDGYTNFSLGTLDSGKRQCKEALQRELGLQVRGDVPLLGFIGRLDGQKGVEIIADAMPWIVSQDVQLVMLGTGRHDLEGMLRHFEREHHDKVRGWVGFSVRLAHRITAGADALLMPSRFEPCGLNQLYAMAYGTVPVVHAVGGLRDTVPPFDPFNHSGLGWTFDRAEAQKLIEALGHCLRTYRDYKESWRGLQERGMSQDFSWEHAAKLYEDVLVKAKYQW

SEQ ID NO: 51 - DNA, 2397 bp, Artificial Sequence
  MS42 ID 275 Mutant Starch Synthase II Genome B CDS
atgtcgtcggcggtcgcgtccgccgcgtccttcctcgcgctcgcgtccgcctcccccgggagatcacggaggaggacgagggtgagcgcgtcgccaccccacaccggggctggcaggttgcactggccgccgtcgccgccgcagcgcacggctcgcgacggagcagtggccgcgcgcgccgccgggaagaaggacgcggggatcgacgacgccgcgcccgcgaggcagccccgcgcactccgcggtggcgccgccaccaaggttgcggagcggagggatcccgtcaagacgctcgatcgcgacgccgcggaaggtggcgcgccgtccccgccggcaccgaggcaggaggacgcccgtctgccgagcatgaacggcatgccggtgaacggtgaaaacaaatctaccggcggcggcggcgcgactaaagacagcgggctgcccgcacccgcacgcgcgccccagccgtcgagccagaacagagtaccggtgaatggtgaaaacaaagctaacgtcgcctcgccgccgacgagcatagccgaggtcgcggctccggatcccgcagctaccatttccatcagtgacaaggcgccagagtccgttgtcccagccgagaaggcgccgccgtcgtccggctcaaatttcgtgccctcggcttctgctcccgggtctgacactgtcagcgacgtggaacttgaactgaagaagggtgcggtcattgtcaaagaagctccaaacccaaaggctctttcgccgcctgcagcacccgctgtacaacaagacctttgggacttcaagaaatacattggtttcgaggagcccgtggaggccaaggatgatggccgggctgttgcaaatgatgcgggctccttcgaacaccaccagaatcacgattccgggcctttggcaggggagaacgtcatgaacgtggtcgtcgtggctgctgaatgttctccctggtgcaaaacaggtggtcttggagatgttgccggtgctttgcccaaggctttggcgaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctctcttccgacaccgccaggaagacatttatgggggcagcagacaggaaattatgaagcgcatgattttgttctgcaaggccgctgtcgaggttccatggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgctcaccagggccgtggcccagtagatgagttcccgttcaccgagttgcctgagcactacctggaacacttcagactgtacgaccccgtgggtggtgaacacgccaactacttcgccgccggcctgaagatggcggaccaggttgtcgtcgtgagcccggggtacctgtgggagctgaagacggtggagggcggctgggggcttcacgacatcatacggcagaacgactggaagacccgcggcatcgtgaacggcatcgacaacatggagtggaaccccgaggtggacgtccacctcaagtcggacggctacaccaacttctccctggggacgctggactccggcaagcggcagtgcaaggaggccctgcagcgggagctgggcctgcaggtccgcggcgacgtgccgctgctcggcttcatcgggcgcctggacgggcagaagggcgtggagatcatcgcggacgcgatgccctggatcgtgagccaggacgtgcagctggtcatgctgggcaccgggcgccacgacctggagggcatgctgcggcacttcgagcgggagcaccacgacaaggtgcgcgggtgggtggggttctccgtgcggctggcgcaccggatcacggccggcgccgacgcgctcctcatgccctcccggttcgagccgtgcggactgaaccagctctacgccatggcctacggcaccgtccccgtcgtgcatgccgtcggcggcctgagggacaccgtgccgccgttcgaccccttcaaccactccgggctcgggtggacgttcgaccgcgcagaggcgcagaagctgatcgaggcgctcgggcactgcctccgcacctaccgggactacaaggagagctggagggggctccaggagcgcggcatgtcgcaggacttcagctgggagcatgccgccaagctctacgaggacgtcctcgtcaaggccaagtaccagtggtga

SEQ ID NO: 52 - protein, 798 aa, Artificial Sequence
  MS42 ID 275 Mutant Starch Synthase II Genome B Protein
MSSAVASAASFLALASASPGRSRRRTRVSASPPHTGAGRLHWPPSPPQRTARDGAVAARAAGKKDAGIDDAAPARQPRALRGGAATKVAERRDPVKTLDRDAAEGGAPSPPAPRQEDARLPSMNGMPVNGENKSTGGGGATKDSGLPAPARAPQPSSQNRVPVNGENKANVASPPTSIAEVAAPDPAATISISDKAPESVVPAEKAPPSSGSNFVPSASAPGSDTVSDVELELKKGAVIVKEAPNPKALSPPAAPAVQQDLWDFKKYIGFEEPVEAKDDGRAVANDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPLFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDVHLKSDGYTNFSLGTLDSGKRQCKEALQRELGLQVRGDVPLLGFIGRLDGQKGVEIIADAMPWIVSQDVQLVMLGTGRHDLEGMLRHFEREHHDKVRGWVGFSVRLAHRITAGADALLMPSRFEPCGLNQLYAMAYGTVPVVHAVGGLRDTVPPFDPFNHSGLGWTFDRAEAQKLIEALGHCLRTYRDYKESWRGLQERGMSQDFSWEHAAKLYEDVLVKAKYQW

SEQ ID NO: 53 - DNA, 2397 bp, Artificial Sequence
  MS42 ID 224 Mutant Starch Synthase II Genome B CDS
atgtcgtcggcggtcgcgtccgccgcgtccttcctcgcgctcgcgtccgcctcccccgggagatcacggaggaggacgagggtgagcgcgtcgccaccccacaccggggctggcaggttgcactggccgccgtcgccgccgcagcgcacggctcgcgacggagcagtggccgcgcgcgccgccgggaagaaggacgcggggatcgacgacgccgcgcccgcgaggcagccccgcgcactccgcggtggcgccgccaccaaggttgcggagcggagggatcccgtcaagacgctcgatcgcgacgccgcggaaggtggcgcgccgtccccgccggcaccgaggcaggaggacgcccgtctgccgagcatgaacggcatgccggtgaacggtgaaaacaaatctaccggcggcggcggcgcgactaaagacagcgggctgcccgcacccgcacgcgcgccccagccgtcgagccagaacagagtaccggtgaatggtgaaaacaaagctaacgtcgcctcgccgccgacgagcatagccgaggtcgcggctccggatcccgcagctaccatttccatcagtgacaaggcgccagagtccgttgtcccagccgagaaggcgccgccgtcgtccggctcaaatttcgtgccctcggcttctgctcccgggtctgacactgtcagcgacgtggaacttgaactgaagaagggtgcggtcattgtcaaagaagctccaaacccaaaggctctttcgccgcctgcagcacccgctgtacaacaagacctttgggacttcaagaaatacattggtttcgaggagcccgtggaggccaaggatgatggccgggctgttgcagatgatgcgggctccttcgaacaccaccagaatcacgattccgggcctttggcaggggagaacgtcatgaacgtggtcgtcgtggctgctgaatgttctccctggtgcaaaacaggtggtcttggagatgttgccgatgctttgcccaaggctttggcgaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctctcttccgacaccgccaggaagacatttatgggggcagcagacaggaaattatgaagcgcatgattttgttctgcaaggccgctgtcgaggttccatggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgctcaccagggccgtggcccagtagatgagttcccgttcaccgagttgcctgagcactacctggaacacttcagactgtacgaccccgtgggtggtgaacacgccaactacttcgccgccggcctgaagatggcggaccaggttgtcgtcgtgagcccggggtacctgtgggagctgaagacggtggagggcggctgggggcttcacgacatcatacggcagaacgactggaagacccgcggcatcgtgaacggcatcgacaacatggagtggaaccccgaggtggacgtccacctcaagtcggacggctacaccaacttctccctggggacgctggactccggcaagcggcagtgcaaggaggccctgcagcgggagctgggcctgcaggtccgcggcgacgtgccgctgctcggcttcatcgggcgcctggacgggcagaagggcgtggagatcatcgcggacgcgatgccctggatcgtgagccaggacgtgcagctggtcatgctgggcaccgggcgccacgacctggagggcatgctgcggcacttcgagcgggagcaccacgacaaggtgcgcgggtgggtggggttctccgtgcggctggcgcaccggatcacggccggcgccgacgcgctcctcatgccctcccggttcgagccgtgcggactgaaccagctctacgccatggcctacggcaccgtccccgtcgtgcatgccgtcggcggcctgagggacaccgtgccgccgttcgaccccttcaaccactccgggctcgggtggacgttcgaccgcgcagaggcgcagaagctgatcgaggcgctcgggcactgcctccgcacctaccgggactacaaggagagctggagggggctccaggagcgcggcatgtcgcaggacttcagctgggagcatgccgccaagctctacgaggacgtcctcgtcaaggccaagtaccagtggtga

SEQ ID NO: 54 - protein, 798 aa, Artificial Sequence
  MS42 ID 224 Mutant Starch Synthase II Genome B Protein
MSSAVASAASFLALASASPGRSRRRTRVSASPPHTGAGRLHWPPSPPQRTARDGAVAARAAGKKDAGIDDAAPARQPRALRGGAATKVAERRDPVKTLDRDAAEGGAPSPPAPRQEDARLPSMNGMPVNGENKSTGGGGATKDSGLPAPARAPQPSSQNRVPVNGENKANVASPPTSIAEVAAPDPAATISISDKAPESVVPAEKAPPSSGSNFVPSASAPGSDTVSDVELELKKGAVIVKEAPNPKALSPPAAPAVQQDLWDFKKYIGFEEPVEAKDDGRAVADDAGSFEHHQNHDSGPLAGENVMNVVVVAAECSPWCKTGGLGDVADALPKALAKRGHRVMVVVPRYGDYEEAYDVGVRKYYKAAGQDMEVNYFHAYIDGVDFVFIDAPLFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDVHLKSDGYTNFSLGTLDSGKRQCKEALQRELGLQVRGDVPLLGFIGRLDGQKGVEIIADAMPWIVSQDVQLVMLGTGRHDLEGMLRHFEREHHDKVRGWVGFSVRLAHRITAGADALLMPSRFEPCGLNQLYAMAYGTVPVVHAVGGLRDTVPPFDPFNHSGLGWTFDRAEAQKLIEALGHCLRTYRDYKESWRGLQERGMSQDFSWEHAAKLYEDVLVKAKYQW

SEQ ID NO: 55 - DNA, 2400 bp, Artificial Sequence
  EMS Divide 1631 Mutant Starch Synthase II Genome A CDS
atgtcgtcggcggtcgcgtccgccgcgtccttcctcgcgctcgcctccgcctcccccgggagatcacgcaggcgggcgagggtgagcgcgccgccaccccacgccggggccggcaggctgcactggccgccgtggccgccgcagcgcacggctcgcgacggaggtgtggccgcgcgcgccgccgggaagaaggacgcgagggtcgacgacgacgccgcgtccgcgaggcagccccgcgcacgccgcggtggcgccgccaccaaggtcgcggagcggagggatcccgtcaagacgctcgatcgcgacgccgcggaaggtggcgcgccggcaccgccggcaccgaggcaggacgccgtccgtccaccgagtatgaacggcacgccggtgaacggtgagaacaaatctaccggcggcggcggcgcgaccaaagacagcgggctgcccgcacccgcacgcgcgccccatccgtcgacccagaacagagtaccagtgaacggtgaaaacaaagctaacgtcgcctcgccgccgacgagcatagccgaggtcgtggctccggattccgcagctaccatttccatcagtgacaaggcgccggagtccgttgtcccagccgagaagccgccgccgtcgtccggctcaaatttcgtggtctcggcttctgctcccaggctggacattgacagcgatgttgaacctgaactgaagaagggtgcggtcatcgtcgaagaagctccaaacccaaaggctctttcgccgcctgcagcccccgctgtacaagaagacctttgggacttcaagaaatacattggcttcgaggagcccgtggaggccaaggatgatggctgggctgttgcagatgatgcgggctcctttgaacatcaccagaaccatgattccggacctttggcaggggagaacgtcatgaacgtggtcgtcgtggctgctgaatgttctccctggtgcaaaacaggtggtcttggagatgttgcgggtgctctgcccaaggctttggcaaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctatcttccgacaccgtcaggaagacatttatgggggcagcagacaggaaattatgaagcgcatgattttgttctgcaaggccgctgtcgaggttccttggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgcgcaccagggccgtggcccagtagatgaattcccgttcaccgagttgcctgagcactacctggaacacttcagactgtacgaccccgtgggtggtgagcacgccaactacttcgccgccggcctgaagatggcggaccaggttgtcgtggtgagccccgggtacctgtgggagctcaagacggtggagggcggctgggggcttcacgacatcatacggcagaacgactggaagacccgcggcatcgtcaacggcatcgacaacatggagtggaaccccgaggtggacgtccacctccagtcggacggctacaccaacttctccctgagcacgctggactccggcaagcggcagtgcaaggaggccctgcagcgcgagctgggcctgcaggtccgcgccgacgtgccgctgctcggcttcatcggccgcctggacgggcagaagggcgtggagatcatcgcggacgccatgccctggatcgtgagccaggacgtgcagctggtcatgctgggcaccggccgccacgacctggagagcatgctgcggcacttcgagcgggagcaccacgacaaggtgcgcgggtgggtggggttctccgtgcgcctggcgcaccggatcacggcgggcgccgacgcgctcctcatgccctcccggttcgagccgtgcgggctgaaccagctctacgccatggcctacggcaccgtccccgtcgtgcacgccgtcggcgggctgagggacaccgtgccgccgttcgaccccttcaaccactccggcctcgggtggacgttcgaccgcgccgaggcgcacaagctgatcgaggcgctcgggcactgcctccgcacctaccgggactacaaggagagctggaggggcctccaggagcgcggcatgtcgcaggacttcagctgggagcatgccgccaagctctacgaggacgtcctcctcaaggccaagtaccagtggtga

SEQ ID NO: 56 - protein, 799 aa, Artificial Sequence
  EMS Divide 1631 Mutant Starch Synthase II Genome A Protein
MSSAVASAASFLALASASPGRSRRRARVSAPPPHAGAGRLHWPPWPPQRTARDGGVAARAAGKKDARVDDDAASARQPRARRGGAATKVAERRDPVKTLDRDAAEGGAPAPPAPRQDAVRPPSMNGTPVNGENKSTGGGGATKDSGLPAPARAPHPSTQNRVPVNGENKANVASPPTSIAEVVAPDSAATISISDKAPESVVPAEKPPPSSGSNFVVSASAPRLDIDSDVEPELKKGAVIVEEAPNPKALSPPAAPAVQEDLWDFKKYIGFEEPVEAKDDGWAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPIFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDVHLQSDGYTNFSLSTLDSGKRQCKEALQRELGLQVRADVPLLGFIGRLDGQKGVEIIADAMPWIVSQDVQLVMLGTGRHDLESMLRHFEREHHDKVRGWVGFSVRLAHRITAGADALLMPSRFEPCGLNQLYAMAYGTVPVVHAVGGLRDTVPPFDPFNHSGLGWTFDRAEAHKLIEALGHCLRTYRDYKESWRGLQERGMSQDFSWEHAAKLYEDVLLKAKYQW

SEQ ID NO: 57 - DNA, 2400 bp, Artificial Sequence
  EMS Divide 1214 Mutant Starch Synthase II Genome A CDS
atgtcgtcggcggtcgcgtccgccgcgtccttcctcgcgctcgcctccgcctcccccgggagatcacgcaggcgggcgagggtgagcgcgccgccaccccacgccggggccggcaggctgcactggccgccgtggccgccgcagcgcacggctcgcgacggaggtgtggccgcgcgcgccgccgggaagaaggacgcgagggtcgacgacgacgccgcgtccgcgaggcagccccgcgcacgccgcggtggcgccgccaccaaggtcgcggagcggagggatcccgtcaagacgctcgatcgcgacgccgcggaaggtggcgcgccggcaccgccggcaccgaggcaggacgccgcccgtccatcgagtatgaacggcacgccggtgaacggtgagaacaaatctaccggcggcggcggcgcgaccaaagacagcgggctgcccgcacccgcacgcgcgccccatccgtcgacccagaacagagtaccagtgaacggtgaaaacaaagctaacgtcgcctcgccgccgacgagcatagccgaggtcgtggctccggattccgcagctaccatttccatcagtgacaaggcgccggagtccgttgtcccagccgagaagccgccgccgtcgtccggctcaaatttcgtggtctcggcttctgctcccaggctggacattgacagcgatgttgaacctgaactgaagaagggtgcggtcatcgtcgaagaagctccaaacccaaaggctctttcgccgcctgcagcccccgctgtacaagaagacctttgggacttcaagaaatacattggcttcgaggagcccgtggaggccaaggatgatggctgggctgttgcagatgatgcgggctcctttgaacatcaccagaaccatgattccggacctttggcaggggagaacgtcatgaacgtggtcgtcgtggctgctgaatgttctccctggtgcaaaacaggtggtcttggagatgttgcgggtgctctgcccaaggctttggcaaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctatcttccgacaccgtcaggaagacatttatgggggcagcagacaggaaattatgaagcgcatgattttgttctgcaaggccgctgtcgaggttccttggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgcgcaccagggccgtggcccagtagatgaattcccgttcaccgagttgcctgagcactacctggaacacttcagactgtacgaccccgtgggtggtgagcacgccaactacttcgccgccggcctgaagatggcggaccaggttgtcgtggtgagccccgggtacctgtgggagctcaagacggtggagggcggctgggggcttcacgacatcatacggcagaacgactggaagacccgcggcatcgtcaacggcatcgacaacatggagtggaaccccgaggtggacgtccacctccagtcggacggctacaccaacttctccctgagcacgctggactccggcaagcggcagtgcaaggaggccctgcagcgcgagctgggcctgcaggtccgcgccgacgtgccgctgctcggcttcatcggccgcctggacgggcagaagggcgtggagatcatcgcggacgccatgccctggatcgtgagccaggacgtgcagctggtcatgctgggcaccggccgccacgacctggagagcatgctgcggcacttcgagcgggagcaccacgacaaggtgcgcgggtgggtggggttctccgtgcgcctggcgcaccggatcacggcgggcgccgacgcgctcctcatgccctcccggttcgagccgtgcgggctgaaccagctctacgccatggcctacggcaccgtccccgtcgtgcacgccgtcggcgggctgagggacaccgtgccgccgttcgaccccttcaaccactccggcctcgggtggacgttcgaccgcgccgaggcgcacaagctgatcgaggcgctcgggcactgcctccgcacctaccgggactacaaggagagctggaggggcctccaggagcgcggcatgtcgcaggacttcagctgggagcatgccgccaagctctacgaggacgtcctcctcaaggccaagtaccagtggtga

SEQ ID NO: 58 - protein, 799 aa, Artificial Sequence
  EMS Divide 1214 Mutant Starch Synthase II Genome A Protein
MSSAVASAASFLALASASPGRSRRRARVSAPPPHAGAGRLHWPPWPPQRTARDGGVAARAAGKKDARVDDDAASARQPRARRGGAATKVAERRDPVKTLDRDAAEGGAPAPPAPRQDAARPSSMNGTPVNGENKSTGGGGATKDSGLPAPARAPHPSTQNRVPVNGENKANVASPPTSIAEVVAPDSAATISISDKAPESVVPAEKPPPSSGSNFVVSASAPRLDIDSDVEPELKKGAVIVEEAPNPKALSPPAAPAVQEDLWDFKKYIGFEEPVEAKDDGWAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPIFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDVHLQSDGYTNFSLSTLDSGKRQCKEALQRELGLQVRADVPLLGFIGRLDGQKGVEIIADAMPWIVSQDVQLVMLGTGRHDLESMLRHFEREHHDKVRGWVGFSVRLAHRITAGADALLMPSRFEPCGLNQLYAMAYGTVPVVHAVGGLRDTVPPFDPFNHSGLGWTFDRAEAHKLIEALGHCLRTYRDYKESWRGLQERGMSQDFSWEHAAKLYEDVLLKAKYQW

SEQ ID NO: 59 - DNA, 2400 bp, Artificial Sequence
  EMS Divide 81 Mutant Starch Synthase II Genome A CDS
atgtcgtcggcggtcgcgtccgccgcgtccttcctcgcgctcgcctccgcctcccccgggagatcacgcaggcgggcgagggtgagcgcgccgccaccccacgccggggccggcaggctgcactggccgccgtggccgccgcagcgcacggctcgcgacggaggtgtggccgcgcgcgccgccgggaagaaggacgcgagggtcgacgacgacgccgcgtccgcgaggcagccccgcgcacgccgcggtggcgccgccaccaaggtcgcggagcggagggatcccgtcaagacgctcgatcgcgacgccgcggaaggtggcgcgccggcaccgccggcaccgaggcaggacgccgcccgtccaccgagtatgaacggcacgccggtgaacggtgagaacaaatctaccggcggcggcggcgcgaccaaagacagcgggctgcccgcacccgcacgcgcgccccattcgtcgacccagaacagagtaccagtgaacggtgaaaacaaagctaacgtcgcctcgccgccgacgagcatagccgaggtcgtggctccggattccgcagctaccatttccatcagtgacaaggcgccggagtccgttgtcccagccgagaagccgccgccgtcgtccggctcaaatttcgtggtctcggcttctgctcccaggctggacattgacagcgatgttgaacctgaactgaagaagggtgcggtcatcgtcgaagaagctccaaacccaaaggctctttcgccgcctgcagcccccgctgtacaagaagacctttgggacttcaagaaatacattggcttcgaggagcccgtggaggccaaggatgatggctgggctgttgcagatgatgcgggctcctttgaacatcaccagaaccatgattccggacctttggcaggggagaacgtcatgaacgtggtcgtcgtggctgctgaatgttctccctggtgcaaaacaggtggtcttggagatgttgcgggtgctctgcccaaggctttggcaaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctatcttccgacaccgtcaggaagacatttatgggggcagcagacaggaaattatgaagcgcatgattttgttctgcaaggccgctgtcgaggttccttggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgcgcaccagggccgtggcccagtagatgaattcccgttcaccgagttgcctgagcactacctggaacacttcagactgtacgaccccgtgggtggtgagcacgccaactacttcgccgccggcctgaagatggcggaccaggttgtcgtggtgagccccgggtacctgtgggagctcaagacggtggagggcggctgggggcttcacgacatcatacggcagaacgactggaagacccgcggcatcgtcaacggcatcgacaacatggagtggaaccccgaggtggacgtccacctccagtcggacggctacaccaacttctccctgagcacgctggactccggcaagcggcagtgcaaggaggccctgcagcgcgagctgggcctgcaggtccgcgccgacgtgccgctgctcggcttcatcggccgcctggacgggcagaagggcgtggagatcatcgcggacgccatgccctggatcgtgagccaggacgtgcagctggtcatgctgggcaccggccgccacgacctggagagcatgctgcggcacttcgagcgggagcaccacgacaaggtgcgcgggtgggtggggttctccgtgcgcctggcgcaccggatcacggcgggcgccgacgcgctcctcatgccctcccggttcgagccgtgcgggctgaaccagctctacgccatggcctacggcaccgtccccgtcgtgcacgccgtcggcgggctgagggacaccgtgccgccgttcgaccccttcaaccactccggcctcgggtggacgttcgaccgcgccgaggcgcacaagctgatcgaggcgctcgggcactgcctccgcacctaccgggactacaaggagagctggaggggcctccaggagcgcggcatgtcgcaggacttcagctgggagcatgccgccaagctctacgaggacgtcctcctcaaggccaagtaccagtggtga

SEQ ID NO: 60 - protein, 799 aa, Artificial Sequence
  EMS Divide 81 Mutant Starch Synthase II Genome A Protein
MSSAVASAASFLALASASPGRSRRRARVSAPPPHAGAGRLHWPPWPPQRTARDGGVAARAAGKKDARVDDDAASARQPRARRGGAATKVAERRDPVKTLDRDAAEGGAPAPPAPRQDAARPPSMNGTPVNGENKSTGGGGATKDSGLPAPARAPHSSTQNRVPVNGENKANVASPPTSIAEVVAPDSAATISISDKAPESVVPAEKPPPSSGSNFVVSASAPRLDIDSDVEPELKKGAVIVEEAPNPKALSPPAAPAVQEDLWDFKKYIGFEEPVEAKDDGWAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPIFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDVHLQSDGYTNFSLSTLDSGKRQCKEALQRELGLQVRADVPLLGFIGRLDGQKGVEIIADAMPWIVSQDVQLVMLGTGRHDLESMLRHFEREHHDKVRGWVGFSVRLAHRITAGADALLMPSRFEPCGLNQLYAMAYGTVPVVHAVGGLRDTVPPFDPFNHSGLGWTFDRAEAHKLIEALGHCLRTYRDYKESWRGLQERGMSQDFSWEHAAKLYEDVLLKAKYQW

SEQ ID NO: 61 - DNA, 2400 bp, Artificial Sequence
  EMS Divide 904 Mutant Starch Synthase II Genome A CDS
atgtcgtcggcggtcgcgtccgccgcgtccttcctcgcgctcgcctccgcctcccccgggagatcacgcaggcgggcgagggtgagcgcgccgccaccccacgccggggccggcaggctgcactggccgccgtggccgccgcagcgcacggctcgcgacggaggtgtggccgcgcgcgccgccgggaagaaggacgcgagggtcgacgacgacgccgcgtccgcgaggcagccccgcgcacgccgcggtggcgccgccaccaaggtcgcggagcggagggatcccgtcaagacgctcgatcgcgacgccgcggaaggtggcgcgccggcaccgccggcaccgaggcaggacgccgcccgtccaccgagtatgaacggcacgccggtgaacggtgagaacaaatctaccggcggcggcggcgcgaccaaagacagcgggctgcccgcacccgcacgcgcgccccatccgtcgacccagaacagagtaccagtgaacggtgaaaacaaagctaacgtcgcctcgccgccgacgagcatagccgaggtcgtggctctggattccgcagctaccatttccatcagtgacaaggcgccggagtccgttgtcccagccgagaagccgccgccgtcgtccggctcaaatttcgtggtctcggcttctgctcccaggctggacattgacagcgatgttgaacctgaactgaagaagggtgcggtcatcgtcgaagaagctccaaacccaaaggctctttcgccgcctgcagcccccgctgtacaagaagacctttgggacttcaagaaatacattggcttcgaggagcccgtggaggccaaggatgatggctgggctgttgcagatgatgcgggctcctttgaacatcaccagaaccatgattccggacctttggcaggggagaacgtcatgaacgtggtcgtcgtggctgctgaatgttctccctggtgcaaaacaggtggtcttggagatgttgcgggtgctctgcccaaggctttggcaaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctatcttccgacaccgtcaggaagacatttatgggggcagcagacaggaaattatgaagcgcatgattttgttctgcaaggccgctgtcgaggttccttggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgcgcaccagggccgtggcccagtagatgaattcccgttcaccgagttgcctgagcactacctggaacacttcagactgtacgaccccgtgggtggtgagcacgccaactacttcgccgccggcctgaagatggcggaccaggttgtcgtggtgagccccgggtacctgtgggagctcaagacggtggagggcggctgggggcttcacgacatcatacggcagaacgactggaagacccgcggcatcgtcaacggcatcgacaacatggagtggaaccccgaggtggacgtccacctccagtcggacggctacaccaacttctccctgagcacgctggactccggcaagcggcagtgcaaggaggccctgcagcgcgagctgggcctgcaggtccgcgccgacgtgccgctgctcggcttcatcggccgcctggacgggcagaagggcgtggagatcatcgcggacgccatgccctggatcgtgagccaggacgtgcagctggtcatgctgggcaccggccgccacgacctggagagcatgctgcggcacttcgagcgggagcaccacgacaaggtgcgcgggtgggtggggttctccgtgcgcctggcgcaccggatcacggcgggcgccgacgcgctcctcatgccctcccggttcgagccgtgcgggctgaaccagctctacgccatggcctacggcaccgtccccgtcgtgcacgccgtcggcgggctgagggacaccgtgccgccgttcgaccccttcaaccactccggcctcgggtggacgttcgaccgcgccgaggcgcacaagctgatcgaggcgctcgggcactgcctccgcacctaccgggactacaaggagagctggaggggcctccaggagcgcggcatgtcgcaggacttcagctgggagcatgccgccaagctctacgaggacgtcctcctcaaggccaagtaccagtggtga

SEQ ID NO: 62 - protein, 799 aa, Artificial Sequence
  EMS Divide 904 Mutant Starch Synthase II Genome A Protein
MSSAVASAASFLALASASPGRSRRRARVSAPPPHAGAGRLHWPPWPPQRTARDGGVAARAAGKKDARVDDDAASARQPRARRGGAATKVAERRDPVKTLDRDAAEGGAPAPPAPRQDAARPPSMNGTPVNGENKSTGGGGATKDSGLPAPARAPHPSTQNRVPVNGENKANVASPPTSIAEVVALDSAATISISDKAPESVVPAEKPPPSSGSNFVVSASAPRLDIDSDVEPELKKGAVIVEEAPNPKALSPPAAPAVQEDLWDFKKYIGFEEPVEAKDDGWAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPIFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDVHLQSDGYTNFSLSTLDSGKRQCKEALQRELGLQVRADVPLLGFIGRLDGQKGVEIIADAMPWIVSQDVQLVMLGTGRHDLESMLRHFEREHHDKVRGWVGFSVRLAHRITAGADALLMPSRFEPCGLNQLYAMAYGTVPVVHAVGGLRDTVPPFDPFNHSGLGWTFDRAEAHKLIEALGHCLRTYRDYKESWRGLQERGMSQDFSWEHAAKLYEDVLLKAKYQW

SEQ ID NO: 63 - DNA, 2400 bp, Artificial Sequence
  EMS Divide 280 Mutant Starch Synthase II Genome A CDS
atgtcgtcggcggtcgcgtccgccgcgtccttcctcgcgctcgcctccgcctcccccgggagatcacgcaggcgggcgagggtgagcgcgccgccaccccacgccggggccggcaggctgcactggccgccgtggccgccgcagcgcacggctcgcgacggaggtgtggccgcgcgcgccgccgggaagaaggacgcgagggtcgacgacgacgccgcgtccgcgaggcagccccgcgcacgccgcggtggcgccgccaccaaggtcgcggagcggagggatcccgtcaagacgctcgatcgcgacgccgcggaaggtggcgcgccggcaccgccggcaccgaggcaggacgccgcccgtccaccgagtatgaacggcacgccggtgaacggtgagaacaaatctaccggcggcggcggcgcgaccaaagacagcgggctgcccgcacccgcacgcgcgccccatccgtcgacccagaacagagtaccagtgaacggtgaaaacaaagctaacgtcgcctcgccgccgacgagcatagccgaggtcgtggctccggattccgcagctaccatttccatcagtgacaaggcgccggagtccgttgtcccagccgagaagccgccgccgtcgtccggctcaaatttcgtggtctcggcttctgctcccaggctggacattgacagcgatgttgaacctgaactgaagaagggtgcggtcatcgtcgaagaagctccaaacccaaaggctctttcgccgcctgcagcccccgctgtacaagaagacctttgggacttcaagaaatacattggcttcgaggagcccgtggaggccaaggatgatggctgggctgttgcagatgatgcgggctcctttgaacatcaccagaaccatgattccggacctttggcaggggagaacgtcatgaacgtggtcgtcgtggctgctgaatgttctccctggtgcaaaacaggtggtcttggagatgttgcgggtgctctgcccaaggctttggcaaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctatcttccgacaccgtcaggaagacatttatgggggcagcagacaggaaattatgaagcgcatgattttgttctgcaaggccgctgtcgaggttccttggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgcgcaccagggccgtggcccagtagatgaattcccgttcaccgagttgcctgagcactacctggaacacttcagactgtacgaccccgtgggtggtgagcacgccaactacttcgccgccggcctgaagatggcggaccaggttgtcgtggtgagccccgggtacctgtgggagctcaagacggtggagggcggctgggggcttcacgacatcatacggcagaacgactggaagacccgcggcatcgtcaacggcatcgacaacatggagtggaaccccgaggtggacgtccacctccagtcggacggctacaccaacttctccctgagcacgctggactccggcaagcggcagtgcaaggaggccctgcagcgcgagctgggcctgcagatccgcgccgacgtgccgctgctcggcttcatcggccgcctggacgggcagaagggcgtggagatcatcgcggacgccatgccctggatcgtgagccaggacgtgcagctggtcatgctgggcaccggccgccacgacctggagagcatgctgcggcacttcgagcgggagcaccacgacaaggtgcgcgggtgggtggggttctccgtgcgcctggcgcaccggatcacggcgggcgccgacgcgctcctcatgccctcccggttcgagccgtgcgggctgaaccagctctacgccatggcctacggcaccgtccccgtcgtgcacgccgtcggcgggctgagggacaccgtgccgccgttcgaccccttcaaccactccggcctcgggtggacgttcgaccgcgccgaggcgcacaagctgatcgaggcgctcgggcactgcctccgcacctaccgggactacaaggagagctggaggggcctccaggagcgcggcatgtcgcaggacttcagctgggagcatgccgccaagctctacgaggacgtcctcctcaaggccaagtaccagtggtga

SEQ ID NO: 64 - protein, 799 aa, Artificial Sequence
  EMS Divide 280 Mutant Starch Synthase II Genome A Protein
MSSAVASAASFLALASASPGRSRRRARVSAPPPHAGAGRLHWPPWPPQRTARDGGVAARAAGKKDARVDDDAASARQPRARRGGAATKVAERRDPVKTLDRDAAEGGAPAPPAPRQDAARPPSMNGTPVNGENKSTGGGGATKDSGLPAPARAPHPSTQNRVPVNGENKANVASPPTSIAEVVAPDSAATISISDKAPESVVPAEKPPPSSGSNFVVSASAPRLDIDSDVEPELKKGAVIVEEAPNPKALSPPAAPAVQEDLWDFKKYIGFEEPVEAKDDGWAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPIFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDVHLQSDGYTNFSLSTLDSGKRQCKEALQRELGLQIRADVPLLGFIGRLDGQKGVEIIADAMPWIVSQDVQLVMLGTGRHDLESMLRHFEREHHDKVRGWVGFSVRLAHRITAGADALLMPSRFEPCGLNQLYAMAYGTVPVVHAVGGLRDTVPPFDPFNHSGLGWTFDRAEAHKLIEALGHCLRTYRDYKESWRGLQERGMSQDFSWEHAAKLYEDVLLKAKYQW

SEQ ID NO: 65 - DNA, 2400 bp, Artificial Sequence
  EMS Divide 674 Mutant Starch Synthase II Genome A CDS
atgtcgtcggcggtcgcgtccgccgcgtccttcctcgcgctcgcctccgcctcccccgggagatcacgcaggcgggcgagggtgagcgcgccgccaccccacgccggggccggcaggctgcactggccgccgtggccgccgcagcgcacggctcgcgacggaggtgtggccgcgcgcgccgccgggaagaaggacgcgagggtcgacgacgacgccgcgtccgcgaggcagccccgcgcacgccgcggtggcgccgccaccaaggtcgcggagcggagggatcccgtcaagacgctcgatcgcgacgccgcggaaggtggcgcgccggcaccgccggcaccgaggcaggacgccgcccgtccaccgagtatgaacggcacgccggtgaacggtgagaacaaatctaccggcggcggcggcgcgaccaaagacagcgggctgcccgcacccgcacgcgcgccccatccgtcgacccagaacagagtaccagtgaacggtgaaaacaaagctaacgtcgcctcgccgccgacgagcatagccgaggtcgtggctccggattccgcagctaccatttccatcagtgacaaggcgccggagtccgttgtcccagccgagaagccgccgccgtcgtccggctcaaatttcgtggtctcggcttctgctcccaggctggacattgacagcgatgttgaacctgaactgaagaagggtgcggtcatcgtcgaagaagctccaaacccaaaggctctttcgccgcctgcagcccccgctgtacaagaagacctttgggacttcaagaaatacattggcttcgaggagcccgtggaggccaaggatgatggctgggctgttgcagatgatgcgggctcctttgaacatcaccagaaccatgattccggacctttggcaggggagaacgtcatgaacgtggtcgtcgtggctgctgaatgttctccctggtgcaaaacaggtggtcttggagatgttgcgggtgctctgcccaaggctttggcaaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctatcttccgacaccgtcaggaagacatttatgggggcagcagacaggaaattatgaagcgcatgattttgttctgcaaggccgctgtcgaggttccttggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgcgcaccagggccgtggcccagtagatgaattcccgttcaccgagttgcctgagcactacctggaacacttcagactgtacgaccccgtgggtggtgagcacgccaactacttcgccgccggcctgaagatggcggaccaggttgtcgtggtgagccccgggtacctgtgggagctcaagacggtggagggcggctgggggcttcacgacatcatacggcagaacgactggaagacccgcggcatcgtcaacggcatcgacaacatggagtggaaccccgaggtggacgtccacctccagtcggacggctacaccaacttctccctgagcacgctggactccggcaagcggcagtgcaaggaggccctgcagcgcgagctgggcctgcaggtccgcgccgacgtgccgctgctcggcttcatcggccgcctggacgggcagaagggcgtggagatcatcgcggacgccatgccctggatcgtgagccaggacgtgcagctgatcatgctgggcaccggccgccacgacctggagagcatgctgcggcacttcgagcgggagcaccacgacaaggtgcgcgggtgggtggggttctccgtgcgcctggcgcaccggatcacggcgggcgccgacgcgctcctcatgccctcccggttcgagccgtgcgggctgaaccagctctacgccatggcctacggcaccgtccccgtcgtgcacgccgtcggcgggctgagggacaccgtgccgccgttcgaccccttcaaccactccggcctcgggtggacgttcgaccgcgccgaggcgcacaagctgatcgaggcgctcgggcactgcctccgcacctaccgggactacaaggagagctggaggggcctccaggagcgcggcatgtcgcaggacttcagctgggagcatgccgccaagctctacgaggacgtcctcctcaaggccaagtaccagtggtga

SEQ ID NO: 66 - protein, 799 aa, Artificial Sequence
  EMS Divide 674 Mutant Starch Synthase II Genome A Protein
MSSAVASAASFLALASASPGRSRRRARVSAPPPHAGAGRLHWPPWPPQRTARDGGVAARAAGKKDARVDDDAASARQPRARRGGAATKVAERRDPVKTLDRDAAEGGAPAPPAPRQDAARPPSMNGTPVNGENKSTGGGGATKDSGLPAPARAPHPSTQNRVPVNGENKANVASPPTSIAEVVAPDSAATISISDKAPESVVPAEKPPPSSGSNFVVSASAPRLDIDSDVEPELKKGAVIVEEAPNPKALSPPAAPAVQEDLWDFKKYIGFEEPVEAKDDGWAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPIFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDVHLQSDGYTNFSLSTLDSGKRQCKEALQRELGLQVRADVPLLGFIGRLDGQKGVEIIADAMPWIVSQDVQLIMLGTGRHDLESMLRHFEREHHDKVRGWVGFSVRLAHRITAGADALLMPSRFEPCGLNQLYAMAYGTVPVVHAVGGLRDTVPPFDPFNHSGLGWTFDRAEAHKLIEALGHCLRTYRDYKESWRGLQERGMSQDFSWEHAAKLYEDVLLKAKYQW

SEQ ID NO: 67 - DNA, 2400 bp, Artificial Sequence
  EMS Divide 1174 Mutant Starch Synthase II Genome A CDS
atgtcgtcggcggtcgcgtccgccgcgtccttcctcgcgctcgcctccgcctcccccgggagatcacgcaggcgggcgagggtgagcgcgccgccaccccacgccggggccggcaggctgcactggccgccgtggccgccgcagcgcacggctcgcgacggaggtgtggccgcgcgcgccgccgggaagaaggacgcgagggtcgacgacgacgccgcgtccgcgaggcagccccgcgcacgccgcggtggcgccgccaccaaggtcgcggagcggagggatcccgtcaagacgctcgatcgcgacgccgcggaaggtggcgcgccggcaccgccggcaccgaggcaggacgccgcccgtccaccgagtatgaacggcacgccggtgaacggtgagaacaaatctaccggcggcggcggcgcgaccaaagacagcgggctgcccgcacccgcacgcgcgccccatccgtcgacccagaacagagtaccagtgaacggtgaaaacaaagctaacgtcgcctcgccgccgacgagcatagccgaggtcgtggctccggattccgcagctaccatttccatcagtgacaaggcgccggagtccgttgtcccagccgagaagccgccgccgtcgtccggctcaaatttcgtggtctcggcttctgctcccaggctggacattgacagcgatgttgaacctgaactgaagaagggtgcggtcatcgtcgaagaagctccaaacccaaaggctctttcgccgcctgcagcccccgctgtacaagaagacctttgggacttcaagaaatacattggcttcgaggagcccgtggaggccaaggatgatggctgggctgttgcagatgatgcgggctcctttgaacatcaccagaaccatgattccggacctttggcaggggagaacgtcatgaacgtggtcgtcgtggctgctgaatgttctccctggtgcaaaacaggtggtcttggagatgttgcgggtgctctgcccaaggctttggcaaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctatcttccgacaccgtcaggaagacatttatgggggcagcagacaggaaattatgaagcgcatgattttgttctgcaaggccgctgtcgaggttccttggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgcgcaccagggccgtggcccagtagatgaattcccgttcaccgagttgcctgagcactacctggaacacttcagactgtacgaccccgtgggtggtgagcacgccaactacttcgccgccggcctgaagatggcggaccaggttgtcgtggtgagccccgggtacctgtgggagctcaagacggtggagggcggctgggggcttcacgacatcatacggcagaacgactggaagacccgcggcatcgtcaacggcatcgacaacatggagtggaaccccgaggtggacgtccacctccagtcggacggctacaccaacttctccctgagcacgctggactccggcaagcggcagtgcaaggaggccctgcagcgcgagctgggcctgcaggtccgcgccgacgtgccgctgctcggcttcatcggccgcctggacgggcagaagggcgtggagatcatcgcggacgccatgccctggatcgtgagccaggacgtgcagctggtcatgctgggcaccggccgccacgacctggagagcatgctgcggcacttcaagcgggagcaccacgacaaggtgcgcgggtgggtggggttctccgtgcgcctggcgcaccggatcacggcgggcgccgacgcgctcctcatgccctcccggttcgagccgtgcgggctgaaccagctctacgccatggcctacggcaccgtccccgtcgtgcacgccgtcggcgggctgagggacaccgtgccgccgttcgaccccttcaaccactccggcctcgggtggacgttcgaccgcgccgaggcgcacaagctgatcgaggcgctcgggcactgcctccgcacctaccgggactacaaggagagctggaggggcctccaggagcgcggcatgtcgcaggacttcagctgggagcatgccgccaagctctacgaggacgtcctcctcaaggccaagtaccagtggtga

SEQ ID NO: 68 - protein, 799 aa, Artificial Sequence
  EMS Divide 1174 Mutant Starch Synthase II Genome A Protein
MSSAVASAASFLALASASPGRSRRRARVSAPPPHAGAGRLHWPPWPPQRTARDGGVAARAAGKKDARVDDDAASARQPRARRGGAATKVAERRDPVKTLDRDAAEGGAPAPPAPRQDAARPPSMNGTPVNGENKSTGGGGATKDSGLPAPARAPHPSTQNRVPVNGENKANVASPPTSIAEVVAPDSAATISISDKAPESVVPAEKPPPSSGSNFVVSASAPRLDIDSDVEPELKKGAVIVEEAPNPKALSPPAAPAVQEDLWDFKKYIGFEEPVEAKDDGWAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPIFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDVHLQSDGYTNFSLSTLDSGKRQCKEALQRELGLQVRADVPLLGFIGRLDGQKGVEIIADAMPWIVSQDVQLVMLGTGRHDLESMLRHFKREHHDKVRGWVGFSVRLAHRITAGADALLMPSRFEPCGLNQLYAMAYGTVPVVHAVGGLRDTVPPFDPFNHSGLGWTFDRAEAHKLIEALGHCLRTYRDYKESWRGLQERGMSQDFSWEHAAKLYEDVLLKAKYQW

SEQ ID NO: 69 - DNA, 2400 bp, Artificial Sequence
  EMS Divide 1513 Mutant Starch Synthase II Genome A CDS
atgtcgtcggcggtcgcgtccgccgcgtccttcctcgcgctcgcctccgcctcccccgggagatcacgcaggcgggcgagggtgagcgcgccgccaccccacgccggggccggcaggctgcactggccgccgtggccgccgcagcgcacggctcgcgacggaggtgtggccgcgcgcgccgccgggaagaaggacgcgagggtcgacgacgacgccgcgtccgcgaggcagccccgcgcacgccgcggtggcgccgccaccaaggtcgcggagcggagggatcccgtcaagacgctcgatcgcgacgccgcggaaggtggcgcgccggcaccgccggcaccgaggcaggacgccgcccgtccaccgagtatgaacggcacgccggtgaacggtgagaacaaatctaccggcggcggcggcgcgaccaaagacagcgggctgcccgcacccgcacgcgcgccccatccgtcgacccagaacagagtaccagtgaacggtgaaaacaaagctaacgtcgcctcgccgccgacgagcatagccgaggtcgtggctccggattccgcagctaccatttccatcagtgacaaggcgccggagtccgttgtcccagccgagaagccgccgccgtcgtccggctcaaatttcgtggtctcggcttctgctcccaggctggacattgacagcgatgttgaacctgaactgaagaagggtgcggtcatcgtcgaagaagctccaaacccaaaggctctttcgccgcctgcagcccccgctgtacaagaagacctttgggacttcaagaaatacattggcttcgaggagcccgtggaggccaaggatgatggctgggctgttgcagatgatgcgggctcctttgaacatcaccagaaccatgattccggacctttggcaggggagaacgtcatgaacgtggtcgtcgtggctgctgaatgttctccctggtgcaaaacaggtggtcttggagatgttgcgggtgctctgcccaaggctttggcaaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctatcttccgacaccgtcaggaagacatttatgggggcagcagacaggaaattatgaagcgcatgattttgttctgcaaggccgctgtcgaggttccttggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgcgcaccagggccgtggcccagtagatgaattcccgttcaccgagttgcctgagcactacctggaacacttcagactgtacgaccccgtgggtggtgagcacgccaactacttcgccgccggcctgaagatggcggaccaggttgtcgtggtgagccccgggtacctgtgggagctcaagacggtggagggcggctgggggcttcacgacatcatacggcagaacgactggaagacccgcggcatcgtcaacggcatcgacaacatggagtggaaccccgaggtggacgtccacctccagtcggacggctacaccaacttctccctgagcacgctggactccggcaagcggcagtgcaaggaggccctgcagcgcgagctgggcctgcaggtccgcgccgacgtgccgctgctcggcttcatcggccgcctggacgggcagaagggcgtggagatcatcgcggacgccatgccctggatcgtgagccaggacgtgcagctggtcatgctgggcaccggccgccacgacctggagagcatgctgcggcacttcgagcgggagcaccacgacaaggtgcgcgggtgggtggggttctccgtgcgcctgacgcaccggatcacggcgggcgccgacgcgctcctcatgccctcccggttcgagccgtgcgggctgaaccagctctacgccatggcctacggcaccgtccccgtcgtgcacgccgtcggcgggctgagggacaccgtgccgccgttcgaccccttcaaccactccggcctcgggtggacgttcgaccgcgccgaggcgcacaagctgatcgaggcgctcgggcactgcctccgcacctaccgggactacaaggagagctggaggggcctccaggagcgcggcatgtcgcaggacttcagctgggagcatgccgccaagctctacgaggacgtcctcctcaaggccaagtaccagtggtga

SEQ ID NO: 70 - protein, 799 aa, Artificial Sequence
  EMS Divide 1513 Mutant Starch Synthase II Genome A Protein
MSSAVASAASFLALASASPGRSRRRARVSAPPPHAGAGRLHWPPWPPQRTARDGGVAARAAGKKDARVDDDAASARQPRARRGGAATKVAERRDPVKTLDRDAAEGGAPAPPAPRQDAARPPSMNGTPVNGENKSTGGGGATKDSGLPAPARAPHPSTQNRVPVNGENKANVASPPTSIAEVVAPDSAATISISDKAPESVVPAEKPPPSSGSNFVVSASAPRLDIDSDVEPELKKGAVIVEEAPNPKALSPPAAPAVQEDLWDFKKYIGFEEPVEAKDDGWAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPIFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDVHLQSDGYTNFSLSTLDSGKRQCKEALQRELGLQVRADVPLLGFIGRLDGQKGVEIIADAMPWIVSQDVQLVMLGTGRHDLESMLRHFEREHHDKVRGWVGFSVRLTHRITAGADALLMPSRFEPCGLNQLYAMAYGTVPVVHAVGGLRDTVPPFDPFNHSGLGWTFDRAEAHKLIEALGHCLRTYRDYKESWRGLQERGMSQDFSWEHAAKLYEDVLLKAKYQW

SEQ ID NO: 71 - DNA, 2400 bp, Artificial Sequence
  EMS Divide 134 Mutant Starch Synthase II Genome A CDS
atgtcgtcggcggtcgcgtccgccgcgtccttcctcgcgctcgcctccgcctcccccgggagatcacgcaggcgggcgagggtgagcgcgccgccaccccacgccggggccggcaggctgcactggccgccgtggccgccgcagcgcacggctcgcgacggaggtgtggccgcgcgcgccgccgggaagaaggacgcgagggtcgacgacgacgccgcgtccgcgaggcagccccgcgcacgccgcggtggcgccgccaccaaggtcgcggagcggagggatcccgtcaagacgctcgatcgcgacgccgcggaaggtggcgcgccggcaccgccggcaccgaggcaggacgccgcccgtccaccgagtatgaacggcacgccggtgaacggtgagaacaaatctaccggcggcggcggcgcgaccaaagacagcgggctgcccgcacccgcacgcgcgccccatccgtcgacccagaacagagtaccagtgaacggtgaaaacaaagctaacgtcgcctcgccgccgacgagcatagccgaggtcgtggctccggattccgcagctaccatttccatcagtgacaaggcgccggagtccgttgtcccagccgagaagccgccgccgtcgtccggctcaaatttcgtggtctcggcttctgctcccaggctggacattgacagcgatgttgaacctgaactgaagaagggtgcggtcatcgtcgaagaagctccaaacccaaaggctctttcgccgcctgcagcccccgctgtacaagaagacctttgggacttcaagaaatacattggcttcgaggagcccgtggaggccaaggatgatggctgggctgttgcagatgatgcgggctcctttgaacatcaccagaaccatgattccggacctttggcaggggagaacgtcatgaacgtggtcgtcgtggctgctgaatgttctccctggtgcaaaacaggtggtcttggagatgttgcgggtgctctgcccaaggctttggcaaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctatcttccgacaccgtcaggaagacatttatgggggcagcagacaggaaattatgaagcgcatgattttgttctgcaaggccgctgtcgaggttccttggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgcgcaccagggccgtggcccagtagatgaattcccgttcaccgagttgcctgagcactacctggaacacttcagactgtacgaccccgtgggtggtgagcacgccaactacttcgccgccggcctgaagatggcggaccaggttgtcgtggtgagccccgggtacctgtgggagctcaagacggtggagggcggctgggggcttcacgacatcatacggcagaacgactggaagacccgcggcatcgtcaacggcatcgacaacatggagtggaaccccgaggtggacgtccacctccagtcggacggctacaccaacttctccctgagcacgctggactccggcaagcggcagtgcaaggaggccctgcagcgcgagctgggcctgcaggtccgcgccgacgtgccgctgctcggcttcatcggccgcctggacgggcagaagggcgtggagatcatcgcggacgccatgccctggatcgtgagccaggacgtgcagctggtcatgctgggcaccggccgccacgacctggagagcatgctgcggcacttcgagcgggagcaccacgacaaggtgcgcgggtgggtggggttctccgtgcgcctggcgcaccggatcacggcgggcgccgacgcgctcctcatgccctcccggttcgagccgtgcgggctgaaccagctctacgccatggcctacggcaccgtccccgtcgtgcacgccgtcggcgagctgagggacaccgtgccgccgttcgaccccttcaaccactccggcctcgggtggacgttcgaccgcgccgaggcgcacaagctgatcgaggcgctcgggcactgcctccgcacctaccgggactacaaggagagctggaggggcctccaggagcgcggcatgtcgcaggacttcagctgggagcatgccgccaagctctacgaggacgtcctcctcaaggccaagtaccagtggtga

SEQ ID NO: 72 - protein, 799 aa, Artificial Sequence
  EMS Divide 134 Mutant Starch Synthase II Genome A Protein
MSSAVASAASFLALASASPGRSRRRARVSAPPPHAGAGRLHWPPWPPQRTARDGGVAARAAGKKDARVDDDAASARQPRARRGGAATKVAERRDPVKTLDRDAAEGGAPAPPAPRQDAARPPSMNGTPVNGENKSTGGGGATKDSGLPAPARAPHPSTQNRVPVNGENKANVASPPTSIAEVVAPDSAATISISDKAPESVVPAEKPPPSSGSNFVVSASAPRLDIDSDVEPELKKGAVIVEEAPNPKALSPPAAPAVQEDLWDFKKYIGFEEPVEAKDDGWAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPIFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDVHLQSDGYTNFSLSTLDSGKRQCKEALQRELGLQVRADVPLLGFIGRLDGQKGVEIIADAMPWIVSQDVQLVMLGTGRHDLESMLRHFEREHHDKVRGWVGFSVRLAHRITAGADALLMPSRFEPCGLNQLYAMAYGTVPVVHAVGELRDTVPPFDPFNHSGLGWTFDRAEAHKLIEALGHCLRTYRDYKESWRGLQERGMSQDFSWEHAAKLYEDVLLKAKYQW

SEQ ID NO: 73 - DNA, 2397 bp, Artificial Sequence
  EMS Divide 90 Mutant Starch Synthase II Genome B CDS
atgtcgtcggcggtcgcgtccgccgcgtccttcctcgcgctcgcgtccgcctcccccgggagatcacggaggaggacgagggtgagcgcgtcgccaccccacaccggggctggcaggttgcactggccgccgtcgccgccgcagcgcacggctcgcgacggagcagtggccgcgcgcgccgccgggaagaaggacgcggggatcgacgacgccgcgcccgcgaggcagccccgcgcactccgcggtggcgccgccaccaaggttgcggagcggagggatcccgtcaagacgctcgatcgcgacgccgcggaaggtggcgcgccgtccccgccggcaccgaggcaggaggacgcccgtctgccgagcatgaacggcatgccggtgaacggtgaaaacaaatctaccggcggcggcggcgcgactaaagacagcgggctgcccgcacccgcacgcgcgccccagccgtcgagccagaacagagtaccggtgaatggtgaaaacaaagctaacgtcgcctcgccgccgacgagcatagccgaggtcgcggctccggatcccgcagctaccatttccatcagtgacaaggcgccagagtccgttgtcccagccgagaaggcgccgccgtcgtccggctcaaatttcgtgccctcggcttctgctcccgggtctgacactgtcagcgacgtggaacttgaactgaagaagggtgcggtcattgtcaaagaagctccaaacccaaaggctctttcgccgcctgcagcacccgctgtacaacaagacctttgggacttcaagaaatacattggtttcgaggagcccgtggaggccaaggatgatggccgggctgttgcagatgatgcgggctccttcgaacaccaccagaatcacgattccgggcctttggcaggggagaacgtcatgaacgtggtcgtcgtggctgctgaatgttctccctggtgcaaaacaggtggtcttggagatgttgccggtgctttgcccaaggctttggcgaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctctcttccgacaccgccaggaagacatttatgggggcagcagacaggaaattatgaagcgcatgattttgttctgcaaggccgctgtcgaggttccatggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgctcaccagggccgtggcccagtagatgagttcccgttcaccgagttgcctgagcactacctggaacacttcagactgtacgaccccgtgggtggtgaacacgccaactacttcgccgccggcctgaagatggcgaaccaggttgtcgtcgtgagcccggggtacctgtgggagctgaagacggtggagggcggctgggggcttcacgacatcatacggcagaacgactggaagacccgcggcatcgtgaacggcatcgacaacatggagtggaaccccgaggtggacgtccacctcaagtcggacggctacaccaacttctccctggggacgctggactccggcaagcggcagtgcaaggaggccctgcagcgggagctgggcctgcaggtccgcggcgacgtgccgctgctcggcttcatcgggcgcctggacgggcagaagggcgtggagatcatcgcggacgcgatgccctggatcgtgagccaggacgtgcagctggtcatgctgggcaccgggcgccacgacctggagggcatgctgcggcacttcgagcgggagcaccacgacaaggtgcgcgggtgggtggggttctccgtgcggctggcgcaccggatcacggccggcgccgacgcgctcctcatgccctcccggttcgagccgtgcggactgaaccagctctacgccatggcctacggcaccgtccccgtcgtgcatgccgtcggcggcctgagggacaccgtgccgccgttcgaccccttcaaccactccgggctcgggtggacgttcgaccgcgcagaggcgcagaagctgatcgaggcgctcgggcactgcctccgcacctaccgggactacaaggagagctggagggggctccaggagcgcggcatgtcgcaggacttcagctgggagcatgccgccaagctctacgaggacgtcctcgtcaaggccaagtaccagtggtga

SEQ ID NO: 74 - protein, 798 aa, Artificial Sequence
  EMS Divide 90 Mutant Starch Synthase II Genome B Protein
MSSAVASAASFLALASASPGRSRRRTRVSASPPHTGAGRLHWPPSPPQRTARDGAVAARAAGKKDAGIDDAAPARQPRALRGGAATKVAERRDPVKTLDRDAAEGGAPSPPAPRQEDARLPSMNGMPVNGENKSTGGGGATKDSGLPAPARAPQPSSQNRVPVNGENKANVASPPTSIAEVAAPDPAATISISDKAPESVVPAEKAPPSSGSNFVPSASAPGSDTVSDVELELKKGAVIVKEAPNPKALSPPAAPAVQQDLWDFKKYIGFEEPVEAKDDGRAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPLFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMANQVVVVSPGYLWELKTVEGGWGLHDIIRQNDWKTRGIVNGIDNMEWNPEVDVHLKSDGYTNFSLGTLDSGKRQCKEALQRELGLQVRGDVPLLGFIGRLDGQKGVEIIADAMPWIVSQDVQLVMLGTGRHDLEGMLRHFEREHHDKVRGWVGFSVRLAHRITAGADALLMPSRFEPCGLNQLYAMAYGTVPVVHAVGGLRDTVPPFDPFNHSGLGWTFDRAEAQKLIEALGHCLRTYRDYKESWRGLQERGMSQDFSWEHAAKLYEDVLVKAKYQW

SEQ ID NO: 75 - DNA, 2397 bp, Artificial Sequence
  EMS Divide 145 Mutant Starch Synthase II Genome B CDS
atgtcgtcggcggtcgcgtccgccgcgtccttcctcgcgctcgcgtccgcctcccccgggagatcacggaggaggacgagggtgagcgcgtcgccaccccacaccggggctggcaggttgcactggccgccgtcgccgccgcagcgcacggctcgcgacggagcagtggccgcgcgcgccgccgggaagaaggacgcggggatcgacgacgccgcgcccgcgaggcagccccgcgcactccgcggtggcgccgccaccaaggttgcggagcggagggatcccgtcaagacgctcgatcgcgacgccgcggaaggtggcgcgccgtccccgccggcaccgaggcaggaggacgcccgtctgccgagcatgaacggcatgccggtgaacggtgaaaacaaatctaccggcggcggcggcgcgactaaagacagcgggctgcccgcacccgcacgcgcgccccagccgtcgagccagaacagagtaccggtgaatggtgaaaacaaagctaacgtcgcctcgccgccgacgagcatagccgaggtcgcggctccggatcccgcagctaccatttccatcagtgacaaggcgccagagtccgttgtcccagccgagaaggcgccgccgtcgtccggctcaaatttcgtgccctcggcttctgctcccgggtctgacactgtcagcgacgtggaacttgaactgaagaagggtgcggtcattgtcaaagaagctccaaacccaaaggctctttcgccgcctgcagcacccgctgtacaacaagacctttgggacttcaagaaatacattggtttcgaggagcccgtggaggccaaggatgatggccgggctgttgcagatgatgcgggctccttcgaacaccaccagaatcacgattccgggcctttggcaggggagaacgtcatgaacgtggtcgtcgtggctgctgaatgttctccctggtgcaaaacaggtggtcttggagatgttgccggtgctttgcccaaggctttggcgaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctctcttccgacaccgccaggaagacatttatgggggcagcagacaggaaattatgaagcgcatgattttgttctgcaaggccgctgtcgaggttccatggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgctcaccagggccgtggcccagtagatgagttcccgttcaccgagttgcctgagcactacctggaacacttcagactgtacgaccccgtgggtggtgaacacgccaactacttcgccgccggcctgaagatggcggaccaggttgtcgtcgtgagcccggggtacctgtgggagctgaagacggtggagggcggctgggggcttcacgacatcatacggcagaacgactggaagacccgcggcatcgtgaacggcatcgacaacatggagtggaaccccgaggtggacgtccacctcaagtcggacggctacaccaacttctccctggggacgctggactccggcaagcggcagtgcaaggaggccctgcagcgggagctgggcctgcaggtccgcggcgacgtgccgctgctcggcttcatcgggcgcctggacgagcagaagggcgtggagatcatcgcggacgcgatgccctggatcgtgagccaggacgtgcagctggtcatgctgggcaccgggcgccacgacctggagggcatgctgcggcacttcgagcgggagcaccacgacaaggtgcgcgggtgggtggggttctccgtgcggctggcgcaccggatcacggccggcgccgacgcgctcctcatgccctcccggttcgagccgtgcggactgaaccagctctacgccatggcctacggcaccgtccccgtcgtgcatgccgtcggcggcctgagggacaccgtgccgccgttcgaccccttcaaccactccgggctcgggtggacgttcgaccgcgcagaggcgcagaagctgatcgaggcgctcgggcactgcctccgcacctaccgggactacaaggagagctggagggggctccaggagcgcggcatgtcgcaggacttcagctgggagcatgccgccaagctctacgaggacgtcctcgtcaaggccaagtaccagtggtga

SEQ ID NO: 76 - protein, 798 aa, Artificial Sequence
  EMS Divide 145 Mutant Starch Synthase II Genome B Protein
MSSAVASAASFLALASASPGRSRRRTRVSASPPHTGAGRLHWPPSPPQRTARDGAVAARAAGKKDAGIDDAAPARQPRALRGGAATKVAERRDPVKTLDRDAAEGGAPSPPAPRQEDARLPSMNGMPVNGENKSTGGGGATKDSGLPAPARAPQPSSQNRVPVNGENKANVASPPTSIAEVAAPDPAATISISDKAPESVVPAEKAPPSSGSNFVPSASAPGSDTVSDVELELKKGAVIVKEAPNPKALSPPAAPAVQQDLWDFKKYIGFEEPVEAKDDGRAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPLFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDVHLKSDGYTNFSLGTLDSGKRQCKEALQRELGLQVRGDVPLLGFIGRLDEQKGVEIIADAMPWIVSQDVQLVMLGTGRHDLEGMLRHFEREHHDKVRGWVGFSVRLAHRITAGADALLMPSRFEPCGLNQLYAMAYGTVPVVHAVGGLRDTVPPFDPFNHSGLGWTFDRAEAQKLIEALGHCLRTYRDYKESWRGLQERGMSQDFSWEHAAKLYEDVLVKAKYQW

SEQ ID NO: 77 - DNA, 2397 bp, Artificial Sequence
  EMS Divide 1664 Mutant Starch Synthase II Genome B CDS
atgtcgtcggcggtcgcgtccgccgcgtccttcctcgcgctcgcgtccgcctcccccgggagatcacggaggaggacgagggtgagcgcgtcgccaccccacaccggggctggcaggttgcactggccgccgtcgccgccgcagcgcacggctcgcgacggagcagtggccgcgcgcgccgccgggaagaaggacgcggggatcgacgacgccgcgcccgcgaggcagccccgcgcactccgcggtggcgccgccaccaaggttgcggagcggagggatcccgtcaagacgctcgatcgcgacgccgcggaaggtggcgcgccgtccccgccggcaccgaggcaggaggacgcccgtctgccgagcatgaacggcatgccggtgaacggtgaaaacaaatctaccggcggcggcggcgcgactaaagacagcgggctgcccgcacccgcacgcgcgccccagccgtcgagccagaacagagtaccggtgaatggtgaaaacaaagctaacgtcgcctcgccgccgacgagcatagccgaggtcgcggctccggatcccgcagctaccatttccatcagtgacaaggcgccagagtccgttgtcccagccgagaaggcgccgccgtcgtccggctcaaatttcgtgccctcggcttctgctcccgggtctgacactgtcagcgacgtggaacttgaactgaagaagggtgcggtcattgtcaaagaagctccaaacccaaaggctctttcgccgcctgcagcacccgctgtacaacaagacctttgggacttcaagaaatacattggtttcgaggagcccgtggaggccaaggatgatggccgggctgttgcagatgatgcgggctccttcgaacaccaccagaatcacgattccgggcctttggcaggggagaacgtcatgaacgtggtcgtcgtggctgctgaatgttctccctggtgcaaaacaggtggtcttggagatgttgccggtgctttgcccaaggctttggcgaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctctcttccgacaccgccaggaagacatttatgggggcagcagacaggaaattatgaagcgcatgattttgttctgcaaggccgctgtcgaggttccatggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgctcaccagggccgtggcccagtagatgagttcccgttcaccgagttgcctgagcactacctggaacacttcagactgtacgaccccgtgggtggtgaacacgccaactacttcgccgccggcctgaagatggcggaccaggttgtcgtcgtgagcccggggtacctgtgggagctgaagacggtggagggcggctgggggcttcacgacatcatacggcagaacgactggaagacccgcggcatcgtgaacggcatcgacaacatggagtggaaccccgaggtggacgtccacctcaagtcggacggctacaccaacttctccctggggacgctggactccggcaagcggcagtgcaaggaggccctgcagcgggagctgggcctgcaggtccgcggcgacgtgccgctgctcggcttcatcgggcgcctggacgggcagaagggcgtggagatcatcgtggacgcgatgccctggatcgtgagccaggacgtgcagctggtcatgctgggcaccgggcgccacgacctggagggcatgctgcggcacttcgagcgggagcaccacgacaaggtgcgcgggtgggtggggttctccgtgcggctggcgcaccggatcacggccggcgccgacgcgctcctcatgccctcccggttcgagccgtgcggactgaaccagctctacgccatggcctacggcaccgtccccgtcgtgcatgccgtcggcggcctgagggacaccgtgccgccgttcgaccccttcaaccactccgggctcgggtggacgttcgaccgcgcagaggcgcagaagctgatcgaggcgctcgggcactgcctccgcacctaccgggactacaaggagagctggagggggctccaggagcgcggcatgtcgcaggacttcagctgggagcatgccgccaagctctacgaggacgtcctcgtcaaggccaagtaccagtggtga

SEQ ID NO: 78 - protein, 798 aa, Artificial Sequence
  EMS Divide 1664 Mutant Starch Synthase II Genome B Protein
MSSAVASAASFLALASASPGRSRRRTRVSASPPHTGAGRLHWPPSPPQRTARDGAVAARAAGKKDAGIDDAAPARQPRALRGGAATKVAERRDPVKTLDRDAAEGGAPSPPAPRQEDARLPSMNGMPVNGENKSTGGGGATKDSGLPAPARAPQPSSQNRVPVNGENKANVASPPTSIAEVAAPDPAATISISDKAPESVVPAEKAPPSSGSNFVPSASAPGSDTVSDVELELKKGAVIVKEAPNPKALSPPAAPAVQQDLWDFKKYIGFEEPVEAKDDGRAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPLFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDVHLKSDGYTNFSLGTLDSGKRQCKEALQRELGLQVRGDVPLLGFIGRLDGQKGVEIIVDAMPWIVSQDVQLVMLGTGRHDLEGMLRHFEREHHDKVRGWVGFSVRLAHRITAGADALLMPSRFEPCGLNQLYAMAYGTVPVVHAVGGLRDTVPPFDPFNHSGLGWTFDRAEAQKLIEALGHCLRTYRDYKESWRGLQERGMSQDFSWEHAAKLYEDVLVKAKYQW

SEQ ID NO: 79 - DNA, 2397 bp, Artificial Sequence
  EMS Divide 93 Mutant Starch Synthase II Genome B CDS
atgtcgtcggcggtcgcgtccgccgcgtccttcctcgcgctcgcgtccgcctcccccgggagatcacggaggaggacgagggtgagcgcgtcgccaccccacaccggggctggcaggttgcactggccgccgtcgccgccgcagcgcacggctcgcgacggagcagtggccgcgcgcgccgccgggaagaaggacgcggggatcgacgacgccgcgcccgcgaggcagccccgcgcactccgcggtggcgccgccaccaaggttgcggagcggagggatcccgtcaagacgctcgatcgcgacgccgcggaaggtggcgcgccgtccccgccggcaccgaggcaggaggacgcccgtctgccgagcatgaacggcatgccggtgaacggtgaaaacaaatctaccggcggcggcggcgcgactaaagacagcgggctgcccgcacccgcacgcgcgccccagccgtcgagccagaacagagtaccggtgaatggtgaaaacaaagctaacgtcgcctcgccgccgacgagcatagccgaggtcgcggctccggatcccgcagctaccatttccatcagtgacaaggcgccagagtccgttgtcccagccgagaaggcgccgccgtcgtccggctcaaatttcgtgccctcggcttctgctcccgggtctgacactgtcagcgacgtggaacttgaactgaagaagggtgcggtcattgtcaaagaagctccaaacccaaaggctctttcgccgcctgcagcacccgctgtacaacaagacctttgggacttcaagaaatacattggtttcgaggagcccgtggaggccaaggatgatggccgggctgttgcagatgatgcgggctccttcgaacaccaccagaatcacgattccgggcctttggcaggggagaacgtcatgaacgtggtcgtcgtggctgctgaatgttctccctggtgcaaaacaggtggtcttggagatgttgccggtgctttgcccaaggctttggcgaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctctcttccgacaccgccaggaagacatttatgggggcagcagacaggaaattatgaagcgcatgattttgttctgcaaggccgctgtcgaggttccatggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgctcaccagggccgtggcccagtagatgagttcccgttcaccgagttgcctgagcactacctggaacacttcagactgtacgaccccgtgggtggtgaacacgccaactacttcgccgccggcctgaagatggcggaccaggttgtcgtcgtgagcccggggtacctgtgggagctgaagacggtggagggcggctgggggcttcacgacatcatacggcagaacgactggaagacccgcggcatcgtgaacggcatcgacaacatggagtggaaccccgaggtggacgtccacctcaagtcggacggctacaccaacttctccctggggacgctggactccggcaagcggcagtgcaaggaggccctgcagcgggagctgggcctgcaggtccgcggcgacgtgccgctgctcggcttcatcgggcgcctggacgggcagaagggcgtggagatcatcgcggacgcgatgccctggatcgtgagccagaacgtgcagctggtcatgctgggcaccgggcgccacgacctggagggcatgctgcggcacttcgagcgggagcaccacgacaaggtgcgcgggtgggtggggttctccgtgcggctggcgcaccggatcacggccggcgccgacgcgctcctcatgccctcccggttcgagccgtgcggactgaaccagctctacgccatggcctacggcaccgtccccgtcgtgcatgccgtcggcggcctgagggacaccgtgccgccgttcgaccccttcaaccactccgggctcgggtggacgttcgaccgcgcagaggcgcagaagctgatcgaggcgctcgggcactgcctccgcacctaccgggactacaaggagagctggagggggctccaggagcgcggcatgtcgcaggacttcagctgggagcatgccgccaagctctacgaggacgtcctcgtcaaggccaagtaccagtggtga

SEQ ID NO: 80 - protein, 798 aa, Artificial Sequence
  EMS Divide 93 Mutant Starch Synthase II Genome B Protein
MSSAVASAASFLALASASPGRSRRRTRVSASPPHTGAGRLHWPPSPPQRTARDGAVAARAAGKKDAGIDDAAPARQPRALRGGAATKVAERRDPVKTLDRDAAEGGAPSPPAPRQEDARLPSMNGMPVNGENKSTGGGGATKDSGLPAPARAPQPSSQNRVPVNGENKANVASPPTSIAEVAAPDPAATISISDKAPESVVPAEKAPPSSGSNFVPSASAPGSDTVSDVELELKKGAVIVKEAPNPKALSPPAAPAVQQDLWDFKKYIGFEEPVEAKDDGRAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPLFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDVHLKSDGYTNFSLGTLDSGKRQCKEALQRELGLQVRGDVPLLGFIGRLDGQKGVEIIADAMPWIVSQNVQLVMLGTGRHDLEGMLRHFEREHHDKVRGWVGFSVRLAHRITAGADALLMPSRFEPCGLNQLYAMAYGTVPVVHAVGGLRDTVPPFDPFNHSGLGWTFDRAEAQKLIEALGHCLRTYRDYKESWRGLQERGMSQDFSWEHAAKLYEDVLVKAKYQW

SEQ ID NO: 81 - DNA, 2397 bp, Artificial Sequence
  EMS Divide 1237 Mutant Starch Synthase II Genome B CDS
atgtcgtcggcggtcgcgtccgccgcgtccttcctcgcgctcgcgtccgcctcccccgggagatcacggaggaggacgagggtgagcgcgtcgccaccccacaccggggctggcaggttgcactggccgccgtcgccgccgcagcgcacggctcgcgacggagcagtggccgcgcgcgccgccgggaagaaggacgcggggatcgacgacgccgcgcccgcgaggcagccccgcgcactccgcggtggcgccgccaccaaggttgcggagcggagggatcccgtcaagacgctcgatcgcgacgccgcggaaggtggcgcgccgtccccgccggcaccgaggcaggaggacgcccgtctgccgagcatgaacggcatgccggtgaacggtgaaaacaaatctaccggcggcggcggcgcgactaaagacagcgggctgcccgcacccgcacgcgcgccccagccgtcgagccagaacagagtaccggtgaatggtgaaaacaaagctaacgtcgcctcgccgccgacgagcatagccgaggtcgcggctccggatcccgcagctaccatttccatcagtgacaaggcgccagagtccgttgtcccagccgagaaggcgccgccgtcgtccggctcaaatttcgtgccctcggcttctgctcccgggtctgacactgtcagcgacgtggaacttgaactgaagaagggtgcggtcattgtcaaagaagctccaaacccaaaggctctttcgccgcctgcagcacccgctgtacaacaagacctttgggacttcaagaaatacattggtttcgaggagcccgtggaggccaaggatgatggccgggctgttgcagatgatgcgggctccttcgaacaccaccagaatcacgattccgggcctttggcaggggagaacgtcatgaacgtggtcgtcgtggctgctgaatgttctccctggtgcaaaacaggtggtcttggagatgttgccggtgctttgcccaaggctttggcgaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctctcttccgacaccgccaggaagacatttatgggggcagcagacaggaaattatgaagcgcatgattttgttctgcaaggccgctgtcgaggttccatggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgctcaccagggccgtggcccagtagatgagttcccgttcaccgagttgcctgagcactacctggaacacttcagactgtacgaccccgtgggtggtgaacacgccaactacttcgccgccggcctgaagatggcggaccaggttgtcgtcgtgagcccggggtacctgtgggagctgaagacggtggagggcggctgggggcttcacgacatcatacggcagaacgactggaagacccgcggcatcgtgaacggcatcgacaacatggagtggaaccccgaggtggacgtccacctcaagtcggacggctacaccaacttctccctggggacgctggactccggcaagcggcagtgcaaggaggccctgcagcgggagctgggcctgcaggtccgcggcgacgtgccgctgctcggcttcatcgggcgcctggacgggcagaagggcgtggagatcatcgcggacgcgatgccctggatcgtgagccaggacgtgcagctggtcatgctgggcaccgggcgccacgacctggagggcatgctgtggcacttcgagcgggagcaccacgacaaggtgcgcgggtgggtggggttctccgtgcggctggcgcaccggatcacggccggcgccgacgcgctcctcatgccctcccggttcgagccgtgcggactgaaccagctctacgccatggcctacggcaccgtccccgtcgtgcatgccgtcggcggcctgagggacaccgtgccgccgttcgaccccttcaaccactccgggctcgggtggacgttcgaccgcgcagaggcgcagaagctgatcgaggcgctcgggcactgcctccgcacctaccgggactacaaggagagctggagggggctccaggagcgcggcatgtcgcaggacttcagctgggagcatgccgccaagctctacgaggacgtcctcgtcaaggccaagtaccagtggtga

SEQ ID NO: 82 - protein, 798 aa, Artificial Sequence
  EMS Divide 1237 Mutant Starch Synthase II Genome B Protein
MSSAVASAASFLALASASPGRSRRRTRVSASPPHTGAGRLHWPPSPPQRTARDGAVAARAAGKKDAGIDDAAPARQPRALRGGAATKVAERRDPVKTLDRDAAEGGAPSPPAPRQEDARLPSMNGMPVNGENKSTGGGGATKDSGLPAPARAPQPSSQNRVPVNGENKANVASPPTSIAEVAAPDPAATISISDKAPESVVPAEKAPPSSGSNFVPSASAPGSDTVSDVELELKKGAVIVKEAPNPKALSPPAAPAVQQDLWDFKKYIGFEEPVEAKDDGRAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPLFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDVHLKSDGYTNFSLGTLDSGKRQCKEALQRELGLQVRGDVPLLGFIGRLDGQKGVEIIADAMPWIVSQDVQLVMLGTGRHDLEGMLWHFEREHHDKVRGWVGFSVRLAHRITAGADALLMPSRFEPCGLNQLYAMAYGTVPVVHAVGGLRDTVPPFDPFNHSGLGWTFDRAEAQKLIEALGHCLRTYRDYKESWRGLQERGMSQDFSWEHAAKLYEDVLVKAKYQW

SEQ ID NO: 83 - DNA, 2397 bp, Artificial Sequence
  EMS Divide 57 Mutant Starch Synthase II Genome B CDS
atgtcgtcggcggtcgcgtccgccgcgtccttcctcgcgctcgcgtccgcctcccccgggagatcacggaggaggacgagggtgagcgcgtcgccaccccacaccggggctggcaggttgcactggccgccgtcgccgccgcagcgcacggctcgcgacggagcagtggccgcgcgcgccgccgggaagaaggacgcggggatcgacgacgccgcgcccgcgaggcagccccgcgcactccgcggtggcgccgccaccaaggttgcggagcggagggatcccgtcaagacgctcgatcgcgacgccgcggaaggtggcgcgccgtccccgccggcaccgaggcaggaggacgcccgtctgccgagcatgaacggcatgccggtgaacggtgaaaacaaatctaccggcggcggcggcgcgactaaagacagcgggctgcccgcacccgcacgcgcgccccagccgtcgagccagaacagagtaccggtgaatggtgaaaacaaagctaacgtcgcctcgccgccgacgagcatagccgaggtcgcggctccggatcccgcagctaccatttccatcagtgacaaggcgccagagtccgttgtcccagccgagaaggcgccgccgtcgtccggctcaaatttcgtgccctcggcttctgctcccgggtctgacactgtcagcgacgtggaacttgaactgaagaagggtgcggtcattgtcaaagaagctccaaacccaaaggctctttcgccgcctgcagcacccgctgtacaacaagacctttgggacttcaagaaatacattggtttcgaggagcccgtggaggccaaggatgatggccgggctgttgcagatgatgcgggctccttcgaacaccaccagaatcacgattccgggcctttggcaggggagaacgtcatgaacgtggtcgtcgtggctgctgaatgttctccctggtgcaaaacaggtggtcttggagatgttgccggtgctttgcccaaggctttggcgaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctctcttccgacaccgccaggaagacatttatgggggcagcagacaggaaattatgaagcgcatgattttgttctgcaaggccgctgtcgaggttccatggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgctcaccagggccgtggcccagtagatgagttcccgttcaccgagttgcctgagcactacctggaacacttcagactgtacgaccccgtgggtggtgaacacgccaactacttcgccgccggcctgaagatggcggaccaggttgtcgtcgtgagcccggggtacctgtgggagctgaagacggtggagggcggctgggggcttcacgacatcatacggcagaacgactggaagacccgcggcatcgtgaacggcatcgacaacatggagtggaaccccgaggtggacgtccacctcaagtcggacggctacaccaacttctccctggggacgctggactccggcaagcggcagtgcaaggaggccctgcagcgggagctgggcctgcaggtccgcggcgacgtgccgctgctcggcttcatcgggcgcctggacgggcagaagggcgtggagatcatcgcggacgcgatgccctggatcgtgagccaggacgtgcagctggtcatgctgggcaccgggcgccacgacctggagggcatgctgcggcacttcgagcgggagcaccacgacaaggtgcgcgggtggatggggttctccgtgcggctggcgcaccggatcacggccggcgccgacgcgctcctcatgccctcccggttcgagccgtgcggactgaaccagctctacgccatggcctacggcaccgtccccgtcgtgcatgccgtcggcggcctgagggacaccgtgccgccgttcgaccccttcaaccactccgggctcgggtggacgttcgaccgcgcagaggcgcagaagctgatcgaggcgctcgggcactgcctccgcacctaccgggactacaaggagagctggagggggctccaggagcgcggcatgtcgcaggacttcagctgggagcatgccgccaagctctacgaggacgtcctcgtcaaggccaagtaccagtggtga

SEQ ID NO: 84 - protein, 798 aa, Artificial Sequence
  EMS Divide 57 Mutant Starch Synthase II Genome B Protein
MSSAVASAASFLALASASPGRSRRRTRVSASPPHTGAGRLHWPPSPPQRTARDGAVAARAAGKKDAGIDDAAPARQPRALRGGAATKVAERRDPVKTLDRDAAEGGAPSPPAPRQEDARLPSMNGMPVNGENKSTGGGGATKDSGLPAPARAPQPSSQNRVPVNGENKANVASPPTSIAEVAAPDPAATISISDKAPESVVPAEKAPPSSGSNFVPSASAPGSDTVSDVELELKKGAVIVKEAPNPKALSPPAAPAVQQDLWDFKKYIGFEEPVEAKDDGRAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPLFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDVHLKSDGYTNFSLGTLDSGKRQCKEALQRELGLQVRGDVPLLGFIGRLDGQKGVEIIADAMPWIVSQDVQLVMLGTGRHDLEGMLRHFEREHHDKVRGWMGFSVRLAHRITAGADALLMPSRFEPCGLNQLYAMAYGTVPVVHAVGGLRDTVPPFDPFNHSGLGWTFDRAEAQKLIEALGHCLRTYRDYKESWRGLQERGMSQDFSWEHAAKLYEDVLVKAKYQW

SEQ ID NO: 85 - DNA, 2397 bp, Artificial Sequence
  EMS Divide 1704 Mutant Starch Synthase II Genome B CDS
atgtcgtcggcggtcgcgtccgccgcgtccttcctcgcgctcgcgtccgcctcccccgggagatcacggaggaggacgagggtgagcgcgtcgccaccccacaccggggctggcaggttgcactggccgccgtcgccgccgcagcgcacggctcgcgacggagcagtggccgcgcgcgccgccgggaagaaggacgcggggatcgacgacgccgcgcccgcgaggcagccccgcgcactccgcggtggcgccgccaccaaggttgcggagcggagggatcccgtcaagacgctcgatcgcgacgccgcggaaggtggcgcgccgtccccgccggcaccgaggcaggaggacgcccgtctgccgagcatgaacggcatgccggtgaacggtgaaaacaaatctaccggcggcggcggcgcgactaaagacagcgggctgcccgcacccgcacgcgcgccccagccgtcgagccagaacagagtaccggtgaatggtgaaaacaaagctaacgtcgcctcgccgccgacgagcatagccgaggtcgcggctccggatcccgcagctaccatttccatcagtgacaaggcgccagagtccgttgtcccagccgagaaggcgccgccgtcgtccggctcaaatttcgtgccctcggcttctgctcccgggtctgacactgtcagcgacgtggaacttgaactgaagaagggtgcggtcattgtcaaagaagctccaaacccaaaggctctttcgccgcctgcagcacccgctgtacaacaagacctttgggacttcaagaaatacattggtttcgaggagcccgtggaggccaaggatgatggccgggctgttgcagatgatgcgggctccttcgaacaccaccagaatcacgattccgggcctttggcaggggagaacgtcatgaacgtggtcgtcgtggctgctgaatgttctccctggtgcaaaacaggtggtcttggagatgttgccggtgctttgcccaaggctttggcgaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctctcttccgacaccgccaggaagacatttatgggggcagcagacaggaaattatgaagcgcatgattttgttctgcaaggccgctgtcgaggttccatggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgctcaccagggccgtggcccagtagatgagttcccgttcaccgagttgcctgagcactacctggaacacttcagactgtacgaccccgtgggtggtgaacacgccaactacttcgccgccggcctgaagatggcggaccaggttgtcgtcgtgagcccggggtacctgtgggagctgaagacggtggagggcggctgggggcttcacgacatcatacggcagaacgactggaagacccgcggcatcgtgaacggcatcgacaacatggagtggaaccccgaggtggacgtccacctcaagtcggacggctacaccaacttctccctggggacgctggactccggcaagcggcagtgcaaggaggccctgcagcgggagctgggcctgcaggtccgcggcgacgtgccgctgctcggcttcatcgggcgcctggacgggcagaagggcgtggagatcatcgcggacgcgatgccctggatcgtgagccaggacgtgcagctggtcatgctgggcaccgggcgccacgacctggagggcatgctgcggcacttcgagcgggagcaccacgacaaggtgcgcgggtgggtggggttctccgtgcggctggcgcaccggatcacggccggcgccgacgcgctcctcatgtcctcccggttcgagccgtgcggactgaaccagctctacgccatggcctacggcaccgtccccgtcgtgcatgccgtcggcggcctgagggacaccgtgccgccgttcgaccccttcaaccactccgggctcgggtggacgttcgaccgcgcagaggcgcagaagctgatcgaggcgctcgggcactgcctccgcacctaccgggactacaaggagagctggagggggctccaggagcgcggcatgtcgcaggacttcagctgggagcatgccgccaagctctacgaggacgtcctcgtcaaggccaagtaccagtggtga

SEQ ID NO: 86 - protein, 798 aa, Artificial Sequence
  EMS Divide 1704 Mutant Starch Synthase II Genome B Protein
MSSAVASAASFLALASASPGRSRRRTRVSASPPHTGAGRLHWPPSPPQRTARDGAVAARAAGKKDAGIDDAAPARQPRALRGGAATKVAERRDPVKTLDRDAAEGGAPSPPAPRQEDARLPSMNGMPVNGENKSTGGGGATKDSGLPAPARAPQPSSQNRVPVNGENKANVASPPTSIAEVAAPDPAATISISDKAPESVVPAEKAPPSSGSNFVPSASAPGSDTVSDVELELKKGAVIVKEAPNPKALSPPAAPAVQQDLWDFKKYIGFEEPVEAKDDGRAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPLFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDVHLKSDGYTNFSLGTLDSGKRQCKEALQRELGLQVRGDVPLLGFIGRLDGQKGVEIIADAMPWIVSQDVQLVMLGTGRHDLEGMLRHFEREHHDKVRGWVGFSVRLAHRITAGADALLMSSRFEPCGLNQLYAMAYGTVPVVHAVGGLRDTVPPFDPFNHSGLGWTFDRAEAQKLIEALGHCLRTYRDYKESWRGLQERGMSQDFSWEHAAKLYEDVLVKAKYQW

SEQ ID NO: 87 - DNA, 2397 bp, Artificial Sequence
  EMS Divide 47 Mutant Starch Synthase II Genome B CDS
atgtcgtcggcggtcgcgtccgccgcgtccttcctcgcgctcgcgtccgcctcccccgggagatcacggaggaggacgagggtgagcgcgtcgccaccccacaccggggctggcaggttgcactggccgccgtcgccgccgcagcgcacggctcgcgacggagcagtggccgcgcgcgccgccgggaagaaggacgcggggatcgacgacgccgcgcccgcgaggcagccccgcgcactccgcggtggcgccgccaccaaggttgcggagcggagggatcccgtcaagacgctcgatcgcgacgccgcggaaggtggcgcgccgtccccgccggcaccgaggcaggaggacgcccgtctgccgagcatgaacggcatgccggtgaacggtgaaaacaaatctaccggcggcggcggcgcgactaaagacagcgggctgcccgcacccgcacgcgcgccccagccgtcgagccagaacagagtaccggtgaatggtgaaaacaaagctaacgtcgcctcgccgccgacgagcatagccgaggtcgcggctccggatcccgcagctaccatttccatcagtgacaaggcgccagagtccgttgtcccagccgagaaggcgccgccgtcgtccggctcaaatttcgtgccctcggcttctgctcccgggtctgacactgtcagcgacgtggaacttgaactgaagaagggtgcggtcattgtcaaagaagctccaaacccaaaggctctttcgccgcctgcagcacccgctgtacaacaagacctttgggacttcaagaaatacattggtttcgaggagcccgtggaggccaaggatgatggccgggctgttgcagatgatgcgggctccttcgaacaccaccagaatcacgattccgggcctttggcaggggagaacgtcatgaacgtggtcgtcgtggctgctgaatgttctccctggtgcaaaacaggtggtcttggagatgttgccggtgctttgcccaaggctttggcgaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctctcttccgacaccgccaggaagacatttatgggggcagcagacaggaaattatgaagcgcatgattttgttctgcaaggccgctgtcgaggttccatggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgctcaccagggccgtggcccagtagatgagttcccgttcaccgagttgcctgagcactacctggaacacttcagactgtacgaccccgtgggtggtgaacacgccaactacttcgccgccggcctgaagatggcggaccaggttgtcgtcgtgagcccggggtacctgtgggagctgaagacggtggagggcggctgggggcttcacgacatcatacggcagaacgactggaagacccgcggcatcgtgaacggcatcgacaacatggagtggaaccccgaggtggacgtccacctcaagtcggacggctacaccaacttctccctggggacgctggactccggcaagcggcagtgcaaggaggccctgcagcgggagctgggcctgcaggtccgcggcgacgtgccgctgctcggcttcatcgggcgcctggacgggcagaagggcgtggagatcatcgcggacgcgatgccctggatcgtgagccaggacgtgcagctggtcatgctgggcaccgggcgccacgacctggagggcatgctgcggcacttcgagcgggagcaccacgacaaggtgcgcgggtgggtggggttctccgtgcggctggcgcaccggatcacggccggcgccgacgcgctcctcatgccctcccggttcgagccgtgcggactgaaccagctctacgccatggcctacggcaccgtccccgtcgtgcatgccgtcggcggcctgagggacaccgtgccgccgttcgaccccttcaaccactccgggctcgggtggacgttcgaccgcgcagaggcgcagaagctgatcgaggcgttcgggcactgcctccgcacctaccgggactacaaggagagctggagggggctccaggagcgcggcatgtcgcaggacttcagctgggagcatgccgccaagctctacgaggacgtcctcgtcaaggccaagtaccagtggtga

SEQ ID NO: 88 - protein, 798 aa, Artificial Sequence
  EMS Divide 47 Mutant Starch Synthase II Genome B Protein
MSSAVASAASFLALASASPGRSRRRTRVSASPPHTGAGRLHWPPSPPQRTARDGAVAARAAGKKDAGIDDAAPARQPRALRGGAATKVAERRDPVKTLDRDAAEGGAPSPPAPRQEDARLPSMNGMPVNGENKSTGGGGATKDSGLPAPARAPQPSSQNRVPVNGENKANVASPPTSIAEVAAPDPAATISISDKAPESVVPAEKAPPSSGSNFVPSASAPGSDTVSDVELELKKGAVIVKEAPNPKALSPPAAPAVQQDLWDFKKYIGFEEPVEAKDDGRAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPLFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDVHLKSDGYTNFSLGTLDSGKRQCKEALQRELGLQVRGDVPLLGFIGRLDGQKGVEIIADAMPWIVSQDVQLVMLGTGRHDLEGMLRHFEREHHDKVRGWVGFSVRLAHRITAGADALLMPSRFEPCGLNQLYAMAYGTVPVVHAVGGLRDTVPPFDPFNHSGLGWTFDRAEAQKLIEAFGHCLRTYRDYKESWRGLQERGMSQDFSWEHAAKLYEDVLVKAKYQW

SEQ ID NO: 89 - DNA, 2397 bp, Artificial Sequence
  EMS Divide 887 Mutant Starch Synthase II Genome B CDS
atgtcgtcggcggtcgcgtccgccgcgtccttcctcgcgctcgcgtccgcctcccccgggagatcacggaggaggacgagggtgagcgcgtcgccaccccacaccggggctggcaggttgcactggccgccgtcgccgccgcagcgcacggctcgcgacggagcagtggccgcgcgcgccgccgggaagaaggacgcggggatcgacgacgccgcgcccgcgaggcagccccgcgcactccgcggtggcgccgccaccaaggttgcggagcggagggatcccgtcaagacgctcgatcgcgacgccgcggaaggtggcgcgccgtccccgccggcaccgaggcaggaggacgcccgtctgccgagcatgaacggcatgccggtgaacggtgaaaacaaatctaccggcggcggcggcgcgactaaagacagcgggctgcccgcacccgcacgcgcgccccagccgtcgagccagaacagagtaccggtgaatggtgaaaacaaagctaacgtcgcctcgccgccgacgagcatagccgaggtcgcggctccggatcccgcagctaccatttccatcagtgacaaggcgccagagtccgttgtcccagccgagaaggcgccgccgtcgtccggctcaaatttcgtgccctcggcttctgctcccgggtctgacactgtcagcgacgtggaacttgaactgaagaagggtgcggtcattgtcaaagaagctccaaacccaaaggctctttcgccgcctgcagcacccgctgtacaacaagacctttgggacttcaagaaatacattggtttcgaggagcccgtggaggccaaggatgatggccgggctgttgcagatgatgcgggctccttcgaacaccaccagaatcacgattccgggcctttggcaggggagaacgtcatgaacgtggtcgtcgtggctgctgaatgttctccctggtgcaaaacaggtggtcttggagatgttgccggtgctttgcccaaggctttggcgaagagaggacatcgtgttatggttgtggtaccaaggtatggggactatgaggaagcctacgatgtcggagtccgaaaatactacaaggctgctggacaggatatggaagtgaattatttccatgcttatatcgatggagttgattttgtgttcattgacgctcctctcttccgacaccgccaggaagacatttatgggggcagcagacaggaaattatgaagcgcatgattttgttctgcaaggccgctgtcgaggttccatggcacgttccatgcggcggtgtcccttatggggatggaaatctggtgtttattgcaaatgattggcacacggcactcctgcctgtctatctgaaagcatattacagggaccatggtttgatgcagtacactcggtccattatggtgatacataacatcgctcaccagggccgtggcccagtagatgagttcccgttcaccgagttgcctgagcactacctggaacacttcagactgtacgaccccgtgggtggtgaacacgccaactacttcgccgccggcctgaagatggcggaccaggttgtcgtcgtgagcccggggtacctgtgggagctgaagacggtggagggcggctgggggcttcacgacatcatacggcagaacgactggaagacccgcggcatcgtgaacggcatcgacaacatggagtggaaccccgaggtggacgtccacctcaagtcggacggctacaccaacttctccctggggacgctggactccggcaagcggcagtgcaaggaggccctgcagcgggagctgggcctgcaggtccgcggcgacgtgccgctgctcggcttcatcgggcgcctggacgggcagaagggcgtggagatcatcgcggacgcgatgccctggatcgtgagccaggacgtgcagctggtcatgctgggcaccgggcgccacgacctggagggcatgctgcggcacttcgagcgggagcaccacgacaaggtgcgcgggtgggtggggttctccgtgcggctggcgcaccggatcacggccggcgccgacgcgctcctcatgccctcccggttcgagccgtgcggactgaaccagctctacgccatggcctacggcaccgtccccgtcgtgcatgccgtcggcggcctgagggacaccgtgccgccgttcgaccccttcaaccactccgggctcgggtggacgttcgaccgcgcagaggcgcagaagctgatcgaggcgctcgggcactgcctctgcacctaccgggactacaaggagagctggagggggctccaggagcgcggcatgtcgcaggacttcagctgggagcatgccgccaagctctacgaggacgtcctcgtcaaggccaagtaccagtggtga

SEQ ID NO: 90 - protein, 798 aa, Artificial Sequence
  EMS Divide 887 Mutant Starch Synthase II Genome B Protein
MSSAVASAASFLALASASPGRSRRRTRVSASPPHTGAGRLHWPPSPPQRTARDGAVAARAAGKKDAGIDDAAPARQPRALRGGAATKVAERRDPVKTLDRDAAEGGAPSPPAPRQEDARLPSMNGMPVNGENKSTGGGGATKDSGLPAPARAPQPSSQNRVPVNGENKANVASPPTSIAEVAAPDPAATISISDKAPESVVPAEKAPPSSGSNFVPSASAPGSDTVSDVELELKKGAVIVKEAPNPKALSPPAAPAVQQDLWDFKKYIGFEEPVEAKDDGRAVADDAGSFEHHQNHDSGPLAGENVMNVVVVAAECSPWCKTGGLGDVAGALPKALAKRGHRVMVVVPRYGDYEEAYDVGVRKYYKAAGQDMEVNYFHAYIDGVDFVFIDAPLFRHRQEDIYGGSRQEIMKRMILFCKAAVEVPWHVPCGGVPYGDGNLVFIANDWHTALLPVYLKAYYRDHGLMQYTRSIMVIHNIAHQGRGPVDEFPFTELPEHYLEHFRLYDPVGGEHANYFAAGLKMADQVVVVSPGYLWELKTVEGGWGLHDIIRQNDWKTRGIVNGIDNMEWNPEVDVHLKSDGYTNFSLGTLDSGKRQCKEALQRELGLQVRGDVPLLGFIGRLDGQKGVEIIADAMPWIVSQDVQLVMLGTGRHDLEGMLRHFEREHHDKVRGWVGFSVRLAHRITAGADALLMPSRFEPCGLNQLYAMAYGTVPVVHAVGGLRDTVPPFDPFNHSGLGWTFDRAEAQKLIEALGHCLCTYRDYKESWRGLQERGMSQDFSWEHAAKLYEDVLVKAKYQW